SEQUENCE LISTING (847 sequences)

SEQ ID NO: 1 - protein, 75 aa, Severe acute respiratory syndrome coronavirus 2
MYSFVSEETGTLIVNSVLLFLAFVVFLLVTLAILTALRLCAYCCNIVNVSLVKPSFYVYSRVKNLNSSRVPDLLV

SEQ ID NO: 2 - protein, 222 aa, Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 3 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 25 aa, Severe acute respiratory syndrome coronavirus 2
WSFNPETNILLNVPLHGTILTRPLL

SEQ ID NO: 5 - protein, 25 aa, Severe acute respiratory syndrome coronavirus 2
ATSRTLSYYKLNTDHSSSSDNIALL

SEQ ID NO: 6 - protein, 47 aa, Severe acute respiratory syndrome coronavirus 2
SFVSEETGTLIVNSVLLFLAFVVFLLVTLAILTALRLCAYCCNIVNV

SEQ ID NO: 7 - protein, 22 aa, Severe acute respiratory syndrome coronavirus 2
PSFYVYSRVKNLNSSRVPDLLV

SEQ ID NO: 8 - protein, 30 aa, Severe acute respiratory syndrome coronavirus 2
GVYYPDKVFRSSVLHSTQDLFLPFFSNVTW

SEQ ID NO: 9 - protein, 25 aa, Severe acute respiratory syndrome coronavirus 2
KIYSKHTPINLVRDLPQGFSALEPL

SEQ ID NO: 10 - protein, 25 aa, Severe acute respiratory syndrome coronavirus 2
VDLPIGINITRFQTLLALHRSYLTP

SEQ ID NO: 11 - protein, 25 aa, Severe acute respiratory syndrome coronavirus 2
GAAAYYVGYLQPRTFLLKYNENGTI

SEQ ID NO: 12 - protein, 25 aa, Severe acute respiratory syndrome coronavirus 2
VGYQPYRVVVLSFELLHAPATVCGP

SEQ ID NO: 13 - protein, 25 aa, Severe acute respiratory syndrome coronavirus 2
SKRSFIEDLLFNKVTLADAGFIKQY

SEQ ID NO: 14 - protein, 25 aa, Severe acute respiratory syndrome coronavirus 2
ICAQKFNGLTVLPPLLTDEMIAQYT

SEQ ID NO: 15 - protein, 36 aa, Severe acute respiratory syndrome coronavirus 2
NQKLIANQFNSAIGKIQDSLSSTASALGKLQDVVNQ

SEQ ID NO: 16 - protein, 25 aa, Severe acute respiratory syndrome coronavirus 2
LVKQLSSNFGAISSVLNDILSRLDK

SEQ ID NO: 17 - protein, 30 aa, Severe acute respiratory syndrome coronavirus 2
QTYVTQQLIRAAEIRASANLAATKMSECVL

SEQ ID NO: 18 - protein, 18 aa, Severe acute respiratory syndrome coronavirus 2
EETGTLIVNSVLLFLAFV

SEQ ID NO: 19 - protein, 12 aa, Severe acute respiratory syndrome coronavirus 2
GTLIVNSVLLFL

SEQ ID NO: 20 - protein, 19 aa, Severe acute respiratory syndrome coronavirus 2
VNSVLLFLAFVVFLLVTLA

SEQ ID NO: 21 - protein, 15 aa, Severe acute respiratory syndrome coronavirus 2
VLLFLAFVVFLLVTL

SEQ ID NO: 22 - protein, 18 aa, Artificial Sequence
  Synthetic construct
KNSVLLFLAFVVFLLVTK

SEQ ID NO: 23 - protein, 12 aa, Artificial Sequence
  Synthetic construct
VLLFLAFVVFLL

SEQ ID NO: 24 - protein, 18 aa, Artificial Sequence
  Synthetic construct
GAHVVFLLVTLAILTAKR

SEQ ID NO: 25 - protein, 12 aa, Severe acute respiratory syndrome coronavirus 2
VVFLLVTLAILT

SEQ ID NO: 26 - protein, 15 aa, Artificial Sequence
  Synthetic construct
TAAILTALRLSAYSA

SEQ ID NO: 27 - protein, 9 aa, Severe acute respiratory syndrome coronavirus 2
ILTALRLSA

SEQ ID NO: 28 - protein, 16 aa, Artificial Sequence
  Synthetic construct
AGSRVKNLNSSRVPDN

SEQ ID NO: 29 - protein, 10 aa, Severe acute respiratory syndrome coronavirus 2
RVKNLNSSRV

SEQ ID NO: 30 - protein, 13 aa, Severe acute respiratory syndrome coronavirus 2
SRVKNLNSSRVPD

SEQ ID NO: 31 - protein, 10 aa, Severe acute respiratory syndrome coronavirus 2
RVKNLNSSRV

SEQ ID NO: 32 - protein, 19 aa, Severe acute respiratory syndrome coronavirus 2
RNRFLYIIKLIFLWLLWPV

SEQ ID NO: 33 - protein, 13 aa, Severe acute respiratory syndrome coronavirus 2
FLYIIKLIFLWLL

SEQ ID NO: 34 - protein, 15 aa, Severe acute respiratory syndrome coronavirus 2
ETNILLNVPLHGTIL

SEQ ID NO: 35 - protein, 9 aa, Severe acute respiratory syndrome coronavirus 2
ILLNVPLHG

SEQ ID NO: 36 - protein, 16 aa, Severe acute respiratory syndrome coronavirus 2
TLSYYKLGASQRVAGD

SEQ ID NO: 37 - protein, 10 aa, Severe acute respiratory syndrome coronavirus 2
YYKLGASQRV

SEQ ID NO: 38 - protein, 16 aa, Artificial Sequence
  Synthetic construct
MLSYYKLGASQRVAGD

SEQ ID NO: 39 - protein, 10 aa, Severe acute respiratory syndrome coronavirus 2
YYKLGASQRV

SEQ ID NO: 40 - protein, 15 aa, Artificial Sequence
  Synthetic construct
LSVYKLGASQRVAGD

SEQ ID NO: 41 - protein, 9 aa, Severe acute respiratory syndrome coronavirus 2
YKLGASQRV

SEQ ID NO: 42 - protein, 16 aa, Severe acute respiratory syndrome coronavirus 2
MFVFLVLLPLVSSQCV

SEQ ID NO: 43 - protein, 12 aa, Severe acute respiratory syndrome coronavirus 2
FVFLVLLPLVSS

SEQ ID NO: 44 - protein, 16 aa, Artificial Sequence
  Synthetic construct
MFVFLVLLPLVSSQKV

SEQ ID NO: 45 - protein, 12 aa, Severe acute respiratory syndrome coronavirus 2
FVFLVLLPLVSS

SEQ ID NO: 46 - protein, 15 aa, Severe acute respiratory syndrome coronavirus 2
DKVFRSSVLHSTQDL

SEQ ID NO: 47 - protein, 9 aa, Severe acute respiratory syndrome coronavirus 2
FRSSVLHST

SEQ ID NO: 48 - protein, 15 aa, Artificial Sequence
  Synthetic construct
DKTFRSSVLHSTQDL

SEQ ID NO: 49 - protein, 9 aa, Severe acute respiratory syndrome coronavirus 2
FRSSVLHST

SEQ ID NO: 50 - protein, 15 aa, Severe acute respiratory syndrome coronavirus 2
SSVLHSTQDLFLPFF

SEQ ID NO: 51 - protein, 9 aa, Severe acute respiratory syndrome coronavirus 2
LHSTQDLFL

SEQ ID NO: 52 - protein, 15 aa, Severe acute respiratory syndrome coronavirus 2
HTPINLVRDLPQGFS

SEQ ID NO: 53 - protein, 9 aa, Severe acute respiratory syndrome coronavirus 2
INLVRDLPQ

SEQ ID NO: 54 - protein, 15 aa, Artificial Sequence
  Synthetic construct
HTPINLVRDLPQGRS

SEQ ID NO: 55 - protein, 9 aa, Severe acute respiratory syndrome coronavirus 2
INLVRDLPQ

SEQ ID NO: 56 - protein, 16 aa, Severe acute respiratory syndrome coronavirus 2
GINITRFQTLLALHRS

SEQ ID NO: 57 - protein, 12 aa, Severe acute respiratory syndrome coronavirus 2
ITRFQTLLALHR

SEQ ID NO: 58 - protein, 17 aa, Severe acute respiratory syndrome coronavirus 2
GINITRFQTLLALHRSY

SEQ ID NO: 59 - protein, 12 aa, Severe acute respiratory syndrome coronavirus 2
ITRFQTLLALHR

SEQ ID NO: 60 - protein, 15 aa, Artificial Sequence
  Synthetic construct
YAGYLQPRTFLLKYN

SEQ ID NO: 61 - protein, 9 aa, Severe acute respiratory syndrome coronavirus 2
YLQPRTFLL

SEQ ID NO: 62 - protein, 15 aa, Severe acute respiratory syndrome coronavirus 2
YRVVVLSFELLHAPA

SEQ ID NO: 63 - protein, 9 aa, Severe acute respiratory syndrome coronavirus 2
VVLSFELLH

SEQ ID NO: 64 - protein, 15 aa, Artificial Sequence
  Synthetic construct
KRVVVLSFELLHKPA

SEQ ID NO: 65 - protein, 9 aa, Severe acute respiratory syndrome coronavirus 2
VVLSFELLH

SEQ ID NO: 66 - protein, 14 aa, Severe acute respiratory syndrome coronavirus 2
IEDLLFNKVTLADA

SEQ ID NO: 67 - protein, 9 aa, Severe acute respiratory syndrome coronavirus 2
LLFNKVTLA

SEQ ID NO: 68 - protein, 15 aa, Artificial Sequence
  Synthetic construct
IEDLLFNKVTLADNG

SEQ ID NO: 69 - protein, 9 aa, Severe acute respiratory syndrome coronavirus 2
LLFNKVTLA

SEQ ID NO: 70 - protein, 15 aa, Severe acute respiratory syndrome coronavirus 2
ANGLTVLPPLLTDEM

SEQ ID NO: 71 - protein, 9 aa, Severe acute respiratory syndrome coronavirus 2
LTVLPPLLT

SEQ ID NO: 72 - protein, 14 aa, Severe acute respiratory syndrome coronavirus 2
NGLTVLPPLLTDEM

SEQ ID NO: 73 - protein, 9 aa, Severe acute respiratory syndrome coronavirus 2
LTVLPPLLT

SEQ ID NO: 74 - protein, 15 aa, Artificial Sequence
  Synthetic construct
KAQYTSALLAGTITS

SEQ ID NO: 75 - protein, 9 aa, Severe acute respiratory syndrome coronavirus 2
YTSALLAGT

SEQ ID NO: 76 - protein, 15 aa, Severe acute respiratory syndrome coronavirus 2
ANQFNSAIGKIQDSL

SEQ ID NO: 77 - protein, 9 aa, Severe acute respiratory syndrome coronavirus 2
FNSAIGKIQ

SEQ ID NO: 78 - protein, 16 aa, Severe acute respiratory syndrome coronavirus 2
IQDSLSSTASALGKLQ

SEQ ID NO: 79 - protein, 10 aa, Severe acute respiratory syndrome coronavirus 2
SLSSTASALG

SEQ ID NO: 80 - protein, 16 aa, Artificial Sequence
  Synthetic construct
KQDSLSSTASALGKLQ

SEQ ID NO: 81 - protein, 10 aa, Severe acute respiratory syndrome coronavirus 2
SLSSTASALG

SEQ ID NO: 82 - protein, 16 aa, Artificial Sequence
  Synthetic construct
RNTLVKQLSSNFGAIK

SEQ ID NO: 83 - protein, 10 aa, Severe acute respiratory syndrome coronavirus 2
LVKQLSSNFG

SEQ ID NO: 84 - protein, 15 aa, Severe acute respiratory syndrome coronavirus 2
SSNFGAISSVLNDIL

SEQ ID NO: 85 - protein, 9 aa, Severe acute respiratory syndrome coronavirus 2
FGAISSVLN

SEQ ID NO: 86 - protein, 15 aa, Severe acute respiratory syndrome coronavirus 2
QQLIRAAEIRASANL

SEQ ID NO: 87 - protein, 9 aa, Severe acute respiratory syndrome coronavirus 2
IRAAEIRAS

SEQ ID NO: 88 - protein, 15 aa, Severe acute respiratory syndrome coronavirus 2
AAEIRASANLAATKM

SEQ ID NO: 89 - protein, 9 aa, Severe acute respiratory syndrome coronavirus 2
IRASANLAA

SEQ ID NO: 90 - protein, 15 aa, Severe acute respiratory syndrome coronavirus 2
WYIWLGFIAGLIAIV

SEQ ID NO: 91 - protein, 9 aa, Severe acute respiratory syndrome coronavirus 2
WLGFIAGLI

SEQ ID NO: 92 - protein, 15 aa, Artificial Sequence
  Synthetic construct
WGIWLGFIAGLIAIK

SEQ ID NO: 93 - protein, 9 aa, Severe acute respiratory syndrome coronavirus 2
WLGFIAGLI

SEQ ID NO: 94 - protein, 15 aa, Severe acute respiratory syndrome coronavirus 2
NAAIVLQLPQGTTLP

SEQ ID NO: 95 - protein, 9 aa, Severe acute respiratory syndrome coronavirus 2
IVLQLPQGT

SEQ ID NO: 96 - protein, 17 aa, Severe acute respiratory syndrome coronavirus 2
PFGWLIVGVALLAVFQS

SEQ ID NO: 97 - protein, 11 aa, Severe acute respiratory syndrome coronavirus 2
WLIVGVALLAV

SEQ ID NO: 98 - protein, 16 aa, Severe acute respiratory syndrome coronavirus 2
KRWQLALSKGVHFVCN

SEQ ID NO: 99 - protein, 11 aa, Severe acute respiratory syndrome coronavirus 2
WQLALSKGVHF

SEQ ID NO: 100 - protein, 33 aa, Severe acute respiratory syndrome coronavirus 2
SKGVHFVCNLLLLFVTVYSHLLLVAAGLEAPFL

SEQ ID NO: 101 - protein, 27 aa, Severe acute respiratory syndrome coronavirus 2
VHFVCNLLLLFVTVYSHLLLVAAGLEA

SEQ ID NO: 102 - protein, 23 aa, Severe acute respiratory syndrome coronavirus 2
IWNLDYIINLIIKNLSKSLTENK

SEQ ID NO: 103 - protein, 17 aa, Severe acute respiratory syndrome coronavirus 2
LDYIINLIIKNLSKSLT

SEQ ID NO: 104 - protein, 16 aa, Severe acute respiratory syndrome coronavirus 2
MKIILFLALITLATCE

SEQ ID NO: 105 - protein, 13 aa, Severe acute respiratory syndrome coronavirus 2
MKIILFLALITLA

SEQ ID NO: 106 - protein, 18 aa, Severe acute respiratory syndrome coronavirus 2
YSPIFLIVAAIVFITLCF

SEQ ID NO: 107 - protein, 12 aa, Severe acute respiratory syndrome coronavirus 2
IFLIVAAIVFIT

SEQ ID NO: 108 - protein, 17 aa, Severe acute respiratory syndrome coronavirus 2
QTPFEIKLAKKFDTFNG

SEQ ID NO: 109 - protein, 11 aa, Severe acute respiratory syndrome coronavirus 2
FEIKLAKKFDT

SEQ ID NO: 110 - protein, 18 aa, Severe acute respiratory syndrome coronavirus 2
DNLLEILQKEKVNINIVG

SEQ ID NO: 111 - protein, 12 aa, Severe acute respiratory syndrome coronavirus 2
LEILQKEKVNIN

SEQ ID NO: 112 - protein, 18 aa, Severe acute respiratory syndrome coronavirus 2
NEEIAIILASFSASTSAF

SEQ ID NO: 113 - protein, 13 aa, Severe acute respiratory syndrome coronavirus 2
IAIILASFSASTS

SEQ ID NO: 114 - protein, 16 aa, Severe acute respiratory syndrome coronavirus 2
FGTVYEKLKPVLDWLE

SEQ ID NO: 115 - protein, 10 aa, Severe acute respiratory syndrome coronavirus 2
VYEKLKPVLD

SEQ ID NO: 116 - protein, 18 aa, Severe acute respiratory syndrome coronavirus 2
NQHEVLLAPLLSAGIFGA

SEQ ID NO: 117 - protein, 12 aa, Severe acute respiratory syndrome coronavirus 2
EVLLAPLLSAGI

SEQ ID NO: 118 - protein, 15 aa, Severe acute respiratory syndrome coronavirus 2
VSSFLEMKSEKQVEQ

SEQ ID NO: 119 - protein, 9 aa, Severe acute respiratory syndrome coronavirus 2
FLEMKSEKQ

SEQ ID NO: 120 - protein, 16 aa, Severe acute respiratory syndrome coronavirus 2
ETKFLTENLLLYIDIN

SEQ ID NO: 121 - protein, 10 aa, Severe acute respiratory syndrome coronavirus 2
FLTENLLLYI

SEQ ID NO: 122 - protein, 16 aa, Severe acute respiratory syndrome coronavirus 2
AKTVLKKCKSAFYILP

SEQ ID NO: 123 - protein, 10 aa, Severe acute respiratory syndrome coronavirus 2
VLKKCKSAFY

SEQ ID NO: 124 - protein, 20 aa, Severe acute respiratory syndrome coronavirus 2
VITFDNLKTLLSLREVRTIK

SEQ ID NO: 125 - protein, 14 aa, Severe acute respiratory syndrome coronavirus 2
FDNLKTLLSLREVR

SEQ ID NO: 126 - protein, 17 aa, Severe acute respiratory syndrome coronavirus 2
VGDIILKPANNSLKITE

SEQ ID NO: 127 - protein, 11 aa, Severe acute respiratory syndrome coronavirus 2
IILKPANNSLK

SEQ ID NO: 128 - protein, 17 aa, Severe acute respiratory syndrome coronavirus 2
PNELSRVLGLKTLATHG

SEQ ID NO: 129 - protein, 12 aa, Severe acute respiratory syndrome coronavirus 2
LSRVLGLKTLAT

SEQ ID NO: 130 - protein, 20 aa, Severe acute respiratory syndrome coronavirus 2
YMPYFFTLLLQLCTFTRSTN

SEQ ID NO: 131 - protein, 14 aa, Severe acute respiratory syndrome coronavirus 2
YFFTLLLQLCTFTR

SEQ ID NO: 132 - protein, 21 aa, Severe acute respiratory syndrome coronavirus 2
LINIIIWFLLLSVCLGSLIYS

SEQ ID NO: 133 - protein, 15 aa, Severe acute respiratory syndrome coronavirus 2
IIIWFLLLSVCLGSL

SEQ ID NO: 134 - protein, 15 aa, Severe acute respiratory syndrome coronavirus 2
QPILLLDQALVSDVG

SEQ ID NO: 135 - protein, 9 aa, Severe acute respiratory syndrome coronavirus 2
LLLDQALVS

SEQ ID NO: 136 - protein, 30 aa, Severe acute respiratory syndrome coronavirus 2
KIVNNWLKQLIKVTLVFLFVAAIFYLITPV

SEQ ID NO: 137 - protein, 25 aa, Severe acute respiratory syndrome coronavirus 2
VNNWLKQLIKVTLVFLFVAAIFYLI

SEQ ID NO: 138 - protein, 14 aa, Severe acute respiratory syndrome coronavirus 2
MRFRRAFGEYSHVV

SEQ ID NO: 139 - protein, 9 aa, Severe acute respiratory syndrome coronavirus 2
FRRAFGEYS

SEQ ID NO: 140 - protein, 18 aa, Severe acute respiratory syndrome coronavirus 2
KEMYLKLRSDVLLPLTQY

SEQ ID NO: 141 - protein, 12 aa, Severe acute respiratory syndrome coronavirus 2
YLKLRSDVLLPL

SEQ ID NO: 142 - protein, 27 aa, Severe acute respiratory syndrome coronavirus 2
KRTIKGTHHWLLLTILTSLLVLVQSTQ

SEQ ID NO: 143 - protein, 21 aa, Severe acute respiratory syndrome coronavirus 2
IKGTHHWLLLTILTSLLVLVQ

SEQ ID NO: 144 - protein, 20 aa, Severe acute respiratory syndrome coronavirus 2
KHAFLCLFLLPSLATVAYFN

SEQ ID NO: 145 - protein, 14 aa, Severe acute respiratory syndrome coronavirus 2
FLCLFLLPSLATVA

SEQ ID NO: 146 - protein, 17 aa, Severe acute respiratory syndrome coronavirus 2
KDCVMYASAVVLLILMT

SEQ ID NO: 147 - protein, 11 aa, Severe acute respiratory syndrome coronavirus 2
VMYASAVVLLI

SEQ ID NO: 148 - protein, 19 aa, Severe acute respiratory syndrome coronavirus 2
IDAFKLNIKLLGVGGKPCI

SEQ ID NO: 149 - protein, 13 aa, Severe acute respiratory syndrome coronavirus 2
FKLNIKLLGVGGK

SEQ ID NO: 150 - protein, 26 aa, Severe acute respiratory syndrome coronavirus 2
CTSVVLLSVLQQLRVESSSKLWAQCV

SEQ ID NO: 151 - protein, 20 aa, Severe acute respiratory syndrome coronavirus 2
VVLLSVLQQLRVESSSKLWA

SEQ ID NO: 152 - protein, 25 aa, Severe acute respiratory syndrome coronavirus 2
TEAFEKMVSLLSVLLSMQGAVDINK

SEQ ID NO: 153 - protein, 19 aa, Severe acute respiratory syndrome coronavirus 2
FEKMVSLLSVLLSMQGAVD

SEQ ID NO: 154 - protein, 21 aa, Severe acute respiratory syndrome coronavirus 2
DSEVVLKKLKKSLNVAKSEFD

SEQ ID NO: 155 - protein, 15 aa, Severe acute respiratory syndrome coronavirus 2
VVLKKLKKSLNVAKS

SEQ ID NO: 156 - protein, 18 aa, Severe acute respiratory syndrome coronavirus 2
TKGGRFVLALLSDLQDLK

SEQ ID NO: 157 - protein, 12 aa, Severe acute respiratory syndrome coronavirus 2
GRFVLALLSDLQ

SEQ ID NO: 158 - protein, 14 aa, Severe acute respiratory syndrome coronavirus 2
LYFIKGLNNLNRGM

SEQ ID NO: 159 - protein, 10 aa, Severe acute respiratory syndrome coronavirus 2
FIKGLNNLNR

SEQ ID NO: 160 - protein, 16 aa, Severe acute respiratory syndrome coronavirus 2
NPDILRVYANLGERVR

SEQ ID NO: 161 - protein, 10 aa, Severe acute respiratory syndrome coronavirus 2
ILRVYANLGE

SEQ ID NO: 162 - protein, 20 aa, Severe acute respiratory syndrome coronavirus 2
NRQFHQKLLKSIAATRGATV

SEQ ID NO: 163 - protein, 14 aa, Severe acute respiratory syndrome coronavirus 2
FHQKLLKSIAATRG

SEQ ID NO: 164 - protein, 18 aa, Severe acute respiratory syndrome coronavirus 2
MPNMLRIMASLVLARKHT

SEQ ID NO: 165 - protein, 12 aa, Severe acute respiratory syndrome coronavirus 2
MLRIMASLVLAR

SEQ ID NO: 166 - protein, 17 aa, Severe acute respiratory syndrome coronavirus 2
CYDHVISTSHKLVLSVN

SEQ ID NO: 167 - protein, 11 aa, Severe acute respiratory syndrome coronavirus 2
HVISTSHKLVL

SEQ ID NO: 168 - protein, 17 aa, Severe acute respiratory syndrome coronavirus 2
FTGYRVTKNSKVQIGEY

SEQ ID NO: 169 - protein, 11 aa, Severe acute respiratory syndrome coronavirus 2
YRVTKNSKVQI

SEQ ID NO: 170 - protein, 14 aa, Severe acute respiratory syndrome coronavirus 2
DKFKVNSTLEQYVF

SEQ ID NO: 171 - protein, 9 aa, Severe acute respiratory syndrome coronavirus 2
FKVNSTLEQ

SEQ ID NO: 172 - protein, 23 aa, Severe acute respiratory syndrome coronavirus 2
ATNYDLSVVNARLRAKHYVYIGD

SEQ ID NO: 173 - protein, 17 aa, Severe acute respiratory syndrome coronavirus 2
YDLSVVNARLRAKHYVY

SEQ ID NO: 174 - protein, 16 aa, Severe acute respiratory syndrome coronavirus 2
FDTRVLSNLNLPGCDG

SEQ ID NO: 175 - protein, 10 aa, Severe acute respiratory syndrome coronavirus 2
RVLSNLNLPG

SEQ ID NO: 176 - protein, 22 aa, Severe acute respiratory syndrome coronavirus 2
PETYFTQSRNLQEFKPRSQMEI

SEQ ID NO: 177 - protein, 16 aa, Severe acute respiratory syndrome coronavirus 2
YFTQSRNLQEFKPRSQ

SEQ ID NO: 178 - protein, 17 aa, Severe acute respiratory syndrome coronavirus 2
DDFVEIIKSQDLSVVSK

SEQ ID NO: 179 - protein, 11 aa, Severe acute respiratory syndrome coronavirus 2
VEIIKSQDLSV

SEQ ID NO: 180 - protein, 18 aa, Severe acute respiratory syndrome coronavirus 2
ICGFIQQKLALGGSVAIK

SEQ ID NO: 181 - protein, 12 aa, Severe acute respiratory syndrome coronavirus 2
FIQQKLALGGSV

SEQ ID NO: 182 - protein, 18 aa, Severe acute respiratory syndrome coronavirus 2
TAFVTNVNASSSEAFLIG

SEQ ID NO: 183 - protein, 13 aa, Severe acute respiratory syndrome coronavirus 2
FVTNVNASSSEAF

SEQ ID NO: 184 - protein, 19 aa, Severe acute respiratory syndrome coronavirus 2
INDMILSLLSKGRLIIREN

SEQ ID NO: 185 - protein, 13 aa, Severe acute respiratory syndrome coronavirus 2
MILSLLSKGRLII

SEQ ID NO: 186 - protein, 9 aa, Severe acute respiratory syndrome coronavirus 2
EETGTLIVN

SEQ ID NO: 187 - protein, 9 aa, Severe acute respiratory syndrome coronavirus 2
ETGTLIVNS

SEQ ID NO: 188 - protein, 9 aa, Severe acute respiratory syndrome coronavirus 2
TGTLIVNSV

SEQ ID NO: 189 - protein, 9 aa, Severe acute respiratory syndrome coronavirus 2
GTLIVNSVL

SEQ ID NO: 190 - protein, 9 aa, Severe acute respiratory syndrome coronavirus 2
TLIVNSVLL

SEQ ID NO: 191 - protein, 9 aa, Severe acute respiratory syndrome coronavirus 2
LIVNSVLLF

SEQ ID NO: 192 - protein, 9 aa, Severe acute respiratory syndrome coronavirus 2
LIVNSVLLF

SEQ ID NO: 193 - protein, 9 aa, Severe acute respiratory syndrome coronavirus 2
IVNSVLLFL

SEQ ID NO: 194 - protein, 9 aa, Severe acute respiratory syndrome coronavirus 2
VNSVLLFLA

SEQ ID NO: 195 - protein, 9 aa, Severe acute respiratory syndrome coronavirus 2
NSVLLFLAF

SEQ ID NO: 196 - protein, 9 aa, Severe acute respiratory syndrome coronavirus 2
SVLLFLAFV

SEQ ID NO: 197 - protein, 9 aa, Severe acute respiratory syndrome coronavirus 2
KNSVLLFLA

SEQ ID NO: 198 - protein, 9 aa, Severe acute respiratory syndrome coronavirus 2
NSVLLFLAF

SEQ ID NO: 199 - protein, 9 aa, Severe acute respiratory syndrome coronavirus 2
SVLLFLAFV

SEQ ID NO: 200 - protein, 9 aa, Severe acute respiratory syndrome coronavirus 2
VLLFLAFVV

SEQ ID NO: 201 - protein, 9 aa, Severe acute respiratory syndrome coronavirus 2
LLFLAFVVF

SEQ ID NO: 202 - protein, 9 aa, Severe acute respiratory syndrome coronavirus 2
LFLAFVVFL

SEQ ID NO: 203 - protein, 9 aa, Severe acute respiratory syndrome coronavirus 2
FLAFVVFLL

SEQ ID NO: 204 - protein, 9 aa, Severe acute respiratory syndrome coronavirus 2
LAFVVFLLV

SEQ ID NO: 205 - protein, 9 aa, Severe acute respiratory syndrome coronavirus 2
AFVVFLLVT

SEQ ID NO: 206 - protein, 9 aa, Severe acute respiratory syndrome coronavirus 2
FVVFLLVTK

SEQ ID NO: 207 - protein, 9 aa, Severe acute respiratory syndrome coronavirus 2
GAHVVFLLV

SEQ ID NO: 208 - protein, 9 aa, Severe acute respiratory syndrome coronavirus 2
AHVVFLLVT

SEQ ID NO: 209 - protein, 9 aa, Severe acute respiratory syndrome coronavirus 2
HVVFLLVTL

SEQ ID NO: 210 - protein, 9 aa, Severe acute respiratory syndrome coronavirus 2
VVFLLVTLA

SEQ ID NO: 211 - protein, 9 aa, Severe acute respiratory syndrome coronavirus 2
VFLLVTLAI

SEQ ID NO: 212 - protein, 9 aa, Severe acute respiratory syndrome coronavirus 2
FLLVTLAIL

SEQ ID NO: 213 - protein, 9 aa, Severe acute respiratory syndrome coronavirus 2
LLVTLAILT

SEQ ID NO: 214 - protein, 9 aa, Severe acute respiratory syndrome coronavirus 2
LVTLAILTA

SEQ ID NO: 215 - protein, 9 aa, Severe acute respiratory syndrome coronavirus 2
VTLAILTAK

SEQ ID NO: 216 - protein, 9 aa, Severe acute respiratory syndrome coronavirus 2
TLAILTAKR

SEQ ID NO: 217 - protein, 9 aa, Severe acute respiratory syndrome coronavirus 2
TAAILTALR

SEQ ID NO: 218 - protein, 9 aa, Severe acute respiratory syndrome coronavirus 2
AAILTALRL

SEQ ID NO: 219 - protein, 9 aa, Severe acute respiratory syndrome coronavirus 2
AILTALRLS

SEQ ID NO: 220 - protein, 9 aa, Severe acute respiratory syndrome coronavirus 2
ILTALRLSA

SEQ ID NO: 221 - protein, 9 aa, Severe acute respiratory syndrome coronavirus 2
LTALRLSAY

SEQ ID NO: 222 - protein, 9 aa, Severe acute respiratory syndrome coronavirus 2
TALRLSAYS

SEQ ID NO: 223 - protein, 9 aa, Severe acute respiratory syndrome coronavirus 2
ALRLSAYSA

SEQ ID NO: 224 - protein, 9 aa, Severe acute respiratory syndrome coronavirus 2
AGSRVKNLN

SEQ ID NO: 225 - protein, 9 aa, Severe acute respiratory syndrome coronavirus 2
GSRVKNLNS

SEQ ID NO: 226 - protein, 9 aa, Severe acute respiratory syndrome coronavirus 2
SRVKNLNSS

SEQ ID NO: 227 - protein, 9 aa, Severe acute respiratory syndrome coronavirus 2
RVKNLNSSR

SEQ ID NO: 228 - protein, 9 aa, Severe acute respiratory syndrome coronavirus 2
VKNLNSSRV

SEQ ID NO: 229 - protein, 9 aa, Severe acute respiratory syndrome coronavirus 2
KNLNSSRVP

SEQ ID NO: 230 - protein, 9 aa, Severe acute respiratory syndrome coronavirus 2
NLNSSRVPD

SEQ ID NO: 231 - protein, 9 aa, Severe acute respiratory syndrome coronavirus 2
LNSSRVPDN

SEQ ID NO: 232 - protein, 9 aa, Severe acute respiratory syndrome coronavirus 2
ETNILLNVP

SEQ ID NO: 233 - protein, 9 aa, Severe acute respiratory syndrome coronavirus 2
TNILLNVPL

SEQ ID NO: 234 - protein, 9 aa, Severe acute respiratory syndrome coronavirus 2
NILLNVPLH

SEQ ID NO: 235 - protein, 9 aa, Severe acute respiratory syndrome coronavirus 2
ILLNVPLHG

SEQ ID NO: 236 - protein, 9 aa, Severe acute respiratory syndrome coronavirus 2
LLNVPLHGT

SEQ ID NO: 237 - protein, 9 aa, Severe acute respiratory syndrome coronavirus 2
LNVPLHGTI

SEQ ID NO: 238 - protein, 9 aa, Severe acute respiratory syndrome coronavirus 2
NVPLHGTIL

SEQ ID NO: 239 - protein, 9 aa, Severe acute respiratory syndrome coronavirus 2
LSVYKLGAS

SEQ ID NO: 240 - protein, 9 aa, Severe acute respiratory syndrome coronavirus 2
SVYKLGASQ

SEQ ID NO: 241 - protein, 9 aa, Severe acute respiratory syndrome coronavirus 2
VYKLGASQR

SEQ ID NO: 242 - protein, 9 aa, Severe acute respiratory syndrome coronavirus 2
YKLGASQRV

SEQ ID NO: 243 - protein, 9 aa, Severe acute respiratory syndrome coronavirus 2
KLGASQRVA

SEQ ID NO: 244 - protein, 9 aa, Severe acute respiratory syndrome coronavirus 2
LGASQRVAG

SEQ ID NO: 245 - protein, 9 aa, Severe acute respiratory syndrome coronavirus 2
GASQRVAGD

SEQ ID NO: 246 - protein, 9 aa, Severe acute respiratory syndrome coronavirus 2
KAQYTSALL

SEQ ID NO: 247 - protein, 9 aa, Severe acute respiratory syndrome coronavirus 2
AQYTSALLA

SEQ ID NO: 248 - protein, 9 aa, Severe acute respiratory syndrome coronavirus 2
QYTSALLAG

SEQ ID NO: 249 - protein, 9 aa, Severe acute respiratory syndrome coronavirus 2
YTSALLAGT

SEQ ID NO: 250 - protein, 9 aa, Severe acute respiratory syndrome coronavirus 2
TSALLAGTI

SEQ ID NO: 251 - protein, 9 aa, Severe acute respiratory syndrome coronavirus 2
SALLAGTIT

SEQ ID NO: 252 - protein, 9 aa, Severe acute respiratory syndrome coronavirus 2
ALLAGTITS

SEQ ID NO: 253 - protein, 9 aa, Severe acute respiratory syndrome coronavirus 2
ANQFNSAIG

SEQ ID NO: 254 - protein, 9 aa, Severe acute respiratory syndrome coronavirus 2
NQFNSAIGK

SEQ ID NO: 255 - protein, 9 aa, Severe acute respiratory syndrome coronavirus 2
QFNSAIGKI

SEQ ID NO: 256 - protein, 9 aa, Severe acute respiratory syndrome coronavirus 2
FNSAIGKIQ

SEQ ID NO: 257 - protein, 9 aa, Severe acute respiratory syndrome coronavirus 2
NSAIGKIQD

SEQ ID NO: 258 - protein, 9 aa, Severe acute respiratory syndrome coronavirus 2
SAIGKIQDS

SEQ ID NO: 259 - protein, 9 aa, Severe acute respiratory syndrome coronavirus 2
AIGKIQDSL

SEQ ID NO: 260 - protein, 9 aa, Severe acute respiratory syndrome coronavirus 2
KQDSLSSTA

SEQ ID NO: 261 - protein, 9 aa, Severe acute respiratory syndrome coronavirus 2
QDSLSSTAS

SEQ ID NO: 262 - protein, 9 aa, Severe acute respiratory syndrome coronavirus 2
DSLSSTASA

SEQ ID NO: 263 - protein, 9 aa, Severe acute respiratory syndrome coronavirus 2
SLSSTASAL

SEQ ID NO: 264 - protein, 9 aa, Severe acute respiratory syndrome coronavirus 2
LSSTASALG

SEQ ID NO: 265 - protein, 9 aa, Severe acute respiratory syndrome coronavirus 2
SSTASALGK

SEQ ID NO: 266 - protein, 9 aa, Severe acute respiratory syndrome coronavirus 2
STASALGKL

SEQ ID NO: 267 - protein, 9 aa, Severe acute respiratory syndrome coronavirus 2
TASALGKLQ

SEQ ID NO: 268 - protein, 9 aa, Severe acute respiratory syndrome coronavirus 2
RNTLVKQLS

SEQ ID NO: 269 - protein, 9 aa, Severe acute respiratory syndrome coronavirus 2
NTLVKQLSS

SEQ ID NO: 270 - protein, 9 aa, Severe acute respiratory syndrome coronavirus 2
TLVKQLSSN

SEQ ID NO: 271 - protein, 9 aa, Severe acute respiratory syndrome coronavirus 2
LVKQLSSNF

SEQ ID NO: 272 - protein, 9 aa, Severe acute respiratory syndrome coronavirus 2
VKQLSSNFG

SEQ ID NO: 273 - protein, 9 aa, Severe acute respiratory syndrome coronavirus 2
KQLSSNFGA

SEQ ID NO: 274 - protein, 9 aa, Severe acute respiratory syndrome coronavirus 2
QLSSNFGAI

SEQ ID NO: 275 - protein, 9 aa, Severe acute respiratory syndrome coronavirus 2
LSSNFGAIK

SEQ ID NO: 276 - protein, 9 aa, Severe acute respiratory syndrome coronavirus 2
SSNFGAISS

SEQ ID NO: 277 - protein, 9 aa, Severe acute respiratory syndrome coronavirus 2
SNFGAISSV

SEQ ID NO: 278 - protein, 9 aa, Severe acute respiratory syndrome coronavirus 2
NFGAISSVL

SEQ ID NO: 279 - protein, 9 aa, Severe acute respiratory syndrome coronavirus 2
FGAISSVLN

SEQ ID NO: 280 - protein, 9 aa, Severe acute respiratory syndrome coronavirus 2
GAISSVLND

SEQ ID NO: 281 - protein, 9 aa, Severe acute respiratory syndrome coronavirus 2
AISSVLNDI

SEQ ID NO: 282 - protein, 9 aa, Severe acute respiratory syndrome coronavirus 2
ISSVLNDIL

SEQ ID NO: 283 - protein, 9 aa, Severe acute respiratory syndrome coronavirus 2
QQLIRAAEI

SEQ ID NO: 284 - protein, 9 aa, Severe acute respiratory syndrome coronavirus 2
QLIRAAEIR

SEQ ID NO: 285 - protein, 9 aa, Severe acute respiratory syndrome coronavirus 2
LIRAAEIRA

SEQ ID NO: 286 - protein, 9 aa, Severe acute respiratory syndrome coronavirus 2
IRAAEIRAS

SEQ ID NO: 287 - protein, 9 aa, Severe acute respiratory syndrome coronavirus 2
RAAEIRASA

SEQ ID NO: 288 - protein, 9 aa, Severe acute respiratory syndrome coronavirus 2
AAEIRASAN

SEQ ID NO: 289 - protein, 9 aa, Severe acute respiratory syndrome coronavirus 2
AAEIRASAN

SEQ ID NO: 290 - protein, 9 aa, Severe acute respiratory syndrome coronavirus 2
AEIRASANL

SEQ ID NO: 291 - protein, 9 aa, Severe acute respiratory syndrome coronavirus 2
AAEIRASAN

SEQ ID NO: 292 - protein, 9 aa, Severe acute respiratory syndrome coronavirus 2
AEIRASANL

SEQ ID NO: 293 - protein, 9 aa, Severe acute respiratory syndrome coronavirus 2
EIRASANLA

SEQ ID NO: 294 - protein, 9 aa, Severe acute respiratory syndrome coronavirus 2
IRASANLAA

SEQ ID NO: 295 - protein, 9 aa, Severe acute respiratory syndrome coronavirus 2
RASANLAAT

SEQ ID NO: 296 - protein, 9 aa, Severe acute respiratory syndrome coronavirus 2
ASANLAATK

SEQ ID NO: 297 - protein, 9 aa, Severe acute respiratory syndrome coronavirus 2
SANLAATKM

SEQ ID NO: 298 - protein, 9 aa, Severe acute respiratory syndrome coronavirus 2
WGIWLGFIA

SEQ ID NO: 299 - protein, 9 aa, Severe acute respiratory syndrome coronavirus 2
GIWLGFIAG

SEQ ID NO: 300 - protein, 9 aa, Severe acute respiratory syndrome coronavirus 2
IWLGFIAGL

SEQ ID NO: 301 - protein, 9 aa, Severe acute respiratory syndrome coronavirus 2
WLGFIAGLI

SEQ ID NO: 302 - protein, 9 aa, Severe acute respiratory syndrome coronavirus 2
LGFIAGLIA

SEQ ID NO: 303 - protein, 9 aa, Severe acute respiratory syndrome coronavirus 2
GFIAGLIAI

SEQ ID NO: 304 - protein, 9 aa, Severe acute respiratory syndrome coronavirus 2
FIAGLIAIK

SEQ ID NO: 305 - protein, 9 aa, Severe acute respiratory syndrome coronavirus 2
MFVFLVLLP

SEQ ID NO: 306 - protein, 9 aa, Severe acute respiratory syndrome coronavirus 2
FVFLVLLPL

SEQ ID NO: 307 - protein, 9 aa, Severe acute respiratory syndrome coronavirus 2
VFLVLLPLV

SEQ ID NO: 308 - protein, 9 aa, Severe acute respiratory syndrome coronavirus 2
FLVLLPLVS

SEQ ID NO: 309 - protein, 9 aa, Severe acute respiratory syndrome coronavirus 2
LVLLPLVSS

SEQ ID NO: 310 - protein, 9 aa, Severe acute respiratory syndrome coronavirus 2
VLLPLVSSQ

SEQ ID NO: 311 - protein, 9 aa, Severe acute respiratory syndrome coronavirus 2
LLPLVSSQK

SEQ ID NO: 312 - protein, 9 aa, Severe acute respiratory syndrome coronavirus 2
LPLVSSQKV

SEQ ID NO: 313 - protein, 9 aa, Severe acute respiratory syndrome coronavirus 2
DKTFRSSVL

SEQ ID NO: 314 - protein, 9 aa, Severe acute respiratory syndrome coronavirus 2
KTFRSSVLH

SEQ ID NO: 315 - protein, 9 aa, Severe acute respiratory syndrome coronavirus 2
TFRSSVLHS

SEQ ID NO: 316 - protein, 9 aa, Severe acute respiratory syndrome coronavirus 2
FRSSVLHST

SEQ ID NO: 317 - protein, 9 aa, Severe acute respiratory syndrome coronavirus 2
RSSVLHSTQ

SEQ ID NO: 318 - protein, 9 aa, Severe acute respiratory syndrome coronavirus 2
SSVLHSTQD

SEQ ID NO: 319 - protein, 9 aa, Severe acute respiratory syndrome coronavirus 2
SVLHSTQDL

SEQ ID NO: 320 - protein, 9 aa, Severe acute respiratory syndrome coronavirus 2
SSVLHSTQD

SEQ ID NO: 321 - protein, 9 aa, Severe acute respiratory syndrome coronavirus 2
SVLHSTQDL

SEQ ID NO: 322 - protein, 9 aa, Severe acute respiratory syndrome coronavirus 2
VLHSTQDLF

SEQ ID NO: 323 - protein, 9 aa, Severe acute respiratory syndrome coronavirus 2
LHSTQDLFL

SEQ ID NO: 324 - protein, 9 aa, Severe acute respiratory syndrome coronavirus 2
HSTQDLFLP

SEQ ID NO: 325 - protein, 9 aa, Severe acute respiratory syndrome coronavirus 2
STQDLFLPF

SEQ ID NO: 326 - protein, 9 aa, Severe acute respiratory syndrome coronavirus 2
TQDLFLPFF

SEQ ID NO: 327 - protein, 9 aa, Severe acute respiratory syndrome coronavirus 2
HTPINLVRD

SEQ ID NO: 328 - protein, 9 aa, Severe acute respiratory syndrome coronavirus 2
TPINLVRDL

SEQ ID NO: 329 - protein, 9 aa, Severe acute respiratory syndrome coronavirus 2
PINLVRDLP

SEQ ID NO: 330 - protein, 9 aa, Severe acute respiratory syndrome coronavirus 2
INLVRDLPQ

SEQ ID NO: 331 - protein, 9 aa, Severe acute respiratory syndrome coronavirus 2
NLVRDLPQG

SEQ ID NO: 332 - protein, 9 aa, Severe acute respiratory syndrome coronavirus 2
LVRDLPQGR

SEQ ID NO: 333 - protein, 9 aa, Severe acute respiratory syndrome coronavirus 2
VRDLPQGRS

SEQ ID NO: 334 - protein, 9 aa, Severe acute respiratory syndrome coronavirus 2
GINITRFQT

SEQ ID NO: 335 - protein, 9 aa, Severe acute respiratory syndrome coronavirus 2
INITRFQTL

SEQ ID NO: 336 - protein, 9 aa, Severe acute respiratory syndrome coronavirus 2
NITRFQTLL

SEQ ID NO: 337 - protein, 9 aa, Severe acute respiratory syndrome coronavirus 2
ITRFQTLLA

SEQ ID NO: 338 - protein, 9 aa, Severe acute respiratory syndrome coronavirus 2
TRFQTLLAL

SEQ ID NO: 339 - protein, 9 aa, Severe acute respiratory syndrome coronavirus 2
RFQTLLALH

SEQ ID NO: 340 - protein, 9 aa, Severe acute respiratory syndrome coronavirus 2
FQTLLALHR

SEQ ID NO: 341 - protein, 9 aa, Severe acute respiratory syndrome coronavirus 2
QTLLALHRS

SEQ ID NO: 342 - protein, 9 aa, Severe acute respiratory syndrome coronavirus 2
YAGYLQPRT

SEQ ID NO: 343 - protein, 9 aa, Severe acute respiratory syndrome coronavirus 2
AGYLQPRTF

SEQ ID NO: 344 - protein, 9 aa, Severe acute respiratory syndrome coronavirus 2
GYLQPRTFL

SEQ ID NO: 345 - protein, 9 aa, Severe acute respiratory syndrome coronavirus 2
YLQPRTFLL

SEQ ID NO: 346 - protein, 9 aa, Severe acute respiratory syndrome coronavirus 2
LQPRTFLLK

SEQ ID NO: 347 - protein, 9 aa, Severe acute respiratory syndrome coronavirus 2
QPRTFLLKY

SEQ ID NO: 348 - protein, 9 aa, Severe acute respiratory syndrome coronavirus 2
PRTFLLKYN

SEQ ID NO: 349 - protein, 9 aa, Severe acute respiratory syndrome coronavirus 2
KRVVVLSFE

SEQ ID NO: 350 - protein, 9 aa, Severe acute respiratory syndrome coronavirus 2
RVVVLSFEL

SEQ ID NO: 351 - protein, 9 aa, Severe acute respiratory syndrome coronavirus 2
VVVLSFELL

SEQ ID NO: 352 - protein, 9 aa, Severe acute respiratory syndrome coronavirus 2
VVLSFELLH

SEQ ID NO: 353 - protein, 9 aa, Severe acute respiratory syndrome coronavirus 2
VLSFELLHK

SEQ ID NO: 354 - protein, 9 aa, Severe acute respiratory syndrome coronavirus 2
LSFELLHKP

SEQ ID NO: 355 - protein, 9 aa, Severe acute respiratory syndrome coronavirus 2
SFELLHKPA

SEQ ID NO: 356 - protein, 9 aa, Severe acute respiratory syndrome coronavirus 2
IEDLLFNKV

SEQ ID NO: 357 - protein, 9 aa, Severe acute respiratory syndrome coronavirus 2
EDLLFNKVT

SEQ ID NO: 358 - protein, 9 aa, Severe acute respiratory syndrome coronavirus 2
DLLFNKVTL

SEQ ID NO: 359 - protein, 9 aa, Severe acute respiratory syndrome coronavirus 2
LLFNKVTLA

SEQ ID NO: 360 - protein, 9 aa, Severe acute respiratory syndrome coronavirus 2
LFNKVTLAD

SEQ ID NO: 361 - protein, 9 aa, Severe acute respiratory syndrome coronavirus 2
FNKVTLADN

SEQ ID NO: 362 - protein, 9 aa, Severe acute respiratory syndrome coronavirus 2
NKVTLADNG

SEQ ID NO: 363 - protein, 9 aa, Severe acute respiratory syndrome coronavirus 2
ANGLTVLPP

SEQ ID NO: 364 - protein, 9 aa, Severe acute respiratory syndrome coronavirus 2
NGLTVLPPL

SEQ ID NO: 365 - protein, 9 aa, Severe acute respiratory syndrome coronavirus 2
GLTVLPPLL

SEQ ID NO: 366 - protein, 9 aa, Severe acute respiratory syndrome coronavirus 2
LTVLPPLLT

SEQ ID NO: 367 - protein, 9 aa, Severe acute respiratory syndrome coronavirus 2
TVLPPLLTD

SEQ ID NO: 368 - protein, 9 aa, Severe acute respiratory syndrome coronavirus 2
VLPPLLTDE

SEQ ID NO: 369 - protein, 9 aa, Severe acute respiratory syndrome coronavirus 2
LPPLLTDEM

SEQ ID NO: 370 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 371 - protein, 275 aa, Severe acute respiratory syndrome coronavirus 2
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 372 - protein, 61 aa, Severe acute respiratory syndrome coronavirus 2
MFHLVDFQVTIAEILLIIMRTFKVSIWNLDYIINLIIKNLSKSLTENKYSQLDEEQPMEID

SEQ ID NO: 373 - protein, 121 aa, Severe acute respiratory syndrome coronavirus 2
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 374 - protein, 638 aa, Severe acute respiratory syndrome coronavirus 2
AYTRYVDNNFCGPDGYPLECIKDLLARAGKASCTLSEQLDFIDTKRGVYCCREHEHEIAWYTERSEKSYELQTPFEIKLAKKFDTFNGECPNFVFPLNSIIKTIQPRVEKKKLDGFMGRIRSVYPVASPNECNQMCLSTLMKCDHCGETSWQTGDFVKATCEFCGTENLTKEGATTCGYLPQNAVVKIYCPACHNSEVGPEHSLAEYHNESGLKTILRKGGRTIAFGGCVFSYVGCHNKCAYWVPRASANIGCNHTGVVGEGSEGLNDNLLEILQKEKVNINIVGDFKLNEEIAIILASFSASTSAFVETVKGLDYKAFKQIVESCGNFKVTKGKAKKGAWNIGEQKSILSPLYAFASEAARVVRSIFSRTLETAQNSVRVLQKAAITILDGISQYSLRLIDAMMFTSDLATNNLVVMAYITGGVVQLTSQWLTNIFGTVYEKLKPVLDWLEEKFKEGVEFLRDGWEIVKFISTCACEIVGGQIVTCAKEIKESVQTFFKLVNKFLALCADSIIIGGAKLKALNLGETFVTHSKGLYRKCVKSREETGLLMPLKAPKEIIFLEGETLPTEVLTEEVVLKTGDLQPLEQPTSEAVEAPLVGTPVCINGLMLLEIKDTEKYCALAPNMMVTNNTFTLKGG

SEQ ID NO: 375 - protein, 1945 aa, Severe acute respiratory syndrome coronavirus 2
APTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGG

SEQ ID NO: 376 - protein, 500 aa, Severe acute respiratory syndrome coronavirus 2
KIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQ

SEQ ID NO: 377 - protein, 290 aa, Severe acute respiratory syndrome coronavirus 2
SAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQ

SEQ ID NO: 378 - protein, 83 aa, Severe acute respiratory syndrome coronavirus 2
SKMSDVKCTSVVLLSVLQQLRVESSSKLWAQCVQLHNDILLAKDTTEAFEKMVSLLSVLLSMQGAVDINKLCEEMLDNRATLQ

SEQ ID NO: 379 - protein, 198 aa, Severe acute respiratory syndrome coronavirus 2
AIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQ

SEQ ID NO: 380 - protein, 113 aa, Severe acute respiratory syndrome coronavirus 2
NNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQ

SEQ ID NO: 381 - protein, 932 aa, Severe acute respiratory syndrome coronavirus 2
SADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 382 - protein, 601 aa, Severe acute respiratory syndrome coronavirus 2
AVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKVGILCIMSDRDLYDKLQFTSLEIPRRNVATLQ

SEQ ID NO: 383 - protein, 527 aa, Severe acute respiratory syndrome coronavirus 2
AENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQ

SEQ ID NO: 384 - protein, 346 aa, Severe acute respiratory syndrome coronavirus 2
SLENVAFNVVNKGHFDGQQGEVPVSIINNTVYTKVDGVDVELFENKTTLPVNVAFELWAKRNIKPVPEVKILNNLGVDIAANTVIWDYKRDAPAHISTIGVCSMTDIAKKPTETICAPLTVFFDGRVDGQVDLFRNARNGVLITEGSVKGLQPSVGPKQASLNGVTLIGEAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQ

SEQ ID NO: 385 - protein, 298 aa, Severe acute respiratory syndrome coronavirus 2
SSQAWQPGVAMPNLYKMQRMLLEKCDLQNYGDSATLPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPKTKNVTKENDSKEGFFTYICGFIQQKLALGGSVAIKITEHSWNADLYKLMGHFAWWTAFVTNVNASSSEAFLIGCNYLGKPREQIDGYVMHANYIFWRNTNPIQLSSYSLFDMSKFPLKLRGTAVMSLKEGQINDMILSLLSKGRLIIRENNRVVISSDVLVNN

SEQ ID NO: 386 - protein, 121 aa, Severe acute respiratory syndrome coronavirus 2
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 387 - protein, 38 aa, Severe acute respiratory syndrome coronavirus 2
MGYINVFAFPFTIYSLLLCRMNSRNYIAQVDVVNFNLT

SEQ ID NO: 388 - protein, 306 aa, Severe acute respiratory syndrome coronavirus 2 [sequence omitted]

SEQ ID NO: 389 - protein, 139 aa, Severe acute respiratory syndrome coronavirus 2
AGNATEVPANSTVLSFCAFAVDAAKAYKDYLASGGQPITNCVKMLCTHTGTGQAITVTPEANMDQESFGGASCCLYCRCHIDHPNPKGFCDLKGKYVQIPTTCANDPVGFTLKNTVCTVCGMWKGYGCSCDQLREPMLQ

SEQ ID NO: 390 - protein, 22 aa, Severe acute respiratory syndrome coronavirus 2
FSNYLKRRVVFNGVSFSTFEEA

SEQ ID NO: 391 - protein, 16 aa, Severe acute respiratory syndrome coronavirus 2
YLKRRVVFNGVSFSTF

SEQ ID NO: 392 - protein, 20 aa, Severe acute respiratory syndrome coronavirus 2
TEAFEKMVSLLSVLLSMQGA

SEQ ID NO: 393 - protein, 9 aa, Severe acute respiratory syndrome coronavirus 2
TEAFEKMVS

SEQ ID NO: 394 - protein, 9 aa, Severe acute respiratory syndrome coronavirus 2
EAFEKMVSL

SEQ ID NO: 395 - protein, 9 aa, Severe acute respiratory syndrome coronavirus 2
AFEKMVSLL

SEQ ID NO: 396 - protein, 9 aa, Severe acute respiratory syndrome coronavirus 2
FEKMVSLLS

SEQ ID NO: 397 - protein, 9 aa, Severe acute respiratory syndrome coronavirus 2
EKMVSLLSV

SEQ ID NO: 398 - protein, 9 aa, Severe acute respiratory syndrome coronavirus 2
KMVSLLSVL

SEQ ID NO: 399 - protein, 9 aa, Severe acute respiratory syndrome coronavirus 2
MVSLLSVLL

SEQ ID NO: 400 - protein, 9 aa, Severe acute respiratory syndrome coronavirus 2
VSLLSVLLS

SEQ ID NO: 401 - protein, 9 aa, Severe acute respiratory syndrome coronavirus 2
SLLSVLLSM

SEQ ID NO: 402 - protein, 9 aa, Severe acute respiratory syndrome coronavirus 2
LLSVLLSMQ

SEQ ID NO: 403 - protein, 9 aa, Severe acute respiratory syndrome coronavirus 2
LSVLLSMQG

SEQ ID NO: 404 - protein, 9 aa, Severe acute respiratory syndrome coronavirus 2
SVLLSMQGA

SEQ ID NO: 405 - protein, 15 aa, Artificial Sequence
  Synthetic construct
AALVLLLLDRLNQLE

SEQ ID NO: 406 - protein, 9 aa, Artificial Sequence
  Synthetic construct
AALVLLLLD

SEQ ID NO: 407 - protein, 9 aa, Artificial Sequence
  Synthetic construct
ALVLLLLDR

SEQ ID NO: 408 - protein, 9 aa, Artificial Sequence
  Synthetic construct
LVLLLLDRL

SEQ ID NO: 409 - protein, 9 aa, Artificial Sequence
  Synthetic construct
VLLLLDRLN

SEQ ID NO: 410 - protein, 9 aa, Severe acute respiratory syndrome coronavirus 2
LLLLDRLNQ

SEQ ID NO: 411 - protein, 9 aa, Severe acute respiratory syndrome coronavirus 2
LLLDRLNQL

SEQ ID NO: 412 - protein, 9 aa, Severe acute respiratory syndrome coronavirus 2
LLDRLNQLE

SEQ ID NO: 413 - protein, 15 aa, Severe acute respiratory syndrome coronavirus 2
AALALLLLDRLNQLE

SEQ ID NO: 414 - protein, 9 aa, Severe acute respiratory syndrome coronavirus 2
AALALLLLD

SEQ ID NO: 415 - protein, 9 aa, Severe acute respiratory syndrome coronavirus 2
ALALLLLDR

SEQ ID NO: 416 - protein, 9 aa, Severe acute respiratory syndrome coronavirus 2
LALLLLDRL

SEQ ID NO: 417 - protein, 9 aa, Severe acute respiratory syndrome coronavirus 2
ALLLLDRLN

SEQ ID NO: 418 - protein, 9 aa, Severe acute respiratory syndrome coronavirus 2
LLLLDRLNQ

SEQ ID NO: 419 - protein, 9 aa, Severe acute respiratory syndrome coronavirus 2
LLLDRLNQL

SEQ ID NO: 420 - protein, 9 aa, Severe acute respiratory syndrome coronavirus 2
LLDRLNQLE

SEQ ID NO: 421 - protein, 18 aa, Severe acute respiratory syndrome coronavirus 2
AQKFNGLTVLPPLLTDEM

SEQ ID NO: 422 - protein, 15 aa, Severe acute respiratory syndrome coronavirus 2
NTLVKQLSSNFGAIS

SEQ ID NO: 423 - protein, 16 aa, Severe acute respiratory syndrome coronavirus 2
TSDYYQLYSTQLSTDT

SEQ ID NO: 424 - protein, 19 aa, Severe acute respiratory syndrome coronavirus 2
PDGVKHVYQLRARSVSPKL

SEQ ID NO: 425 - protein, 17 aa, Severe acute respiratory syndrome coronavirus 2
MKFLVFLGIITTVAAFH

SEQ ID NO: 426 - protein, 15 aa, Severe acute respiratory syndrome coronavirus 2
VQTFFKLVNKFLALC

SEQ ID NO: 427 - protein, 19 aa, Severe acute respiratory syndrome coronavirus 2
VTAYNGYLTSSSKTPEEHF

SEQ ID NO: 428 - protein, 17 aa, Severe acute respiratory syndrome coronavirus 2
TRFFYVLGLAAIMQLFF

SEQ ID NO: 429 - protein, 15 aa, Severe acute respiratory syndrome coronavirus 2
RGMVLGSLAATVRLQ

SEQ ID NO: 430 - protein, 17 aa, Severe acute respiratory syndrome coronavirus 2
MPILTLTRALTAESHVD

SEQ ID NO: 431 - protein, 15 aa, Artificial Sequence
  Synthetic construct
HTPINLVRGLPQGFS

SEQ ID NO: 432 - protein, 16 aa, Artificial Sequence
  Synthetic construct
MPNLYKMQRMLLEKDD

SEQ ID NO: 433 - protein, 25 aa, Artificial Sequence
  Synthetic construct
KIYSKHTPINLGRDLPEGFSALEPL

SEQ ID NO: 434 - protein, 25 aa, Artificial Sequence
  Synthetic construct
VDLPIGINTTRFQTLLALHRSYLTP

SEQ ID NO: 435 - protein, 25 aa, Artificial Sequence
  Synthetic construct
VGYQPYRVGVLSFELLHAPATVCGP

SEQ ID NO: 436 - protein, 25 aa, Artificial Sequence
  Synthetic construct
SKRSPIEDALFNKVTLADAGFIKQY

SEQ ID NO: 437 - protein, 25 aa, Artificial Sequence
  Synthetic construct
SKRSPIEDGLFNKVTLADAGFIKQY

SEQ ID NO: 438 - protein, 30 aa, Artificial Sequence
  Synthetic construct
QTYVTQQLGRAAEGRASANLAATKMSECVL

SEQ ID NO: 439 - protein, 50 aa, Severe acute respiratory syndrome coronavirus 2
ATSRTLSYYKLGASQRVAGDSGFAAYSRYRIGNYKLNTDHSSSSDNIALL

SEQ ID NO: 440 - protein, 1213 aa, Artificial Sequence
  Synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVGVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 441 - protein, 1213 aa, Artificial Sequence
  Synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVGVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLGRAAEGRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 442 - protein, 1273 aa, Artificial Sequence
  Synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVGVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 443 - protein, 1273 aa, Artificial Sequence
  Synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVGVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLGRAAEGRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 444 - protein, 1257 aa, Artificial Sequence
  Synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHH

SEQ ID NO: 445 - protein, 1257 aa, Artificial Sequence
  Synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVGVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHH

SEQ ID NO: 446 - protein, 1257 aa, Artificial Sequence
  Synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLGRAAEGRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHH

SEQ ID NO: 447 - protein, 9 aa, Severe acute respiratory syndrome coronavirus 2
LIVNSVLLF

SEQ ID NO: 448 - protein, 9 aa, Severe acute respiratory syndrome coronavirus 2
IVNSVLLFL

SEQ ID NO: 449 - protein, 10 aa, Severe acute respiratory syndrome coronavirus 2
NSVLLFLAFV

SEQ ID NO: 450 - protein, 9 aa, Severe acute respiratory syndrome coronavirus 2
SVLLFLAFV

SEQ ID NO: 451 - protein, 9 aa, Severe acute respiratory syndrome coronavirus 2
VLLFLAFVV

SEQ ID NO: 452 - protein, 9 aa, Severe acute respiratory syndrome coronavirus 2
FLAFVVFLL

SEQ ID NO: 453 - protein, 9 aa, Severe acute respiratory syndrome coronavirus 2
LAFVVFLLV

SEQ ID NO: 454 - protein, 9 aa, Severe acute respiratory syndrome coronavirus 2
FVVFLLVTL

SEQ ID NO: 455 - protein, 10 aa, Severe acute respiratory syndrome coronavirus 2
VVFLLVTLAI

SEQ ID NO: 456 - protein, 9 aa, Severe acute respiratory syndrome coronavirus 2
VTLAILTAL

SEQ ID NO: 457 - protein, 9 aa, Severe acute respiratory syndrome coronavirus 2
LAILTALRL

SEQ ID NO: 458 - protein, 9 aa, Severe acute respiratory syndrome coronavirus 2
TITVEELKK

SEQ ID NO: 459 - protein, 9 aa, Severe acute respiratory syndrome coronavirus 2
VEELKKLLE

SEQ ID NO: 460 - protein, 9 aa, Severe acute respiratory syndrome coronavirus 2
WNLVIGFLF

SEQ ID NO: 461 - protein, 9 aa, Severe acute respiratory syndrome coronavirus 2
LVIGFLFLT

SEQ ID NO: 462 - protein, 9 aa, Severe acute respiratory syndrome coronavirus 2
VIGFLFLTW

SEQ ID NO: 463 - protein, 9 aa, Severe acute respiratory syndrome coronavirus 2
IKLIFLWLL

SEQ ID NO: 464 - protein, 9 aa, Severe acute respiratory syndrome coronavirus 2
ACFVLAAVY

SEQ ID NO: 465 - protein, 9 aa, Severe acute respiratory syndrome coronavirus 2
ITGGIAIAM

SEQ ID NO: 466 - protein, 9 aa, Severe acute respiratory syndrome coronavirus 2
RLFARTRSM

SEQ ID NO: 467 - protein, 9 aa, Severe acute respiratory syndrome coronavirus 2
SFNPETNIL

SEQ ID NO: 468 - protein, 9 aa, Severe acute respiratory syndrome coronavirus 2
PETNILLNV

SEQ ID NO: 469 - protein, 9 aa, Severe acute respiratory syndrome coronavirus 2
VATSRTLSY

SEQ ID NO: 470 - protein, 9 aa, Severe acute respiratory syndrome coronavirus 2
ATSRTLSYY

SEQ ID NO: 471 - protein, 9 aa, Severe acute respiratory syndrome coronavirus 2
NTDHSSSSD

SEQ ID NO: 472 - protein, 9 aa, Severe acute respiratory syndrome coronavirus 2
SSDNIALLV

SEQ ID NO: 473 - protein, 9 aa, Severe acute respiratory syndrome coronavirus 2
APRITFGGP

SEQ ID NO: 474 - protein, 9 aa, Severe acute respiratory syndrome coronavirus 2
GPSDSTGSN

SEQ ID NO: 475 - protein, 9 aa, Severe acute respiratory syndrome coronavirus 2
YYLGTGPEA

SEQ ID NO: 476 - protein, 10 aa, Severe acute respiratory syndrome coronavirus 2
ASSRSSSRSR

SEQ ID NO: 477 - protein, 9 aa, Severe acute respiratory syndrome coronavirus 2
SSRSSSRSR

SEQ ID NO: 478 - protein, 9 aa, Severe acute respiratory syndrome coronavirus 2
SSRSRNSSR

SEQ ID NO: 479 - protein, 9 aa, Severe acute respiratory syndrome coronavirus 2
NSTPGSSRG

SEQ ID NO: 480 - protein, 9 aa, Severe acute respiratory syndrome coronavirus 2
TPGSSRGTS

SEQ ID NO: 481 - protein, 9 aa, Severe acute respiratory syndrome coronavirus 2
SSRGTSPAR

SEQ ID NO: 482 - protein, 9 aa, Severe acute respiratory syndrome coronavirus 2
GDAALALLL

SEQ ID NO: 483 - protein, 9 aa, Severe acute respiratory syndrome coronavirus 2
KPRQKRTAT

SEQ ID NO: 484 - protein, 9 aa, Severe acute respiratory syndrome coronavirus 2
QFAPSASAF

SEQ ID NO: 485 - protein, 9 aa, Severe acute respiratory syndrome coronavirus 2
AYKTFPPTE

SEQ ID NO: 486 - protein, 9 aa, Severe acute respiratory syndrome coronavirus 2
FSKQLQQSM

SEQ ID NO: 487 - protein, 9 aa, Severe acute respiratory syndrome coronavirus 2
FTIYSLLLC

SEQ ID NO: 488 - protein, 9 aa, Severe acute respiratory syndrome coronavirus 2
IYSLLLCRM

SEQ ID NO: 489 - protein, 9 aa, Severe acute respiratory syndrome coronavirus 2
GTTTLNGLW

SEQ ID NO: 490 - protein, 9 aa, Severe acute respiratory syndrome coronavirus 2
QLRVIGHSM

SEQ ID NO: 491 - protein, 9 aa, Severe acute respiratory syndrome coronavirus 2
IQPGQTFSV

SEQ ID NO: 492 - protein, 9 aa, Severe acute respiratory syndrome coronavirus 2
GTDTTITVN

SEQ ID NO: 493 - protein, 9 aa, Severe acute respiratory syndrome coronavirus 2
NLVAMKYNY

SEQ ID NO: 494 - protein, 9 aa, Severe acute respiratory syndrome coronavirus 2
GMNGRTILG

SEQ ID NO: 495 - protein, 9 aa, Severe acute respiratory syndrome coronavirus 2
CSKVITGLH

SEQ ID NO: 496 - protein, 9 aa, Severe acute respiratory syndrome coronavirus 2
TQAPTHLSV

SEQ ID NO: 497 - protein, 9 aa, Severe acute respiratory syndrome coronavirus 2
ITREEAIRH

SEQ ID NO: 498 - protein, 9 aa, Severe acute respiratory syndrome coronavirus 2
VQMLSDTLK

SEQ ID NO: 499 - protein, 9 aa, Severe acute respiratory syndrome coronavirus 2
VDRYPANSI

SEQ ID NO: 500 - protein, 9 aa, Severe acute respiratory syndrome coronavirus 2
PGCDGGSLY

SEQ ID NO: 501 - protein, 9 aa, Severe acute respiratory syndrome coronavirus 2
GCDGGSLYV

SEQ ID NO: 502 - protein, 9 aa, Severe acute respiratory syndrome coronavirus 2
KSAFVNLKQ

SEQ ID NO: 503 - protein, 9 aa, Severe acute respiratory syndrome coronavirus 2
QVVSDIDYV

SEQ ID NO: 504 - protein, 9 aa, Severe acute respiratory syndrome coronavirus 2
KQFDTYNLW

SEQ ID NO: 505 - protein, 9 aa, Severe acute respiratory syndrome coronavirus 2
RNARNGVLI

SEQ ID NO: 506 - protein, 9 aa, Severe acute respiratory syndrome coronavirus 2
SVGPKQASL

SEQ ID NO: 507 - protein, 9 aa, Severe acute respiratory syndrome coronavirus 2
TQSRNLQEF

SEQ ID NO: 508 - protein, 9 aa, Severe acute respiratory syndrome coronavirus 2
LAMDEFIER

SEQ ID NO: 509 - protein, 9 aa, Severe acute respiratory syndrome coronavirus 2
SQLGGLHLL

SEQ ID NO: 510 - protein, 9 aa, Severe acute respiratory syndrome coronavirus 2
GLHLLIGLA

SEQ ID NO: 511 - protein, 9 aa, Severe acute respiratory syndrome coronavirus 2
RFKESPFEL

SEQ ID NO: 512 - protein, 9 aa, Severe acute respiratory syndrome coronavirus 2
TDAQTGSSK

SEQ ID NO: 513 - protein, 9 aa, Severe acute respiratory syndrome coronavirus 2
LLLDDFVEI

SEQ ID NO: 514 - protein, 9 aa, Severe acute respiratory syndrome coronavirus 2
LVLSVNPYV

SEQ ID NO: 515 - protein, 9 aa, Severe acute respiratory syndrome coronavirus 2
NTCTERLKL

SEQ ID NO: 516 - protein, 9 aa, Severe acute respiratory syndrome coronavirus 2
AETLKATEE

SEQ ID NO: 517 - protein, 9 aa, Severe acute respiratory syndrome coronavirus 2
KATEETFKL

SEQ ID NO: 518 - protein, 9 aa, Severe acute respiratory syndrome coronavirus 2
SDRELHLSW

SEQ ID NO: 519 - protein, 9 aa, Severe acute respiratory syndrome coronavirus 2
EVGKPRPPL

SEQ ID NO: 520 - protein, 9 aa, Severe acute respiratory syndrome coronavirus 2
YTFEKGDYG

SEQ ID NO: 521 - protein, 9 aa, Severe acute respiratory syndrome coronavirus 2
GDYGDAVVY

SEQ ID NO: 522 - protein, 9 aa, Severe acute respiratory syndrome coronavirus 2
TVMPLSAPT

SEQ ID NO: 523 - protein, 9 aa, Severe acute respiratory syndrome coronavirus 2
MPLSAPTLV

SEQ ID NO: 524 - protein, 9 aa, Severe acute respiratory syndrome coronavirus 2
ISDEFSSNV

SEQ ID NO: 525 - protein, 9 aa, Severe acute respiratory syndrome coronavirus 2
VGMQKYSTL

SEQ ID NO: 526 - protein, 10 aa, Severe acute respiratory syndrome coronavirus 2
TLQGPPGTGK

SEQ ID NO: 527 - protein, 9 aa, Severe acute respiratory syndrome coronavirus 2
LQGPPGTGK

SEQ ID NO: 528 - protein, 9 aa, Severe acute respiratory syndrome coronavirus 2
GPPGTGKSH

SEQ ID NO: 529 - protein, 9 aa, Severe acute respiratory syndrome coronavirus 2
FAIGLALYY

SEQ ID NO: 530 - protein, 9 aa, Severe acute respiratory syndrome coronavirus 2
ALCEKALKY

SEQ ID NO: 531 - protein, 9 aa, Severe acute respiratory syndrome coronavirus 2
CEKALKYLP

SEQ ID NO: 532 - protein, 9 aa, Severe acute respiratory syndrome coronavirus 2
RIIPARARV

SEQ ID NO: 533 - protein, 9 aa, Severe acute respiratory syndrome coronavirus 2
VVFDEISMA

SEQ ID NO: 534 - protein, 9 aa, Severe acute respiratory syndrome coronavirus 2
VVNARLRAK

SEQ ID NO: 535 - protein, 9 aa, Severe acute respiratory syndrome coronavirus 2
LPAPRTLLT

SEQ ID NO: 536 - protein, 9 aa, Severe acute respiratory syndrome coronavirus 2
AEIVDTVSA

SEQ ID NO: 537 - protein, 9 aa, Severe acute respiratory syndrome coronavirus 2
IVDTVSALV

SEQ ID NO: 538 - protein, 9 aa, Severe acute respiratory syndrome coronavirus 2
VDTVSALVY

SEQ ID NO: 539 - protein, 9 aa, Severe acute respiratory syndrome coronavirus 2
ITHDVSSAI

SEQ ID NO: 540 - protein, 9 aa, Severe acute respiratory syndrome coronavirus 2
GVVREFLTR

SEQ ID NO: 541 - protein, 9 aa, Severe acute respiratory syndrome coronavirus 2
LEIPRRNVA

SEQ ID NO: 542 - protein, 9 aa, Severe acute respiratory syndrome coronavirus 2
QAWQPGVAM

SEQ ID NO: 543 - protein, 9 aa, Severe acute respiratory syndrome coronavirus 2
CQYLNTLTL

SEQ ID NO: 544 - protein, 9 aa, Severe acute respiratory syndrome coronavirus 2
WLPTGTLLV

SEQ ID NO: 545 - protein, 9 aa, Severe acute respiratory syndrome coronavirus 2
KLALGGSVA

SEQ ID NO: 546 - protein, 9 aa, Severe acute respiratory syndrome coronavirus 2
LALGGSVAI

SEQ ID NO: 547 - protein, 9 aa, Severe acute respiratory syndrome coronavirus 2
VMHANYIFW

SEQ ID NO: 548 - protein, 9 aa, Severe acute respiratory syndrome coronavirus 2
IQLSSYSLF

SEQ ID NO: 549 - protein, 9 aa, Severe acute respiratory syndrome coronavirus 2
LLSKGRLII

SEQ ID NO: 550 - protein, 9 aa, Severe acute respiratory syndrome coronavirus 2
VVISSDVLV

SEQ ID NO: 551 - protein, 9 aa, Severe acute respiratory syndrome coronavirus 2
YLASGGQPI

SEQ ID NO: 552 - protein, 9 aa, Severe acute respiratory syndrome coronavirus 2
MLCTHTGTG

SEQ ID NO: 553 - protein, 9 aa, Severe acute respiratory syndrome coronavirus 2
TPFEIKLAK

SEQ ID NO: 554 - protein, 9 aa, Severe acute respiratory syndrome coronavirus 2
FEIKLAKKF

SEQ ID NO: 555 - protein, 9 aa, Severe acute respiratory syndrome coronavirus 2
RKGGRTIAF

SEQ ID NO: 556 - protein, 9 aa, Severe acute respiratory syndrome coronavirus 2
VPRASANIG

SEQ ID NO: 557 - protein, 9 aa, Severe acute respiratory syndrome coronavirus 2
GEGSEGLND

SEQ ID NO: 558 - protein, 9 aa, Severe acute respiratory syndrome coronavirus 2
GLNDNLLEI

SEQ ID NO: 559 - protein, 9 aa, Severe acute respiratory syndrome coronavirus 2
ILASFSAST

SEQ ID NO: 560 - protein, 9 aa, Severe acute respiratory syndrome coronavirus 2
KVTKGKAKK

SEQ ID NO: 561 - protein, 9 aa, Severe acute respiratory syndrome coronavirus 2
QKSILSPLY

SEQ ID NO: 562 - protein, 9 aa, Severe acute respiratory syndrome coronavirus 2
ILSPLYAFA

SEQ ID NO: 563 - protein, 9 aa, Severe acute respiratory syndrome coronavirus 2
FASEAARVV

SEQ ID NO: 564 - protein, 9 aa, Severe acute respiratory syndrome coronavirus 2
ISQYSLRLI

SEQ ID NO: 565 - protein, 9 aa, Severe acute respiratory syndrome coronavirus 2
TNNLVVMAY

SEQ ID NO: 566 - protein, 9 aa, Severe acute respiratory syndrome coronavirus 2
IFGTVYEKL

SEQ ID NO: 567 - protein, 9 aa, Severe acute respiratory syndrome coronavirus 2
TVYEKLKPV

SEQ ID NO: 568 - protein, 9 aa, Severe acute respiratory syndrome coronavirus 2
VTHSKGLYR

SEQ ID NO: 569 - protein, 9 aa, Severe acute respiratory syndrome coronavirus 2
FLEGETLPT

SEQ ID NO: 570 - protein, 9 aa, Severe acute respiratory syndrome coronavirus 2
PTEVLTEEV

SEQ ID NO: 571 - protein, 9 aa, Severe acute respiratory syndrome coronavirus 2
VLTEEVVLK

SEQ ID NO: 572 - protein, 9 aa, Severe acute respiratory syndrome coronavirus 2
LTEEVVLKT

SEQ ID NO: 573 - protein, 9 aa, Severe acute respiratory syndrome coronavirus 2
EEVVLKTGD

SEQ ID NO: 574 - protein, 9 aa, Severe acute respiratory syndrome coronavirus 2
LEIKDTEKY

SEQ ID NO: 575 - protein, 9 aa, Severe acute respiratory syndrome coronavirus 2
KSVNITFEL

SEQ ID NO: 576 - protein, 9 aa, Severe acute respiratory syndrome coronavirus 2
KTLQPVSEL

SEQ ID NO: 577 - protein, 9 aa, Severe acute respiratory syndrome coronavirus 2
TLQPVSELL

SEQ ID NO: 578 - protein, 10 aa, Severe acute respiratory syndrome coronavirus 2
QPVSELLTPL

SEQ ID NO: 579 - protein, 9 aa, Severe acute respiratory syndrome coronavirus 2
VSELLTPLG

SEQ ID NO: 580 - protein, 9 aa, Severe acute respiratory syndrome coronavirus 2
SELLTPLGI

SEQ ID NO: 581 - protein, 9 aa, Severe acute respiratory syndrome coronavirus 2
TIVEVQPQL

SEQ ID NO: 582 - protein, 9 aa, Severe acute respiratory syndrome coronavirus 2
MELTPVVQT

SEQ ID NO: 583 - protein, 9 aa, Severe acute respiratory syndrome coronavirus 2
ELTPVVQTI

SEQ ID NO: 584 - protein, 9 aa, Severe acute respiratory syndrome coronavirus 2
TDNVYIKNA

SEQ ID NO: 585 - protein, 9 aa, Severe acute respiratory syndrome coronavirus 2
KPTVVVNAA

SEQ ID NO: 586 - protein, 9 aa, Severe acute respiratory syndrome coronavirus 2
YIATNGPLK

SEQ ID NO: 587 - protein, 9 aa, Severe acute respiratory syndrome coronavirus 2
EDIQLLKSA

SEQ ID NO: 588 - protein, 9 aa, Severe acute respiratory syndrome coronavirus 2
HEVLLAPLL

SEQ ID NO: 589 - protein, 9 aa, Severe acute respiratory syndrome coronavirus 2
VLLAPLLSA

SEQ ID NO: 590 - protein, 9 aa, Severe acute respiratory syndrome coronavirus 2
AVFDKNLYD

SEQ ID NO: 591 - protein, 9 aa, Severe acute respiratory syndrome coronavirus 2
NLYDKLVSS

SEQ ID NO: 592 - protein, 9 aa, Severe acute respiratory syndrome coronavirus 2
ITESKPSVE

SEQ ID NO: 593 - protein, 9 aa, Severe acute respiratory syndrome coronavirus 2
CVEEVTTTL

SEQ ID NO: 594 - protein, 9 aa, Severe acute respiratory syndrome coronavirus 2
VEEVTTTLE

SEQ ID NO: 595 - protein, 9 aa, Severe acute respiratory syndrome coronavirus 2
FLTENLLLY

SEQ ID NO: 596 - protein, 9 aa, Severe acute respiratory syndrome coronavirus 2
TENLLLYID

SEQ ID NO: 597 - protein, 9 aa, Severe acute respiratory syndrome coronavirus 2
HPDSATLVS

SEQ ID NO: 598 - protein, 9 aa, Severe acute respiratory syndrome coronavirus 2
DIDITFLKK

SEQ ID NO: 599 - protein, 9 aa, Severe acute respiratory syndrome coronavirus 2
LTAVVIPTK

SEQ ID NO: 600 - protein, 9 aa, Severe acute respiratory syndrome coronavirus 2
TEMLAKALR

SEQ ID NO: 601 - protein, 9 aa, Severe acute respiratory syndrome coronavirus 2
KSAFYILPS

SEQ ID NO: 602 - protein, 9 aa, Severe acute respiratory syndrome coronavirus 2
ETKAIVSTI

SEQ ID NO: 603 - protein, 9 aa, Severe acute respiratory syndrome coronavirus 2
VPATVSVSS

SEQ ID NO: 604 - protein, 9 aa, Severe acute respiratory syndrome coronavirus 2
TPEEHFIET

SEQ ID NO: 605 - protein, 9 aa, Severe acute respiratory syndrome coronavirus 2
ETISLAGSY

SEQ ID NO: 606 - protein, 10 aa, Severe acute respiratory syndrome coronavirus 2
FDNLKTLLSL

SEQ ID NO: 607 - protein, 9 aa, Severe acute respiratory syndrome coronavirus 2
LSLREVRTI

SEQ ID NO: 608 - protein, 9 aa, Severe acute respiratory syndrome coronavirus 2
TTVDNINLH

SEQ ID NO: 609 - protein, 9 aa, Severe acute respiratory syndrome coronavirus 2
NSHEGKTFY

SEQ ID NO: 610 - protein, 9 aa, Severe acute respiratory syndrome coronavirus 2
HTTDPSFLG

SEQ ID NO: 611 - protein, 9 aa, Severe acute respiratory syndrome coronavirus 2
YPQVNGLTS

SEQ ID NO: 612 - protein, 9 aa, Severe acute respiratory syndrome coronavirus 2
CYLATALLT

SEQ ID NO: 613 - protein, 9 aa, Severe acute respiratory syndrome coronavirus 2
YLATALLTL

SEQ ID NO: 614 - protein, 9 aa, Severe acute respiratory syndrome coronavirus 2
NFCALILAY

SEQ ID NO: 615 - protein, 9 aa, Severe acute respiratory syndrome coronavirus 2
MYMGTLSYE

SEQ ID NO: 616 - protein, 9 aa, Severe acute respiratory syndrome coronavirus 2
YELKHGTFT

SEQ ID NO: 617 - protein, 9 aa, Severe acute respiratory syndrome coronavirus 2
KLDGVVCTE

SEQ ID NO: 618 - protein, 9 aa, Severe acute respiratory syndrome coronavirus 2
FTEQPIDLV

SEQ ID NO: 619 - protein, 9 aa, Severe acute respiratory syndrome coronavirus 2
KKGAKLLHK

SEQ ID NO: 620 - protein, 9 aa, Severe acute respiratory syndrome coronavirus 2
YVDNSSLTI

SEQ ID NO: 621 - protein, 9 aa, Severe acute respiratory syndrome coronavirus 2
KPNELSRVL

SEQ ID NO: 622 - protein, 10 aa, Severe acute respiratory syndrome coronavirus 2
KPNELSRVLG

SEQ ID NO: 623 - protein, 10 aa, Severe acute respiratory syndrome coronavirus 2
YMPYFFTLLL

SEQ ID NO: 624 - protein, 10 aa, Severe acute respiratory syndrome coronavirus 2
MPYFFTLLLQ

SEQ ID NO: 625 - protein, 10 aa, Severe acute respiratory syndrome coronavirus 2
PYFFTLLLQL

SEQ ID NO: 626 - protein, 9 aa, Severe acute respiratory syndrome coronavirus 2
LKSPNFSKL

SEQ ID NO: 627 - protein, 9 aa, Severe acute respiratory syndrome coronavirus 2
NIIIWFLLL

SEQ ID NO: 628 - protein, 10 aa, Severe acute respiratory syndrome coronavirus 2
IIIWFLLLSV

SEQ ID NO: 629 - protein, 10 aa, Severe acute respiratory syndrome coronavirus 2
IWFLLLSVCL

SEQ ID NO: 630 - protein, 10 aa, Severe acute respiratory syndrome coronavirus 2
FLLLSVCLGS

SEQ ID NO: 631 - protein, 10 aa, Severe acute respiratory syndrome coronavirus 2
LSVCLGSLIY

SEQ ID NO: 632 - protein, 10 aa, Severe acute respiratory syndrome coronavirus 2
SVCLGSLIYS

SEQ ID NO: 633 - protein, 10 aa, Severe acute respiratory syndrome coronavirus 2
LIYSTAALGV

SEQ ID NO: 634 - protein, 10 aa, Severe acute respiratory syndrome coronavirus 2
YSTAALGVLM

SEQ ID NO: 635 - protein, 10 aa, Severe acute respiratory syndrome coronavirus 2
SYCTGYREGY

SEQ ID NO: 636 - protein, 10 aa, Severe acute respiratory syndrome coronavirus 2
FKWDLTAFGL

SEQ ID NO: 637 - protein, 10 aa, Severe acute respiratory syndrome coronavirus 2
LAYILFTRFF

SEQ ID NO: 638 - protein, 10 aa, Severe acute respiratory syndrome coronavirus 2
TIVNGVRRSF

SEQ ID NO: 639 - protein, 10 aa, Severe acute respiratory syndrome coronavirus 2
YANGGKGFCK

SEQ ID NO: 640 - protein, 10 aa, Severe acute respiratory syndrome coronavirus 2
QPILLLDQAL

SEQ ID NO: 641 - protein, 10 aa, Severe acute respiratory syndrome coronavirus 2
MFDAYVNTFS

SEQ ID NO: 642 - protein, 10 aa, Severe acute respiratory syndrome coronavirus 2
AYVNTFSSTF

SEQ ID NO: 643 - protein, 9 aa, Severe acute respiratory syndrome coronavirus 2
TFNVPMEKL

SEQ ID NO: 644 - protein, 9 aa, Severe acute respiratory syndrome coronavirus 2
MEKLKTLVA

SEQ ID NO: 645 - protein, 9 aa, Severe acute respiratory syndrome coronavirus 2
ATAEAELAK

SEQ ID NO: 646 - protein, 9 aa, Severe acute respiratory syndrome coronavirus 2
AEAELAKNV

SEQ ID NO: 647 - protein, 9 aa, Severe acute respiratory syndrome coronavirus 2
NVLSTFISA

SEQ ID NO: 648 - protein, 10 aa, Severe acute respiratory syndrome coronavirus 2
KLSHQSDIEV

SEQ ID NO: 649 - protein, 9 aa, Severe acute respiratory syndrome coronavirus 2
VENMTPRDL

SEQ ID NO: 650 - protein, 10 aa, Severe acute respiratory syndrome coronavirus 2
LSEQLRKQIR

SEQ ID NO: 651 - protein, 10 aa, Severe acute respiratory syndrome coronavirus 2
SAAKKNNLPF

SEQ ID NO: 652 - protein, 9 aa, Severe acute respiratory syndrome coronavirus 2
AKKNNLPFK

SEQ ID NO: 653 - protein, 9 aa, Severe acute respiratory syndrome coronavirus 2
LTCATTRQV

SEQ ID NO: 654 - protein, 10 aa, Severe acute respiratory syndrome coronavirus 2
NVVTTKIALK

SEQ ID NO: 655 - protein, 10 aa, Severe acute respiratory syndrome coronavirus 2
QLIKVTLVFL

SEQ ID NO: 656 - protein, 10 aa, Severe acute respiratory syndrome coronavirus 2
LIKVTLVFLF

SEQ ID NO: 657 - protein, 10 aa, Severe acute respiratory syndrome coronavirus 2
IKVTLVFLFV

SEQ ID NO: 658 - protein, 10 aa, Severe acute respiratory syndrome coronavirus 2
TLVFLFVAAI

SEQ ID NO: 659 - protein, 10 aa, Severe acute respiratory syndrome coronavirus 2
FLFVAAIFYL

SEQ ID NO: 660 - protein, 10 aa, Severe acute respiratory syndrome coronavirus 2
YTNDKACPLI

SEQ ID NO: 661 - protein, 10 aa, Severe acute respiratory syndrome coronavirus 2
REVGFVVPGL

SEQ ID NO: 662 - protein, 10 aa, Severe acute respiratory syndrome coronavirus 2
GLPGTILRTT

SEQ ID NO: 663 - protein, 10 aa, Severe acute respiratory syndrome coronavirus 2
TTNGDFLHFL

SEQ ID NO: 664 - protein, 10 aa, Severe acute respiratory syndrome coronavirus 2
DFLHFLPRVF

SEQ ID NO: 665 - protein, 10 aa, Severe acute respiratory syndrome coronavirus 2
RVVTTFDSEY

SEQ ID NO: 666 - protein, 10 aa, Severe acute respiratory syndrome coronavirus 2
VLNNDYYRSL

SEQ ID NO: 667 - protein, 10 aa, Severe acute respiratory syndrome coronavirus 2
DYYRSLPGVF

SEQ ID NO: 668 - protein, 10 aa, Severe acute respiratory syndrome coronavirus 2
VFCGVDAVNL

SEQ ID NO: 669 - protein, 10 aa, Severe acute respiratory syndrome coronavirus 2
LLTNMFTPLI

SEQ ID NO: 670 - protein, 10 aa, Severe acute respiratory syndrome coronavirus 2
NMFTPLIQPI

SEQ ID NO: 671 - protein, 10 aa, Severe acute respiratory syndrome coronavirus 2
SIVAGGIVAI

SEQ ID NO: 672 - protein, 10 aa, Severe acute respiratory syndrome coronavirus 2
VVAFNTLLFL

SEQ ID NO: 673 - protein, 10 aa, Severe acute respiratory syndrome coronavirus 2
VAFNTLLFLM

SEQ ID NO: 674 - protein, 10 aa, Severe acute respiratory syndrome coronavirus 2
LFLMSFTVLC

SEQ ID NO: 675 - protein, 10 aa, Severe acute respiratory syndrome coronavirus 2
TPVYSFLPGV

SEQ ID NO: 676 - protein, 10 aa, Severe acute respiratory syndrome coronavirus 2
VIYLYLTFYL

SEQ ID NO: 677 - protein, 10 aa, Severe acute respiratory syndrome coronavirus 2
QWMVMFTPLV

SEQ ID NO: 678 - protein, 10 aa, Severe acute respiratory syndrome coronavirus 2
EEAALCTFLL

SEQ ID NO: 679 - protein, 10 aa, Severe acute respiratory syndrome coronavirus 2
TQYNRYLALY

SEQ ID NO: 680 - protein, 10 aa, Severe acute respiratory syndrome coronavirus 2
REAACCHLAK

SEQ ID NO: 681 - protein, 9 aa, Severe acute respiratory syndrome coronavirus 2
HLAKALNDF

SEQ ID NO: 682 - protein, 10 aa, Severe acute respiratory syndrome coronavirus 2
QPPQTSITSA

SEQ ID NO: 683 - protein, 9 aa, Severe acute respiratory syndrome coronavirus 2
PPQTSITSA

SEQ ID NO: 684 - protein, 10 aa, Severe acute respiratory syndrome coronavirus 2
HWLLLTILTS

SEQ ID NO: 685 - protein, 10 aa, Severe acute respiratory syndrome coronavirus 2
LLLTILTSLL

SEQ ID NO: 686 - protein, 10 aa, Severe acute respiratory syndrome coronavirus 2
LLTILTSLLV

SEQ ID NO: 687 - protein, 10 aa, Severe acute respiratory syndrome coronavirus 2
TILTSLLVLV

SEQ ID NO: 688 - protein, 10 aa, Severe acute respiratory syndrome coronavirus 2
TQWSLFFFLY

SEQ ID NO: 689 - protein, 10 aa, Severe acute respiratory syndrome coronavirus 2
FAMGIIAMSA

SEQ ID NO: 690 - protein, 9 aa, Severe acute respiratory syndrome coronavirus 2
MGIIAMSAF

SEQ ID NO: 691 - protein, 10 aa, Severe acute respiratory syndrome coronavirus 2
KHKHAFLCLF

SEQ ID NO: 692 - protein, 10 aa, Severe acute respiratory syndrome coronavirus 2
HKHAFLCLFL

SEQ ID NO: 693 - protein, 10 aa, Severe acute respiratory syndrome coronavirus 2
KHAFLCLFLL

SEQ ID NO: 694 - protein, 9 aa, Severe acute respiratory syndrome coronavirus 2
HAFLCLFLL

SEQ ID NO: 695 - protein, 9 aa, Severe acute respiratory syndrome coronavirus 2
FLCLFLLPS

SEQ ID NO: 696 - protein, 10 aa, Severe acute respiratory syndrome coronavirus 2
FLCLFLLPSL

SEQ ID NO: 697 - protein, 10 aa, Severe acute respiratory syndrome coronavirus 2
LFLLPSLATV

SEQ ID NO: 698 - protein, 10 aa, Severe acute respiratory syndrome coronavirus 2
VYMPASWVMR

SEQ ID NO: 699 - protein, 10 aa, Severe acute respiratory syndrome coronavirus 2
WLDMVDTSLS

SEQ ID NO: 700 - protein, 10 aa, Severe acute respiratory syndrome coronavirus 2
VMYASAVVLL

SEQ ID NO: 701 - protein, 10 aa, Severe acute respiratory syndrome coronavirus 2
MYASAVVLLI

SEQ ID NO: 702 - protein, 10 aa, Severe acute respiratory syndrome coronavirus 2
WTLMNVLTLV

SEQ ID NO: 703 - protein, 9 aa, Severe acute respiratory syndrome coronavirus 2
KVYYGNALD

SEQ ID NO: 704 - protein, 10 aa, Severe acute respiratory syndrome coronavirus 2
TCYFGLFCLL

SEQ ID NO: 705 - protein, 10 aa, Severe acute respiratory syndrome coronavirus 2
CYFGLFCLLN

SEQ ID NO: 706 - protein, 10 aa, Severe acute respiratory syndrome coronavirus 2
GLFCLLNRYF

SEQ ID NO: 707 - protein, 10 aa, Severe acute respiratory syndrome coronavirus 2
RYMNSQGLLP

SEQ ID NO: 708 - protein, 10 aa, Severe acute respiratory syndrome coronavirus 2
KCTSVVLLSV

SEQ ID NO: 709 - protein, 9 aa, Severe acute respiratory syndrome coronavirus 2
CTSVVLLSV

SEQ ID NO: 710 - protein, 10 aa, Severe acute respiratory syndrome coronavirus 2
RVESSSKLWA

SEQ ID NO: 711 - protein, 10 aa, Severe acute respiratory syndrome coronavirus 2
AQCVQLHNDI

SEQ ID NO: 712 - protein, 10 aa, Severe acute respiratory syndrome coronavirus 2
QLHNDILLAK

SEQ ID NO: 713 - protein, 10 aa, Severe acute respiratory syndrome coronavirus 2
EAFEKMVSLL

SEQ ID NO: 714 - protein, 10 aa, Severe acute respiratory syndrome coronavirus 2
EKMVSLLSVL

SEQ ID NO: 715 - protein, 10 aa, Severe acute respiratory syndrome coronavirus 2
KMVSLLSVLL

SEQ ID NO: 716 - protein, 9 aa, Severe acute respiratory syndrome coronavirus 2
MVSLLSVLL

SEQ ID NO: 717 - protein, 10 aa, Severe acute respiratory syndrome coronavirus 2
SMQGAVDINK

SEQ ID NO: 718 - protein, 10 aa, Severe acute respiratory syndrome coronavirus 2
ASEFSSLPSY

SEQ ID NO: 719 - protein, 10 aa, Severe acute respiratory syndrome coronavirus 2
DSEVVLKKLK

SEQ ID NO: 720 - protein, 9 aa, Severe acute respiratory syndrome coronavirus 2
RSEDKRAKV

SEQ ID NO: 721 - protein, 9 aa, Severe acute respiratory syndrome coronavirus 2
CVPLNIIPL

SEQ ID NO: 722 - protein, 10 aa, Severe acute respiratory syndrome coronavirus 2
NTCDGTTFTY

SEQ ID NO: 723 - protein, 10 aa, Severe acute respiratory syndrome coronavirus 2
NLAWPLIVTA

SEQ ID NO: 724 - protein, 10 aa, Severe acute respiratory syndrome coronavirus 2
WPLIVTALRA

SEQ ID NO: 725 - protein, 10 aa, Severe acute respiratory syndrome coronavirus 2
YYNTTKGGRF

SEQ ID NO: 726 - protein, 10 aa, Severe acute respiratory syndrome coronavirus 2
GLNNLNRGMV

SEQ ID NO: 727 - protein, 9 aa, Severe acute respiratory syndrome coronavirus 2
GMVLGSLAA

SEQ ID NO: 728 - protein, 10 aa, Severe acute respiratory syndrome coronavirus 2
GMVLGSLAAT

SEQ ID NO: 729 - protein, 10 aa, Severe acute respiratory syndrome coronavirus 2
MVLGSLAATV

SEQ ID NO: 730 - protein, 9 aa, Severe acute respiratory syndrome coronavirus 2
RVCGVSAAR

SEQ ID NO: 731 - protein, 10 aa, Severe acute respiratory syndrome coronavirus 2
FFKFRIDGDM

SEQ ID NO: 732 - protein, 9 aa, Severe acute respiratory syndrome coronavirus 2
VYANLGERV

SEQ ID NO: 733 - protein, 10 aa, Severe acute respiratory syndrome coronavirus 2
LTLDNQDLNG

SEQ ID NO: 734 - protein, 9 aa, Severe acute respiratory syndrome coronavirus 2
TTPGSGVPV

SEQ ID NO: 735 - protein, 10 aa, Severe acute respiratory syndrome coronavirus 2
FSTVFPPTSF

SEQ ID NO: 736 - protein, 9 aa, Severe acute respiratory syndrome coronavirus 2
TSFGPLVRK

SEQ ID NO: 737 - protein, 10 aa, Severe acute respiratory syndrome coronavirus 2
KIFVDGVPFV

SEQ ID NO: 738 - protein, 10 aa, Severe acute respiratory syndrome coronavirus 2
VNLHSSRLSF

SEQ ID NO: 739 - protein, 10 aa, Severe acute respiratory syndrome coronavirus 2
NLHSSRLSFK

SEQ ID NO: 740 - protein, 9 aa, Severe acute respiratory syndrome coronavirus 2
DPAMHAASG

SEQ ID NO: 741 - protein, 10 aa, Severe acute respiratory syndrome coronavirus 2
AMHAASGNLL

SEQ ID NO: 742 - protein, 10 aa, Severe acute respiratory syndrome coronavirus 2
AASGNLLLDK

SEQ ID NO: 743 - protein, 9 aa, Severe acute respiratory syndrome coronavirus 2
AAISDYDYY

SEQ ID NO: 744 - protein, 10 aa, Severe acute respiratory syndrome coronavirus 2
VVEVVDKYFD

SEQ ID NO: 745 - protein, 9 aa, Severe acute respiratory syndrome coronavirus 2
IAATRGATV

SEQ ID NO: 746 - protein, 9 aa, Severe acute respiratory syndrome coronavirus 2
AATRGATVV

SEQ ID NO: 747 - protein, 9 aa, Severe acute respiratory syndrome coronavirus 2
LRIMASLVL

SEQ ID NO: 748 - protein, 10 aa, Severe acute respiratory syndrome coronavirus 2
MVMCGGSLYV

SEQ ID NO: 749 - protein, 10 aa, Severe acute respiratory syndrome coronavirus 2
IADKYVRNLQ

SEQ ID NO: 750 - protein, 10 aa, Severe acute respiratory syndrome coronavirus 2
YVRNLQHRLY

SEQ ID NO: 751 - protein, 10 aa, Severe acute respiratory syndrome coronavirus 2
EFYAYLRKHF

SEQ ID NO: 752 - protein, 9 aa, Severe acute respiratory syndrome coronavirus 2
STYASQGLV

SEQ ID NO: 753 - protein, 10 aa, Severe acute respiratory syndrome coronavirus 2
SIKNFKSVLY

SEQ ID NO: 754 - protein, 10 aa, Severe acute respiratory syndrome coronavirus 2
SRILGAGCFV

SEQ ID NO: 755 - protein, 10 aa, Severe acute respiratory syndrome coronavirus 2
IFTIGTVTLK

SEQ ID NO: 756 - protein, 10 aa, Severe acute respiratory syndrome coronavirus 2
SDFVRATATI

SEQ ID NO: 757 - protein, 9 aa, Severe acute respiratory syndrome coronavirus 2
ATIPIQASL

SEQ ID NO: 758 - protein, 10 aa, Severe acute respiratory syndrome coronavirus 2
PIQASLPFGW

SEQ ID NO: 759 - protein, 10 aa, Severe acute respiratory syndrome coronavirus 2
GWLIVGVALL

SEQ ID NO: 760 - protein, 10 aa, Severe acute respiratory syndrome coronavirus 2
LLAVFQSASK

SEQ ID NO: 761 - protein, 9 aa, Severe acute respiratory syndrome coronavirus 2
KRWQLALSK

SEQ ID NO: 762 - protein, 10 aa, Severe acute respiratory syndrome coronavirus 2
VHFVCNLLLL

SEQ ID NO: 763 - protein, 10 aa, Severe acute respiratory syndrome coronavirus 2
HFVCNLLLLF

SEQ ID NO: 764 - protein, 10 aa, Severe acute respiratory syndrome coronavirus 2
FVCNLLLLFV

SEQ ID NO: 765 - protein, 9 aa, Severe acute respiratory syndrome coronavirus 2
VCNLLLLFV

SEQ ID NO: 766 - protein, 10 aa, Severe acute respiratory syndrome coronavirus 2
VTVYSHLLLV

SEQ ID NO: 767 - protein, 10 aa, Severe acute respiratory syndrome coronavirus 2
VYSHLLLVAA

SEQ ID NO: 768 - protein, 10 aa, Severe acute respiratory syndrome coronavirus 2
YLYALVYFLQ

SEQ ID NO: 769 - protein, 10 aa, Severe acute respiratory syndrome coronavirus 2
DYCIPYNSVT

SEQ ID NO: 770 - protein, 10 aa, Severe acute respiratory syndrome coronavirus 2
IPYNSVTSSI

SEQ ID NO: 771 - protein, 10 aa, Severe acute respiratory syndrome coronavirus 2
IYDEPTTTTS

SEQ ID NO: 772 - protein, 10 aa, Severe acute respiratory syndrome coronavirus 2
YDEPTTTTSV

SEQ ID NO: 773 - protein, 10 aa, Severe acute respiratory syndrome coronavirus 2
EPTTTTSVPL

SEQ ID NO: 774 - protein, 10 aa, Severe acute respiratory syndrome coronavirus 2
VDFQVTIAEI

SEQ ID NO: 775 - protein, 10 aa, Severe acute respiratory syndrome coronavirus 2
MKIILFLALI

SEQ ID NO: 776 - protein, 10 aa, Severe acute respiratory syndrome coronavirus 2
IILFLALITL

SEQ ID NO: 777 - protein, 10 aa, Severe acute respiratory syndrome coronavirus 2
LFLALITLAT

SEQ ID NO: 778 - protein, 10 aa, Severe acute respiratory syndrome coronavirus 2
QECVRGTTVL

SEQ ID NO: 779 - protein, 10 aa, Severe acute respiratory syndrome coronavirus 2
ADNKFALTCF

SEQ ID NO: 780 - protein, 10 aa, Severe acute respiratory syndrome coronavirus 2
FLIVAAIVFI

SEQ ID NO: 781 - protein, 9 aa, Severe acute respiratory syndrome coronavirus 2
VAAIVFITL

SEQ ID NO: 782 - protein, 9 aa, Severe acute respiratory syndrome coronavirus 2
TLCFTLKRK

SEQ ID NO: 783 - protein, 9 aa, Severe acute respiratory syndrome coronavirus 2
MKFLVFLGI

SEQ ID NO: 784 - protein, 10 aa, Severe acute respiratory syndrome coronavirus 2
FLVFLGIITT

SEQ ID NO: 785 - protein, 10 aa, Severe acute respiratory syndrome coronavirus 2
LGIITTVAAF

SEQ ID NO: 786 - protein, 9 aa, Severe acute respiratory syndrome coronavirus 2
IITTVAAFH

SEQ ID NO: 787 - protein, 10 aa, Severe acute respiratory syndrome coronavirus 2
RVGARKSAPL

SEQ ID NO: 788 - protein, 9 aa, Severe acute respiratory syndrome coronavirus 2
CVDEAGSKS

SEQ ID NO: 789 - protein, 10 aa, Severe acute respiratory syndrome coronavirus 2
YTVSCLPFTI

SEQ ID NO: 790 - protein, 9 aa, Severe acute respiratory syndrome coronavirus 2
FTINCQEPK

SEQ ID NO: 791 - protein, 9 aa, Severe acute respiratory syndrome coronavirus 2
QEPKLGSLV

SEQ ID NO: 792 - protein, 10 aa, Severe acute respiratory syndrome coronavirus 2
YHDVRVVLDF

SEQ ID NO: 793 - protein, 10 aa, Severe acute respiratory syndrome coronavirus 2
MFVFLVLLPL

SEQ ID NO: 794 - protein, 10 aa, Severe acute respiratory syndrome coronavirus 2
FVFLVLLPLV

SEQ ID NO: 795 - protein, 9 aa, Severe acute respiratory syndrome coronavirus 2
VFLVLLPLV

SEQ ID NO: 796 - protein, 10 aa, Severe acute respiratory syndrome coronavirus 2
YPDKVFRSSV

SEQ ID NO: 797 - protein, 10 aa, Severe acute respiratory syndrome coronavirus 2
VLHSTQDLFL

SEQ ID NO: 798 - protein, 10 aa, Severe acute respiratory syndrome coronavirus 2
NIIRGWIFGT

SEQ ID NO: 799 - protein, 10 aa, Severe acute respiratory syndrome coronavirus 2
FEYVSQPFLM

SEQ ID NO: 800 - protein, 10 aa, Severe acute respiratory syndrome coronavirus 2
GINITRFQTL

SEQ ID NO: 801 - protein, 10 aa, Severe acute respiratory syndrome coronavirus 2
YLTPGDSSSG

SEQ ID NO: 802 - protein, 9 aa, Severe acute respiratory syndrome coronavirus 2
TPGDSSSGW

SEQ ID NO: 803 - protein, 10 aa, Severe acute respiratory syndrome coronavirus 2
LYNSASFSTF

SEQ ID NO: 804 - protein, 9 aa, Severe acute respiratory syndrome coronavirus 2
PTKLNDLCF

SEQ ID NO: 805 - protein, 10 aa, Severe acute respiratory syndrome coronavirus 2
QPYRVVVLSF

SEQ ID NO: 806 - protein, 9 aa, Severe acute respiratory syndrome coronavirus 2
RVVVLSFEL

SEQ ID NO: 807 - protein, 10 aa, Severe acute respiratory syndrome coronavirus 2
APATVCGPKK

SEQ ID NO: 808 - protein, 10 aa, Severe acute respiratory syndrome coronavirus 2
IGAEHVNNSY

SEQ ID NO: 809 - protein, 10 aa, Severe acute respiratory syndrome coronavirus 2
YECDIPIGAG

SEQ ID NO: 810 - protein, 10 aa, Severe acute respiratory syndrome coronavirus 2
TNSPRRARSV

SEQ ID NO: 811 - protein, 10 aa, Severe acute respiratory syndrome coronavirus 2
SFCTQLNRAL

SEQ ID NO: 812 - protein, 9 aa, Severe acute respiratory syndrome coronavirus 2
FCTQLNRAL

SEQ ID NO: 813 - protein, 10 aa, Severe acute respiratory syndrome coronavirus 2
DPSKPSKRSF

SEQ ID NO: 814 - protein, 10 aa, Severe acute respiratory syndrome coronavirus 2
AQKFNGLTVL

SEQ ID NO: 815 - protein, 9 aa, Severe acute respiratory syndrome coronavirus 2
IAQYTSALL

SEQ ID NO: 816 - protein, 10 aa, Severe acute respiratory syndrome coronavirus 2
YTSALLAGTI

SEQ ID NO: 817 - protein, 10 aa, Severe acute respiratory syndrome coronavirus 2
GWTFGAGAAL

SEQ ID NO: 818 - protein, 10 aa, Severe acute respiratory syndrome coronavirus 2
LIANQFNSAI

SEQ ID NO: 819 - protein, 9 aa, Severe acute respiratory syndrome coronavirus 2
QFNSAIGKI

SEQ ID NO: 820 - protein, 9 aa, Severe acute respiratory syndrome coronavirus 2
KIQDSLSST

SEQ ID NO: 821 - protein, 9 aa, Severe acute respiratory syndrome coronavirus 2
IQDSLSSTA

SEQ ID NO: 822 - protein, 10 aa, Severe acute respiratory syndrome coronavirus 2
LSSTASALGK

SEQ ID NO: 823 - protein, 9 aa, Severe acute respiratory syndrome coronavirus 2
ALNTLVKQL

SEQ ID NO: 824 - protein, 9 aa, Severe acute respiratory syndrome coronavirus 2
AISSVLNDI

SEQ ID NO: 825 - protein, 10 aa, Severe acute respiratory syndrome coronavirus 2
RLITGRLQSL

SEQ ID NO: 826 - protein, 10 aa, Severe acute respiratory syndrome coronavirus 2
FPREGVFVSN

SEQ ID NO: 827 - protein, 9 aa, Severe acute respiratory syndrome coronavirus 2
ITTDNTFVS

SEQ ID NO: 828 - protein, 10 aa, Severe acute respiratory syndrome coronavirus 2
FKEELDKYFK

SEQ ID NO: 829 - protein, 10 aa, Severe acute respiratory syndrome coronavirus 2
KYFKNHTSPD

SEQ ID NO: 830 - protein, 9 aa, Severe acute respiratory syndrome coronavirus 2
VDLGDISGI

SEQ ID NO: 831 - protein, 10 aa, Severe acute respiratory syndrome coronavirus 2
SLIDLQELGK

SEQ ID NO: 832 - protein, 10 aa, Severe acute respiratory syndrome coronavirus 2
GLIAIVMVTI

SEQ ID NO: 833 - protein, 1257 aa, Artificial Sequence
  Synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVGVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLGRAAEGRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHH

SEQ ID NO: 834 - DNA, 32 bp, Artificial Sequence
  Synthetic Construct
gttaactagttcagctggaccacagccgcagc

SEQ ID NO: 835 - DNA, 39 bp, Artificial Sequence
  Synthetic Construct
cgggttaactagttcagaaatcctttctcttgaccatgg

SEQ ID NO: 836 - DNA, 25 bp, Artificial Sequence
  Synthetic Construct
ctagcctctggaatcctttctcttg

SEQ ID NO: 837 - protein, 1273 aa, Artificial Sequence
  Synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLGFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 838 - protein, 1273 aa, Artificial Sequence
  Synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLGRAAEGRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 839 - protein, 1273 aa, Artificial Sequence
  Synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLGFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLGRAAEGRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 840 - protein, 1213 aa, Artificial Sequence
  Synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLGFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 841 - protein, 1213 aa, Artificial Sequence
  Synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLGRAAEGRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 842 - protein, 1213 aa, Artificial Sequence
  Synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLGFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLGRAAEGRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 843 - protein, 1273 aa, Artificial Sequence; identical to SEQ ID NO: 838
  Full Length Spike Opt 2

SEQ ID NO: 844 - protein, 1212 aa, Artificial Sequence
  Full Length Spike Opt 3
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLGRAAEGRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVKNLNESLIDLQELGKYEQYIKWP

SEQ ID NO: 845 - protein, 1212 aa, Artificial Sequence
  Ectodomain Spike Opt 1
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLGFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVKNLNESLIDLQELGKYEQYIKWP

SEQ ID NO: 846 - protein, 1212 aa, Artificial Sequence; identical to SEQ ID NO: 844
  Ectodomain Spike Opt 2

SEQ ID NO: 847 - protein, 1213 aa, Artificial Sequence; identical to SEQ ID NO: 842
  Ectodomain Spike Opt 3

(19) United States
(12) Patent Application Publication
(10) Pub. No.: US 2023/0242591 A1
(43) Pub. Date: Aug. 3, 2023

(54) REGULATORY T CELL EPITOPES AND DETOLERIZED SARS-COV-2 ANTIGENS

(71) Applicant: EPIVAX, INC., Providence, RI (US)

(72) Inventors: Anne De Groot, Providence, RI (US); William Martin, Providence, RI (US)

(21) Appl. No.: 17/795,261

(22) PCT Filed: Feb. 12, 2021
(86) PCT No.: PCT/US2021/017782

§ 371 Date: Jul. 26, 2022

Related U.S. Application Data
(60) Provisional application No. 62/976,715, filed on Feb. 14, 2020.
(60) Provisional application No. 62/983,012, filed on Feb. 28, 2020.
(60) Provisional application No. 62/991,790, filed on Mar. 19, 2020.
(60) Provisional application No. 62/991,814, filed on Mar. 19, 2020.
(60) Provisional application No. 63/001,632, filed on Mar. 30, 2020.
(60) Provisional application No. 63/001,624, filed on Mar. 30, 2020.
(60) Provisional application No. 63/004,729, filed on Apr. 3, 2020.
(60) Provisional application No. 63/006,962, filed on Apr. 8, 2020.
(60) Provisional application No. 63/065,135, filed on Aug. 13, 2020.
(60) Provisional application No. 63/065,161, filed on Aug. 13, 2020.
(60) Provisional application No. 63/065,152, filed on Aug. 13, 2020.
(60) Provisional application No. 63/065,129, filed on Aug. 13, 2020.
(60) Provisional application No. 63/065,163, filed on Aug. 13, 2020.
(60) Provisional application No. 63/073,161, filed on Sep. 1, 2020.
(60) Provisional application No. 63/073,156, filed on Sep. 1, 2020.
(60) Provisional application No. 63/081,062, filed on Sep. 21, 2020.
(60) Provisional application No. 63/081,055, filed on Sep. 21, 2020.
(60) Provisional application No. 63/083,389, filed on Sep. 25, 2020.
(60) Provisional application No. 63/092,229, filed on Oct. 15, 2020.

(51) Int. Cl. C07K 14/005; C12N 15/63; A61K 39/12; A61P 31/14

(52) U.S. Cl. CPC C07K 14/005; C12N 15/63; A61K 39/12; A61P 31/14; C12N 2770/20022; C12N 2770/20034

(57) ABSTRACT

The present is directed to compositions comprising regulatory T cell epitopes, wherein said epitopes comprises a polypeptide comprising at least a portion of SEQ ID NOS: 4-370, 391-440, and 448-833 (and/or fragments and variants thereof), and optionally 1 to 12 additional amino acids distributed in any ratio on the N-terminus and/or C-terminus of the polypeptide of SEQ ID NOS: 4-370, 391-440, and 448-833, as well as methods of producing and using the same. The present is further directed to detolerized antigens to the regulatory T cell epitopes, including proteins or polypeptides of SARS-CoV-2 wherein one or more of the identified T cell epitopes are deleted, partially deleted and/or mutated.

### CROSS-REFERENCE TO RELATED APPLICATION

This application is a U.S. national stage under 35 U.S.C. § 371 of international application number PCT/US2021/017782 filed Feb. 12, 2021, and which depends from and claims priority to U.S. Provisional Application No. 62/976,715 filed Feb. 14, 2020, U.S. Provisional Application No. 62/991,790 filed Mar. 19, 2020, U.S. Provisional Application No. 63/001,632 filed Mar. 30, 2020, U.S. Provisional Application No. 63/065,129 filed Aug. 13, 2020, U.S. Provisional Application No. 63/073,161 filed Sep. 1, 2020, U.S. Provisional Application No. 63/083,389 filed Sep. 25, 2020, U.S. Provisional Application No. 63/092,229 filed Oct. 15, 2020, U.S. Provisional Application No. 62/983,012 filed Feb. 28, 2020, U.S. Provisional Application No. 62/991,814 filed Mar. 19, 2020, U.S. Provisional Application No. 63/065,161 filed Aug. 13, 2020, U.S. Provisional Application No. 63/081,062 filed Sep. 21, 2020, U.S. Provisional Application No. 63/001,624 filed Mar. 30, 2020, U.S. Provisional Application No. 63/065,135 filed Aug. 13, 2020, U.S. Provisional Application No. 63/004,729 filed Apr. 3, 2020, U.S. Provisional Application No. 63/065,152 filed Aug. 13, 2020, U.S. Provisional Application No. 63/006,962 filed Apr. 8, 2020, U.S. Provisional Application No. 63/065,163 filed Aug. 13, 2020, U.S. Provisional Application No. 63/073,156 filed Sep. 1, 2020, and U.S. Provisional Application No. 63/081,055 filed Sep. 21, 2020, the entire contents of each of which are incorporated herein by reference in their entirety.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created Feb. 8, 2023, is named “2023-02-08 Sequence Listing Corrected EPV0036PA_ST25” and is 453,601 bytes in size.

### FIELD

The present disclosure generally relates to immune-suppressive peptides and detolerized antigens from the SARS-CoV-2 virus. The disclosure concerns a novel class of regulatory T cell activating epitope (termed “Tregitope”, “Treg activating regulatory T-cell epitope”, “Tregitope peptide”, or “T-cell epitope polypeptide”) and/or detolerized antigens thereof, including compositions and methods of producing and using the same. Such compounds and compositions include Tregitope polypeptides or detolerized antigens thereof (including concatemeric polypeptides and chimeric or fusion polypeptides), as well as nucleic acids, plasmids, vectors (including expression vectors), and cells which express the polypeptides, pharmaceutical compositions, and vaccines. The present disclosure also generally relates to methods, assays, and kits against severe acute respiratory syndrome coronavirus 2 (SARS-CoV-2) or a related coronavirus, as well as methods, assays, and kits for the diagnosis of a SARS-CoV-2 infection or related coronaviruses infection.

### BACKGROUND

Severe acute respiratory syndrome coronavirus 2 (SARS-CoV-2) is a positive-sense single-stranded ribonucleic (RNA) virus belonging to the Coronaviridae family. SARS-CoV-2 (which may also be referred to herein as “COVID-19 virus”) was first identified in Wuhan, China in late 2019 and is the cause of the highly contagious coronavirus disease 2019 (which has been termed and may be referred to herein as “COVID-19”, “2019 novel coronavirus”, or “2019-nCoV”). SARS-CoV-2 infection causes a broad range of disease, known as coronavirus disease 2019 (COVID-19), from mild or no symptoms to serious complications that may be rapidly fatal, often in adults over 65 years old and individuals with underlying medical conditions including cardiovascular disease, type 2 diabetes, and obesity. The global spread of COVID-19 was declared a pandemic by the World Health Organization (WHO) on Mar. 11, 2020. As of Dec. 25, 2020, the global spread of severe acute respiratory syndrome coronavirus 2 (SARS-CoV-2) has resulted in over 79 million cases of COVID-19, 1.7 million deaths and global economic disruption in less than 10 months since the first case appeared in Wuhan, China. Recovery from natural infection in non-severe disease, and resistance to severe disease in younger individuals suggests that the immune system can be harnessed to help bring an end to the COVID-19 pandemic by vaccination strategies that recapitulate protective immune responses.

While immune correlates of COVID-19 protection are not yet defined, several studies show cellular adaptive immune mechanisms contribute to SARS-CoV-2 control. Humoral immune responses also contribute to protection and have been the focus of current vaccine development efforts. Virus-specific IgM and IgG antibodies are found in nearly all infections. Seroconversion is observed 7 to 14 days after onset of symptoms and persists for weeks after virus clearance. Antibody levels wane four to five months after infection, but durable memory B cell immunity has been described in mild and severe disease. Antibodies are found against the surface spike glycoprotein and the internal nucleocapsid protein. Neutralizing antibodies target the receptor binding domain of spike, preventing cell entry via the angiotensin-converting enzyme 2 (ACE2) host receptor. Neutralizing antibodies are found in more than 90% of persons who seroconvert. In a prospective study of exposed healthcare workers, anti-COVID-19 IgG titers were correlated with protection from subsequent PCR test positivity, suggesting that either antibodies, or T cell response (responsible for driving higher Ab titers) or both were correlates of protection from subsequent infection. In other studies, spike-specific follicular helper CD4 T cells (Tfh) frequencies correlate with neutralizing antibody responses. Although much of the current COVID-19 vaccine focus has been on generating antibody responses, this latter finding identifies a critical role for T cells in generating immunity.

More recently, correlations between a wide range of T cell responses and protection from infection have begun to emerge. A large prospective study showed numbers of SARS-CoV-2-specific T cells indirectly correlate with disease risk. Individuals with low T cell responses to spike, membrane and nucleocapsid proteins develop COVID-19 while high responders do not, even if seronegative. T cell breadth is another key feature of the protected response, as patients with mild disease have higher TCR clonality in blood and bronchoalveolar lavage in comparison with severe disease.

T cell phenotype and function may also help to predict mild versus severe cases. Poor outcomes are associated with multiple signs of T cell impairment including enhanced expression of PD-1 and TIM-3 exhaustion markers, higher inhibitory molecule levels including CTLA-4 and TIGIT, and low frequencies of polyfunctional CD4 and CD8 T cells, as well as low GzmB-producing CD8 T cells. In contrast, non-severe patients present with lower levels of inhibitory molecules and higher GzmA, GzmB, and perforin effectors. Moreover, in recovered patients, Tfh are found in the periphery at the time of viral clearance and persist into convalescence in contrast with an absence of lymph node Tfh found in patients who died of COVID-19. These findings underscore the importance of defining T cell epitope specificities to better understand COVID-19 immunity and to develop antibody- and T cell-directed vaccines that exploit T cell immunity.

Thus, there is an urgent need for the identification of CD4+ and CD8+ effector T cell epitopes, as well as tolerance-inducing regulatory T-cell epitopes, contained in SARS-CoV-2 and for their use in the development and design of effective pharmaceuticals and vaccines.

Further, artificial induction of tolerance to self or to foreign antigens is the goal of therapy for autoimmunity, transplantation, allergy and other diseases. Immune response targeting autologous and non-autologous therapeutic proteins often limits clinical efficacy. Immune-modulating treatments, inducing tolerance to therapeutic proteins compositions, may reduce the formation of anti-drug antibodies (ADA), which improve clinical outcomes. Until recently, therapeutic tolerance induction relied on broad-based immune cell depleting therapies. These broad-based approaches weaken the immune system in general and leave many subjects vulnerable to opportunistic infections, autoimmune attack, and cancer. There is a need in the art for less aggressive and more targeted approaches to the induction of immune tolerance.

Immune tolerance is regulated by a complex interplay between antigen presenting cells (APC), T cells, B cells, cytokines, chemokines, and surface receptors. Initial self/non-self discrimination occurs in the thymus during neonatal development where medullary epithelial cells express specific self-protein epitopes to immature T cells. T cells recognizing self-antigens with high affinity are deleted, but autoreactive T cells with moderate affinity sometimes avoid deletion and can be converted to ‘natural’ regulatory T cells (T_{Reg}) cells. These natural T_{Reg} cells are exported to the periphery and help to control latent autoimmune response.

A second form of tolerance develops in the periphery. In this case, activated T cells are converted to an ‘adaptive’ T_{Reg} phenotype through the action of certain immune-suppressive cytokines and chemokines such as IL-10, TGF-β, and CCL19. The possible roles for these ‘adaptive’ T_{Reg} cells include dampening immune response following the successful clearance of an invading pathogen, controlling excessive inflammation caused by an allergic reaction, controlling excessive inflammation caused by low level or chronic infection, or possibly controlling inflammatory response targeting beneficial symbiotic bacteria.

Naturally occurring T_{Regs} (including both natural T_{Regs} and adaptive T_{Regs}) are a critical component of immune regulation in the periphery. For example, upon activation of natural T_{Regs} through their TCR, natural T_{Regs} express immune-modulating cytokines and chemokines. Activated natural T_{Regs} may suppress nearby effector T cells through contact-dependent and independent mechanisms. In addition, the cytokines released by these cells including, but not limited to, IL-10 and TGF-β, are capable of inducing antigen-specific adaptive T_{Regs}. Despite extensive efforts, with few exceptions, the antigen specificity of natural T_{Regs}, and more importantly natural T_{Regs} circulating in clinically significant volumes, is still unknown. Further, pathogens, such as viruses, appear to have epitopes that are highly cross-conserved with self epitopes (“tolerized” antigens or epitopes) and can significantly expand T regulatory cell activities and lead to an immune camouflage that dampens or even escapes the adaptive immune response. For example, in the context of natural infection or un-adjuvanted vaccination using H7N9 HA influenza, Treg responses are induced by these epitopes, and humoral immune responses may be diminished and delayed, as has been reported in H7N9 infection (Guo L et al., Emerg. Infect. Dis., 20:192-200, 2013). As chronic-disease viruses appear to have many such epitopes that are highly cross-conserved with self and significantly expands T regulatory cells (Tregs), immune camouflage may be an important method by which certain human pathogens, including SARS-CoV-2, dampen or even escape adaptive immune response. To counter such, the detolerization of the immune system to these antigens by mutation of their anchoring and/or TCR recognition offers a way to counter the apparent camouflaged activity.

There is therefore need in the art for the identification of regulatory T cell epitopes (“Tregitopes”) and detolerized antigens thereof, compositions containing such, and for methods related to their preparation and use.

### SUMMARY OF THE INVENTION

In aspects, the present disclosure is directed to novel, therapeutic regulatory T cell epitope compositions. Such compositions include one or more of e.g., polypeptides (which may be termed herein as “Tregitope”) having a sequence comprising, consisting of, or consisting essentially of one or more of SEQ ID NOS: 4-370, 391-440, and 448-833 and/or fragments and variants thereof, and optionally 1 to 12 additional amino acids distributed in any ratio on the N-terminus and/or C-terminus of the polypeptide of SEQ ID NOS: 4-370, 391-440, and 448-833 (in aspects, the polypeptides may be isolated, synthetic, or recombinant) as disclosed herein; nucleic acids, expression cassettes, plasmids, expression vectors, recombinant viruses, or cells (all of which in aspects may be isolated, synthetic, or recombinant) as disclosed herein; chimeric or fusion polypeptide compositions as disclosed herein (which in aspects may be isolated, synthetic, or recombinant); and/or pharmaceutical compositions or formulations as disclosed herein, and use of the same, e.g., to suppress an immune response in the body or more specifically to suppress an immune response in the body caused by the administration of a therapeutic agent to treat a medical condition.

In further aspects, the present disclosure concerns detolerized antigens. As set forth herein, a detolerized antigen may include an absence within a composition of a Tregitope or one or more mutations thereto such that engagement and/or activation of TReg cells is negatively impacted. In some aspects, detolerized antigens include removal of the Tregitopes discussed herein. In some aspects, the present disclosure concerns immunogenic compositions of Sars-CoV-2 wherein the Tregitopes identified herein are removed and/or absent such that they cannot activate or engage a T_{Reg} cell. For example, the present disclosure concerns detolerized antigen compositions absent of one or more of the Tregitopes of SEQ ID NOS: 4-370, 391-440, and 448-833. In further aspects, a detolerized antigen includes a mutation or mutations to a Tregitope as set forth herein. In aspects, a detolerized antigen concerns a mutation or mutations to a Tregitope as identified herein such that the efficiency of binding to the MHC and/or T cell receptor is negatively affected, thereby diminishing or cancelling engagement and/or activation of T_{Reg} cells.

The selective engagement and activation of naturally occurring T_{Regs} (in aspects, including natural T_{Regs} and/or adaptive T_{Regs}) through the use of Tregitope compositions and detolerized antigen compositions as disclosed herein is therapeutically valuable as a means of treatment for any disease or condition marked by the presence of an unwanted immune response. Examples of such an unwanted immune response include the following: autoimmune disease such as type 1 diabetes, MS, Lupus, and RA; transplant related disorders such as Graft vs. Host disease (GVHD) and Host vs. Graft disease (HVGD); allergic reactions; immune rejection of biologic medicines such as monoclonal antibodies; immune response targeting replacement proteins; immune response targeting therapeutic toxins such as Botulinum toxin; and immune response to infectious disease whether acute or chronic. It will be further appreciated that providing a composition to stimulate an immune response to Sars-CoV-2 that features one or more detolerized antigens will be of benefit in avoiding T_{Reg} activation or an immune camouflage as set forth herein. Accordingly, compositions with the absence and/or mutations of Tregitopes identified herein can provide an enhanced and/or more effective immune response.

In aspects, the present disclosure harnesses the functions of naturally occurring T_{Regs} (in aspects, including natural T_{Regs} and/or adaptive T_{Regs}), and in particular aspects, those cells that already regulate immune responses to foreign and self-proteins in the periphery (pre-existing or natural T_{Reg}). In aspects, the present disclosure provides Tregitope compositions, with such compositions including one or more of polypeptides having a sequence comprising, consisting of, or consisting essentially of one or more of SEQ ID NOS: 4-370, 391-440, and 448-833 and/or fragments and variants thereof, and optionally 1 to 12 additional amino acids distributed in any ratio on the N-terminus and/or C-terminus as disclosed herein; nucleic acids, expression cassettes, plasmids, expression vectors, recombinant viruses, or cells (all of which in aspects may be isolated, synthetic, or recombinant) as disclosed herein; chimeric or fusion polypeptide compositions as disclosed herein (which in aspects may be isolated, synthetic, or recombinant); and/or pharmaceutical compositions or formulations as disclosed herein. In aspects, a Tregitope or a detolerized antigen thereof composition of the present disclosure may be either covalently bound, non-covalently bound, or in admixture with a specific target antigen.

In aspects, the present disclosure is directed to a polypeptide having a sequence comprising, consisting of, or consisting essentially of one or more of SEQ ID NOS: 4-370, 391-440, and 448-833. The phrase “consisting essentially of” is intended to mean that a polypeptide according to the present disclosure, in addition to the sequence according to any of SEQ ID NOS: 4-370, 391-440, and 448-833 or a variant thereof, contains additional amino acids or residues that may be present at either terminus of the peptide and/or on a side chain that are not necessarily forming part of the peptide that functions as an MHC ligand and provided they do not substantially impair the activity of the peptide to function as a Tregitope. In certain aspects, the Tregitopes can be capped with an N-terminal acetyl and/or C-terminal amino group.

In aspects, the instant disclosure is directed to a peptide or polypeptide comprising, consisting, or consisting essentially of an amino acid sequence of SEQ ID NOS: 4-370, 391-440, and 448-833 (and/or fragments or variants thereof), and optionally 1 to 12 additional amino acids distributed in any ratio on the N-terminus and/or C-terminus of the polypeptide of SEQ ID NOS: 4-370, 391-440, and 448-833. In aspects, the instant disclosure is directed to a peptide or polypeptide have a core amino acid sequence comprising, consisting of, or consisting essentially of one or more peptides or polypeptides having an amino acid sequence of SEQ ID NOS: 4-370, 391-440, and 448-833, and optionally having extensions of 1 to 12 amino acids on the C-terminal and/or the N-terminal of the core amino acid sequence, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio). In aspects, the instant disclosure is directed to a peptide or polypeptide having a core sequence comprising, consisting of, or consisting essentially of one or more peptides or polypeptides having an amino acid sequence of SEQ ID NOS: 4-370, 391-440, and 448-833 (and/or fragments and variants thereof), optionally with extensions of 1 to 12 amino acids on the C-terminal and/or the N-terminal, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio), provided that the polypeptide with the flanking amino acids is still able to bind to the same HLA molecule (i.e., retain MHC binding propensity) as said polypeptide core sequence without said flanking amino acids. In aspects, said polypeptide with the flanking amino acids is still able to bind to the same HLA molecule (i.e., retain MHC binding propensity) and/or retain the same TCR specificity as said polypeptide core sequence without said flanking amino acids. In aspects, said flanking amino acid sequences are those that also flank the peptides or polypeptides included therein in the naturally occurring protein.

For example, for a peptide or polypeptide have a core sequence comprising, consisting of, or consisting essentially of one or more peptides or polypeptides having an amino acid sequence of SEQ ID NOS: 6, 7, 18-31, 186-231, and 448-459 (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 6, 7, 18-31, 186-231, and 448-459 in the amino acid sequence of the envelope (SEQ ID NO: 1) of SARS-CoV-2. For a peptide or polypeptide have a core sequence comprising, consisting of, or consisting essentially of one or more peptides or polypeptides having an amino acid sequence of SEQ ID NOS: 4, 5, 17, 32-41, 232-245, 440, and 450-471 (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS. 4, 5, 17, 32-41, 232-245, 440, and 450-471 in the amino acid sequence of the membrane (SEQ ID NO: 2) of SARS-CoV-2. For a peptide or polypeptide have a core sequence comprising, consisting of, or consisting essentially of one or more peptides or polypeptides having an amino acid sequence of SEQ ID NOS. 8-17, 42-93, 246-370, 422, 423, 432, 434-439, and 794-833 (and/or fragments and variants thereof), optionally with extensions of 1 to 12 amino acids on the C-terminal and/or the N-terminal, the extensions of 1 to 12 amino acids are those found flanking the amino acid sequence of 8-17, 42-93, 246-370, 422, 423, 432, 434-439, and 794-833 in the amino acid sequence of the spike (SEQ ID NO: 3) of SARS-CoV-2. In aspects, said flanking amino acid sequences as described herein may serve as a MHC stabilizing region. The use of a longer peptide may allow endogenous processing by patient cells and may lead to more effective antigen presentation and induction of T cell responses. In aspects, the peptides or polypeptides can be in either neutral (uncharged) or salt forms, and may be either free of or include modifications such as glycosylation, side chain oxidation, or phosphorylation. In certain aspects, the Tregitopes can be capped with an N-terminal acetyl and/or C-terminal amino group.

In further aspects, the present disclosure concerns detolerized antigens (in which the identified Tregitopes discussed herein are removed from an antigen or in which the Tregitopes are altered, such as through deletion, partial deletion or amino acid mutation(s), to no longer function as tolerizing epitopes). For example, in aspects, removal of one or more of the identified regulatory T cell epitopes from an antigen or polypeptide sequence comprises deletion of all or some of the amino acids of the one or more regulatory T cell epitopes. In aspects, said removal of the one or more regulatory T cell epitopes from an antigen or polypeptide sequence comprises deletion of some or all of the amino acids of the one or more regulatory T cell epitopes and adding one or more amino acids at the site of deletion of the regulatory T cell epitope amino acids. In aspects, said removal of the one or more regulatory T cell epitopes from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said removal of the one or more regulatory T cell epitopes from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of removal need not correspond to the number of amino acids deleted from the previously existing regulatory T cell epitope amino acids. As such, the present disclosure comprises a Sars-CoV-2 antigen or derived polypeptide wherein one or more of the Tregitopes as set forth herein is deleted, partially deleted or mutated, such that the antigen is detolerized and does not engage or stimulate T_{Reg} cells when administered. Such may include the absence, deletion, partial deletion or mutation to one or more of the amino acid sequences set forth in SEQ ID NOS: 4-370, 391-440, and 448-833 from an antigen or polypeptide sequence comprising such. In aspects, with the tolerizing epitopes (Tregitopes) identified, binding of such epitopes can be disrupted by deletion, partial deletion and/or mutating anchoring residues included therein. Such mutations for the anchoring residues must select replacement amino acids which are dis-favorable for binding. Any amino acid substitution identified by EpiMatrix as disruptive (reduces predicted T cell epitope content) is viable. Targeted anchoring residues and specific mutations to improve immunogenicity and decease tolerogenicity are disclosed herein. Further, by mutating TCR contacts, T cell recognition of such tolerizing epitopes can be disrupted. Any amino acid substitution to TCR contacts can disrupt TCR recognition. Targeted TCR contacts for such epitopes are disclosed herein. As further explained in the Detailed Description, in some aspects, the detolerized antigens comprise, consist of, or consist essentially of one or more peptides or polypeptides with amino acid sequence as set forth in SEQ ID NO: 6, 7, 18-31, 186-231, and/or 448-459 with a mutation at one of positions V62, L65, S67, V70, K63, N64, N66, S68, R69, T11, L12, V14, N15, S16, V17, L19, F20, A22, F23, V24, V25, F26, L27, L28, V29, T30, L31, A32, I33, L34, A36, R38, A41, I13, L18, F20, L21, T35, L37, L39, and/or C40 in relation to SEQ ID NO: 1. In further aspects, the mutation comprises one or more of V62A, V62G, V62N, V62Q, V62S, V62T, and/or S67Q in relation to SEQ ID NO: 1.

As further explained in the Detailed Description, in some aspects, the detolerized antigens comprise, consist of, or consist essentially of one or more peptides or polypeptides with amino acid sequence as set forth in SEQ ID NO: 4, 5, 17, 32-41, 232-245, 440, and 450-471 with a mutation at one of positions I118, N121, P123, G126, L119, L120, V122, L124, H125, Y179, G182, S184, V187, K180, L181, A183, Q185, and/or R186 in relation to SEQ ID NO: 2. In further aspects, the mutation comprises one or more of I118A, I118G, I118N, I118Q, I118S, I118T, N121P, P123Q, P123G, G126P, Y179A, Y179N, Y1790, Y1795, Y179T, S184G, S184Q, and/or S184T in relation to SEQ ID NO: 2.

As further explained in the Detailed Description, in some aspects, the detolerized antigens comprise, consist of, or consist essentially of one or more peptides or polypeptides with amino acid sequence as set forth in SEQ ID NO: 8-17, 42-93, 246-370, 422, 423, 432, 434-439, and 794-833 with a mutation at one of positions F28, S31, L33, T36, D38, L41, R29, S30, V32, H34, S35, Q37, L39, F40, I195, V198, D200, Q203, N196, L197, R199, L201, P202, I220, F223, T225, A228, T221, R222, Q224, L226, L227, Y254, P257, Y259, L262, L255, Q256, R258, F260, L261, V496, S499, E501, H504, V497, L498, F500, L502, L503, L806, N809, V811, A814, L807, F808, K810, T812, L813, L843, L846, P848, T851, T844, V845, P847, L849, L850 F912, A915, G917, Q920, L923, T926, S928, G931, N913, S914, I916, K918, I919, S924, S925, A927, A929, L930, F955, I958, I960, N963, G956, A957, S959, V961, L962, I998, A1001, I1003, S1006, N1008, A1011, R999, A1000, E1002, R104, A1005, A1007, L1009, and/or A1010 in relation to SEQ ID NO: 3. In further aspects, the mutation comprises one or more of F28G, F28A, F28N, F28T, F28S, F28Q, S31G, S31T, L33Q, I195A, I195G, I195N, I195S, I195T, I195Q, V198G, V198T, V198N, Q203E, Q203G, Q203T, I220A, I220G, I220N, I2200, I220S, I220T, T225Q, Y254A, Y254G, Y254N, Y254Q, Y254S, Y254T, T259G, T259Q, V496A, V496G, V496N, V496Q, V496S, V496T, S499G, S499Q, S499T, L806A, L806G, L806N, L806Q, L806S, L806T, N809G, L843A, L843G, L843N, L843Q, L843S, L843T, L846G, L846T, P848Q, F912A, F912G, F912N, F912Q, F912S, F912T, A915G, L923A, L923G, L923N, L923Q, L923S, L923T, T926G, F955A, F955G, F955N, F955Q, F955S, F955T, I958G, S960G, S960Q, S960T, I998A, I998G, I998N, 1998Q, I998S, I998T, A1001G, A1001T, I1003A, I1003G, I1003N, I1003Q, I1003S, I1003T and/or N1008Q in relation to SEQ ID NO: 3.

In aspects, one or more peptides or polypeptides of the instant disclosure (e.g., but not limited to, a peptide or polypeptide comprising, consisting, or consisting essentially of an amino acid sequence of SEQ ID NOS: 4-370, 391-440, and 448-833 (and/or fragments or variants thereof), and optionally 1 to 12 additional amino acids distributed in any ratio on the N terminus and/or C-terminus of the polypeptide of SEQ ID NOS: 391-440, and 448-833, as well as detolerized antigens as disclosed herein), may be joined to, linked to (e.g., fused in-frame, chemically linked, or otherwise bound), and/or inserted into a heterologous polypeptide. In aspects, the one or more peptides or polypeptides of the instant disclosure may be joined to, linked to (e.g., fused in-frame, chemically linked, or otherwise bound), and/or inserted into a heterologous polypeptide as a whole, although it may be made up from a joined to, linked to (e.g., fused in-frame, chemically linked, or otherwise bound), and/or inserted amino acid sequence, together with flanking amino acids of the heterologous polypeptide. In aspects, the peptides or polypeptides can be in either neutral (uncharged) or salt forms, and may be either free of or include modifications such as glycosylation, side chain oxidation, or phosphorylation. In certain aspects, the Tregitopes can be capped with an N-terminal acetyl and/or C-terminal amino group.

In aspects, the present disclosure is directed to polypeptide having a sequence comprising one or more of SEQ ID NOS: 4-370, 391-440, and 448-833 (and fragments or variants thereof), and optionally 1 to 12 additional amino acids distributed in any ratio on the N terminus and/or C-terminus of the polypeptide of SEQ ID NOS: 4-370, 391-440, and 448-833), as well as detolerized antigens as disclosed herein, wherein said one or more of SEQ ID NOS: 4-370, 391-440, and 448-833 or said detolerized antigen is not naturally included in the polypeptide and/or said one or more of SEQ ID NOS: 4-370, 391-440, and 448-833 or detolerized antigen is not located at its natural position in the polypeptide. In aspects of the above-described polypeptides, the polypeptides may be isolated, synthetic, or recombinant. In aspects, the peptides or polypeptides can be in either neutral (uncharged) or salt forms, and may be either free of or include modifications such as glycosylation, side chain oxidation, or phosphorylation. In certain aspects, the polypeptides can be capped with an N-terminal acetyl and/or C-terminal amino group.

In aspects, the present disclosure is directed to a concatemeric polypeptide or peptide that comprises at one or more of the instantly-disclosed polypeptides or peptides (e.g., but not limited to, a peptide or polypeptide comprising, consisting, or consisting essentially of an amino acid sequence of SEQ ID NOS: 4-370, 391-440, and 448-833 (and/or fragments or variants thereof), and optionally 1 to 12 additional amino acids distributed in any ratio on the N-terminus and/or C-terminus of the polypeptide of SEQ ID NOS: 4-370, 391-440, and 448-833, as well as detolerized antigens as disclosed herein) linked, fused, or joined together (e.g., fused in-frame, chemically linked, or otherwise bound) to an additional peptide or polypeptide. Such additional peptide or polypeptide may be one or more of the instantly disclosed polypeptides or peptides, or may be an additional peptide or polypeptide of interest. In aspects a concatemeric peptide is composed of 1 or more, 2 or more, 3 or more, 4 or more, 5 or more 6 or more 7 or more, 8 or more, 9 or more of the instantly disclosed peptides or polypeptides. In other aspects, the concatemeric peptides or polypeptides include 1000 or more, 1000 or less, 900 or less, 500 or less, 100 or less, 75 or less, 50 or less, 40 or less, 30 or less, 20 or less, or 10 or less peptide epitopes. In yet other embodiments, a concatemeric peptide has 3-100, 5-100, 10-100, 15-100, 20-100, 25-100, 30-100, 35-100, 40-100, 45-100, 50-100, 55-100, 60-100, 65-100, 70-100, 75-100, 80-100, 90-100, 5-50, 10-50, 15-50, 20-50, 25-50, 30-50, 35-50, 40-50, 45-50, 100-150, 100-200, 100-300, 100-400, 100-500, 50-500, 50-800, 50-1,000, or 100-1,000 of the instantly-disclosed peptides or polypeptides linked, fused, or joined together. Each peptide or polypeptide of the concatemeric polypeptide may optionally have one or more linkers, which may optionally be cleavage sensitive sites, adjacent to their N-terminal and/or C-terminal end. In such a concatemeric peptide, two or more of the peptide epitopes may have a cleavage sensitive site between them. Alternatively, two or more of the peptide epitopes may be connected directly to one another or through a linker that is not a cleavage sensitive site. In aspects of above-described concatemeric peptides or polypeptides, the concatemeric peptides or polypeptides may be isolated, synthetic, or recombinant. In aspects, the concatemeric peptides or polypeptides can be in either neutral (uncharged) or salt forms, and may be either free of or include modifications such as glycosylation, side chain oxidation, or phosphorylation. In certain aspects, the concatemeric polypeptides can be capped with an N-terminal acetyl and/or C-terminal amino group.

In aspects, the present disclosure is directed to a chimeric or fusion polypeptide composition comprising one or more polypeptides (e.g., but not limited to, a peptide or polypeptide comprising, consisting, or consisting essentially of an amino acid sequence of SEQ ID NOS: 4-370, 391-440, and 448-833 (and/or fragments or variants thereof), and optionally 1 to 12 additional amino acids distributed in any ratio on the N terminus and/or C-terminus of the polypeptide of SEQ ID NOS: 4-370, 391-440, and 448-833, as well as detolerized antigens as disclosed herein) of the present disclosure. In aspects, a chimeric or fusion polypeptide composition of the present disclosure comprises one or more polypeptides of the present disclosure joined to, linked to (e.g., fused in-frame, chemically-linked, or otherwise bound), and/or inserted into a heterologous polypeptide. In aspects, the one or more polypeptides (e.g., Tregitope) of the present disclosure may be inserted into the heterologous polypeptide, may be added to the C-terminus (with or without the use of linkers, as is known in the art), and/or added to the N-terminus (with or without the use of linkers, as is known in the art) of the heterologous polypeptide. In aspects of the instantly disclosed chimeric or fusion polypeptide compositions, the one or more polypeptides of the present disclosure have a sequence comprising, consisting of, or consisting essentially of one or more of SEQ ID NOS: 4-370, 391-440, and 448-833 and/or a detolerized antigen as disclosed herein. In aspects of the chimeric or fusion polypeptide compositions, the one or more of SEQ ID NOS: 4-370, 391-440, and 448-833 and/or detolerized antigen may be joined to, linked to (e.g., fused in-frame, chemically-linked, or otherwise bound), and/or inserted into a heterologous polypeptide as a whole, although it may be made up from a joined to, linked to (e.g., fused in-frame, chemically-linked, or otherwise bound), and/or inserted amino acid sequence, together with flanking amino acids of the heterologous polypeptide. In aspects, a chimeric or fusion polypeptide composition of the present disclosure comprises a polypeptide, said polypeptide having a sequence comprising one or more of SEQ ID NOS: 4-370, 391-440, and 448-833 and/or a detolerized antigen of the present disclosure, wherein said one or more of SEQ ID NOS: 4-370, 391-440, and 448-833 and/or a detolerized antigen is not naturally included in the polypeptide and/or said of one or more of SEQ ID NOS: 4-370, 391-440, and 448-833 and/or detolerized antigenic not located at its natural position in the polypeptide. In aspects, the one or more of SEQ ID NOS: 4-370, 391-440, and 448-833 and/or a detolerized antigen of the present disclosure can be joined, linked to (e.g., fused in-frame, chemically linked, or otherwise bound), and/or inserted into the polypeptide. In aspects of above-described chimeric or fusion polypeptide compositions, the chimeric or fusion polypeptides may be isolated, synthetic, or recombinant. In aspects, the chimeric or fusion polypeptides can be in either neutral (uncharged) or salt forms, and may be either free of or include modifications such as glycosylation, side chain oxidation, or phosphorylation.

In aspects, the present disclosure is directed to a nucleic acid (e.g., DNA or RNA, including mRNA), encoding peptides, polypeptides (including the detolerized antigens as disclosed herein), concatemeric peptides, and/or chimeric or fusion polypeptides as described herein. For example, in aspects, the instant disclosure is directed to a nucleic acid encoding a peptide or polypeptide comprising, consisting of, or consisting essentially of one or more peptides or polypeptides as set forth herein, including optionally 1 to 12 additional amino acids distributed in any ratio on the N-terminus and/or C-terminus of the polypeptide. In aspects, the present disclosure is directed to a vector comprising such a nucleic acid as described. In aspects, the present disclosure is directed to a cell or vaccine comprising such a vector as described. In aspects, the present disclosure is directed to a cell comprising a vector of the present disclosure. In further aspects, the present disclosure is directed to nucleic acids encoding a protein or polypeptide derivative thereof of the SARS-CoV-2 wherein one or more peptides of the Tregitopes as set forth herein are deleted, partially deleted and/or mutated, such that when translated, the resulting peptide is a detolerized antigen and demonstrates reduced, diminished, negligible or negated activity towards engaging or stimulating or activating a T_{Reg} cell.

In aspects, the instant disclosure is directed to a pharmaceutical composition, the pharmaceutical composition comprising one or more peptides, polypeptides (including the detolerized antigens disclosed herein), concatemeric peptides, and/or chimeric or fusion polypeptides as disclosed herein and a pharmaceutically acceptable carrier, excipient, and/or adjuvant. In further aspects, the pharmaceutical composition comprising a SARS-CoV-2 derived peptide wherein one or more Tregitopes therein is deleted, partially deleted and/or mutated, such that the polypeptide exhibits reduced or absent T_{Reg} activation and/or engagement when administered. Another aspect is directed to a pharmaceutical composition, the pharmaceutical composition comprising one or more nucleic acids encoding one or more peptides, polypeptides (including the detolerized antigens disclosed herein), concatemeric peptides, and/or chimeric or fusion polypeptides as disclosed herein, and a pharmaceutically acceptable carrier, excipient, and/or adjuvant. In aspects, the one or more nucleic acids encoding said peptides or polypeptides are DNA, RNA, or mRNA.

In aspects, the present disclosure is directed to a method of affecting, either negatively or positively, regulatory T-cells (in aspects, naturally occurring T_{Regs}, including natural T_{Regs} and/or adaptive T_{Regs}) in a subject in need thereof and/or suppressing an immune response and/or stimulating an immune response in a subject in need thereof by administering to the subject a therapeutically effect amount of a composition of the present disclosure.

In aspects, the present disclosure is directed to a method of treating or preventing a medical condition in a subject in need thereof comprising administering a composition of the present disclosure. In aspects, the medical condition is selected from the group consisting of: an allergy, an autoimmune disease, a transplant related disorder, graft versus host disease, a blood clotting disorder, an enzyme or protein deficiency disorder, a hemostatic disorder, cancer, infertility; and a viral, bacterial or parasitic infection.

In aspects, the present disclosure is directed to a method of stimulating/inducing, regulatory T-cells (e.g., naturally occurring T_{Regs} (in aspects, including natural T_{Regs} and/or adaptive T_{Regs})) to suppress an immune response in a subject in need thereof by administering to the subject a therapeutically effect amount of a Tregitope composition of the present disclosure. In aspects, the immune response is the result of one or more therapeutic treatments with at least one therapeutic protein, treatment with a vaccine or treatment with at least one antigen. In another aspect, the administration of a Tregitope composition of the present disclosure shifts one or more antigen presenting cells to a regulatory phenotype, one or more dendritic cells to a regulatory phenotype, decreases CD11c and HLA-DR expression in the dendritic cells or other antigen presenting cells.

In aspects, the present disclosure is directed to a method for expanding a population of regulatory T cells, comprising: (a) providing a biological sample from a subject; (b) isolating regulatory T-cells from the biological sample; (c) contacting the isolated regulatory T-cells with an effective amount of a composition of the present disclosure, under conditions wherein the T-regulatory cells increase in number to yield an expanded regulatory T-cell composition, thereby expanding the regulatory T-cells in the biological sample; and, additionally, (d) returning the sample to the subject in need of treatment.

In aspects, the present disclosure is directed to a method for stimulating regulatory T cells in a biological sample, comprising: (a) providing a biological sample from a subject; (b) isolating regulatory T-cells from the biological sample; (c) contacting the isolated regulatory T-cells with an effective amount of a composition of the present disclosure, under conditions wherein the T-regulatory cells are stimulated to alter one or more biological function, thereby stimulating the regulatory T-cells in the biological sample; and, additionally, (d) returning cells to the subject in need of treatment.

In aspects, the present disclosure is directed to a method for repressing/suppressing an immune response in a subject, comprising administering a therapeutically effective amount of a composition of the present disclosure, wherein the Tregitope composition represses/suppresses the immune response. In aspects, the Tregitope composition represses/suppresses an innate immune response. In aspects, the Tregitope composition represses/suppresses an adaptive immune response. In aspects, the Tregitope composition represses/suppresses an effector T cell response. In aspects, the Tregitope composition represses/suppresses a memory T cell response. In aspects, the Tregitope composition represses/suppresses helper T cell response. In aspects, the composition represses/suppresses B cell response. In aspects, the Tregitope composition represses/suppresses an NKT cell (natural killer T cell) response.

In aspects, the present disclosure is directed to a method of suppressing an immune response, specifically an antigen specific immune response in a subject, through the administration of a therapeutically effective amount of a composition of the present disclosure, wherein said composition activates naturally occurring T_{Regs} (in aspects, including natural T_{Regs} and/or adaptive T_{Regs}, and in aspects CD4⁺/CD25⁺/FoxP3⁺ regulatory T-cells) or suppresses the activation of CD4⁺ T-cells, the proliferation of CD4⁺ and/or CD8⁺ T-cells, and/or suppresses the activation or proliferation of β-cells or NKT Cells. In aspects, a composition of the present disclosure may be covalently bound, non-covalently bound, or in admixture with a specific target antigen. In aspects, an administered composition of the present disclosure that is covalently bound, non-covalently bound, or in admixture with a specific target antigen results in the diminution of immune response against the target antigen.

In aspects, the target antigen may allogenic protein or protein fragments. In aspects, the target antigen may be a biologic medicine or fragments thereof. In aspects, the suppressive effect is mediated by natural T_{Regs}. In aspects, the suppressive effect is mediated by an adaptive T_{Regs}. In aspects, the one or more Tregitope included in the compositions of the present disclosure suppresses an effector T cell response. In aspects, the one or more Tregitopes of the presently disclosed Tregitope compositions suppresses an innate immune response. In aspects, the one or more Tregitopes of the presently disclosed Tregitope compositions suppresses an adaptive immune response. In aspects, the one or more Tregitopes of the presently disclosed Tregitope compositions suppresses helper T cell response. In aspects, the one or more Tregitopes of the presently disclosed Tregitope compositions suppresses a memory T cell response. In aspects, the one or more Tregitopes of the presently disclosed Tregitope compositions suppresses B cell response. In aspects, the one or more Tregitopes of the presently disclosed Tregitope compositions suppresses NKT cell response.

In aspects, the present disclosure is directed to a kit for preventing or treating a medical condition, in particular, for the suppression of an immune response in a subject, wherein the kit comprises a composition of the present disclosure. In aspects, the kit may further comprise an effective amount of an antigen or allergen or therapeutic agent, such as a replacement protein or peptide.

In aspects, the present disclosure is directed to a method of stimulating and/or diminishing immuno-suppression through the administration of the detolerized antigens (which include detolerized Tregitopes) as set forth herein. In some aspects, the detolerized antigen include one or more SARS-CoV-2 derived peptides wherein one or more of the Tregitopes as identified herein is deleted, partially deleted and/or mutated, such that the polypeptide exhibits reduced or absent T_{Reg} engagement and/or activation. As described herein, in aspects, the detolerized antigens have diminished T cell receptor activation through deletion and/or mutations to the anchoring amino acids between the MHC and the TCR, either in the agretope and/or the epitope of the binding. The diminished binding allows for the T_{Reg} cells to experienced diminished activation and thereby provide for reduced immuno-suppression. In further aspects, the inability or reduced ability to activate T_{Reg} cells also allows for other immuno-stimulatory T cells to interact and generate a positive immune response to the detolerized antigen.

In some aspects, the present disclosure is directed to methods for enhancing an immune response in a subject in need thereof by administering to the subject a therapeutically effective amount of a detolerized antigen as set forth herein. In some aspects, the detolerized antigen polypeptide reduces T_{Reg} activation in the subject. In further aspects, the detolerized antigen polypeptide retains MHC binding. In other aspects, the detolerized antigen polypeptide stimulates CD4+ and/or CD8+ T cells.

### BRIEF DESCRIPTION OF THE FIGURES

The present disclosure may be better understood with reference to the following figures.

FIG. 1 shows the sequences of the envelope (SEQ ID NO: 1) of SARS-CoV-2, the membrane (SEQ ID NO: 2) of SARS-CoV-2, and the spike (SEQ ID NO: 3) of SARS-CoV-2.

FIG. 2 is an overview of MHC class II cluster selection from the envelope (SEQ ID NO: 1) of SARS-CoV-2. The cluster address given the location of the peptide within the sequences that were provided for analysis. The core peptide (middle amino acids in bold, SEQ ID NO: in parentheses) defines the actual cluster that was identified during the analysis. The stabilizing flanks (N-terminal and C-terminal, not bold) are included for use with the core sequence, and are labeled by the SEQ ID NO: not listed in parentheses. The number of hits is the number of EpiMatrix Z-scores above 1.64 or top 5% found within the sequence. The EpiMatrix Cluster Score is derived from the number of hits normalized for the length of the cluster. Cluster Score is thus the excess or shortfall in predicted aggregate immunogenicity relative to a random peptide standard. Hydrophobicity scores of 2 and above are predictive of difficulty synthesizing peptides.

FIGS. 3-7 are EpiMatrix Cluster detail reports for identified MHC class II clusters of the envelope (SEQ ID NO: 1) of SARS-CoV-2. Z-score indicates the potential of a 9-mer frame to bind to a given HLA allele; the strength of the score is indicated by the blue shading as noted in the respective Figures. All scores in the Top 5% (Z-Score≥1.64) are considered “Hits”. *Scores in the top 10% are considered elevated, other scores are grayed out for simplicity. Frames containing 4 or more alleles scoring above 1.64 are referred to as EpiBars and are highlighted in yellow. These frames have an increased likelihood of binding to HLA. Flanking amino acids, added to stabilize the cluster during in-vitro testing, are presented in blue type face and underlined.

FIG. 8 is an overview of MHC class II cluster selection from the membrane (SEQ ID NO: 2) of SARS-CoV-2. The cluster address given the location of the peptide within the sequences that were provided for analysis. The core peptide (middle amino acids in bold, SEQ ID NO: in parentheses) defines the actual cluster that was identified during the analysis. The stabilizing flanks (N-terminal and C-terminal, not bold) are included for use with the core sequence, and are labeled by the SEQ ID NO: not listed in parentheses. The number of hits is the number of EpiMatrix Z-scores above 1.64 or top 5% found within the sequence. The EpiMatrix Cluster Score is derived from the number of hits normalized for the length of the cluster. Cluster Score is thus the excess or shortfall in predicted aggregate immunogenicity relative to a random peptide standard. Hydrophobicity scores of 2 and above are predictive of difficulty synthesizing peptides.

FIGS. 9-10 are EpiMatrix Cluster detail reports for identified MHC class II clusters of the membrane (SEQ ID NO: 2) of SARS-CoV-2. Z-score indicates the potential of a 9-mer frame to bind to a given HLA allele; the strength of the score is indicated by the blue shading as noted in the respective Figures. All scores in the Top 5% (Z-Score≥1.64) are considered “Hits”. *Scores in the top 10% are considered elevated, other scores are grayed out for simplicity. Frames containing 4 or more alleles scoring above 1.64 are referred to as EpiBars and are highlighted in yellow. These frames have an increased likelihood of binding to HLA. Flanking amino acids, added to stabilize the cluster during in-vitro testing, are presented in blue type face and underlined.

FIG. 11 is an overview of MHC class II cluster selection from the spike (SEQ ID NO: 3) of SARS-CoV-2. The cluster address given the location of the peptide within the sequences that were provided for analysis. The core peptide (middle amino acids in bold, SEQ ID NO: in parentheses) defines the actual cluster that was identified during the analysis. The stabilizing flanks (N-terminal and C-terminal, not bold) are included for use with the core sequence, and are labeled by the SEQ ID NO: not listed in parentheses. The number of hits is the number of EpiMatrix Z-scores above 1.64 or top 5% found within the sequence. The EpiMatrix Cluster Score is derived from the number of hits normalized for the length of the cluster. Cluster Score is thus the excess or shortfall in predicted aggregate immunogenicity relative to a random peptide standard. Hydrophobicity scores of 2 and above are predictive of difficulty synthesizing peptides.

FIGS. 12-28 are EpiMatrix Cluster detail reports for identified MHC class II clusters of the spike (SEQ ID NO: 3) of SARS-CoV-2. Z-score indicates the potential of a 9-mer frame to bind to a given HLA allele; the strength of the score is indicated by the blue shading as noted in the respective Figures. All scores in the Top 5% (Z-Score≥1.64) are considered “Hits”. *Scores in the top 10% are considered elevated, other scores are grayed out for simplicity. Frames containing 4 or more alleles scoring above 1.64 are referred to as EpiBars and are highlighted in yellow. These frames have an increased likelihood of binding to HLA. Flanking amino acids, added to stabilize the cluster during in-vitro testing, are presented in blue type face and underlined.

FIGS. 29A-F are the JanusMatrix reports for identified MHC class II clusters of the envelope (SEQ ID NO: 1) of SARS-CoV-2. *Count of HUMAN JanusMatrix matches found in the search database. With respect to a given EpiMatrix Hit (a 9-mer contained within the input sequence which is predicted to bind to a specific allele), a Janus Matrix match is a 9-mer derived from the search database (e.g., the human genome) which is predicted to bind to the same allele as the EpiMatrix Hit and shares TCR facing contacts with the EpiMatrix Hit. ** Janus Homology Score represents the average depth of coverage in the search database for each EpiMatrix hit in the input sequence. For example, an input peptide with eight EpiMatrix hits, all of which have one match in the search database, has a Janus Homology Score of 1. An input peptide with four EpiMatrix Hits, all of which have two matches in the search database, has a Janus Homology Score of 2. The JanusMatrix Homology Score considers all constituent 9-mers in any given peptide, including flanks.

FIGS. 30A-C are the JanusMatrix reports for identified MHC class II clusters of the membrane (SEQ ID NO: 2) of SARS-CoV-2. *Count of HUMAN JanusMatrix matches found in the search database. With respect to a given EpiMatrix Hit (a 9-mer contained within the input sequence which is predicted to bind to a specific allele), a Janus Matrix match is a 9-mer derived from the search database (e.g., the human genome) which is predicted to bind to the same allele as the EpiMatrix Hit and shares TCR facing contacts with the EpiMatrix Hit. ** Janus Homology Score represents the average depth of coverage in the search database for each EpiMatrix hit in the input sequence. For example, an input peptide with eight EpiMatrix hits, all of which have one match in the search database, has a Janus Homology Score of 1. An input peptide with four EpiMatrix Hits, all of which have two matches in the search database, has a Janus Homology Score of 2. The JanusMatrix Homology Score considers all constituent 9-mers in any given peptide, including flanks.

FIGS. 31A-T are the JanusMatrix reports for identified MHC class II clusters of the spike (SEQ ID NO: 3) of SARS-CoV-2. *Count of HUMAN JanusMatrix matches found in the search database. With respect to a given EpiMatrix Hit (a 9-mer contained within the input sequence which is predicted to bind to a specific allele), a Janus Matrix match is a 9-mer derived from the search database (e.g., the human genome) which is predicted to bind to the same allele as the EpiMatrix Hit and shares TCR facing contacts with the EpiMatrix Hit. ** Janus Homology Score represents the average depth of coverage in the search database for each EpiMatrix hit in the input sequence. For example, an input peptide with eight EpiMatrix hits, all of which have one match in the search database, has a Janus Homology Score of 1. An input peptide with four EpiMatrix Hits, all of which have two matches in the search database, has a Janus Homology Score of 2. The JanusMatrix Homology Score considers all constituent 9-mers in any given peptide, including flanks.

FIGS. 32A-32D shows the sequences of the nucleocapsid (SEQ ID NO: 371), ORF3a (SEQ ID NO: 372), ORF6 (SEQ ID NO: 373), ORF7a (SEQ ID NO: 374), ORF8 (SEQ ID NO: 387), ORF10 (SEQ ID NO: 388), ORF1ab non-structural protein 2 (NSP2) (SEQ ID NO: 375), ORF1ab non-structural protein 3 (NSP3) (SEQ ID NO: 376), ORF1ab non-structural protein 4 (NSP4) (SEQ ID NO: 377), ORF1ab 3C-like proteinase (SEQ ID NO: 389), ORF1ab non-structural protein 6 (NSP6) (SEQ ID NO: 378), ORF1ab non-structural protein 7 (NSP7) (SEQ ID NO: 379), ORF1ab non-structural protein 8 (NSP8) (SEQ ID NO: 380), ORF1ab non-structural protein 9 (NSP9) (SEQ ID NO: 381), ORF1ab non-structural protein 10 (NSP10) (SEQ ID NO: 390), ORF1ab RNA-dependent RNA polymerase (SEQ ID NO: 382), ORF1ab helicase (SEQ ID NO: 383), ORF1ab 3′-5′ exonuclease (SEQ ID NO: 384), ORF1ab endoRNase (SEQ ID NO: 385), and ORF1ab 2′O-ribose methyltransferase proteins (SEQ ID NO: 386) of SARS-CoV-2.

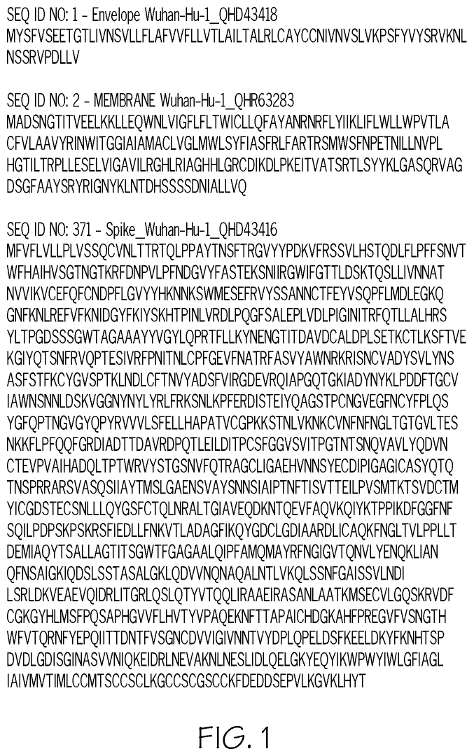

### DETAILED DESCRIPTION

The present disclosure generally relates to Tregitope peptide compounds and compositions and detolerized antigens thereof for use against SARS-CoV-2 infection and related diseases caused by SARS-CoV-2, including COVID-19. The disclosure relates to immunogenic peptides, polypeptides, concatemeric peptides, and chimeric or fusion polypeptides and the uses thereof, particularly in pharmaceutical and vaccine compositions. The present disclosure also relates to nucleic acids, vectors (including expression vectors), and cells which express the peptides, polypeptides, concatemeric peptides, and chimeric or fusion polypeptides and the uses thereof. The peptides, polypeptides, concatemeric peptides, and chimeric or fusion polypeptides of the present disclosure more specifically comprise an agretope predicted to be a ligand of HLA class I and/or HLA class II MHC molecules, as well as an epitope that is predicted to be recognized by T-cell receptors (TCRs) (including CD8+ and/or CD4+ T-cells) in the context of MHC class I and/or class II molecules. In further aspects, the epitopes and/or anchoring amino acids are mutated to form detolerized antigen variants of the Tregitope peptides.

### (i) Definitions

To further facilitate an understanding of the present disclosure, a number of terms and phrases are defined below. Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Ranges provided herein are understood to be shorthand for all of the values within the range. For example, a range of 1 to 25 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, or 25, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 25 may comprise 1 to 5, 1 to 10, 1 to 15, and 1 to 20 in one direction, or 25 to 20, 25 to 15, 25 to 10, and 25 to 5 in the other direction.

As used herein, the term “biological sample” refers to any sample of tissue, cells, or secretions from an organism.

As used herein, the term “medical condition” includes, but is not limited to, any condition or disease manifested as one or more physical and/or psychological symptoms for which treatment and/or prevention is desirable, and includes previously and newly identified diseases and other disorders.

As used herein, the term “immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells, metastatic tumor cells, malignant melanoma, invading pathogens (including a virus), cells or tissues infected with pathogens, or, in cases of autoimmunity or pathological inflammation, normal human cells or tissues. In aspects, an immune response includes a measurable cytotoxic T lymphocyte (CTL) response (e.g., against a virus expressing an immunogenic polypeptide) or a measurable B cell response, such as the production of antibodies, (e.g., against an immunogenic polypeptide). One of ordinary skill would know various assays to determine whether an immune response against a peptide, polypeptide, or related composition was generated, including use of the experiments and assays as disclosed in the Examples herein. Various B lymphocyte and T lymphocyte assays are well known, such as ELISAs, EliSpot assays, cytotoxic T lymphocyte CTL assays, such as chromium release assays, proliferation assays using peripheral blood lymphocytes (PBL), tetramer assays, and other cytokine production assays. See Benjamini et al. (1991), hereby incorporated by reference.

As used herein, the term “effective amount”, “therapeutically effective amount”, or the like of a composition, including a Tregitope or detolerized antigen thereof compound or composition of the present disclosure (including one or more of peptides or polypeptides having a sequence comprising, consisting of, or consisting essentially of one or more of SEQ ID NOS: 4-370, 391-440, and 448-833 and/or fragments and variants thereof, and optionally 1 to 12 additional amino acids distributed in any ratio on the N terminus and/or C-terminus of the polypeptide, or detolerized antigens as disclosed herein; concatemeric peptides as disclosed herein, including concatemeric polypeptides comprising, consisting of, or consisting essentially of one or more of SEQ ID NOS: 4-370, 391-440, and 448-833 and/or fragments and variants thereof; chimeric of fusion polypeptide compositions as disclosed herein; nucleic acids encoding such peptides, polypeptides, concatemeric peptides, or chimeric of fusion polypeptide compositions as disclosed herein; expression cassettes, plasmids, expression vectors, recombinant viruses, or cells which express such peptides, polypeptides, concatemeric peptides or chimeric of fusion polypeptide compositions as disclosed herein; vaccine compositions or formulations, and/or pharmaceutical compositions or formulations as disclosed herein) is a quantity sufficient to achieve a desired therapeutic and/or prophylactic effect. In aspects, such a desired therapeutic and/or prophylactic effect is, e.g., an amount that results in the prevention of, or a decrease in, the symptoms and/or underlying causes associated with a disease that is being treated, such as SARS-CoV-2 infection and related diseases caused by SARS-CoV-2, including COVID-19, or an amount to measurably to inhibit virus (for example, SARS-CoV-2) replication or infectivity. The amount of a composition of the present disclosure administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compounds and compositions of the present invention can also be administered in combination with each other or with one or more additional therapeutic compounds.

As used herein, the term “T-cell epitope” means an MHC ligand or protein determinant, 7 to 30 amino acids in length, and capable of specific binding to human leukocyte antigen (HLA) molecules and interacting with specific T cell receptors (TCRs). As used herein, in the context of a T cell epitope that is known or determined (e.g. predicted) to engage a T cell, the terms “engage”, “engagement” or the like means that when bound to a MHC molecule (e.g. human leukocyte antigen (HLA) molecules), the T cell epitope is capable of interacting with the TCR of the T cell and activating the T cell. Generally, T-cell epitopes are linear and do not express specific three-dimensional characteristics. T-cell epitopes are not affected by the presence of denaturing solvents. The ability to interact with T-cell epitopes can be predicted by in silico methods (De Groot A S et al., (1997), AIDS Res Hum Retroviruses, 13(7):539-41; Schafer J R et al., (1998), Vaccine, 16(19):1880-4; De Groot A S et al., (2001), Vaccine, 19(31):4385-95; De Groot A R et al., (2003), Vaccine, 21(27-30):4486-504, all of which are herein incorporated by reference in their entirety.

As used herein, the term “T-cell epitope cluster” refers to polypeptide that contains between about 4 to about 40 MHC binding motifs. In particular embodiments, the T-cell epitope cluster contains between about 5 to about 35 MHC binding motifs, between about 8 and about 30 MHC binding motifs; and between about 10 and 20 MHC binding motifs.

As used herein, the term “immune-stimulating T-cell epitope polypeptide” refers to a molecule capable of inducing an immune response, e.g., a humoral, T cell-based, or innate immune response.

As used herein, the term “regulatory T cell”, “Treg” or the like, means a subpopulation of T cells that suppress immune effector function, including the suppression or down regulation of CD4+ and/or CD8+ effector T cell (Teff) induction, proliferation, and/or cytokine production, through a variety of different mechanisms including cell-cell contact and suppressive cytokine production. In aspects, CD4+ Tregs are characterized by the presence of certain cell surface markers including but not limited to CD4, CD25, and FoxP3. In aspects, upon activation, CD4+ regulatory T cells secrete immune suppressive cytokines and chemokines including but not limited to IL-10 and/or TGFβ. CD4+ Tregs may also exert immune suppressive effects through direct killing of target cells, characterized by the expression upon activation of effector molecules including but not limited to granzyme B and perforin. In aspects, CD8+ Tregs are characterized by the presence of certain cell surface markers including but not limited to CD8, CD25, and, upon activation, FoxP3. In aspects, upon activation, regulatory CD8+ T cells secrete immune suppressive cytokines and chemokines including but not limited to IFNγ, IL-10, and/or TGFβ. In aspects, CD8⁺ Tregs may also exert immune suppressive effects through direct killing of target cells, characterized by the expression upon activation of effector molecules including but not limited to granzyme B and/or perforin.

As used herein, the term “regulatory T cell epitope” (“Tregitope”) refers to a “T cell epitope” that causes a tolerogenic response (Weber C A et al., (2009), Adv Drug Deliv, 61(11):965-76) and is capable of binding to MHC molecules and engaging (i.e. interacting with and activating) circulating naturally occurring Tregs (in aspects, including natural Tregs and/or adaptive Tregs). In aspects, upon activation, CD4+ regulatory T cells secrete immune suppressive cytokines and chemokines including but not limited to IL-10 and/or TGFβ. CD4+ Tregs may also exert immune suppressive effects through direct killing of target cells, characterized by the expression upon activation of effector molecules including but not limited to granzyme B and perforin, leads to the expression of the immune suppressive cytokines including, but not limited to, IL-10 and TGF-β and TNF-α. In aspects, upon activation, regulatory CD8+ T cells secrete immune suppressive cytokines and chemokines including but not limited to IFNγ, IL-10, and/or TGFβ. In aspects, CD8⁺ Tregs may also exert immune suppressive effects through direct killing of target cells, characterized by the expression upon activation of effector molecules including but not limited to granzyme B and/or perforin.

As used herein, a “detolerized” antigen refers to a variant of a polypeptide sequence comprising, consisting essentially of, or consisting of a Tregitope wherein the recognition of such a Tregitope by the TCR is disrupted through deletion and/or mutation of an anchoring amino acid and/or mutation of a T cell receptor epitope. In some aspects, a detolerized antigen may include a peptide sequence derived from an endogenous protein, such as, e.g., the membrane, spike, envelope etc of SARS-CoV-2, wherein one or more Tregitopes as identified herein is deleted and/or partially deleted. In other aspects, a Tregitope portion of a SARS-CoV-2 derived peptide features a mutation or mutations to amino acids in one or more Tregitopes therein. As set forth herein, a Tregitope interaction leads to a tolerogenic response. Through the deletion, mutation and/or manipulation of the amino acid sequence in the TCR recognition motifs and/or peptide anchoring, the antigen or Tregitopes can become detolerized and avoid an immune-suppressive response.

As used herein, the term “B-cell epitope” means a protein determinant capable of specific binding to an antibody. B-cell epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

As used herein, the terms “the major histocompatibility complex (MHC)”, “MHC molecules”, “MHC proteins” or “HLA proteins” are to be understood as meaning, in particular, proteins capable of binding peptides resulting from the proteolytic cleavage of protein antigens and representing potential T-cell epitopes, transporting them to the cell surface and presenting them there to specific cells, in particular cytotoxic T-lymphocytes or T-helper cells. The major histocompatibility complex in the genome comprises the genetic region whose gene products expressed on the cell surface are important for binding and presenting endogenous and/or foreign antigens and thus for regulating immunological processes. The major histocompatibility complex is classified into two gene groups coding for different proteins, namely molecules of MHC class I and molecules of MHC class II. The molecules of the two MHC classes are specialized for different antigen sources. The molecules of MHC class I present endogenously synthesized antigens, for example viral proteins and tumor antigens. The molecules of MHC class II present protein antigens originating from exogenous sources, for example bacterial products. The cellular biology and the expression patterns of the two MHC classes are adapted to these different roles. MHC molecules of class I consist of a heavy chain and a light chain and are capable of binding a peptide of about 8 to 11 amino acids, but usually 9 or 10 amino acids, if this peptide has suitable binding motifs, and presenting it to cytotoxic T-lymphocytes. The peptide bound by the MHC molecules of class I originates from an endogenous protein antigen. The heavy chain of the MHC molecules of class I is preferably an HLA-A, HLA-B or HLA-C monomer, and the light chain is β-2-microglobulin. MHC molecules of class II consist of an α-chain and a β-chain and are capable of binding a peptide of about 12 to 25 amino acids if this peptide has suitable binding motifs, and presenting it to T-helper cells. The peptide bound by the MHC molecules of class II usually originates from an extracellular of exogenous protein antigen. The α-chain and the β-chain are in particular HLA-DR, HLA-DQ and HLA-DP monomers.

As used herein, the term “MHC complex” refers to a protein complex capable of binding with a specific repertoire of polypeptides known as HLA ligands and transporting said ligands to the cell surface.

As used herein, the term “MHC Ligand” means a polypeptide capable of binding to one or more specific MHC alleles. The term “HLA ligand” is interchangeable with the term “MHC Ligand”. Cells expressing MHC/Ligand complexes on their surface are referred to as “Antigen Presenting Cells” (APCs). Similarly, as used herein, the term “MHC binding peptide” relates to a peptide which binds to an MHC class I and/or an MHC class II molecule. In the case of MHC class I/peptide complexes, the binding peptides are typically 8-10 amino acids long although longer or shorter peptides may be effective. In the case of MHC class II/peptide complexes, the binding peptides are typically 10-25 amino acids long and are in particular 13-18 amino acids long, whereas longer and shorter peptides may also be effective.

As used herein, the term “T Cell Receptor” or “TCR” refers to a protein complex expressed by T cells that is capable of engaging a specific repertoire of MHC/Ligand complexes as presented on the surface of cells, such as antigen presenting cells (APCs).

As used herein, the term “MHC Binding Motif” refers to a pattern of amino acids in a protein sequence that predicts binding to a particular MHC allele.

As used herein, the term “AAY cleavage motif” refers to the short amino acid motif consisting of the sequence “alanine-alanine-tyrosine” capable of promoting proteasome-mediated cleavage of a peptide or protein, promoting the binding of the transporter associated with antigen processing to a peptide or protein, and/or increasing proteasome degradation at specific sites within a peptide or protein.

As used herein, the term “immune synapse” means the protein complex formed by the simultaneous engagement of a given T cell epitope to both a cell surface MHC complex and TCR.

As used herein, the term “polypeptide” refers to a polymer of amino acids, and not to a specific length; thus, peptides, oligopeptides and proteins are included within the definition of a polypeptide. As used herein, a polypeptide is said to be “isolated” or “purified” when it is substantially free of cellular material when it is isolated from recombinant and non-recombinant cells, or free of chemical precursors or other chemicals when it is chemically synthesized. A peptide or polypeptide (e.g., a polypeptide comprising, consisting of, or consisting essentially of one or more of SEQ ID NOS: 4-370, 391-440, and 448-833 or variants and fragments thereof, which in aspects may be isolated, synthetic, or recombinant) of the present disclosure, however, can be joined to, linked to, or inserted into another polypeptide (e.g., a heterologous polypeptide) with which it is not normally associated in a cell and still be “isolated” or “purified.” Additionally, one or more T-cell epitopes of the present disclosure can be joined to, linked to, or inserted into another polypeptide wherein said one or more T-cell epitopes of the present disclosure is not naturally included in the polypeptide and/or said one or more T-cell epitopes of the present disclosure is not located at its natural position in the polypeptide. When a polypeptide is recombinantly produced, it can also be substantially free of culture medium, for example, culture medium represents less than about 20%, less than about 10%, or less than about 5% of the volume of the polypeptide preparation.

As used herein, a “concatemeric” peptide or polypeptide refers to a series of at least two peptides or polypeptides linked together. Such linkages may form of string-of-beads design. In aspects, each of the peptides or polypeptides of concatemeric polypeptide may optionally be spaced by one or more linkers, and in further aspects neutral linkers. The term “linker” may refer to a peptide added between two peptide domains such as epitopes or vaccine sequences to connect said peptide domains. In aspects, a linker sequence is used to reduce steric hindrance between each one or more identified peptides of the instant disclosure, is well translated, and supports or allows processing of the each one or more identified polypeptides of the instant disclosure. In aspects, the linker should have little or no immunogenic sequence elements. In aspects, each peptide or polypeptide of the concatemeric polypeptide may optionally have one or more linkers, which may optionally be cleavage sensitive sites, adjacent to their N and/or C terminal end. In such a concatemeric peptide, two or more of the peptides may have a cleavage sensitive site between them. Alternatively two or more of the peptides may be connected directly to one another or through a linker that is not a cleavage sensitive site.

As used herein, the term “pharmaceutically acceptable” refers to approved or approvable by a regulatory agency of the Federal or a state government or listed in the U.S. Pharmacopeia or other generally recognized pharmacopeia for use in animals, including humans.

As used herein, the term “pharmaceutically acceptable excipient, carrier, or diluent” or the like refer to an excipient, carrier, or diluent that can be administered to a subject, together with an agent, and which does not destroy the pharmacological activity thereof and is nontoxic when administered in doses sufficient to deliver a therapeutic amount of the agent.

As used herein, the term “purpose built computer program” refers to a computer program designed to fulfill a specific purpose; typically to analyze a specific set of raw data and answer a specific scientific question.

As used herein, the term “z-score” indicates how many standard deviations an element is from the mean. A z-score can be calculated from the following formula: z=(X−μ)/σ; where z is the z-score, X is the value of the element, μ is the population mean, and σ is the standard deviation.

As used herein, the term “EpiBar™” refers to a 9-mer peptide that is predicted to be reactive to at least four different HLA alleles. Cluster scores higher than 10 are considered to be significant. A band-like EpiBar pattern is characteristic of promiscuous epitopes. Z score indicates the potential of a 9-mer frame to bind to a given HLA allele. All scores in the top 5% are considered “hits.”

As used herein, the term “native Fc” refers to a molecule or sequence comprising the sequence of a non-antigen-binding fragment resulting from digestion of whole antibody, whether in monomeric or multimeric form, into which a peptide sequence may be added by insertion into or replacement of a loop region. The original immunoglobulin source of the native Fc is preferably of human origin and may be any of the immunoglobulins, including but not limited to IgG1 and IgG2. Native Fc's are made up of monomeric polypeptides that may be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgA1, IgGA2). One example of a native Fc is a disulfide-bonded dimer resulting from papain digestion of an IgG (see Ellison et al. (1982), Nucleic Acids Res. 10: 4071-9). The term “native Fc” as used herein is generic to the monomeric, dimeric, and multimeric forms.

As used herein, the term “Immune Synapse” means the protein complex formed by the simultaneous engagement of a given T cell epitope to both a cell surface MHC complex and TCR.

As used herein, the singular forms “a,” “an,” and “the” are intended to include the plural forms, including “at least one,” unless the content clearly indicates otherwise. “Or” means “and/or.” As used herein, the term “and/or” and “one or more” includes any and all combinations of the associated listed items. For example, the term “one or more” with respect to the “one or more of SEQ ID NOS: 4-370, 391-440, and 448-833 of the present disclosure” includes any and all combinations of SEQ ID NOS: 4-370, 391-440, and 448-833. The term “or a combination thereof” means a combination including at least one of the foregoing elements.

As used herein, a “variant” peptide or polypeptide (including a variant Tregitope or detolerized antigen) can differ in amino acid sequence by one or more substitutions, deletions, insertions, inversions, fusions, and truncations or a combination of any of these. In aspects, a variant peptide or polypeptide (including a variant T-cell epitope) can differ in amino acid sequence by one or more substitutions, deletions, insertions, inversions, fusions, and truncations or a combination of any of these provided said variants retain MHC binding propensity and/or TCR specificity, and/or SARS-CoV-2 activity.

Although any methods and materials similar or equivalent to those described herein can be used in the practice or testing of the present disclosure, the preferred methods and materials are described. Other features, objects, and advantages of the present disclosure will be apparent from the description and the Claims. In the Specification and the appended Claims, the singular forms include plural referents unless the context clearly dictates otherwise. Unless defined otherwise, all technical and scientific terms used herein have the same meaning as commonly understood by one of ordinary skill in the art to which this invention belongs. All references cited herein are incorporated herein by reference in their entirety and for all purposes to the same extent as if each individual publication, patent, or patent application was specifically and individually indicated to be incorporated by reference in its entirety for all purposes.

### (ii) General

The adaptive immune cascade begins when soluble protein antigens are taken up by Antigen Presenting Cells (APCs) and processed through the Class II antigen presentation pathway. Protein antigens in the Class II presentation pathway are degraded by various proteases found in the Endoplasmic Reticulum. Some of the resulting protein fragments are bound to Class II MHC molecules. Peptide-loaded MHC molecules are trafficked to the cell surface where they are interrogated by CD4+ T cells. Peptide fragments that are capable of binding to an MHC molecule and mediating the cell-to-cell interaction between APCs and circulating T cells are referred to as T cell epitopes. Recognition of these peptide-MHC complexes by CD4+ T cells can lead to either an immune activating or an immune suppressing response based on the phenotype of the responding T cells and the local cytokine/chemokine milieu. In general, engagement between the MHC/peptide complex and the T cell receptor (TCR) of T effector cells leads to activation and the subsequent secretion of pro-inflammatory cytokines such as IL-4, and IFN-γ. On the other hand, the activation of natural T regulatory cells (T_{Regs}) leads to the expression of the immune suppressive cytokines IL-10 and TGF-β, among others (Shevach E, (2002), Nat Rev Immunol, 2(6):389-400). These cytokines act directly on nearby effector T cells leading in some cases to anergy or apoptosis. In other cases, regulatory cytokines and chemokines convert effector T cells to T regulatory phenotypes; this process is referred here as “induced” or “adaptive” tolerance. T cell epitopes that are capable of binding to MHC molecules and engaging and/or activating circulating naturally occurring T_{Regs} (in aspects, including natural T_{Regs} and/or adaptive T_{Regs}), are referred to as “Tregitopes.” In aspects, the instantly disclosed Tregitopes are T cell epitope clusters, which are epitopes capable of binding to multiple MHC alleles and multiple TCRs.

Initial self/non-self discrimination occurs in the thymus during neonatal development where cortical and medullary epithelial cells express specific self-protein epitopes to immature T cells. T cells recognizing self-antigens with high affinity are deleted, but autoreactive T cells with moderate affinity sometimes avoid deletion and can be converted to natural regulatory T cells (T_{Reg}) cells. These natural T_{Reg} cells are exported to the periphery and help to control a latent autoimmune response. Natural regulatory T cells are a critical component of immune regulation and self-tolerance.

Self-tolerance is regulated by a complex interplay between T cells, B cells, cytokines and surface receptors. T regulatory immune responses counterbalance T effector immune response to protein antigens (whether self or foreign). A tilt of the balance toward the autoreactive side, either by increasing the number or function of autoreactive T effector cells or by diminishing the number or function of T regulatory cells, is manifested as autoimmunity.

A second form of tolerance occurs in the periphery where mature T cells are converted to an ‘adaptive’ T_{Reg} phenotype upon activation via their T cell receptor in the presence of IL-10 and TGF-β, which are usually supplied by bystander T regulatory cells. The possible roles for these ‘adaptive’ T_{Reg} cells include dampening immune response following the successful clearance of an invading pathogen, controlling excessive inflammation caused by an allergic reaction, controlling excessive inflammation caused by low-level or chronic infection, or possibly controlling inflammatory response targeting beneficial symbiotic bacteria and viruses. ‘Adaptive’ T_{Regs} may also play a role in suppressing immune response targeting human antibodies that have undergone somatic hypermutation (Chaudhry A et al., (2011), Immunity, 34(4):566-78).

T_{Reg} cells are also instrumental in B cell tolerance. B cells express a single low affinity Fc receptor, FcγRIIB on their cell surface (Ravetch J V et al., (1986), Science, 234(4777):718-25). This receptor contains the immunoreceptor tyrosine-based inhibition motif sequence (ITIM) in its cytoplasmic domain. Co-ligation of FcγRIIB and the B-cell receptor (BCR) by immune complexes act to trigger the tyrosine phosphorylation of the ITIM leading to the recruitment of the inositol phosphatase, SHIP, which inhibits BCR-triggered proliferation by interfering with the activation of MAP kinases and blocks phagocytosis by the dissociation of Burton's tyrosine kinase (Btk) from the cell membrane, which inhibits calcium influx into the cell. FcγRIIB can also induce apoptosis independent of the ITIM. Upon homo-aggregation of FcRIIB by ICs, the association of Btk with the cell membrane is enhanced, thereby triggering an apoptotic response (Pearse R, et al., (1999), Immunity, 10(6):753-60). Expression of FcγRIIB is highly variable and cytokine dependent. IL-4 and IL-10, which are expressed by activated Th2 and T_{Reg} cells, have been shown to act synergistically to enhance FcγRIIB expression (Joshi T et al., (2006), Mol Immuno., 43(7):839-50), thus aiding in the suppression of a humoral response.

Further, certain viruses contain a significantly high number of T cell receptor (TCR) ‘cross-conservation’ with human sequences (i.e., JanusMatrix-defined ‘human-like’ T cell epitopes) and are able to establish chronic infections in humans. As certain viruses appear to have many such epitopes that are highly cross-conserved with self and significantly expand T regulatory cells (T_{Regs}), immune camouflage may be an important method by which certain human pathogens, including SARS-CoV-2, dampen or even escape adaptive immune response. As such, SARS-CoV-2 may minimize a host response by adopting ‘immune camouflage’ through expression of Tregitopes and recruitment and activation of T_{Regs}. It was hypothesized that SARS-CoV-2 may contain highly cross-conserved epitopes that exhibit low immunogenicity and stimulate functional T_{Regs}, and accordingly dampen or even escape any adaptive immune response. Such epitopes (i.e. Tregitopes) accordingly further serve as important targets for modification and/or removal, including specific modifications, to create detolerized antigen/peptides with increased immunogenicity.

Accordingly, some aspects of the present disclosure concern peptides derived from one or more proteins of SARS-CoV-2 wherein a Tregitope or Tregitopes therein are compromised to inhibit, reduce or abolish engagement and/or activation of T_{Reg} cells. Tregitopes can be compromised through deletion of the Tregitope from the peptide, partial deletion of the Tregitope from the peptide or mutation of the Tregitope. For example, in aspects, removal of one or more of the identified regulatory T cell epitopes from an antigen or polypeptide sequence comprises deletion of all or some of the amino acids of the one or more regulatory T cell epitopes. In aspects, said removal of the one or more regulatory T cell epitopes from an antigen or polypeptide sequence comprises deletion of some or all of the amino acids of the one or more regulatory T cell epitopes and adding one or more amino acids at the site of deletion of the regulatory T cell epitope amino acids. In aspects, said removal of the one or more regulatory T cell epitopes from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said removal of the one or more regulatory T cell epitopes from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of removal need not correspond to the number of amino acids deleted from the previously existing regulatory T cell epitope amino acids. In aspects, with the tolerizing epitopes (Tregitopes) identified, binding of such epitopes can be disrupted by mutating anchoring residues and/or TCR epitope residues included therein. Such mutations for the anchoring residues must select replacement amino acids which are dis-favorable for binding. Any amino acid substitution identified by EpiMatrix as disruptive (reduces predicted T cell epitope content) is viable. Targeted anchoring residues and specific mutations to improve immunogenicity and decease tolerogenicity are disclosed herein. Further, by mutating TCR contacts, T cell recognition of such tolerizing epitopes can be disrupted. Any amino acid substitution to TCR contacts can disrupt TCR recognition. Targeted TCR contacts for such epitopes are disclosed herein.

The identification of Tregitopes therefore offer approaches to harness the innate immune system, as well in providing targets for deletion, partial deletion and/or mutation to detolerize and avoid unwanted adaptive immune camouflage. For example, it is possible to exploit Tregitope specific T_{Reg} cells to suppress unwanted immune responses and also to induce adaptive T_{Reg} to co-delivered proteins. This discovery has implications for the design of therapeutic regimens and antigen-specific therapies for transplantation, protein therapeutics, allergy, chronic infection, autoimmunity and vaccine design. Similarly, the Tregitopes presented herein can further be targets for deletion and/or mutation to avoid T_{Reg} binding and the subsequent immune suppression, while enlisting and/or stimulating an immune response and allowing for detolerization of disguised “self” epitopes or antigens.

The present disclosure accordingly concerns both Tregitope compositions of the peptides identified herein and detolerized antigenic compositions thereof. The administration of a drug, a protein, or an allergen in conjunction with compositions herein, including a Tregitope composition of the present disclosure can suppress an effector immune response. Tregitopes, including Tregitope compositions of the present disclosure, can be used to deliberately manipulate the immune system toward tolerance. The identification of Tregitopes further allows for the design of peptides that avoid activation of T_{Reg} cells. In certain aspects, the detolerized antigenic compositions can be utilized as immunostimulatory compositions with other antigenic compositions to stimulate an immune response to SARS-CoV-2. For example, including a detolerized antigen with of deleted Tregitope(s), partially deleted Tregitopes and/or mutation(s) to a Tregitope provides peptide compositions with reduced and/or abolished TReg engagement and/or activation, thereby allowing the administered composition to avoid immune camouflage. The administration of an antigen comprising, consisting, or consisting essentially of a deleted and/or mutated Tregitope of a detolerized antigen can alternatively disrupt binding and allow for the adaptive immune response to recognize the epitope and circumvent the immune camouflage response. The removal, including removal by mutation, of the Tregitopes set forth herein from a polypeptide or antigen provide for detolerizing antigens to provoke the immune system and/or prevent a pathogen from recognition as self by the immune system.

The compositions of the present disclosure include Tregitope compositions and detolerized antigens thereof including peptides with deleted, partially deleted and/or mutated Tregitopes. The compositions may include one or more polypeptides (which may be termed herein as “Treg activating regulatory T-cell epitope”, “Tregitope”, “Tregitope peptide”, or “T-cell epitope polypeptide”) that comprise, consist, or consist essentially of an amino acid sequence as set forth in any of SEQ ID NOS: 4-370, 391-440, and 448-833 and/or a detolerized antigen variant thereof, including a peptide with a deleted Tregitope, a partially elected Tregitope and/or one or more amino acid mutations therein. The compositions may include polypeptide fragments and/or variants thereof, including optionally 1 to 12 additional amino acids distributed in any ratio on the N-terminus and/or C-terminus of a polypeptide as set forth in SEQ ID NOS: 4-370, 391-440, and 448-833 or a detolerized antigen variants thereof as described herein. The compositions may include nucleic acids, expression cassettes, plasmids, expression vectors, recombinant viruses, or cells (all of which may be isolated, synthetic, recombinant, or combinations thereof) as disclosed herein. The compositions may include chimeric or fusion polypeptide compositions as disclosed herein (which in aspects may be isolated, synthetic, recombinant, or combinations thereof). The compositions may be part of a pharmaceutical composition and/or formulation. The compositions may be utilized in the selective engagement and activation of regulatory T cells, as well as in avoiding activation thereof in the case of the detolerized antigens as set forth herein. The compositions may be one or more of a polypeptide (or peptide), a nucleic acid, a chimeric polypeptide and/or a pharmaceutical composition.

Presented herein is a demonstration that severe acute respiratory syndrome coronavirus 2 (SARS-CoV-2) contains T cell epitopes that relate to naturally occurring populations of regulatory T cells (including natural T_{Regs} and/or adaptive T_{Regs}). In the course of normal immune surveillance, these proteins are taken up by professional APCs, such as dendritic cells or macrophages, and degraded. During the degradation process, some of the epitopes contained in these proteins are bound to MHC molecules, transported to the cell surface presented to regulatory T cells. Those cells, once activated by the APC, release cytokines and chemokines help to suppress autoimmune responses that would otherwise hinder the function of the extracellular proteins.

By using the Tregitope compositions of the present disclosure to selectively activate naturally occurring T_{Regs} (in aspects, including natural T_{Regs} and/or adaptive T_{Regs}), it is herein shown that the Tregitope compositions of the present disclosure can be used to suppress a variety of unwanted immune responses. In its simplest form, systemic application of the Tregitope compositions of the present disclosure can be used as a generalized immune suppressant useful for controlling severe autoimmune reactions such as, for example, MS flare-ups, allergic reactions, transplant reactions, or uncontrolled response to infection.

In a more controlled application, the Tregitope compositions of the present disclosure can be used to suppress localized autoimmune responses. In a targeted application, such as might be achieved through the fusion, bonding or admixture of the Tregitope compositions of the present disclosure to certain other T cell epitopes, the Tregitope compositions can suppress highly specific immune reactions to the fused, bonded, or admixed T cell epitopes while leaving the balance of the immune system intact. For example, through the delivery of a Tregitope composition of the present disclosure fused to an autoimmune antigen, an allergen, or an antigenic protein such as an antibody (which can be IgG, IgM, IgA, IgD or IgE molecules or antigen-specific antibody fragments thereof (including, but not limited to, a Fab, F(ab′)₂, Fv, disulfide linked Fv, scFv, single domain antibody, closed conformation multispecific antibody, disulfide-linked scfv, diabody) or replacement enzyme, the immune system can be trained to “tolerate” the co-delivered antigen by, e.g., inducing naturally occurring T_{Regs} (in aspects, including natural T_{Regs} and/or adaptive T_{Regs}) and/or converting the phenotype of responding effector T cells to that of adaptive regulatory T cells.

The Tregitopes of the present disclosure, as well as detolerized antigens thereof, are derived from severe acute respiratory syndrome coronavirus 2 (SARS-CoV-2) and the proteins expressed therein, which is a positive-sense single-stranded ribonucleic (RNA) virus belonging to the Coronaviridae family. SARS-CoV-2 (which may also be referred to herein as “COVID-19 virus”) was first identified in Wuhan, China in late 2019 and is the cause of the highly contagious coronavirus disease 2019 (which has been termed and may be referred to herein as “COVID-19”, “2019 novel coronavirus”, or “2019-nCoV”). To be useful, these Tregitopes should be true T cell epitopes (i.e., capable of binding to both MHC molecules and TCRs). In aspects, the Tregitopes should be related to a pre-existing population of regulatory T cells that is sufficiently large to have a therapeutic effect. T cell epitope clusters, which are epitopes capable of binding to multiple MHC alleles and multiple TCRs, are key to satisfying this latter qualification.

As is further set forth herein, the identification of the true Tregitopes allows for the ability to remove and/or mutate such Tregitopes from polypeptides or antigens to provide detolerized antigens. For example, mutations to affect the anchoring and/or TCR binding within the “true” T cell epitope allow for the MHC binding through a retained agretope, while avoiding T_{Reg} recognition and binding or diminished MHC binding through agretope mutation to have the detolerized antigen fail to be effectively presented at all. Such can then allow for immune stimulation and/or avoidance of the immune suppression by activating the T_{Regs}.

With respect to the Tregitopes, the instantly disclosed treatments provide the following advantages:

1. Treatment with the Tregitope compositions of the present disclosure is highly antigen specific (e.g., treatment with the Tregitope compositions can, e.g., expand and/or stimulate corresponding naturally occurring T_{Reg} populations (in aspects, including natural T_{Regs} and/or adaptive T_{Regs}) in a highly antigen specific manner);

2. An efficient and less expensive treatment regimen when compared to current antigen specific therapies wherein subjects are treated over a prolonged period of time with frequent high dose antigen preparations; and

3. A second line of defense when induction of tolerance through high dose therapy fails to induce immune tolerance in the treated subject.

With respect to the detolerized antigens derived from the Tregitopes, the disclosed treatments offer efficient and low-cost treatment regimen that minimize T_{Reg} activation in an immunization step and allows for a more pronounced and effective outcome. The detolerized antigens may include a protein or a peptide derived from SARS-CoV-2 wherein a Tregitope or Tregitopes therein are deleted and/or partially deleted and/or mutated. A detolerized antigen may further include a mutated Tregitope or a peptide comprised of a mutated Tregitope.

In aspects, the present disclosure is directed to therapeutic Tregitope compositions that are safely administered to a patient experiencing an autoimmune response. The mechanism of action of the claimed Tregitopes (and Tregitopes included in said Tregitope compositions) is natural, supporting their efficacy and safety.

In addition to the Tregitope compositions, the present disclosure also concerns mutations to the Tregitope peptides or antigens such that binding is disrupted and the antigens are detolerized by the immune system. It was hypothesized that SARS-CoV-2 contains highly cross-conserved epitopes that exhibit low immunogenicity and stimulate functional Tregs, which may dampen or even escape adaptive immune response. Such epitopes can therefore also serve as important targets for specific modifications, including specific mutations, to create detolerized peptides with increased immunogenicity. By identification of the Tregitopes set forth herein, the mutagenesis therein provides for detolerized antigens. With the tolerizing epitopes identified, binding of such epitopes can be disrupted by deleting the Tregitope or mutating anchoring residues included therein. Such mutations for the anchoring residues must select replacement amino acids which are dis-favorable for binding. Any amino acid substitution identified by EpiMatrix as disruptive (reduces predicted T cell epitope content) is viable. Targeted anchoring residues and specific mutations to improve immunogenicity and decease tolerogenicity are disclosed herein. Further, by mutating TCR contacts, T cell recognition of such tolerizing epitopes can be disrupted. Any amino acid substitution to TCR contacts can disrupt TCR recognition. Targeted TCR contacts for such epitopes are disclosed herein.

As such, the instant disclosure further provides detolerized SARS-CoV-2 antigens (e.g., peptides and polypeptides), and methods of producing and using the same. Aspects include methods for improving the efficacy of vaccine antigens against SARS-CoV-2 infection and related diseases caused by SARS-CoV-2, including COVID-19, comprising the steps of: (a) identifying constituent T cell epitopes within a SARS-CoV-2 vaccine antigen which share TCR contacts with proteins derived from either the human proteome or the human microbiome; (b) deleting/removing part or whole of the identified Tregitope from the antigen and/or introducing mutated amino acids therein that negatively impact anchoring or epitope recognition to provide a detolerized antigen and (c) administering the detolerized antigen to a subject as part of a vaccine or immunostimulatory composition, either alone or in combination with other antigenic peptides an/or other detolerized antigens. It will be appreciated that the detolerized antigen can be produced by manipulations to the nucleic acids encoding the antigen to remove and/or mutate such. It will also be appreciated that nucleic acids compositions comprised of sequences encoding the detolerized antigens can be utilized in the compositions as well and/or instead of a detolerized antigen polypeptide.

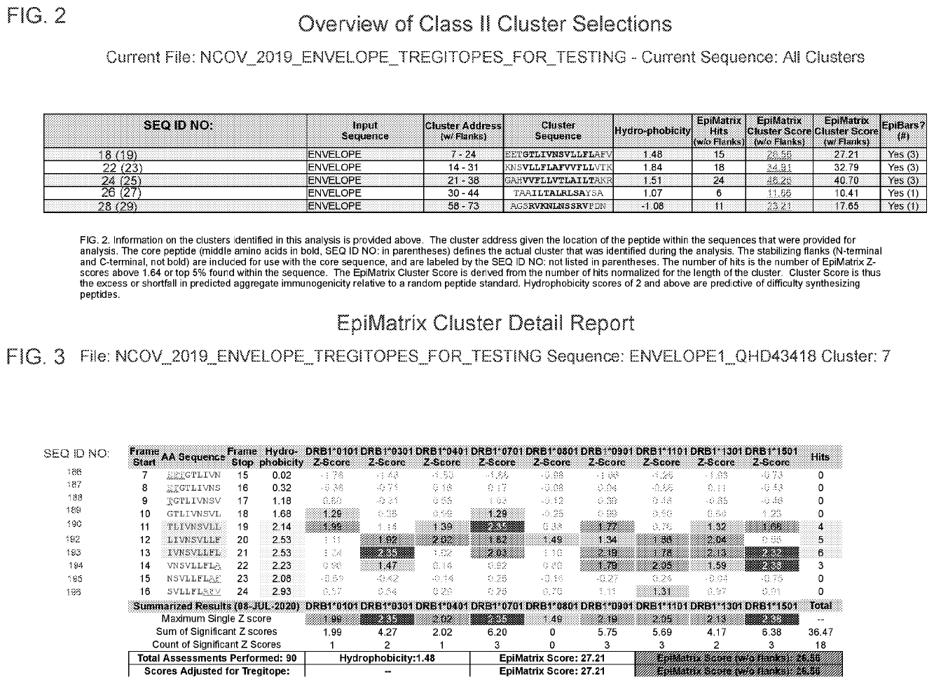

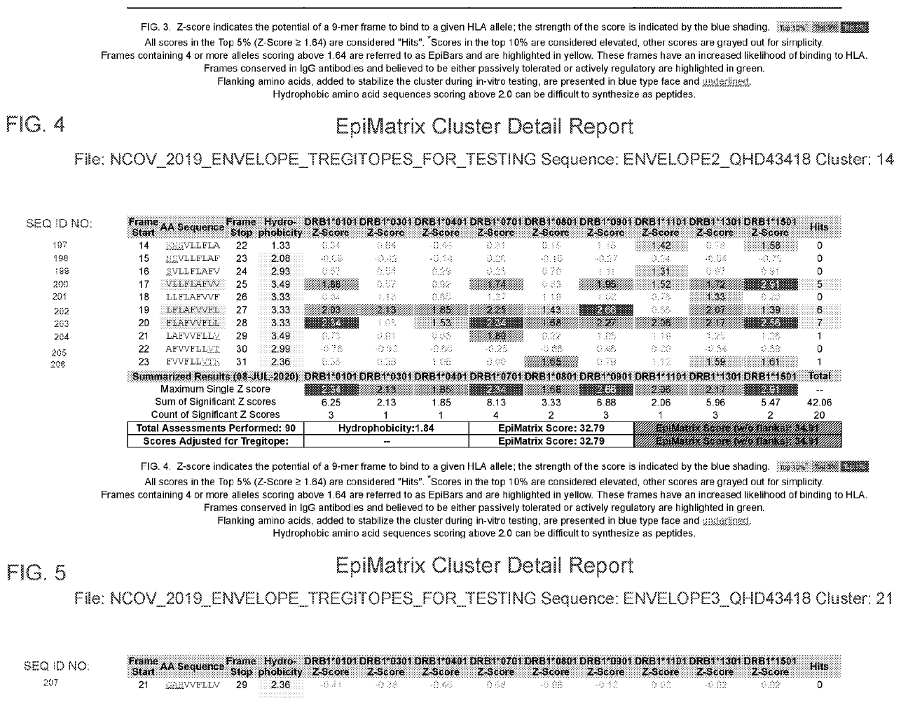

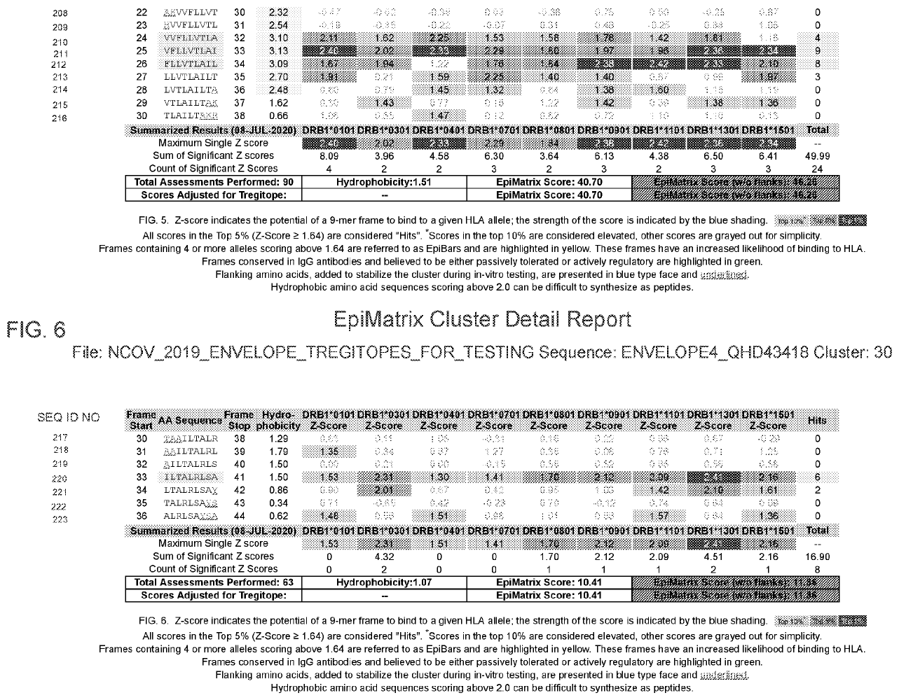

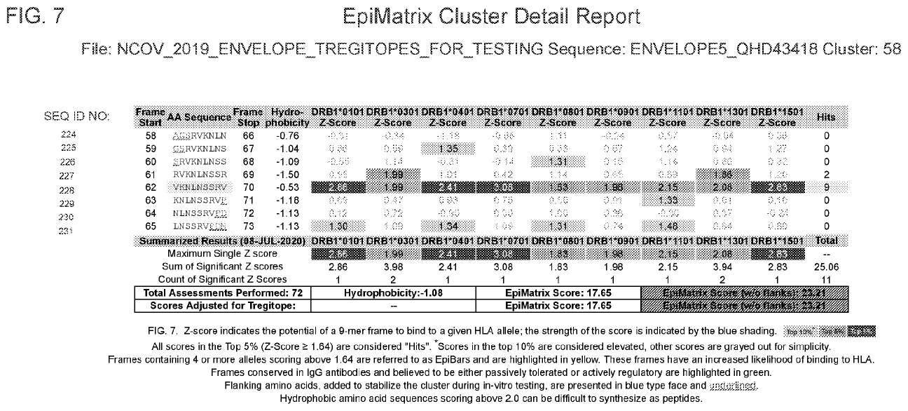

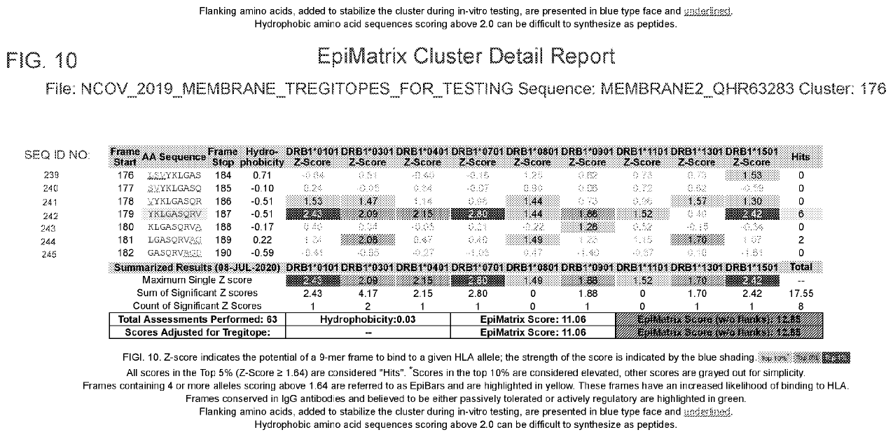

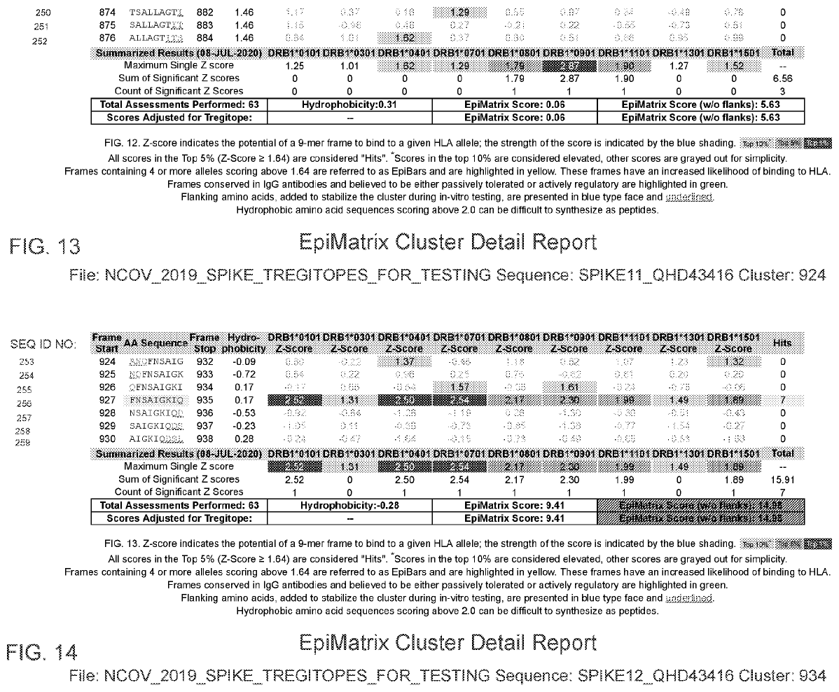

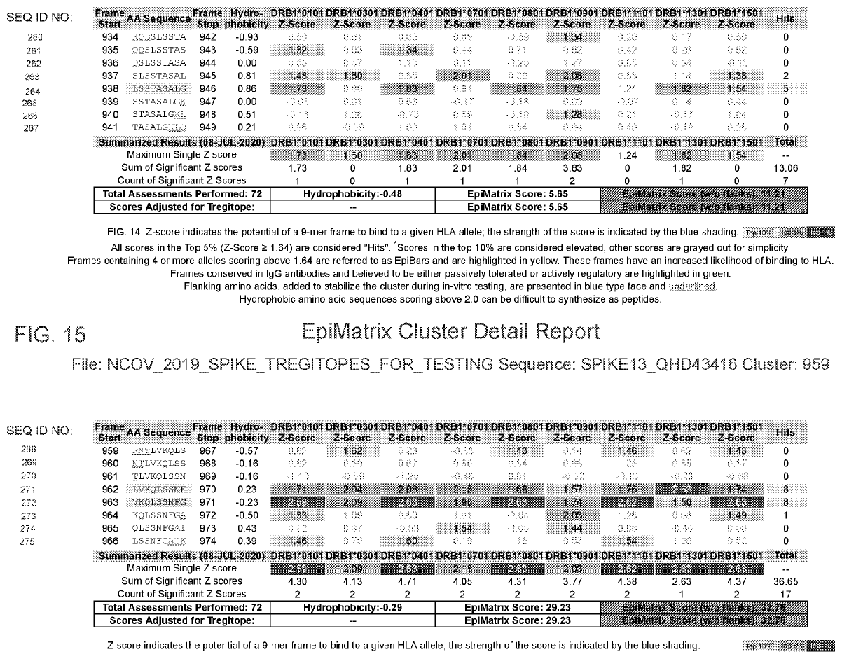

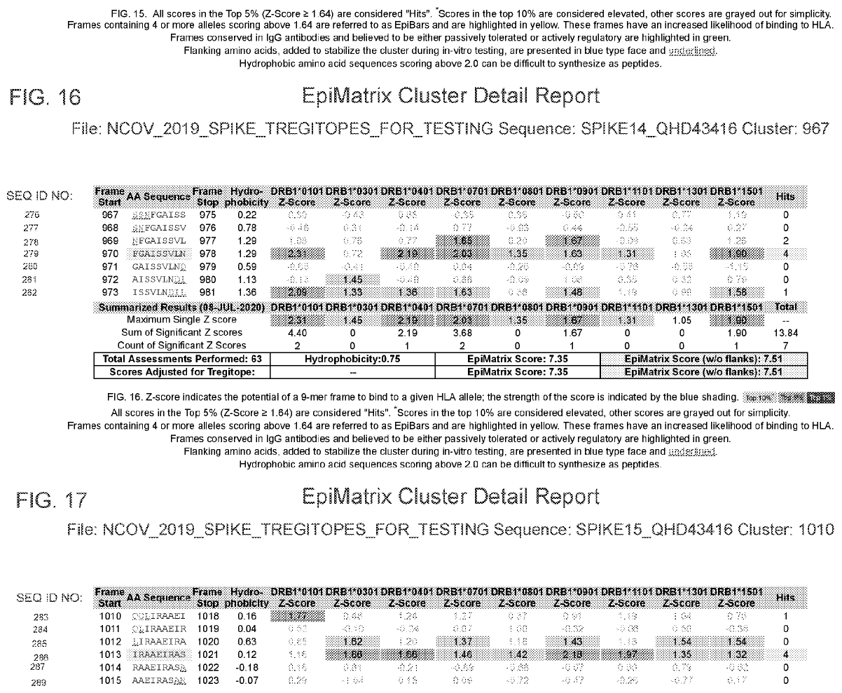

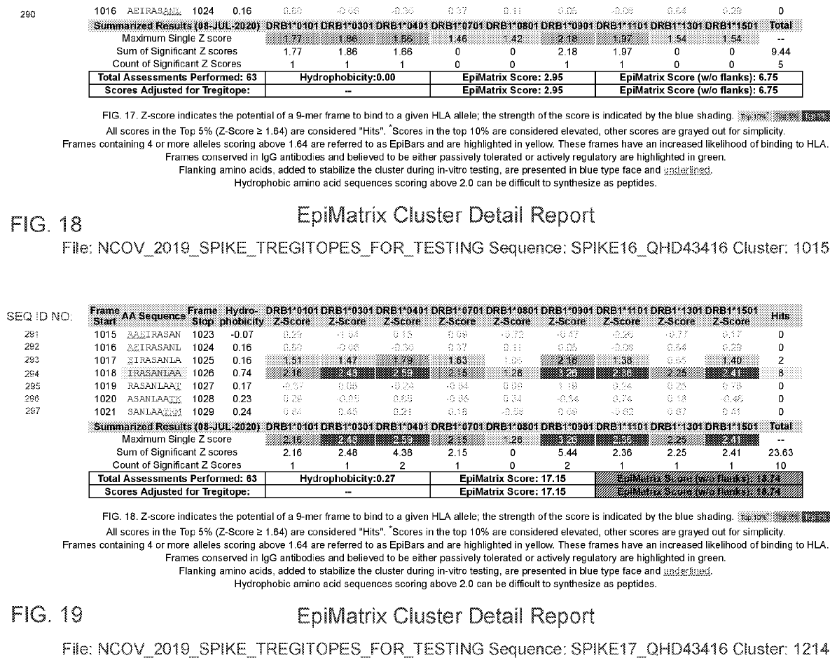

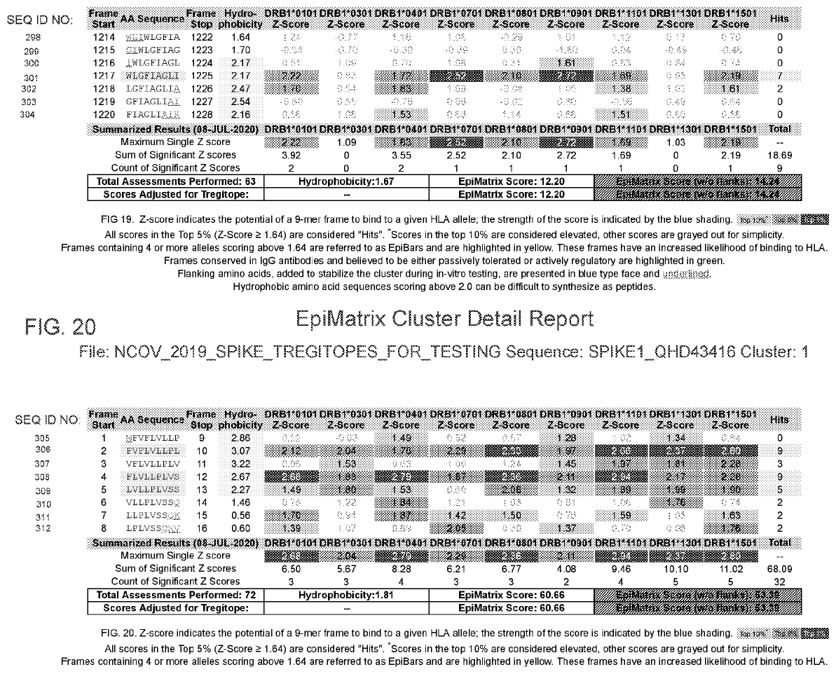

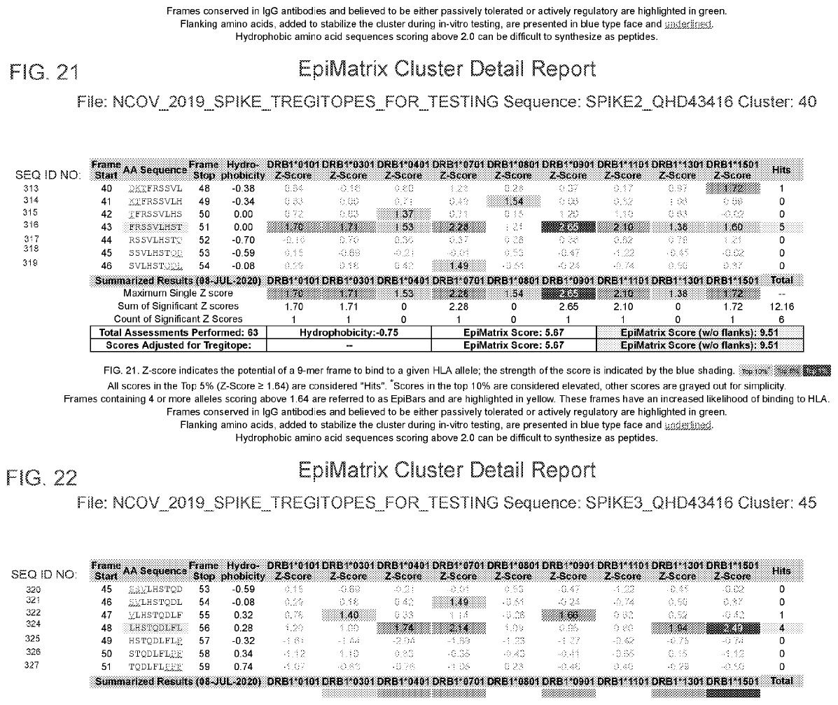

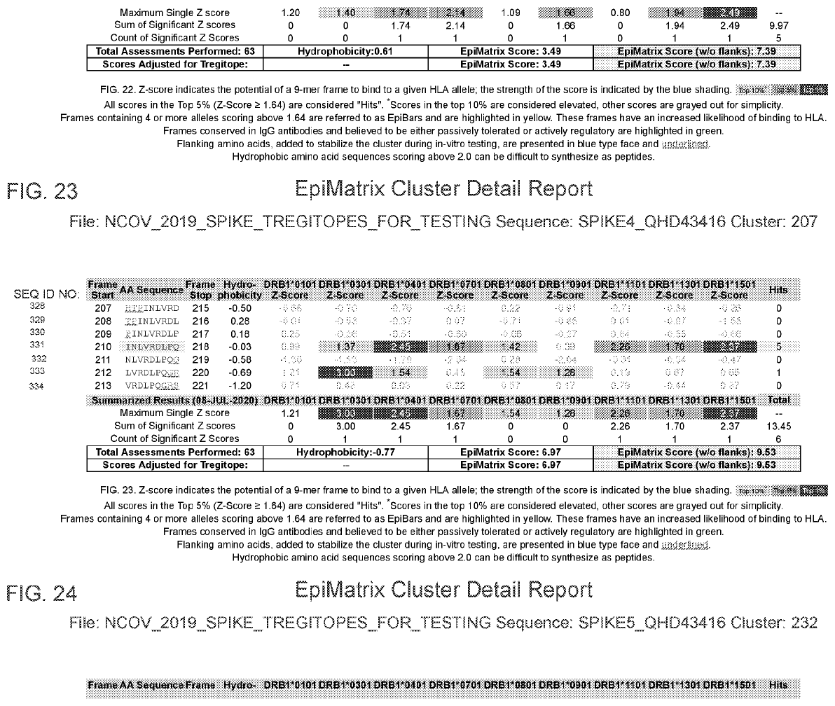

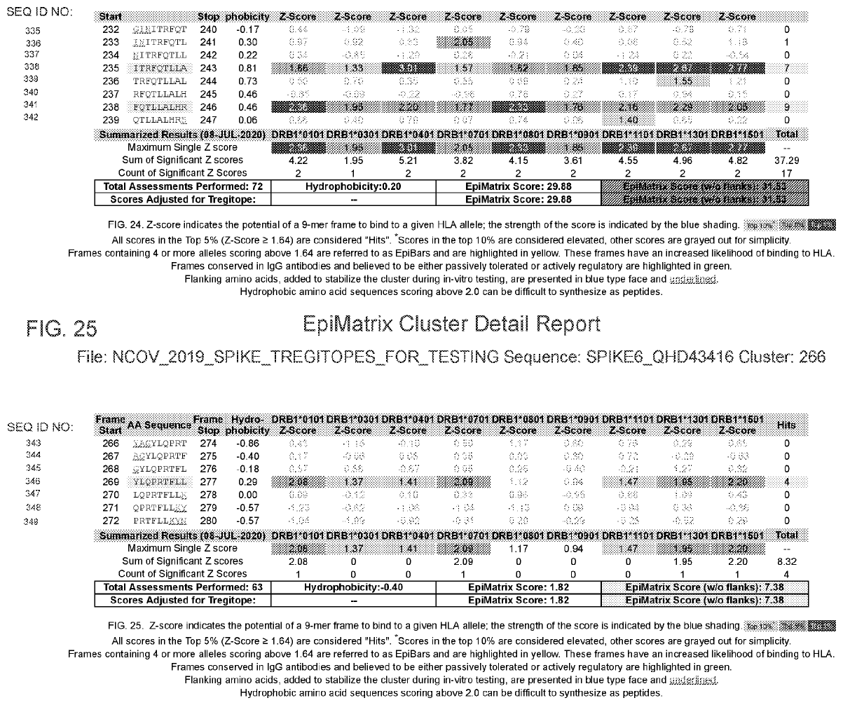

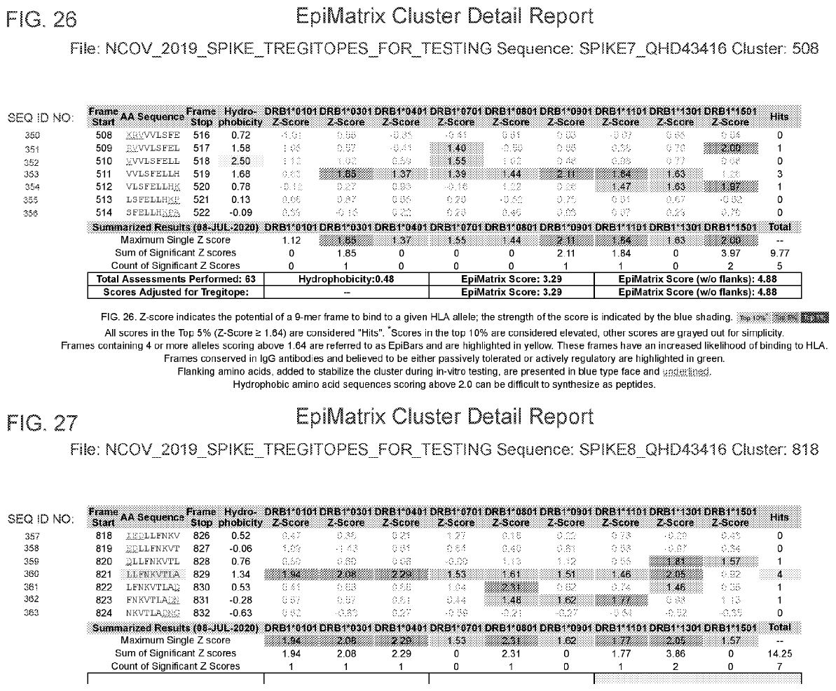

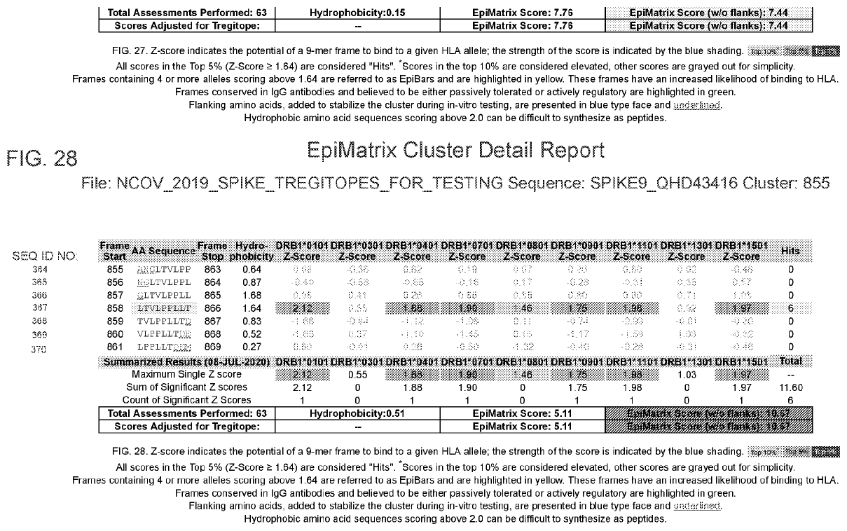

In aspects, the present disclosure is directed to Tregitope compositions that include one or more of the regulatory Tregitopes (or mutations thereof) set forth in Table 1, Table 2, and FIGS. 2-31, as well as fragments thereof (including 9-mers), variants thereof, and fragments of such variants thereof. Similarly, in other aspects, the present disclosure concerns detolerized antigenic peptides. In some aspects, the detolerized antigens include a protein or a peptide derived from SARSS-CoV-2 wherein one or more of the Tregitopes as set forth in Table 1, Table 2 and FIGS. 2-31 are deleted. In other aspects, the detolerized antigens include a SARS-CoV-2 protein or peptide, wherein one or more Tregiopes therein as set forth in Table 1, Table 2 and FIGS. 2-31 are partially deleted, deleted and/or mutated at one or more amino acids therein. In further aspects, the detolerized antigens include a polypeptide compositions that includes a Tregitope as set forth in Table 1, Table 2 and FIGS. 2-31 wherein one or more amino acids therein is mutated. For the Tregitope compositions it is significant that the peptide or fragment or variant thereof retains MHC binding propensity and/or TCR specificity. For the detolerizing compositions, the peptides include modification and/or mutations to either reduce MHC binding and/or reduce homologies between TCR contacts of said target T cell epitope and the human proteome or the human microbiome.

Table 2 describes the core peptide (middle amino acids in bold, SEQ ID NO: in parentheses), which defines the actual cluster that was identified during the analysis. The stabilizing flanks (N-terminal and C-terminal, not bold) are optionally included for use with the core sequence, and are labeled in Table 2 by the SEQ ID NO: not listed in parentheses. In certain aspects, the Tregitopes of the instant disclosure can be capped with an N-terminal acetyl and/or C-terminal amino group.

TABLE 1
Regulatory Tregitopes

| SEQUENCE | SEQUENCE ID NO: |
|---|---|
| WSFNPETNILLNVPLHGTILTRPLL | SEQ ID NO: 4 |
| ATSRTLSYYKLNTDHSSSSDNIALL | SEQ ID NO: 5 |
| ATSRTLSYYKLGASQRVAGDSGFAAYSRYRIGNY | SEQ ID NO: 440 |
| KLNTDHSSSSNIALL | |
| SFVSEETGTLIVNSVLLFLAFVVFLLVTLAILTA | SEQ ID NO: 6 |
| LRLCAYCCNIVNV | |
| PSFYVYSRVKNLNSSRVPDLLV | SEQ ID NO: 7 |
| GVYYPDKVFRSSVLHSTQDLFLPFFSNVTW | SEQ ID NO: 8 |
| KIYSKHTPINLVRDLPQGFSALEPL | SEQ ID NO: 9 |
| VDLPIGINITRFQTLLALHRSYLTP | SEQ ID NO: 10 |
| GAAAYYVGYLOPRTFLLKYNENGTI | SEQ ID NO: 11 |
| VGYQPYRVVVLSFELLHAPATVCGP | SEQ ID NO: 12 |
| SKRSFIEDLLFNKVTLADAGFIKQY | SEQ ID NO: 13 |
| ICAQKFNGLTVLPPLLTDEMIAQYT | SEQ ID NO: 14 |
| NQKLIANQFNSAIGKQDSLSSTASALGKLQDVVN | SEQ ID NO: 15 |
| Q | |
| LVKQLSSNFGAISSVLNDILSRLDK | SEQ ID NO: 16 |
| QTYVTQQLIRAAEIRASANLAATKMSECVL | SEQ ID NO: 17 |

TABLE 2
Regulatory Tregitopes

| | | | Sequence | | | | | |
|---|---|---|---|---|---|---|---|---|
| | | | (cluster | | | | | |
| | | | sequence is | | | | | |
| | | | bolded, while | | | | | |
| | | | flanks are | | | | | |
| | | | not bolded, | | EpiMatrix | EpiMatrix | | |
| | | Sequence | mutations | | Hits | Cluster | Number | Janus |
| SEQ | Input | Address | from WT are | Class | (Class II | Score | of Human | Homology |
| ID NO: | Sequence | (w/Flanks) | italicized) | I Hits | w/o Flanks) | (w/o Flanks) | Sequences | Score |
| 18 | ENVELOPE | 0007-0024 | EET: GTLIVNSV | 11 | 15 | 26.56 | 22 | 2.72 |
| (19) | (SEQ ID | | LLFL: AFV | | | | | |
| | NO: 1) | | | | | | | |
| 20 | ENVELOPE | 0014-0036 | VNS: VLLFLAFV | 26 | 22 | 40.15 | 46 | 3.48 |
| (21) | (SEQ ID | | VFLLVTL: A | | | | | |
| | NO: 1) | | | | | | | |
| 22 | ENVELOPE | 0014-0031 | KNS: VLLFLAFV | — | 18 | 34.91 | 33 | 3.95 |
| (23) | (SEQ ID | | VFLL: VTK | | | | | |
| | NO: 1) | | | | | | | |
| 24 | ENVELOPE | 0021-0038 | GAH: VVFLLVTL | — | 24 | 46.26 | 47 | 5.25 |
| (25) | (SEQ ID | | AILT: AKR | | | | | |
| | NO: 1) | | | | | | | |
| 26 | ENVELOPE | 0030-0044 | TAA: ILTALRL | — | 6 | 11.86 | 28 | 5.75 |
| (27) | (SEQ ID | | [symbol] A: YSA | | | | | |
| | NO: 1) | | | | | | | |
| 28 | ENVELOPE | 0058-0073 | AGS: RVKNLNSS | — | 11 | 23.21 | 15 | 3.00 |
| (29) | (SEQ ID | | RV: PD | | | | | |
| | NO: 1) | | | | | | | |
| 30 | ENVELOPE | 0060-0072 | S: RVKNLNSSR | 1 | 11 | 23.21 | 15 | 3.00 |
| (31) | (SEQ ID | | V: PD | | | | | |
| | NO: 1) | | | | | | | |
| 32 | MEMBRANE | 0042-0060 | RNR: RLYIIKLI | 29 | 15 | 27.53 | 8 | 1.56 |
| (33) | (SEQ ID | | FLWLL: WPV | | | | | |
| | NO: 2) | | | | | | | |
| 34 | MEMBRANE | 0115-0129 | ETN: ILLNVPLH | 3 | 8 | 16.48 | 9 | 2.45 |
| (35) | (SEQ ID | | G: TIL | | | | | |
| | NO: 2) | | | | | | | |
| 36 | MEMBRANE | 0175-0190 | TLS: YYKLGASQ | 4 | 9 | 17.64 | 8 | 1.83 |
| (37) | (SEQ ID | | RV: AGD | | | | | |
| | NO: 2) | | | | | | | |
| 38 | MEMBRANE | 0175-0190 | MLS: YYKLGASQ | — | | 17.64 | | 2.20 |
| (39) | (SEQ ID | | RV: AGD | | | | | |
| | NO: 2) | | | | | | | |
| 40 | MEMBRANE | 0176-0190 | LSV: YKLGASQR | — | 6 | 12.85 | 8 | 2.75 |
| (41) | (SEQ ID | | V: AGD | | | | | |
| | NO: 2) | | | | | | | |
| 42 | SPIKE | 0001-0016 | M: FVFLVLLPLV | 10 | 26 | 53.39 | 63 | 4.03 |
| (43) | (SEQ ID | | SS: QCV | | | | | |
| | NO: 3) | | | | | | | |
| 44 | SPIKE | 0001-0016 | M: FVFLVLLPLV | — | 26 | 53.39 | 60 | 3.94 |
| (45) | (SEQ ID | | SS: QKV | | | | | |
| | NO: 3) | | | | | | | |
| 46 | SPIKE | 0040-0054 | DKV: FRSSVLHS | 3 | 5 | 9.51 | 11 | 1.92 |
| (47) | (SEQ ID | | T: QDL | | | | | |
| | NO: 3) | | | | | | | |
| 48 | SPIKE | 0040-0054 | DKT: FRSSVLHS | — | 5 | 9.51 | 7 | 2.67 |
| (49) | (SEQ ID | | T: QDL | | | | | |
| | NO: 3) | | | | | | | |
| 50 | SPIKE | 0045-0059 | SSV: LHSTQDLF | — | 4 | 7.38 | 5 | 2.00 |
| (51) | (SEQ ID | | L: PFF | | | | | |
| | NO: 3) | | | | | | | |
| 52 | SPIKE | 0207-0221 | HTP: INLVRDLQ | 3 | 5 | 9.53 | 17 | 4.25 |
| (53) | (SEQ ID | | Q: GFS | | | | | |
| | NO: 3) | | | | | | | |
| 54 | SPIKE | 0207-02221 | HTP: INLVRDLQ | — | 5 | 9.53 | 17 | 5.67 |
| (55) | (SEQ ID | | Q: GRS | | | | | |
| | NO: 3) | | | | | | | |
| 56 | SPIKE | 0232-0247 | GIN: ITRFQTLL | — | 16 | 31.53 | 11 | 1.47 |
| (57) | (SEQ ID | | ALHR: S | | | | | |
| | NO: 3) | | | | | | | |
| 58 | SPIKE | 0232-0248 | GIN: ITRFQTLL | 10 | 16 | 31.53 | 11 | 1.47 |
| (59) | (SEQ ID | | ALHR: SY | | | | | |
| | NO: 3) | | | | | | | |
| 60 | SPIKE | 0266-0280 | YAG: YLQPRTFL | — | 4 | 7.38 | 6 | 2.75 |
| (61) | (SEQ ID | | L: KYN | | | | | |
| | NO: 3) | | | | | | | |
| 62 | SPIKE | 0508-0522 | YRV: VVLSFELL | 5 | 3 | 4.88 | 30 | 7.57 |
| (63) | (SEQ ID | | H: APA | | | | | |
| | NO: 3) | | | | | | | |
| 64 | SPIKE | 0508-0522 | KRV: VVLSFELL | — | 3 | 4.88 | 29 | 10.00 |
| (65) | (SEQ ID | | H: KPA | | | | | |
| | NO: 3) | | | | | | | |
| 66 | SPIKE | 0818-0831 | IED: LLFNKVTL | 3 | 4 | 7.44 | 13 | 2.22 |
| (67) | (SEQ ID | | A: DA | | | | | |
| | NO: 3) | | | | | | | |
| 68 | SPIKE | 0818-0832 | IED: LLFNKVTL | — | 4 | 7.44 | 13 | 2.71 |
| (69) | (SEQ ID | | A: DNG | | | | | |
| | NO: 3) | | | | | | | |
| 70 | SPIKE | 0855-0869 | ANG: LTVLPPLL | — | 6 | 10.67 | 5 | 1.83 |
| (71) | (SEQ ID | | T: DEM | | | | | |
| | NO: 3) | | | | | | | |
| 72 | SPIKE | 0856-0869 | NG: LTVLPPLL | 3 | 6 | 10.67 | 5 | 1.83 |
| (73) | (SEQ ID | | T: DEM | | | | | |
| | NO: 3) | | | | | | | |
| 74 | SPIKE | 0870-0884 | KAQ: YTSALLAG | — | 3 | 5.63 | 8 | 3.33 |
| (75) | (SEQ ID | | T: ITS | | | | | |
| | NO: 3) | | | | | | | |
| 76 | SPIKE | 0924-0938 | ANQ: FNSAIGKI | — | 7 | 14.98 | 5 | 2.0 |
| (77) | (SEQ ID | | Q: DSL | | | | | |
| | NO: 3) | | | | | | | |
| 78 | SPIKE | 0934-0949 | IQD: SLSSTASA | 6 | 7 | 11.21 | 17 | 3.11 |
| (79) | (SEQ ID | | LG: KLQ | | | | | |
| | NO: 3) | | | | | | | |
| 80 | SPIKE | 0934-0949 | KQD: SLSSTASA | — | 7 | 11.21 | 17 | 4.0 |
| (81) | (SEQ ID | | LG: KLQ | | | | | |
| | NO: 3) | | | | | | | |
| 82 | SPIKE | 0959-0974 | RNT: LVKQLSSN | — | 16 | 32.76 | 9 | 1.65 |
| (83) | (SEQ ID | | FG: AIK | | | | | |
| | NO: 3) | | | | | | | |
| 84 | SPIKE | 0967-0981 | SSN: FGAISSVL | — | 4 | 7.51 | 11 | 2.29 |
| (85) | (SEQ ID | | N: DIL | | | | | |
| | NO: 3) | | | | | | | |
| 86 | SPIKE | 1010-1024 | QQL: IRAAEIRA | 2 | 4 | 6.75 | 6 | 1.40 |
| (87) | (SEQ ID | | S: ANL | | | | | |
| | NO: 3) | | | | | | | |
| 88 | SPIKE | 1015-1029 | AAE: IRASANLA | 6 | 8 | 18.74 | 14 | 3.40 |
| (89) | (SEQ ID | | A: TKM | | | | | |
| | NO: 3) | | | | | | | |
| 90 | SPIKE | 1214-1228 | WYI: WLGFIAGL | 9 | 7 | 14.24 | 12 | 2.42 |
| (91) | (SEQ ID | | I: AIV | | | | | |
| | NO: 3) | | | | | | | |
| 92 | SPIKE | 1214-1228 | WGI: WLGFIAGL | — | 7 | 14.24 | 9 | 2.67 |
| (93) | (SEQ ID | | I: AIK | | | | | |
| | NO: 3) | | | | | | | |
| 94 | NUCLEOCAPSID | 154-168 | NAA: IVLQLPQG | 2 | 6 | 10.05 | 8 | 4.00 |
| (95) | (SEQ ID | | T: TLP | | | | | |
| | NO: 371) | | | | | | | |
| 96 | ORF3A | 042-058 | PFG: WLIVGVAL | 1 | 13 | 24.28 | 24 | 4.23 |
| (97) | (SEQ ID | | LAV: FQS | | | | | |
| | NO: 372) | | | | | | | |
| 98 | ORF3A | 067-082 | KR: WQLALSKGV | 6 | 11 | 23.09 | 9 | 2.42 |
| (99) | (SEQ ID | | HF: VCN | | | | | |
| | NO: 372) | | | | | | | |
| 100 | ORF3A | 074-106 | SKG: VHFVCNLL | 38 | 53 | 95.68 | 91 | 3.26 |
| (101) | (SEQ ID | | LLFVTVYSHLLL | | | | | |
| | NO: 372) | | VAAGLEA: PFL | | | | | |
| 102 | ORF6 | 026-048 | IWN: LDYIINLI | 39 | 39 | 77.11 | 44 | 2.88 |
| (103) | (SEQ ID | | IKNLSKSLT: EN | | | | | |
| | NO: 373) | | K | | | | | |
| 104 | ORF7A | 001-016 | MKIILFLALITL | 12 | 17 | 31.91 | 56 | 6.52 |
| (105) | (SEQ ID | | A: TCE | | | | | |
| | NO: 374) | | | | | | | |
| 106 | ORF7A | 097-114 | YSP: IFLIVAAI | 12 | 23 | 41.55 | 19 | 2.09 |
| (107) | (SEQ ID | | VFIT: LCF | | | | | |
| | NO: 374) | | | | | | | |
| 108 | ORF1AB_NSP2 | 072-088 | QTP: FEIKLAKK | 7 | 10 | 18.15 | 8 | 1.10 |
| (109) | (SEQ ID | | FDT: FNG | | | | | |
| | NO: 375) | | | | | | | |
| 110 | ORF1AB_NSP2 | 268-285 | DNL: LEILQKEK | 2 | 9 | 14.52 | 10 | 1.90 |
| (111) | (SEQ ID | | VNIN: IVG | | | | | |
| | NO: 375) | | | | | | | |
| 112 | ORF1AB_NSP2 | 290-307 | NEE: IAIILASF | 9 | 16 | 28.92 | 30 | 3.29 |
| (113) | (SEQ ID | | SASTS: AF | | | | | |
| | NO: 375) | | | | | | | |
| 114 | ORF1AB_NSP2 | 437-452 | FGT: VYEKLKPV | 6 | 6 | 10.63 | 8 | 2.00 |
| (115) | (SEQ ID | | LD: WLE | | | | | |
| | NO: 375) | | | | | | | |
| 116 | ORF1AB_NSP3 | 317-334 | NQH: EVLLAPLL | 8 | 11 | 17.99 | 81 | 13.17 |
| (117) | (SEQ ID | | SAGI: FGA | | | | | |
| | NO: 376) | | | | | | | |
| 118 | ORF1AB_NSP3 | 369-383 | VSS: FLEMKSEK | 5 | 8 | 17.97 | 9 | 2.22 |
| (119) | (SEQ ID | | Q: VEQ | | | | | |
| | NO: 376) | | | | | | | |
| 120 | ORF1AB_NSP3 | 427-442 | ETK: FLTENLLL | 13 | 4 | 6.31 | 15 | 3.14 |
| (121) | (SEQ ID | | YI: DIN | | | | | |
| | NO: 376) | | | | | | | |
| 122 | ORF1AB_NSP3 | 524-539 | AKT: VLKKCKSA | 5 | 8 | 14.57 | 11 | 2 |
| (123) | (SEQ ID | | FY: ILP | | | | | |
| | NO: 376) | | | | | | | |
| 124 | ORF1AB_NSP3 | 732-751 | VIT: FDNLKTLL | 9 | 16 | 29.14 | 21 | 1.95 |
| (125) | (SEQ ID | | SLREVR: TIK | | | | | |
| | NO: 376) | | | | | | | |
| 126 | ORF1AB_NSP3 | 1254-1270 | VGD: IILKPANN | 7 | 18 | 33.29 | 10 | 2 |
| (127) | (SEQ ID | | SLK: ITE | | | | | |
| | NO: 376) | | | | | | | |
| 128 | ORF1AB_NSP3 | 1292-1308 | PNE: LSRVLGLK | 2 | 12 | 21.59 | 23 | 3 |
| (129) | (SEQ ID | | TLAT: HG | | | | | |
| | NO: 376) | | | | | | | |
| 130 | ORF1AB_NSP3 | 1350-1369 | YMP: YFFTLLLQ | 23 | 18 | 28.49 | 18 | 2.15 |
| (131) | (SEQ ID | | LCTFTR: STN | | | | | |
| | NO: 376) | | | | | | | |
| 132 | ORF1AB_NSP3 | 1408-1428 | LIN: IIIWFLLL | 32 | 27 | 54.71 | 65 | 6.13 |
| (133) | (SEQ ID | | SVCLGSL: IYS | | | | | |
| | NO: 376) | | | | | | | |
| 134 | ORF1AB_NSP3 | 1743-1757 | QPI: LLLDQALV | 5 | 4 | 8.56 | 23 | 6.86 |
| (135) | (SEQ ID | | S: DVG | | | | | |
| | NO: 376) | | | | | | | |
| 136 | ORF1AB_NSP4 | 1-30 | KI: VNNWLKQLK | 32 | 49 | 81.67 | 28 | 0.96 |
| (137) | (SEQ ID | | KVTLVFLFVAAIF | | | | | |
| | NO: 377) | | YLI: TPV | | | | | |
| 138 | ORF1AB_NSP4 | 302-315 | MR: FRRAFGEY | 0 | 8 | 16.53 | 4 | 1.75 |
| (139) | (SEQ ID | | S: HVV | | | | | |
| | NO: 377) | | | | | | | |
| 391 | ORF1AB_NSP4 | 394-415 | FSN: YLKRRVVF | 13 | 11 | 13.16 | 13 | 1.13 |
| (392) | (SEQ ID | | NGVSFSTF: EEA | | | | | |
| | NO: 377) | | | | | | | |
| 140 | ORF1AB_NSP4 | 424-441 | KEM: YLKLRSDV | 12 | 11 | 20.85 | 16 | 1.85 |
| (141) | (SEQ ID | | LLPL: TQY | | | | | |
| | NO: 377) | | | | | | | |
| 142 | ORF1AB_NSP6 | 4-30 | KRT: IKGTHHWL | 27 | 39 | 71.82 | 85 | 6.41 |
| (143) | (SEQ ID | | LLTILTSLLVLV | | | | | |
| | NO: 378) | | Q: STQ | | | | | |
| 144 | ORF1AB_NSP6 | 63-82 | KHA: FLCLFLLP | 19 | 20 | 37.07 | 29 | 1.9 |
| (145) | (SEQ ID | | SLATVA: YFN | | | | | |
| | NO: 378) | | | | | | | |
| 146 | ORF1AB_NSP6 | 111-127 | KDC: VMYASAVV | 12 | 18 | 34.15 | 26 | 2.44 |
| (147) | (SEQ ID | | LLLI: LMT | | | | | |
| | NO: 378) | | | | | | | |
| 148 | ORF1AB_NSP6 | 266-284 | IDA: FKLNIKLL | 5 | 13 | 27.78 | 15 | 3.2 |
| (149) | (SEQ ID | | GVGGK: PCI | | | | | |
| | NO: 378) | | | | | | | |
| 150 | ORF1AB_NSP7 | 8-33 | CTS: VVLLSVLQ | 11 | 33 | 56.25 | 54 | 3.09 |
| (151) | (SEQ ID | | QLRVESSSKLWA: | | | | | |
| | NO: 379) | | QCV | | | | | |
| 152 | ORF1AB_NSP7 | 46-70 | TEA: FEKMVSLL | 17 | 39 | 78.38 | 118 | 7.00 |
| (153) | (SEQ ID | | SVLLSMQGAVD: | | | | | |
| | NO: 379) | | INK | | | | | |
| 154 | ORF1AB_NSP8 | 030-050 | DSE: VVLKKLKK | 5 | 28 | 53.65 | 51 | 4 |
| (155) | (SEQ ID | | SLNVAKS: EFD | | | | | |
| | NO: 380) | | | | | | | |
| 156 | ORF1AB_NSP9 | 035-052 | TKG: GRFVLALL | 5 | 14 | 25.78 | 18 | 2.21 |
| (157) | (SEQ ID | | SDLQ: DLK | | | | | |
| | NO: 381) | | | | | | | |
| 158 | ORF1AB_NSP9 | 088-101 | LY: FIKGLNNLN | 3 | 16 | 34.47 | 9 | 1.90 |
| (159) | (SEQ ID | | R: GM | | | | | |
| | NO: 381) | | | | | | | |
| 160 | ORF1AB_RNA- | 168-183 | NPD: ILRVYANL | 3 | 11 | 23.92 | 10 | 2.25 |
| (161) | DEPENDENT_ | | GE: RVR | | | | | |
| | RNA_ | | | | | | | |
| | POLYMERASE | | | | | | | |
| | (SEQ ID | | | | | | | |
| | NO: 382) | | | | | | | |
| 162 | ORF1AB_RNA- | 568-587 | NRQ: FHQKLLKS | 6 | 20 | 35.77 | 20 | 2.14 |
| (163) | DEPENDENT_ | | IAATRG: ATV | | | | | |
| | RNA_ | | | | | | | |
| | POLYMERASE | | | | | | | |
| | (SEQ ID | | | | | | | |
| | NO: 382) | | | | | | | |
| 164 | ORF1AB_RNA- | 626-643 | MPN: MLRIMASL | 15 | 20 | 42.54 | 16 | 1.71 |
| (165) | DEPENDENT_ | | VLAR: KHT | | | | | |
| | RNA_ | | | | | | | |
| | POLYMERASE | | | | | | | |
| | (SEQ ID | | | | | | | |
| | NO: 382) | | | | | | | |
| 166 | ORF1AB_ | 030-046 | CYD: HVISTSHK | 4 | 10 | 17.06 | 16 | 1.62 |
| (167) | HELICASE | | LVL: SVN | | | | | |
| | (SEQ ID | | | | | | | |
| | NO: 383) | | | | | | | |
| 168 | ORF1AB_ | 182-198 | FTG: YRVTKNSK | 2 | 10 | 19.77 | 10 | 2.00 |
| (169) | HELICASE | | VQI: GEY | | | | | |
| | (SEQ ID | | | | | | | |
| | NO: 383) | | | | | | | |
| 170 | ORF1AB_ | 344-357 | DK: FKVNSTLE | 7 | 7 | 14.65 | 5 | 2.38 |
| (171) | HELICASE | | Q: YVF | | | | | |
| | (SEQ ID | | | | | | | |
| | NO: 383) | | | | | | | |
| 172 | ORF1AB_ | 379-401 | ATN: YDLSVVNA | 5 | 11 | 13.62 | 12 | 1.36 |
| (173) | HELICASE | | RLRAKHYVY: IG | | | | | |
| | (SEQ ID | | D | | | | | |
| | NO: 383) | | | | | | | |
| 174 | ORF1AB_3- | 401-416 | FDT: RVLSNLNL | 0 | 9 | 18.48 | 44 | 11.55 |
| (175) | TO-5_ | | PG: CDG | | | | | |
| | EXONUCLEASE | | | | | | | |
| | (SEQ ID | | | | | | | |
| | NO: 384) | | | | | | | |
| 176 | ORF1AB_ | 190-211 | PET: YFTQSRNL | 8 | 14 | 20.99 | 11 | 1.93 |
| (177) | ENDORNASE | | QEFKPRSQ: MEI | | | | | |
| | (SEQ ID | | | | | | | |
| | NO: 385) | | | | | | | |
| 178 | ORF1AB_ | 300-316 | DDF: VEIIKSQD | 4 | 8 | 13.64 | 6 | 1.2 |
| (179) | ENDORNASE | | LSV: VSK | | | | | |
| | (SEQ ID | | | | | | | |
| | NO: 385) | | | | | | | |
| 180 | ORF1AB_2- | 153-170 | ICG: FIQQKLAL | 5 | 14 | 26.33 | 20 | 2.67 |
| (181) | O-RIBOSE_ | | GGSV: AIK | | | | | |
| | METHYL- | | | | | | | |
| | TRANSFERASE | | | | | | | |
| | (SEQ ID | | | | | | | |
| | NO: 386) | | | | | | | |
| 182 | ORF1AB_2- | 191-208 | TA: FVTNVNASS | 1 | 18 | 34.78 | 8 | 1.22 |
| (183) | O-RIBOSE_ | | SEAF: LIG | | | | | |
| | METHYL- | | | | | | | |
| | TRANSFERASE | | | | | | | |
| | (SEQ ID | | | | | | | |
| | NO: 386) | | | | | | | |
| 184 | ORF1AB_2- | 267-285 | IND: MILSLLSK | 11 | 27 | 50.22 | 38 | 2.80 |
| (185) | O-RIBOSE_ | | GRLII: REN | | | | | |
| | METHYL- | | | | | | | |
| | TRANSFERASE | | | | | | | |
| | (SEQ ID | | | | | | | |
| | NO: 386) | | | | | | | |

The present disclosure also includes polypeptide fragments of the Tregitopes and detolerized antigens as described herein. The present disclosure also encompasses fragments of the variants of the Tregitopes and detolerized antigens described herein, provided said fragments and/or variants at least in part retain MHC binding propensity and/or TCR specificity.

The present disclosure also provides chimeric or fusion polypeptides (which in aspects may be isolated, synthetic, or recombinant) wherein one or more of the instantly disclosed Tregitopes or detolerized antigens is a part thereof. In aspects, a chimeric or fusion polypeptide composition comprises one or more polypeptides (e.g. Tregitope or detolerized antigen) of the instant disclosure linked to a heterologous polypeptide (e.g., but not limited to, IgG, IgM, IgA, IgD or IgE molecules or antigen-specific antibody fragments thereof (including, but not limited to, a Fab, F(ab′)₂, Fv, disulfide linked Fv, scFv, single domain antibody, closed conformation multispecific antibody, disulfide-linked scfv, diabody)). As previously stated, the term “heterologous polypeptide” is intended to mean that the one or more Tregitopes or detolerized antigens of the instant disclosure (e.g., one or more of SEQ ID NOS: 4-370, 391-440, and 448-833 or a peptide lacking one or more thereof or mutated therein) are heterologous to, or not included naturally, in the heterologous polypeptide. In aspects, the one or more Tregitopes or detolerized antigens may be inserted into the heterologous polypeptide (e.g., through mutagenesis or other known means in the art), may be added to the C-terminus (with or without the use of linkers, as is known in the art), and/or added to the N-terminus (with or without the use of linkers, as is known in the art) of the heterologous polypeptide. In aspects, the one or more Tregitopes or detolerized antigens may be inserted into or replace amino acids in a Fc domain as disclosed in U.S. Pat. Nos. 7,442,778, 7,645,861, 7,655,764, 7,655,765, and/or 7,750,128 (each of which are herein incorporated by reference in their entirety). For example, protein engineering by mutagenesis can be performed using site-directed mutagenesis techniques, or other mutagenesis techniques known in the art (see e.g., James A. Brannigan and Anthony J. Wilkinson, 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety). In aspects, chimeric or fusion polypeptides comprise one or more Tregitopes and/or detolerized antigens of the present disclosure operatively linked to a heterologous polypeptide. “Operatively linked” indicates that the polypeptide (e.g., Tregitope or detolerized antigen of the present disclosure) and the heterologous protein are fused in-frame or chemically linked or otherwise bound. For example, in aspects, the one or more Tregitopes or detolerized antigens may be covalently bound to one or more internal conjugation site(s) in an Fc domain as disclosed in U.S. Pat. Nos. 8,008,453, 9,114,175, and/or 10,188,740 (each of which are herein incorporated by reference in their entirety). In aspects, an isolated, synthetic, or recombinant chimeric or fusion polypeptide composition comprises a polypeptide, said polypeptide having a sequence comprising one or more of SEQ ID NOS: 4-370, 391-440, and 448-833 of the present disclosure, wherein said one or more of SEQ ID NOS: 4-370, 391-440, and 448-833 is not naturally included in the polypeptide and/or said of one or more of SEQ ID NOS: 4-370, 391-440, and 448-833 is not located at its natural position in the polypeptide. In aspects, the one or more of SEQ ID NOS: 4-370, 391-440, and 448-833 of the present disclosure can be joined, linked to (e.g., fused in-frame, chemically linked, or otherwise bound), and/or inserted into the polypeptide. In aspects, the one or more of SEQ ID NOS: 4-370, 391-440, and 448-833 of the present disclosure can be joined or linked to (e.g., fused in-frame, chemically-linked, or otherwise bound) to a small molecule, drug, or drag fragment, for example but not limited to, a drug or drug fragment that is binds with high affinity to defined HLAs. Similarly with the detolerized antigens, a peptide either deleted for, partially deleted for or mutated in one or more of SEQ ID NOS: 4-370, 391-440, and 448-833 can be joined or linked to (e.g., fused in-frame, chemically-linked, or otherwise bound) to a small molecule, drug, or drag fragment, for example but not limited to, a drug or drug fragment that is binds with high affinity to defined HLAs. In aspects of the above chimeric or fusion polypeptide compositions, the one or more polypeptides (Treg activating regulatory T-cell epitope, Tregitope, Tregitope peptide, or T-cell epitope polypeptide) of the present disclosure have a sequence comprising, consisting of, or consisting essentially of one or more of SEQ ID NOS: 4-370, 391-440, and 448-833, and optionally 1 to 12 additional amino acids distributed in any ratio on the N-terminus and/or C-terminus of the polypeptide of SEQ ID NOS: 4-370, 391-440, and 448-833. In further aspects, a detolerized antigen such as a SARS-CoV-2 derived protein or polypeptide deleted or mutated in one or more of the TRegitopes as set forth in SEQ ID NOS: 4-370, 391-440, and 448-833 can optionally include 1 to 12 additional amino acids distributed in any ratio on the N-terminus and/or C-terminus.

### (iii) Detolerized Mutagenesis

The present disclosure includes polypeptide fragments of SARS-CoV-2 polypeptides that are detolerized as described herein. The present disclosure also encompasses fragments of the variants of the detolerized SARS-CoV-2 polypeptides described herein, and in aspects, provided said fragments and/or variants at least in part retain MHC binding propensity and/or TCR specificity. In addition to the identification of the Tregitope polypeptides as set forth in SEQ ID NOS: 4-370, 391-440, and 448-833, the present disclosure further includes detolerized antigenic compounds thereof. Some aspects of the present disclosure concern peptides or proteins derived from one or more proteins of SARS-CoV-2 wherein a Tregitope or Tregitopes therein are compromised to inhibit, reduce or abolish engagement and/or activation of T_{Reg} cells. Tregitopes can be compromised through deletion of a Tregitope therein, partial deletion of a Tregitope therein or amino acid mutation of a Tregitope therein. For example, in aspects, removal of one or more of the identified regulatory T cell epitopes from an antigen or polypeptide sequence comprises deletion of all or some of the amino acids of the one or more regulatory T cell epitopes (Tregitopes). In aspects, said removal of the one or more regulatory T cell epitopes from an antigen or polypeptide sequence comprises deletion of some or all of the amino acids of the one or more regulatory T cell epitopes and adding one or more amino acids at the site of deletion of the regulatory T cell epitope amino acids. In aspects, said removal of the one or more regulatory T cell epitopes from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said removal of the one or more regulatory T cell epitopes from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of removal need not correspond to the number of amino acids deleted from the previously existing regulatory T cell epitope amino acids.

In addition to deletion or partial deletion of a Tregitope, detolerization may include site-directed mutagenesis of one or more amino acids within a Tregitope sequence of an antigenic peptide. Particularly, sited directed mutagenesis to provide detolerized antigens may include mutated anchoring residues (e.g. agretope mutations) and/or mutated TCR epitopes within the identified Tregitopes as set forth herein. Any amino acid substitution identified by EpiMatrix as disruptive (reduces predicted T cell epitope content) is viable. Targeted anchoring residues and specific TCR binding mutations to improve immunogenicity and decease tolerogenicity are disclosed herein. Further, by mutating TCR contacts, T cell recognition of such tolerizing epitopes can be disrupted.

Accordingly, the present disclosure encompasses any and/or all SARS-CoV-2 expressed proteins or peptide fragments thereto, wherein the Tregitopes expressed are deleted, partially deleted or mutated to the point where T_{Reg} cell engagement or activation is negatively impacted and/or abolished. All identified Tregitopes discussed herein provided a JanusMatrix homology score as described herein of 2.0 or greater. The polypeptides of SEQ ID NOS: 6, 7, 18-31, 186-231, and 448-459 were identified in the sequence of the envelope (SEQ ID NO: 1 of SARS-CoV-2). The polypeptides of SEQ ID NOS: 4, 5, 17, 32-41, 232-245, 440, and 450-471 were identified in the sequence of the membrane (SEQ ID NO: 2 of SARS-CoV-2). The polypeptides of SEQ ID NOS. 8-17, 42-93, 246-370, 422, 423, 432, 434-439, and 794-833 were identified in the sequence of the spike (SEQ ID NO: 3 of SARS-CoV-2). The polypeptides of SEQ ID NOS. 94, 95, 406-421, and 474-487 were identified in the sequence of the nucleocapsid (SEQ ID NO: 371) of SARS-CoV-2. The polypeptides of SEQ ID NOS. 96-101, 424, and 756-774 were identified in the sequence of the ORF3a protein (SEQ ID NO: 372) of SARS-CoV-2. The polypeptides of SEQ ID NOS. 102, 103, and 775 were identified in the sequence of the ORF6 protein (SEQ ID NO: 373) of SARS-CoV-2. The polypeptides of SEQ ID NOS. 104-107, 425, and 776-783 were identified in the sequence of the ORF7a protein (SEQ ID NO: 374) of SARS-CoV-2. The polypeptides of SEQ ID NOS. 108-115, 427, and 554-575 were identified in the sequence of the ORF1ab non-structural protein 2 (NSP2) protein (SEQ ID NO: 375) of SARS-CoV-2. The polypeptides of SEQ ID NOS. 116-135, 428, 429, and 576-655 were identified in the sequence of the ORF1ab non-structural protein 3 (NSP3) protein (SEQ ID NO: 376) of SARS-CoV-2. The polypeptides of SEQ ID NOS. 136-141, 391, 392, and 656-684 were identified in the sequence of the ORF1ab non-structural protein 3 (NSP4) protein (SEQ ID NO: 377) of SARS-CoV-2. The polypeptides of SEQ ID NOS. 142-149 and 685-708 were identified in the sequence of the ORF1ab non-structural protein 6 (NSP6) protein (SEQ ID NO: 378) of SARS-CoV-2. The polypeptides of SEQ ID NOS. 150-153, 393-405, and 709-718 were identified in the sequence of the ORF1ab non-structural protein 7 (NSP7) protein (SEQ ID NO: 379) of SARS-CoV-2. The polypeptides of SEQ ID NOS. 154-155 and 719-725 were identified in the sequence of the ORF1ab non-structural protein 8 (NSP8) protein (SEQ ID NO: 380) of SARS-CoV-2. The polypeptides of SEQ ID NOS. 156-159 and 726-730 were identified in the sequence of the ORF1ab non-structural protein 9 (NSP9) protein (SEQ ID NO: 381) of SARS-CoV-2. The polypeptides of SEQ ID NOS. 160-165, 431, and 731-755 were identified in the sequence of the ORF1ab RNA-dependent RNA polymerase protein (SEQ ID NO: 382) of SARS-CoV-2. The polypeptides of SEQ ID NOS. 166-173 and 515-542 were identified in the sequence of the ORF1ab helicase protein (SEQ ID NO: 383) of SARS-CoV-2. The polypeptides of SEQ ID NOS. 174-175 and 496-505 were identified in the sequence of the ORF1ab 3′-to-5′ exonuclease (SEQ ID NO: 384) of SARS-CoV-2. The polypeptides of SEQ ID NOS. 176-179 and 506-514 were identified in the sequence of the ORF1ab endoRNase protein (SEQ ID NO: 385) of SARS-CoV-2. The polypeptides of SEQ ID NOS. 180-185, 433, and 543-551 were identified in the sequence of the ORF1ab 2′O-ribose methyltransferase protein (SEQ ID NO: 386) of SARS-CoV-2. The polypeptides of SEQ ID NOS. 488 and 489 were identified in the sequence of the ORF10 protein (SEQ ID NO: 388) of SARS-CoV-2. The polypeptides of SEQ ID NOS. 490-495 were identified in the sequence of the ORF1ab 3C-like protein (SEQ ID NO: 389) of SARS-CoV-2. The polypeptides of SEQ ID NOS. 552 and 553 were identified in the sequence of the ORF1ab non-structural protein 10 (NSP10) of (SEQ ID NO: 390) of SARS-CoV-2. The polypeptides of SEQ ID NOS. 426 and 784-793 were identified in the sequence of the ORF8 protein (SEQ ID NO: 387) of SARS-CoV-2.

As such, the instant disclosure further provides detolerized SARS-CoV-2 antigens (e.g., peptides and polypeptides), and methods of producing and using the same wherein the Tregitopes therein are deleted, partially deleted, replaced or mutated as described herein. Aspects include methods for improving the efficacy of vaccine antigens against SARS-CoV-2 infection and related diseases caused by SARS-CoV-2, including COVID-19, comprising the steps of: (a) identifying constituent T cell epitopes within a SARS-CoV-2 vaccine antigen which share TCR contacts with proteins derived from either the human proteome or the human microbiome; (b) deleting/removing part or whole of the identified Tregitope from the antigen and/or introducing mutated amino acids therein that negatively impact anchoring or epitope recognition to provide a detolerized antigen and (c) administering the detolerized antigen to a subject as part of a vaccine or immunostimulatory composition, either alone or in combination with other antigenic peptides an/or other detolerized antigens. It will be appreciated that the detolerized antigen can be produced by manipulations to the nucleic acids encoding the antigen to remove and/or mutate such. It will also be appreciated that nucleic acids compositions comprised of sequences encoding the detolerized antigens can be utilized in the compositions as well and/or instead of a detolerized antigen polypeptide. In aspects, such a detolerized antigen can include:
In aspects, a detolerized antigen includes the envelope (SEQ ID NO: 1) of SARS-CoV-2 or a fragment thereof provided that said fragment contains deletion, partial deletion and/or mutation of one amino acid or more therein of one or more of SEQ ID NOS: 6, 7, 18-31, 186-231, and 448-459. In aspects, such an antigen includes polypeptides comprising/consisting/consisting essentially of detolerized antigenic variants of one or more of SEQ ID NOS: 6, 7, 18-31, 186-231, and 448-459.
In aspects, a detolerized antigen includes the membrane (SEQ ID NO: 2) of SARS-CoV-2 or a fragment thereof provided that said fragment contains deletion, partial deletion and/or mutation of one amino acid or more therein of one or more of SEQ ID NO: 4, 5, 17, 32-41, 232-245, 440, and 450-471. In aspects, such an antigen includes polypeptides comprising/consisting/consisting essentially of detolerized antigenic variants of one or more of SEQ ID NOS: 4, 5, 17, 32-41, 232-245, 440, and 450-471.
In aspects, a detolerized antigen includes the spike (SEQ ID NO: 3) of SARS-CoV-2 or a fragment thereof provided that said fragment contains deletion, partial deletion and/or mutation of one amino acid or more therein of one or more of SEQ ID NOS: 8-17, 42-93, 246-370, 422, 423, 432, 434-439, and 794-833. In aspects, such an antigen includes polypeptides comprising/consisting/consisting essentially of detolerized antigenic variants of one or more of SEQ ID NOS: 8-17, 42-93, 246-370, 422, 423, 432, 434-439, and 794-833.
In aspects, a detolerized antigen includes the nucleocapsid (SEQ ID NO: 371) of SARS-CoV-2 or a fragment thereof provided that said fragment contains deletion, partial deletion and/or mutation of one amino acid or more therein of one or more of SEQ ID NOS: 94, 95, 406-421, and 474-487. In aspects, such an antigen includes polypeptides comprising/consisting/consisting essentially of detolerized antigenic variants of one or more of SEQ ID NOS: 94, 95, 406-421, and 474-487.
In aspects, a detolerized antigen includes the ORF3a protein (SEQ ID NO: 372) of SARS-CoV-2 or a fragment thereof provided that said fragment contains deletion, partial deletion and/or mutation of one amino acid or more therein of one or more of SEQ ID NOS: 96-101, 424, and 756-774. In aspects, such an antigen includes polypeptides comprising/consisting/consisting essentially of detolerized antigenic variants of one or more of SEQ ID NOS: 96-101, 424, and 756-774.
In aspects, a detolerized antigen includes the ORF6 protein (SEQ ID NO: 373) of SARS-CoV-2 or a fragment thereof provided that said fragment contains deletion, partial deletion and/or mutation of one amino acid or more therein of one or more of SEQ ID NOS: 102, 103 and 775. In aspects, such an antigen includes polypeptides comprising/consisting/consisting essentially of detolerized antigenic variants of one or more of SEQ ID NOS: 102, 103, and 775.
In aspects, a detolerized antigen includes the ORF7a protein (SEQ ID NO: 374) of SARS-CoV-2 or a fragment thereof provided that said fragment contains deletion, partial deletion and/or mutation of one amino acid or more therein of one or more of SEQ ID NOS: 104-107, 425, and 776-783. In aspects, such an antigen includes polypeptides comprising/consisting/consisting essentially of detolerized antigenic variants of one or more of SEQ ID NOS: 104-107, 425, and 776-783.
In aspects, a detolerized antigen includes the ORF1ab non-structural protein 2 (NSP2) protein (SEQ ID NO: 375) of SARS-CoV-2 or a fragment thereof provided that said fragment contains deletion, partial deletion and/or mutation of one amino acid or more therein of one or more of SEQ ID NOS: 108-115, 427, and 554-575. In aspects, such an antigen includes polypeptides comprising/consisting/consisting essentially of detolerized antigenic variants of one or more of SEQ ID NOS: 108-115, 427, and 554-575.
In aspects, a detolerized antigen includes the ORF1ab non-structural protein 3 (NSP3) protein (SEQ ID NO: 376) of SARS-CoV-2 or a fragment thereof provided that said fragment contains deletion, partial deletion and/or mutation of one amino acid or more therein of one or more of SEQ ID NOS: 116-135, 428, 429, and 576-655. In aspects, such an antigen includes polypeptides comprising/consisting/consisting essentially of detolerized antigenic variants of one or more of SEQ ID NOS: 116-135, 428, 429, and 576-655.
In aspects, a detolerized antigen includes the ORF1ab non-structural protein 4 (NSP4) protein (SEQ ID NO: 377) of SARS-CoV-2 or a fragment thereof provided that said fragment contains deletion, partial deletion and/or mutation of one amino acid or more therein of one or more of SEQ ID NOS: 136-141, 391, 392, and 656-684. In aspects, such an antigen includes polypeptides comprising/consisting/consisting essentially of detolerized antigenic variants of one or more of SEQ ID NOS: 136-141, 391, 392, and 656-684.
In aspects, a detolerized antigen includes the ORF1ab non-structural protein 6 (NSP6) protein (SEQ ID NO: 378) of SARS-CoV-2 or a fragment thereof provided that said fragment contains deletion, partial deletion and/or mutation of one amino acid or more therein of one or more of SEQ ID NOS: 142-149 and 685-708, In aspects, such an antigen includes polypeptides comprising/consisting/consisting essentially of detolerized antigenic variants of one or more of SEQ ID NOS: 142-149 and 685-708.
In aspects, a detolerized antigen includes the ORF1ab non-structural protein 7 (NSP7) protein (SEQ ID NO: 379) of SARS-CoV-2 or a fragment thereof provided that said fragment contains deletion, partial deletion and/or mutation of one amino acid or more therein of one or more of SEQ ID NOS: 150-153, 393-405, and 709-718. In aspects, such an antigen includes polypeptides comprising/consisting/consisting essentially of detolerized antigenic variants of one or more of SEQ ID NOS: 150-153, 393-405, and 709-718.
In aspects, a detolerized antigen includes the ORF1ab non-structural protein 8 (NSP8) protein (SEQ ID NO: 380) of SARS-CoV-2 or a fragment thereof provided that said fragment contains deletion, partial deletion and/or mutation of one amino acid or more therein of one or more of SEQ ID NOS: 154-155 and 719-725. In aspects, such an antigen includes polypeptides comprising/consisting/consisting essentially of detolerized antigenic variants of one or more of SEQ ID NOS: 154-155 and 719-725.
In aspects, a detolerized antigen includes the ORF1ab non-structural protein 9 (NSP9) protein (SEQ ID NO: 381) of SARS-CoV-2 or a fragment thereof provided that said fragment contains deletion, partial deletion and/or mutation of one amino acid or more therein of one or more of SEQ ID NOS: 156-159 and 726-730. In aspects, such an antigen includes polypeptides comprising/consisting/consisting essentially of detolerized antigenic variants of one or more of SEQ ID NOS: 156-159 and 726-730.
In aspects, a detolerized antigen includes the ORF1ab RNA-dependent RNA polymerase protein (SEQ ID NO: 382) of SARS-CoV-2 or a fragment thereof provided that said fragment contains deletion, partial deletion and/or mutation of one amino acid or more therein of one or more of SEQ ID NOS: 160-165, 431, and 731-755, In aspects, such an antigen includes polypeptides comprising/consisting/consisting essentially of detolerized antigenic variants of one or more of SEQ ID NOS: 160-165, 431, and 731-755.
In aspects, a detolerized antigen includes the ORF1ab helicase protein (SEQ ID NO: 383) of SARS-CoV-2 or a fragment thereof provided that said fragment contains deletion, partial deletion and/or mutation of one amino acid or more therein of one or more of SEQ ID NOS: 166-173 and 515-542. In aspects, such an antigen includes polypeptides comprising/consisting/consisting essentially of detolerized antigenic variants of one or more of SEQ ID NOS: 166-173 and 515-542.
In aspects, a detolerized antigen includes the ORF1ab 3′-to-5′ exonuclease (SEQ ID NO: 384) of SARS-CoV-2 or a fragment thereof provided that said fragment contains deletion, partial deletion and/or mutation of one amino acid or more therein of one or more of SEQ ID NOS: 174-175 and 496-505. In aspects, such an antigen includes polypeptides comprising/consisting/consisting essentially of detolerized antigenic variants of one or more of SEQ ID NOS: 174-175 and 496-505.
In aspects, a detolerized antigen includes the ORF1ab endoRNase protein (SEQ ID NO: 385) of SARS-CoV-2 or a fragment thereof provided that said fragment contains deletion, partial deletion and/or mutation of one amino acid or more therein of one or more of SEQ ID NOS: 176-179 and 506-514. In aspects, such an antigen includes polypeptides comprising/consisting/consisting essentially of detolerized antigenic variants of one or more of SEQ ID NOS: 176-179 and 506-514.
In aspects, a detolerized antigen includes the ORF1ab 2′O-ribose methyltransferase protein (SEQ ID NO: 386) of SARS-CoV-2 or a fragment thereof provided that said fragment contains deletion, partial deletion and/or mutation of one amino acid or more therein of one or more of SEQ ID NOS: 180-185, 433, and 543-551. In aspects, such an antigen includes polypeptides comprising/consisting/consisting essentially of detolerized antigenic variants of one or more of SEQ ID NOS: 180-185, 433, and 543-551.
In aspects, a detolerized antigen includes the ORF10 protein (SEQ ID NO: 388) of SARS-CoV-2 or a fragment thereof provided that said fragment contains deletion, partial deletion and/or mutation of one amino acid or more therein of one or more of SEQ ID NOS: 488 and 489. In aspects, such an antigen includes polypeptides comprising/consisting/consisting essentially of detolerized antigenic variants of one or more of SEQ ID NOS: 488 and 489.
In aspects, a detolerized antigen includes the ORF1ab 3C-like proteinase (SEQ ID NO: 389) of SARS-CoV-2 or a fragment thereof provided that said fragment contains deletion, partial deletion and/or mutation of one amino acid or more therein of one or more of f SEQ ID NOS: 490-495. In aspects, such an antigen includes polypeptides comprising/consisting/consisting essentially of detolerized antigenic variants of one or more of SEQ ID NOS: 490-495.
In aspects, a detolerized antigen includes the ORF1ab non-structural protein 10 (NSP10) (SEQ ID NO: 390) of SARS-CoV-2 or a fragment thereof provided that said fragment contains deletion, partial deletion and/or mutation of one amino acid or more therein of one or more of SEQ ID NOS: 552 and 553. In aspects, such an antigen includes polypeptides comprising/consisting/consisting essentially of detolerized antigenic variants of one or more of SEQ ID NOS: 552 and 553.
In aspects, a detolerized antigen includes the ORFS protein (SEQ ID NO: 387) of SARS-CoV-2 or a fragment thereof provided that said fragment contains deletion, partial deletion and/or mutation of one amino acid or more therein of one or more of SEQ ID NOS: 426 and 784-793. In aspects, such an antigen includes polypeptides comprising/consisting/consisting essentially of detolerized antigenic variants of one or more of SEQ ID NOS: 426 and 784-793.

In aspects, identified Tregitopes or antigens/polypeptides that contain such Tregitopes can be detolerized by methods of deletion and/or mutation. In aspects, such methods further include making modifications (e.g., by recombinant engineering, site-directed mutagenesis) to said identified constituent T cell epitopes. In aspects, such modifications are made so as to either reduce MHC binding and/or reduce homologies between TCR contacts of said target T cell epitope and the human proteome or the human microbiome. For example, in aspects, modification or removal of one or more of the identified regulatory T cell epitopes from an antigen or polypeptide sequence comprises deletion of all or some of the amino acids of the one or more regulatory T cell epitopes. In aspects, said modification or removal of the one or more regulatory T cell epitopes from an antigen or polypeptide sequence comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects w binding of such epitopes can be disrupted by mutating anchoring residues included therein. Such mutations for the anchoring residues must select replacement amino acids which are dis-favorable for binding. Any amino acid substitution identified by EpiMatrix as disruptive (reduces predicted T cell epitope content) is viable. Further, by mutating TCR contacts, T cell recognition of such tolerizing epitopes can be disrupted. Any amino acid substitution to TCR contacts disrupt TCR recognition. Such detolerized antigens can be used to improve the efficacy of pharmaceuticals and vaccines against SARS-CoV-2 infection and related diseases caused by SARS-CoV-2, including COVID-19. The instant disclosure also provides to nucleic acids, vectors, and cells which express the instantly-disclosed detolerized antigens and the uses thereof. The instant disclosure is particularly suited to produce vaccines for humans, particularly for vaccinating against SARS-CoV-2 infection and related diseases caused by SARS-CoV-2, including COVID-19. In aspects, the instantly disclosed detolerized polypeptides, which are described in more detail below, can be produced by recombinant engineering, site-directed mutagenesis, or can be produced by direct chemical synthesis or by other recombinant methods (J Sambrook et al., Molecular Cloning: A Laboratory Manual, (2^{ED}, 1989), Cold Spring Harbor Laboratory Press, Cold Springs Harbor, N.Y. (Publ)).

In aspects, the present disclosure relates to detolerized SARS-CoV-2 envelope polypeptides of SEQ ID NO: 1 or fragments thereof, wherein one or more of SEQ ID NOS: 6, 7, 18-31, 186-231, and 448-459 is modified or removed from said detolerized envelope polypeptide of SEQ ID NO: 1 or fragment thereof. For example, in aspects, modification or removal of one or more of the identified regulatory T cell epitopes (e.g., one or more of SEQ ID NOS: 6, 7, 18-31, 186-231, and 448-459) comprises deletion of all or some of the amino acids of the one or more regulatory T cell epitopes of SEQ ID NOS: 6, 7, 18-31, 186-231, and 448-459. In aspects, said modification or removal of the one or more regulatory T cell epitopes of SEQ ID NOS: 6, 7, 18-31, 186-231, and 448-459 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 6, 7, 18-31, 186-231, and 448-459 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 6, 7, 18-31, 186-231, and 448-459 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 6, 7, 18-31, 186-231, and 448-459), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 6, 7, 18-31, 186-231, and 448-459), T cell recognition of such tolerizing epitopes can be disrupted. Any amino acid substitution to TCR contacts disrupt TCR recognition.

In aspects, mutations can be made to specific anchoring residues of the envelope polypeptide of SEQ ID NO: 1, including mutations to specific anchoring residues of the tolerizing epitopes identified therein, and the binding of such epitopes can be disrupted. In aspects, such mutations include mutating the following amino acids of SEQ ID NO: 1 or a fragment thereof, provided said fragment contains SEQ ID NO: 7: V62, L65, S67, and/or V70, especially V62, (with such amino acid positions labeled based on the full sequence of SEQ ID NO: 1). As such, in aspects the present disclosure is directed to a polypeptide comprising the amino acid sequence of SEQ ID NO: 1 or a fragment thereof, provided said fragment contains SEQ ID NO: 7, and comprising mutations to V62, L65, S67, and/or V70, especially V62 (with such amino acid positions labeled based on the full sequence of SEQ ID NO: 1). In aspects, the mutations of V62, L65, S67, and/or V70 include amino acid substitutions that are disfavorable for binding. In aspects, such mutations can be any amino acid substitution identified by EpiMatrix as disruptive (reduces predicted T cell epitope content). In aspects, such mutations can be determined by HLA binding assays, as is known in the art.

In aspects, specific mutations to the polypeptide of SEQ ID NO: 1 or a fragment thereof, provided said fragment contains SEQ ID NO: 7, include the following: V62A; V62G; V62N; V62Q; V62S; V62T; and/or S67Q, (with such amino acid positions labeled based on the full sequence of SEQ ID NO: 1). In further aspects, mutations are made to specific TCR contacts of the envelope polypeptide of SEQ ID NO: 1, including mutations to specific TCR contacts of the tolerizing epitopes identified therein, and TCR recognition of such epitopes can be disrupted. Such mutations include mutating the following amino acids of SEQ ID NO: 1 or a fragment thereof, provided said fragment contains SEQ ID NO: 7: K63, N64, N66, S68, and/or R69, especially N66 (with such amino acid positions labeled based on the full sequence of SEQ ID NO: 1). In aspects, the mutations of K63, N64, N66, S68, and/or R69 include any amino acid substitutions. As such, in aspects the present disclosure is directed to a polypeptide comprising the amino acid sequence of SEQ ID NO: 1 or a fragment thereof, provided said fragment contains SEQ ID NO: 7 and comprising mutations to K63, N64, N66, S68, and/or R69, especially N66 (with such amino acid positions labeled based on the full sequence of SEQ ID NO: 1). In aspects, the mutations of K63, N64, N66, S68, and/or R69 include any amino acid substitutions.

In other aspects, such mutations include mutating the following amino acids of SEQ ID NO: 1 or a fragment thereof, provided said fragment contains SEQ ID NO: 6: T11, L12, V14, N15, S16, V17, L19, F20, A22, F23, V24, V25, F26, L27, L28, V29, T30, L31, A32, I33, L34, A36, R38, and/or A41 (with such amino acid positions labeled based on the full sequence of SEQ ID NO: 1). As such, in aspects the present disclosure is directed to a polypeptide comprising the amino acid sequence of SEQ ID NO: 1 or a fragment thereof, provided said fragment contains SEQ ID NO: 7, and comprising mutations to T11, L12, V14, N15, S16, V17, L19, F20, A22, F23, V24, V25, F26, L27, L28, V29, T30, L31, A32, I33, L34, A36, R38, and/or A41 (with such amino acid positions labeled based on the full sequence of SEQ ID NO: 1). In some aspects, mutations are made to specific TCR contacts of the envelope polypeptide of SEQ ID NO: 1. In aspects, such mutations can be any amino acid substitution identified by EpiMatrix as disruptive (reduces predicted T cell epitope content). In aspects, such mutations can be determined by HLA binding assays, as is known in the art. Any amino acid substitution to specific TCR contacts can disrupt TCR recognition.

In aspects, such mutations include mutating the following amino acids of SEQ ID NO: 1 or a fragment thereof, provided said fragment contains SEQ ID NO: 6: L12, I13, V14, N15, S16, V17, L18, L19, F20, L21, A22, F23, V24, V25, F26, L27, L28, V29, T30, L31, A32, I33, L34T35, L37, L39, and/or C40 (with such amino acid positions labeled based on the full sequence of SEQ ID NO: 1). In aspects, the mutations of L12, I13, V14, N15, S16, V17, L18, L19, F20, L21, A22, F23, V24, V25, F26, L27, L28, V29, T30, L31, A32, I33, L34, T35, L37, L39, and/or C40 include any amino acid substitutions. As such, in aspects the present disclosure is directed to a polypeptide comprising the amino acid sequence of SEQ ID NO: 1 or a fragment thereof, provided said fragment contains SEQ ID NO: 6 and comprising mutations to K63, N64, N66, S68, and/or R69, especially N66 (with such amino acid positions labeled based on the full sequence of SEQ ID NO: 1). In aspects, the mutations of K63, N64, N66, S68, and/or R69 include any amino acid substitutions.

In further aspects, mutations are made to specific anchoring residues of the envelope polypeptide of SEQ ID NO: 1, or a fragment thereof, provided said fragment contains one or more of SEQ ID NOS: 6, 7, 18-31, 186-231, and 448-459, including mutations to specific anchoring residues of the tolerizing epitopes identified therein (e.g., one or more of SEQ ID NOS: 6, 7, 18-31, 186-231, and 448-459), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. Further, by mutating TCR contacts, including mutations to specific TCR contacts of the tolerizing epitopes identified therein (e.g., one or more of SEQ ID NOS: 6, 7, 18-31, 186-231, and 448-459), T cell recognition of such tolerizing epitopes can be disrupted. Any amino acid substitution to TCR contacts disrupt TCR recognition.

In aspects, the present disclosure relates to detolerized SARS-CoV-2 membrane polypeptides of SEQ ID NO: 2 or fragments thereof, wherein one or more of SEQ ID NOS: 4, 5, 17, 32-41, 232-245, 440, and 450-471 is modified or removed from said detolerized membrane polypeptide of SEQ ID NO: 2 or fragment thereof. For example, in aspects, modification or removal of one or more of the identified regulatory T cell epitopes (e.g., one or more of SEQ ID NOS: 4, 5, 17, 32-41, 232-245, 440, and 450-471) comprises deletion of all or some of the amino acids of the one or more regulatory T cell epitopes of SEQ ID NOS: 4, 5, 17, 32-41, 232-245, 440, and 450-471. In aspects, said modification or removal of the one or more regulatory T cell epitopes of SEQ ID NOS: 4, 5, 17, 32-41, 232-245, 440, and 450-471 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 4, 5, 17, 32-41, 232-245, 440, and 450-471 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 4, 5, 17, 32-41, 232-245, 440, and 450-471 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 4, 5, 17, 32-41, 232-245, 440, and 450-471), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 4, 5, 17, 32-41, 232-245, 440, and 450-471), T cell recognition of such tolerizing epitopes can be disrupted. Any amino acid substitution to TCR contacts disrupt TCR recognition.

In aspects, mutations are made to specific anchoring residues of the membrane polypeptide of SEQ ID NO: 2. In aspects, such mutations include mutating the following amino acids of SEQ ID NO: 2 or a fragment thereof, provided said fragment contains SEQ ID NO: 4: I118, N121, P123, and/or G126 (with such amino acid positions labeled based on the full sequence of SEQ ID NO: 2). As such, in aspects the present disclosure is directed to a polypeptide comprising the amino acid sequence of SEQ ID NO: 2 or a fragment thereof, provided said fragment contains SEQ ID NO: 4, and comprising mutations to I118, N121, P123, and/or G126 (with such amino acid positions labeled based on the full sequence of SEQ ID NO: 2). In aspects, the mutations of I118, N121, P123, and/or G126 include amino acid substitutions that are disfavorable for binding. In aspects, such mutations can be any amino acid substitution identified by EpiMatrix as disruptive (reduces predicted T cell epitope content). In aspects, such mutations can be determined by HLA binding assays, as is known in the art. In aspects, specific mutations to the polypeptide of SEQ ID NO: 2 or a fragment thereof, provided said fragment contains SEQ ID NO: 4, include the following: I118A; I118G; I118N; I118Q; I118S; I118T; N121P; P123Q; P123G; and/or G126P (with such amino acid positions labeled based on the full sequence of SEQ ID NO: 2).

In aspects, mutations are made to specific TCR contacts of the membrane polypeptide of SEQ ID NO: 2, including mutations to specific TCR contacts of the tolerizing epitopes identified therein, and TCR recognition of such epitopes can be disrupted. Any amino acid substitution can disrupt TCR recognition. In aspects, such mutations include mutating the following amino acids of SEQ ID NO: 2 or a fragment thereof, provided said fragment contains SEQ ID NO: 4: L119, L120, V122, L124, and/or H125, especially V122 (with such amino acid positions labeled based on the full sequence of SEQ ID NO: 2). As such, in aspects the present disclosure is directed to a polypeptide comprising the amino acid sequence of SEQ ID NO: 2 or a fragment thereof, provided said fragment contains SEQ ID NO: 4, and comprising mutations to L119, L120, V122, L124, and/or H125, especially V122 (with such amino acid positions labeled based on the full sequence of SEQ ID NO: 2). In aspects, the mutations of L119, L120, V122, L124, and/or H125 include any amino acid substitutions.

In aspects, mutations are made to specific anchoring residues of the membrane polypeptide of SEQ ID NO: 2, including mutations to specific anchoring residues of the tolerizing epitopes identified therein, and the binding of such epitopes can be disrupted. Such mutations for the anchoring residues must select replacement amino acids which are dis-favorable for binding. In aspects, such mutations include mutating the following amino acids of SEQ ID NO: 2 or a fragment thereof, provided said fragment contains SEQ ID NO: 440: Y179, G182, S184, and/or V187 (with such amino acid positions labeled based on the full sequence of SEQ ID NO: 2). As such, in aspects the present disclosure is directed to a polypeptide comprising the amino acid sequence of SEQ ID NO: 2 or a fragment thereof, provided said fragment contains SEQ ID NO: 440 and comprising mutations to Y179, G182, S184, and/or V187 (with such amino acid positions labeled based on the full sequence of SEQ ID NO: 2). In aspects, the mutations of Y179, G182, S184, and/or V187 include amino acid substitutions that are disfavorable for binding. In aspects, such mutations can be any amino acid substitution identified by EpiMatrix as disruptive (reduces predicted T cell epitope content). In aspects, such mutations can be determined by HLA binding assays, as is known in the art. In aspects, specific mutations to the polypeptide of SEQ ID NO: 2 or a fragment thereof, provided said fragment contains SEQ ID NO: 440, include the following: Y179A; Y179N; Y179Q; Y1795; Y179T; S184G; S184Q; and/or S184T (with such amino acid positions labeled based on the full sequence of SEQ ID NO: 2).

In aspects, mutations are made to specific TCR contacts of the membrane polypeptide of SEQ ID NO: 2, including mutations to specific TCR contacts of the tolerizing epitopes identified therein, and TCR recognition of such epitopes can be disrupted. Any amino acid substitution can disrupt TCR recognition. In aspects, such mutations include mutating the following amino acids of SEQ ID NO: 2 or a fragment thereof, provided said fragment contains SEQ ID NO: 440: K180, L181, A183, Q185, and/or R186, especially A183 (with such amino acid positions labeled based on the full sequence of SEQ ID NO: 2). As such, in aspects the present disclosure is directed to a polypeptide comprising the amino acid sequence of SEQ ID NO: 2 or a fragment thereof, provided said fragment contains SEQ ID NO: 440 and comprising mutations to K180, L181, A183, Q185, and/or R186, especially A183 (with such amino acid positions labeled based on the full sequence of SEQ ID NO: 2). In aspects, the mutations of K180, L181, A183, Q185, and/or R186 include any amino acid substitutions.

In aspects, mutations are made to specific anchoring residues of the membrane polypeptide of SEQ ID NO: 2, or a fragment thereof, provided said fragment contains one or more of SEQ ID NOS: 4, 5, 17, 32-41, 232-245, 440, and 450-471, including mutations to specific anchoring residues of the tolerizing epitopes identified therein (e.g., one or more of SEQ ID NOS: 4, 5, 17, 32-41, 232-245, 440, and 450-471), and the binding of such epitopes can be disrupted. Any amino acid substitution identified by EpiMatrix as disruptive (reduces predicted T cell epitope content) is viable. Further, by mutating TCR contacts, including mutations to specific TCR contacts of the tolerizing epitopes identified therein (e.g., one or more of SEQ ID NOS: 4, 5, 17, 32-41, 232-245, 440, and 450-471), T cell recognition of such tolerizing epitopes can be disrupted. Any amino acid substitution to TCR contacts disrupt TCR recognition.

In aspects, the present disclosure relates to detolerized SARS-CoV-2 spike polypeptides of SEQ ID NO: 3 or fragments thereof, wherein one or more of SEQ ID NOS: 8-17, 42-93, 246-370, 422, 423, 432, 434-439, and 794-833 is modified or removed from said detolerized spike polypeptide of SEQ ID NO: 3 or fragment thereof. For example, in aspects, modification or removal of one or more of the identified regulatory T cell epitopes (e.g., one or more of SEQ ID NOS: 8-17, 42-93, 246-370, 422, 423, 432, 434-439, and 794-833) comprises deletion of all or some of the amino acids of the one or more regulatory T cell epitopes of SEQ ID NOS: 8-17, 42-93, 246-370, 422, 423, 432, 434-439, and 794-833. In aspects, said modification or removal of the one or more regulatory T cell epitopes of SEQ ID NOS: 8-17, 42-93, 246-370, 422, 423, 432, 434-439, and 794-833 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 8-17, 42-93, 246-370, 422, 423, 432, 434-439, and 794-833 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 8-17, 42-93, 246-370, 422, 423, 432, 434-439, and 794-833 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 8-17, 42-93, 246-370, 422, 423, 432, 434-439, and 794-833), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 8-17, 42-93, 246-370, 422, 423, 432, 434-439, and 794-833), T cell recognition of such tolerizing epitopes can be disrupted. Any amino acid substitution to TCR contacts disrupt TCR recognition.

In aspects, mutations are made to specific anchoring residues of the spike polypeptide of SEQ ID NO: 3 (or a polypeptide of SEQ ID NO: 3 without the first 15 amino acids of SEQ ID NO: 3), including mutations to specific anchoring residues of the tolerizing epitopes identified therein, and the binding of such epitopes can be disrupted. Such mutations for the anchoring residues must select replacement amino acids which are dis-favorable for binding. In aspects, such mutations include mutating the following amino acids of SEQ ID NO: 3 (or a polypeptide of SEQ ID NO: 3 without the first 15 amino acids of SEQ ID NO: 3) or a fragment thereof, provided said fragment contains SEQ ID NO: 8: F28, S31, L33, T36, D38, and/or L41 (with such amino acid positions labeled based on the full sequence of SEQ ID NO: 3). As such, in aspects the present disclosure is directed to a polypeptide comprising the amino acid sequence of SEQ ID NO: 3 (or a polypeptide of SEQ ID NO: 3 without the first 15 amino acids of SEQ ID NO: 3) or a fragment thereof, provided said fragment contains SEQ ID NO: 8, and comprising mutations to F28, S31, L33, T36, D38, and/or L41 (with such amino acid positions labeled based on the full sequence of SEQ ID NO: 3 In aspects, the mutations of F28, S31, L33, T36, D38, and/or L41 include amino acid substitutions that are disfavorable for binding. In aspects, such mutations can be any amino acid substitution identified by EpiMatrix as disruptive (reduces predicted T cell epitope content). In aspects, such mutations can be determined by HLA binding assays, as is known in the art. In aspects, specific mutations to the polypeptide of SEQ ID NO: 3 (or a polypeptide of SEQ ID NO: 3 without the first 15 amino acids of SEQ ID NO: 3) or a fragment thereof, provided said fragment contains SEQ ID NO: 7, include the following: F28G; F28A; F28N; F28T; F28S; F28Q, S31G; S31T and/or L33Q (with such amino acid positions labeled based on the full sequence of SEQ ID NO: 371).

In aspects, mutations are made to specific TCR contacts of the spike polypeptide of SEQ ID NO: 3 (or a polypeptide of SEQ ID NO: 3 without the first 15 amino acids of SEQ ID NO: 3), including mutations to specific TCR contacts of the tolerizing epitopes identified therein, and TCR recognition of such epitopes can be disrupted. Any amino acid substitution can disrupt TCR recognition. In aspects, such mutations include mutating the following amino acids of SEQ ID NO: 3 (or a polypeptide of SEQ ID NO: 3 without the first 15 amino acids of SEQ ID NO: 3) or a fragment thereof, provided said fragment contains SEQ ID NO: 8: R29, S30, V32, H34, S35, Q37, L39, and/or F40, especially V32 and/or Q37, (with such amino acid positions labeled based on the full sequence of SEQ ID NO: 3). As such, in aspects the present disclosure is directed to a polypeptide comprising the amino acid sequence of SEQ ID NO: 3 (or a polypeptide of SEQ ID NO: 3 without the first 15 amino acids of SEQ ID NO: 3) or a fragment thereof, provided said fragment contains SEQ ID NO: 8, and comprising mutations to R29, S30, V32, H34, S35, Q37, L39, and/or F40, especially V32 and/or Q37, (with such amino acid positions labeled based on the full sequence of SEQ ID NO: 3). In aspects, the mutations of R29, S30, V32, H34, S35, Q37, L39, and/or F40 include any amino acid substitutions.

In aspects, mutations are made to specific anchoring residues of the spike polypeptide of SEQ ID NO: 3 (or a polypeptide of SEQ ID NO: 3 without the first 15 amino acids) or a fragment thereof, provided said fragment contains SEQ ID NO: 9, I195, V198, D200 and/or Q203 (with such amino acid positions labeled based on the full sequence of SEQ ID NO: 3). As such, in aspects the present disclosure is directed to a polypeptide comprising the amino acid sequence of SEQ ID NO: 3 (or a polypeptide of SEQ ID NO: 3 without the first 15 amino acids of SEQ ID NO: 3) or a fragment thereof, provided said fragment contains SEQ ID NO: 9, and comprising mutations to I195, V198, D200 and/or Q203 (with such amino acid positions labeled based on the full sequence of SEQ ID NO: 3). In aspects, the mutations of I195, V198, D200 and/or Q203 include amino acid substitutions that are disfavorable for binding. In aspects, such mutations can be any amino acid substitution identified by EpiMatrix as disruptive (reduces predicted T cell epitope content). In aspects, such mutations can be determined by HLA binding assays, as is known in the art. In aspects, specific mutations to the polypeptide of SEQ ID NO: 3 (or a polypeptide of SEQ ID NO: 3 without the first 15 amino acids of SEQ ID NO: 3) or a fragment thereof, provided said fragment contains SEQ ID NO: 9, include the following: I195A; I195G; I195N; I195S; I195T; I195Q; V198G; V198T; V198N; Q203E; Q203G; and/or Q203T (with such amino acid positions labeled based on the full sequence of SEQ ID NO: 3).

In aspects, mutations are made to specific TCR contacts of the spike polypeptide of SEQ ID NO: 3 (or a polypeptide of SEQ ID NO: 3 without the first 15 amino acids of SEQ ID NO: 3), including mutations to specific TCR contacts of the tolerizing epitopes identified therein, and TCR recognition of such epitopes can be disrupted. Any amino acid substitution can disrupt TCR recognition. In aspects, such mutations include mutating the following amino acids of SEQ ID NO: 3 (or a polypeptide of SEQ ID NO: 3 without the first 15 amino acids of SEQ ID NO: 3) or a fragment thereof, provided said fragment contains SEQ ID NO: 9: N196, L197, R199, L201, and/or P202, especially R199, (with such amino acid positions labeled based on the full sequence of SEQ ID NO: 3). As such, in aspects the present disclosure is directed to a polypeptide comprising the amino acid sequence of SEQ ID NO: 3 (or a polypeptide of SEQ ID NO: 3 without the first 15 amino acids of SEQ ID NO: 3) or a fragment thereof, provided said fragment contains SEQ ID NO: 9, and comprising mutations to N196, L197, R199, L201, and/or P202, especially R199, (with such amino acid positions labeled based on the full sequence of SEQ ID NO: 3). In aspects, the mutations of N196, L197, R199, L201, and/or P202 include any amino acid substitutions. By way of further example, SEQ ID NO: 434 sets forth two identified Tregitope mutations within SEQ ID NO: 9.

In aspects, mutations are made to specific anchoring residues of the spike polypeptide of SEQ ID NO: 3 (or a polypeptide of SEQ ID NO: 3 without the first 15 amino acids of SEQ ID NO: 3), including mutations to specific anchoring residues of the tolerizing epitopes identified therein, and the binding of such epitopes can be disrupted. Such mutations for the anchoring residues must select replacement amino acids which are dis-favorable for binding. In aspects, such mutations include mutating the following amino acids of SEQ ID NO: 3 (or a polypeptide of SEQ ID NO: 3 without the first 15 amino acids of SEQ ID NO: 3) or a fragment thereof, provided said fragment contains SEQ ID NO: 10: I220, F223, T225, and/or A228 (with such amino acid positions labeled based on the full sequence of SEQ ID NO: 3). As such, in aspects the present disclosure is directed to a polypeptide comprising the amino acid sequence of SEQ ID NO: 3 (or a polypeptide of SEQ ID NO: 3 without the first 15 amino acids of SEQ ID NO: 3) or a fragment thereof, provided said fragment contains SEQ ID NO: 10, and comprising mutations to I220, F223, T225, and/or A228 (with such amino acid positions labeled based on the full sequence of SEQ ID NO: 3). In aspects, the mutations of I220, F223, T225, and/or A228 include amino acid substitutions that are disfavorable for binding. In aspects, such mutations can be any amino acid substitution identified by EpiMatrix as disruptive (reduces predicted T cell epitope content). In aspects, such mutations can be determined by HLA binding assays, as is known in the art. In aspects, specific mutations to the polypeptide of SEQ ID NO: 3 (or a polypeptide of SEQ ID NO: 3 without the first 15 amino acids of SEQ ID NO: 3) or a fragment thereof, provided said fragment contains SEQ ID NO: 10, include the following: I220A; I220G; I220N; I220Q; I220S; I220T; and/or T225Q (with such amino acid positions labeled based on the full sequence of SEQ ID NO: 3).

In aspects, mutations are made to specific TCR contacts of the spike polypeptide of SEQ ID NO: 3 (or a polypeptide of SEQ ID NO: 3 without the first 15 amino acids of SEQ ID NO: 3), including mutations to specific TCR contacts of the tolerizing epitopes identified therein, and TCR recognition of such epitopes can be disrupted. Any amino acid substitution can disrupt TCR recognition. In aspects, such mutations include mutating the following amino acids of SEQ ID NO: 3 (or a polypeptide of SEQ ID NO: 3 without the first 15 amino acids of SEQ ID NO: 3) or a fragment thereof, provided said fragment contains SEQ ID NO: 10: T221, R222, Q224, L226, and/or L227, especially Q224, (with such amino acid positions labeled based on the full sequence of SEQ ID NO: 3). As such, in aspects the present disclosure is directed to a polypeptide comprising the amino acid sequence of SEQ ID NO: 3 (or a polypeptide of SEQ ID NO: 3 without the first 15 amino acids of SEQ ID NO: 3) or a fragment thereof, provided said fragment contains SEQ ID NO: 10, and comprising mutations to T221, R222, Q224, L226, and/or L227, especially Q224, (with such amino acid positions labeled based on the full sequence of SEQ ID NO: 3). In aspects, the mutations of T221, R222, Q224, L226, and/or L227 include any amino acid substitutions. By way of further example, SEQ ID NO: 435 sets forth one identified Tregitope mutations within SEQ ID NO: 10.

In aspects, mutations are made to specific anchoring residues of the spike polypeptide of SEQ ID NO: 3 (or a polypeptide of SEQ ID NO: 3 without the first 15 amino acids of SEQ ID NO: 3), including mutations to specific anchoring residues of the tolerizing epitopes identified therein, and the binding of such epitopes can be disrupted. Such mutations for the anchoring residues must select replacement amino acids which are dis-favorable for binding. In aspects, such mutations include mutating the following amino acids of SEQ ID NO: 3 (or a polypeptide of SEQ ID NO: 3 without the first 15 amino acids of SEQ ID NO: 3) or a fragment thereof, provided said fragment contains SEQ ID NO: 11: Y254, P257, Y259, and/or L262 (with such amino acid positions labeled based on the full sequence of SEQ ID NO: 3). As such, in aspects the present disclosure is directed to a polypeptide comprising the amino acid sequence of SEQ ID NO: 3 (or a polypeptide of SEQ ID NO: 3 without the first 15 amino acids of SEQ ID NO: 3) or a fragment thereof, provided said fragment contains SEQ ID NO: 11, and comprising mutations to Y254, P257, Y259, and/or L262 (with such amino acid positions labeled based on the full sequence of SEQ ID NO: 3). In aspects, the mutations of Y254, P257, Y259, and/or L262 include amino acid substitutions that are disfavorable for binding. In aspects, such mutations can be any amino acid substitution identified by EpiMatrix as disruptive (reduces predicted T cell epitope content). In aspects, such mutations can be determined by HLA binding assays, as is known in the art. In aspects, specific mutations to the polypeptide of SEQ ID NO: 3 (or a polypeptide of SEQ ID NO: 3 without the first 15 amino acids of SEQ ID NO: 3) or a fragment thereof, provided said fragment contains SEQ ID NO: 11, include the following: Y254A; Y254G; Y254N; Y254Q; Y254S; Y254T; T259G; and/or T259Q (with such amino acid positions labeled based on the full sequence of SEQ ID NO: 3).

In aspects, mutations are made to specific TCR contacts of the spike polypeptide of SEQ ID NO: 3 (or a polypeptide of SEQ ID NO: 3 without the first 15 amino acids of SEQ ID NO: 3), including mutations to specific TCR contacts of the tolerizing epitopes identified therein, and TCR recognition of such epitopes can be disrupted. Any amino acid substitution can disrupt TCR recognition. In aspects, such mutations include mutating the following amino acids of SEQ ID NO: 3 (or a polypeptide of SEQ ID NO: 3 without the first 15 amino acids of SEQ ID NO: 3) or a fragment thereof, provided said fragment contains SEQ ID NO: 11: L255, Q256, R258, F260, and/or L261, especially R258, (with such amino acid positions labeled based on the full sequence of SEQ ID NO: 3). As such, in aspects the present disclosure is directed to a polypeptide comprising the amino acid sequence of SEQ ID NO: 3 (or a polypeptide of SEQ ID NO: 3 without the first 15 amino acids of SEQ ID NO: 3) or a fragment thereof, provided said fragment contains SEQ ID NO: 11, and comprising mutations to L255, Q256, R258, F260, and/or L261, especially R258, (with such amino acid positions labeled based on the full sequence of SEQ ID NO: 3). In aspects, the mutations of L255, Q256, R258, F260, and/or L261 include any amino acid substitutions.

In aspects, mutations are made to specific anchoring residues of the spike polypeptide of SEQ ID NO: 3 (or a polypeptide of SEQ ID NO: 3 without the first 15 amino acids of SEQ ID NO: 3), including mutations to specific anchoring residues of the tolerizing epitopes identified therein, and the binding of such epitopes can be disrupted. Such mutations for the anchoring residues must select replacement amino acids which are dis-favorable for binding. In aspects, such mutations include mutating the following amino acids of SEQ ID NO: 3 (or a polypeptide of SEQ ID NO: 3 without the first 15 amino acids of SEQ ID NO: 3) or a fragment thereof, provided said fragment contains SEQ ID NO: 12: V496, S499, E501, and/or H504 (with such amino acid positions labeled based on the full sequence of SEQ ID NO: 3). As such, in aspects the present disclosure is directed to a polypeptide comprising the amino acid sequence of SEQ ID NO: 3 (or a polypeptide of SEQ ID NO: 3 without the first 15 amino acids of SEQ ID NO: 3) or a fragment thereof, provided said fragment contains SEQ ID NO: 12, and comprising mutations to V496, S499, E501, and/or H504 (with such amino acid positions labeled based on the full sequence of SEQ ID NO: 3). In aspects, the mutations of V496, S499, E501, and/or H504 include amino acid substitutions that are disfavorable for binding. In aspects, such mutations can be any amino acid substitution identified by EpiMatrix as disruptive (reduces predicted T cell epitope content). In aspects, such mutations can be determined by HLA binding assays, as is known in the art. In aspects, specific mutations to the polypeptide of SEQ ID NO: 3 (or a polypeptide of SEQ ID NO: 3 without the first 15 amino acids of SEQ ID NO: 3) or a fragment thereof, provided said fragment contains SEQ ID NO: 12, include the following: V496A; V496G; V496N; V496Q; V496S; V496T; S499G; S499Q; and/or S499T (with such amino acid positions labeled based on the full sequence of SEQ ID NO: 3).

In aspects, mutations are made to specific TCR contacts of the spike polypeptide of SEQ ID NO: 3 (or a polypeptide of SEQ ID NO: 3 without the first 15 amino acids of SEQ ID NO: 3), including mutations to specific TCR contacts of the tolerizing epitopes identified therein, and TCR recognition of such epitopes can be disrupted. Any amino acid substitution can disrupt TCR recognition. In aspects, such mutations include mutating the following amino acids of SEQ ID NO: 3 (or a polypeptide of SEQ ID NO: 3 without the first 15 amino acids of SEQ ID NO: 3) or a fragment thereof, provided said fragment contains SEQ ID NO: 12: V497, L498, F500, L502, and/or L503, especially F500, (with such amino acid positions labeled based on the full sequence of SEQ ID NO: 3). As such, in aspects the present disclosure is directed to a polypeptide comprising the amino acid sequence of SEQ ID NO: 3 (or a polypeptide of SEQ ID NO: 3 without the first 15 amino acids of SEQ ID NO: 3) or a fragment thereof, provided said fragment contains SEQ ID NO: 12, and comprising mutations to V497, L498, F500, L502, and/or L503, especially F500, (with such amino acid positions labeled based on the full sequence of SEQ ID NO: 3). In aspects, the mutations of V497, L498, F500, L502, and/or L503 include any amino acid substitutions. By way of further example, SEQ ID NO: 436 sets forth one identified Tregitope mutation within SEQ ID NO: 12. In aspects, mutations are made to specific anchoring residues of the spike polypeptide of SEQ ID NO: 3 (or a polypeptide of SEQ ID NO: 3 without the first 15 amino acids of SEQ ID NO: 3), including mutations to specific anchoring residues of the tolerizing epitopes identified therein, and the binding of such epitopes can be disrupted. Such mutations for the anchoring residues must select replacement amino acids which are dis-favorable for binding. In aspects, such mutations include mutating the following amino acids of SEQ ID NO: 3 (or a polypeptide of SEQ ID NO: 3 without the first 15 amino acids of SEQ ID NO: 3) or a fragment thereof, provided said fragment contains SEQ ID NO: 13: L806, N809, V811, and/or A814 (with such amino acid positions labeled based on the full sequence of SEQ ID NO: 3). As such, in aspects the present disclosure is directed to a polypeptide comprising the amino acid sequence of SEQ ID NO: 3 (or a polypeptide of SEQ ID NO: 3 without the first 15 amino acids of SEQ ID NO: 3) or a fragment thereof, provided said fragment contains SEQ ID NO: 13, and comprising mutations to L806, N809, V811, and/or A814 (with such amino acid positions labeled based on the full sequence of SEQ ID NO: 3). In aspects, the mutations of L806, N809, V811, and/or A814 include amino acid substitutions that are disfavorable for binding. In aspects, such mutations can be any amino acid substitution identified by EpiMatrix as disruptive (reduces predicted T cell epitope content). In aspects, such mutations can be determined by HLA binding assays, as is known in the art. In aspects, specific mutations to the polypeptide of SEQ ID NO: 3 (or a polypeptide of SEQ ID NO: 3 without the first 15 amino acids of SEQ ID NO: 3) or a fragment thereof, provided said fragment contains SEQ ID NO: 13, include the following: L806A; L806G; L806N; L806Q; L806S; L806T; and/or N809G (with such amino acid positions labeled based on the full sequence of SEQ ID NO: 3).

In aspects, mutations are made to specific TCR contacts of the spike polypeptide of SEQ ID NO: 3 (or a polypeptide of SEQ ID NO: 3 without the first 15 amino acids of SEQ ID NO: 3), including mutations to specific TCR contacts of the tolerizing epitopes identified therein, and TCR recognition of such epitopes can be disrupted. Any amino acid substitution can disrupt TCR recognition. In aspects, such mutations include mutating the following amino acids of SEQ ID NO: 3 (or a polypeptide of SEQ ID NO: 3 without the first 15 amino acids of SEQ ID NO: 3) or a fragment thereof, provided said fragment contains SEQ ID NO: 13: L807, F808, K810, T812, and/or L813, especially K810, (with such amino acid positions labeled based on the full sequence of SEQ ID NO: 3). As such, in aspects the present disclosure is directed to a polypeptide comprising the amino acid sequence of SEQ ID NO: 3 (or a polypeptide of SEQ ID NO: 3 without the first 15 amino acids of SEQ ID NO: 3) or a fragment thereof, provided said fragment contains SEQ ID NO: 13, and comprising mutations to L807, F808, K810, T812, and/or L813, especially K810, (with such amino acid positions labeled based on the full sequence of SEQ ID NO: 371). In aspects, the mutations of L807, F808, K810, T812, and/or L813 include any amino acid substitutions. By way of further example, SEQ ID NOS: 437 and 438 sets forth two identified Tregitope mutations within SEQ ID NO: 13. In aspects, mutations are made to specific anchoring residues of the spike polypeptide of SEQ ID NO: 3 (or a polypeptide of SEQ ID NO: 3 without the first 15 amino acids of SEQ ID NO: 3), including mutations to specific anchoring residues of the tolerizing epitopes identified therein, and the binding of such epitopes can be disrupted. Such mutations for the anchoring residues must select replacement amino acids which are dis-favorable for binding. In aspects, such mutations include mutating the following amino acids of SEQ ID NO: 3 (or a polypeptide of SEQ ID NO: 371 without the first 15 amino acids of SEQ ID NO: 3) or a fragment thereof, provided said fragment contains SEQ ID NO: 14: L843, L846, P848, and/or T851 (with such amino acid positions labeled based on the full sequence of SEQ ID NO: 3). As such, in aspects the present disclosure is directed to a polypeptide comprising the amino acid sequence of SEQ ID NO: 3 (or a polypeptide of SEQ ID NO: 3 without the first 15 amino acids of SEQ ID NO: 3) or a fragment thereof, provided said fragment contains SEQ ID NO: 14, and comprising mutations to L843, L846, P848, and/or T851 (with such amino acid positions labeled based on the full sequence of SEQ ID NO: 3). In aspects, the mutations of L843, L846, P848, and/or T851 include amino acid substitutions that are disfavorable for binding. In aspects, such mutations can be any amino acid substitution identified by EpiMatrix as disruptive (reduces predicted T cell epitope content). In aspects, such mutations can be determined by HLA binding assays, as is known in the art. In aspects, specific mutations to the polypeptide of SEQ ID NO: 3 (or a polypeptide of SEQ ID NO: 3 without the first 15 amino acids of SEQ ID NO: 3) or a fragment thereof, provided said fragment contains SEQ ID NO: 14, include the following: L843A; L843G; L843N; L843Q; L843S; L843T; L846G; L846T; and/or P848Q (with such amino acid positions labeled based on the full sequence of SEQ ID NO: 3).

In aspects, mutations are made to specific TCR contacts of the spike polypeptide of SEQ ID NO: 3 (or a polypeptide of SEQ ID NO: 3 without the first 15 amino acids of SEQ ID NO: 3), including mutations to specific TCR contacts of the tolerizing epitopes identified therein, and TCR recognition of such epitopes can be disrupted. Any amino acid substitution can disrupt TCR recognition. In aspects, such mutations include mutating the following amino acids of SEQ ID NO: 3 (or a polypeptide of SEQ ID NO: 3 without the first 15 amino acids of SEQ ID NO: 3) or a fragment thereof, provided said fragment contains SEQ ID NO: 14: T844, V845, P847, L849, and/or L850, especially P847, (with such amino acid positions labeled based on the full sequence of SEQ ID NO: 3). As such, in aspects the present disclosure is directed to a polypeptide comprising the amino acid sequence of SEQ ID NO: 3 (or a polypeptide of SEQ ID NO: 3 without the first 15 amino acids of SEQ ID NO: 3) or a fragment thereof, provided said fragment contains SEQ ID NO: 14, and comprising mutations to T844, V845, P847, L849, and/or L850, especially P847, (with such amino acid positions labeled based on the full sequence of SEQ ID NO: 3). In aspects, the mutations T844, V845, P847, L849, and/or L850 include any amino acid substitutions. In aspects, mutations are made to specific anchoring residues of the spike polypeptide of SEQ ID NO: 3 (or a polypeptide of SEQ ID NO: 3 without the first 15 amino acids of SEQ ID NO: 3), including mutations to specific anchoring residues of the tolerizing epitopes identified therein, and the binding of such epitopes can be disrupted. Such mutations for the anchoring residues must select replacement amino acids which are dis-favorable for binding. In aspects, such mutations include mutating the following amino acids of SEQ ID NO: 3 (or a polypeptide of SEQ ID NO: 3 without the first 15 amino acids of SEQ ID NO: 3) or a fragment thereof, provided said fragment contains SEQ ID NO: 15: F912, A915, G917, Q920, L923, T926, S928, and/or G931 (with such amino acid positions labeled based on the full sequence of SEQ ID NO: 3). As such, in aspects the present disclosure is directed to a polypeptide comprising the amino acid sequence of SEQ ID NO: 3 (or a polypeptide of SEQ ID NO: 3 without the first 15 amino acids of SEQ ID NO: 3) or a fragment thereof, provided said fragment contains SEQ ID NO: 15, and comprising mutations to F912, A915, G917, Q920, L923, T926, S928, and/or G931 (with such amino acid positions labeled based on the full sequence of SEQ ID NO: 3). In aspects, the mutations of F912, A915, G917, Q920, L923, T926, S928, and/or G931 include amino acid substitutions that are disfavorable for binding. In aspects, such mutations can be any amino acid substitution identified by EpiMatrix as disruptive (reduces predicted T cell epitope content). In aspects, such mutations can be determined by HLA binding assays, as is known in the art. In aspects, specific mutations to the polypeptide of SEQ ID NO: 3 (or a polypeptide of SEQ ID NO: 3 without the first 15 amino acids of SEQ ID NO: 3) or a fragment thereof, provided said fragment contains SEQ ID NO: 15, include the following: F912A; F912G; F912N; F912Q; F912S; F912T; A915G; L923A; L923G; L923N; L923Q; L923S; L923T, and/or T926G (with such amino acid positions labeled based on the full sequence of SEQ ID NO: 3).

In aspects, mutations are made to specific TCR contacts of the spike polypeptide of SEQ ID NO: 3 (or a polypeptide of SEQ ID NO: 3 without the first 15 amino acids of SEQ ID NO: 3), including mutations to specific TCR contacts of the tolerizing epitopes identified therein, and TCR recognition of such epitopes can be disrupted. Any amino acid substitution can disrupt TCR recognition. In aspects, such mutations include mutating the following amino acids of SEQ ID NO: 3 (or a polypeptide of SEQ ID NO: 3 without the first 15 amino acids of SEQ ID NO: 3) or a fragment thereof, provided said fragment contains SEQ ID NO: 15: N913, S914, I916, K918, I919, S924, S925, A927, A929, and/or L930, especially I916 and/or A927, (with such amino acid positions labeled based on the full sequence of SEQ ID NO: 3). As such, in aspects the present disclosure is directed to a polypeptide comprising the amino acid sequence of SEQ ID NO: 3 (or a polypeptide of SEQ ID NO: 3 without the first 15 amino acids of SEQ ID NO: 3) or a fragment thereof, provided said fragment contains SEQ ID NO: 15, and comprising mutations to N913, S914, I916, K918, I919, S924, S925, A927, A929, and/or L930, especially I916 and/or A927, (with such amino acid positions labeled based on the full sequence of SEQ ID NO: 3). In aspects, the mutations N913, S914, I916, K918, I919, S924, S925, A927, A929, and/or L930 include any amino acid substitutions. In aspects, mutations are made to specific anchoring residues of the spike polypeptide of SEQ ID NO: 3 (or a polypeptide of SEQ ID NO: 3 without the first 15 amino acids of SEQ ID NO: 3), including mutations to specific anchoring residues of the tolerizing epitopes identified therein, and the binding of such epitopes can be disrupted. Such mutations for the anchoring residues must select replacement amino acids which are dis-favorable for binding. In aspects, such mutations include mutating the following amino acids of SEQ ID NO: 3 (or a polypeptide of SEQ ID NO: 3 without the first 15 amino acids of SEQ ID NO: 3) or a fragment thereof, provided said fragment contains SEQ ID NO: 16: F955, I958, S960, and/or N963 (with such amino acid positions labeled based on the full sequence of SEQ ID NO: 3). As such, in aspects the present disclosure is directed to a polypeptide comprising the amino acid sequence of SEQ ID NO: 3 (or a polypeptide of SEQ ID NO: 3 without the first 15 amino acids of SEQ ID NO: 3) or a fragment thereof, provided said fragment contains SEQ ID NO: 16, and comprising mutations to F955, I958, S960, and/or N963 (with such amino acid positions labeled based on the full sequence of SEQ ID NO: 3). In aspects, the mutations of F955, I958, S960, and/or N963 include amino acid substitutions that are disfavorable for binding. In aspects, such mutations can be any amino acid substitution identified by EpiMatrix as disruptive (reduces predicted T cell epitope content). In aspects, such mutations can be determined by HLA binding assays, as is known in the art. In aspects, specific mutations to the polypeptide of SEQ ID NO: 3 (or a polypeptide of SEQ ID NO: 3 without the first 15 amino acids of SEQ ID NO: 3) or a fragment thereof, provided said fragment contains SEQ ID NO: 16, include the following: F955A; F955G; F955N; F955Q; F955S; F955T, I958G; S960G; 5960Q; and/or S960T (with such amino acid positions labeled based on the full sequence of SEQ ID NO: 3).

In aspects, mutations are made to specific TCR contacts of the spike polypeptide of SEQ ID NO: 3 (or a polypeptide of SEQ ID NO: 3 without the first 15 amino acids of SEQ ID NO: 3), including mutations to specific TCR contacts of the tolerizing epitopes identified therein, and TCR recognition of such epitopes can be disrupted. Any amino acid substitution can disrupt TCR recognition. In aspects, such mutations include mutating the following amino acids of SEQ ID NO: 3 (or a polypeptide of SEQ ID NO: 3 without the first 15 amino acids of SEQ ID NO: 3) or a fragment thereof, provided said fragment contains SEQ ID NO: 16: G956, A957, S959, V961, and/or L962, especially S959, (with such amino acid positions labeled based on the full sequence of SEQ ID NO: 3). As such, in aspects the present disclosure is directed to a polypeptide comprising the amino acid sequence of SEQ ID NO: 3 (or a polypeptide of SEQ ID NO: 3 without the first 15 amino acids of SEQ ID NO: 3) or a fragment thereof, provided said fragment contains SEQ ID NO: 16, and comprising mutations to G956, A957, S959, V961, and/or L962, especially S959, (with such amino acid positions labeled based on the full sequence of SEQ ID NO: 3). In aspects, the mutations G956, A957, S959, V961, and/or L962 include any amino acid substitutions. In aspects, mutations are made to specific anchoring residues of the spike polypeptide of SEQ ID NO: 3 (or a polypeptide of SEQ ID NO: 3 without the first 15 amino acids of SEQ ID NO: 3), including mutations to specific anchoring residues of the tolerizing epitopes identified therein, and the binding of such epitopes can be disrupted. Such mutations for the anchoring residues must select replacement amino acids which are dis-favorable for binding. In aspects, such mutations include mutating the following amino acids of SEQ ID NO: 3 (or a polypeptide of SEQ ID NO: 3 without the first 15 amino acids of SEQ ID NO: 3) or a fragment thereof, provided said fragment contains SEQ ID NO: 17: I998, A1001, I1003, S1006, N1008, and/or A1011 (with such amino acid positions labeled based on the full sequence of SEQ ID NO: 3). As such, in aspects the present disclosure is directed to a polypeptide comprising the amino acid sequence of SEQ ID NO: 3 (or a polypeptide of SEQ ID NO: 3 without the first 15 amino acids of SEQ ID NO: 3) or a fragment thereof, provided said fragment contains SEQ ID NO: 17, and comprising mutations to I998, A1001, I1003, S1006, N1008, and/or A1011 (with such amino acid positions labeled based on the full sequence of SEQ ID NO: 3). In aspects, the mutations of I998, A1001, I1003, S1006, N1008, and/or A1011 include amino acid substitutions that are disfavorable for binding. In aspects, such mutations can be any amino acid substitution identified by EpiMatrix as disruptive (reduces predicted T cell epitope content). In aspects, such mutations can be determined by HLA binding assays, as is known in the art. In aspects, specific mutations to the polypeptide of SEQ ID NO: 3 (or a polypeptide of SEQ ID NO: 3 without the first 15 amino acids of SEQ ID NO: 3) or a fragment thereof, provided said fragment contains SEQ ID NO: 17, include the following: I998A; I998G; I998N; I998Q; 1998S; 1998T; A1001G; A1001T; I1003A; I1003G; I1003N; I1003Q; I1003S; I1003T and/or N1008Q (with such amino acid positions labeled based on the full sequence of SEQ ID NO: 3).

In aspects, mutations are made to specific TCR contacts of the spike polypeptide of SEQ ID NO: 3 (or a polypeptide of SEQ ID NO: 3 without the first 15 amino acids of SEQ ID NO: 3), including mutations to specific TCR contacts of the tolerizing epitopes identified therein, and TCR recognition of such epitopes can be disrupted. Any amino acid substitution can disrupt TCR recognition. In aspects, such mutations include mutating the following amino acids of SEQ ID NO: 3 (or a polypeptide of SEQ ID NO: 3 without the first 15 amino acids of SEQ ID NO: 3) or a fragment thereof, provided said fragment contains SEQ ID NO: 17: R999, A1000, E1002, R104, A1005, A1007, L1009, and/or A1010, especially E1002 and/or A1007, (with such amino acid positions labeled based on the full sequence of SEQ ID NO: 3). As such, in aspects the present disclosure is directed to a polypeptide comprising the amino acid sequence of SEQ ID NO: 3 (or a polypeptide of SEQ ID NO: 3 without the first 15 amino acids of SEQ ID NO: 3) or a fragment thereof, provided said fragment contains SEQ ID NO: 17, and comprising mutations to R999, A1000, E1002, R104, A1005, A1007, L1009, and/or A1010, especially E1002 and/or A1007, (with such amino acid positions labeled based on the full sequence of SEQ ID NO: 3). In aspects, the mutations R999, A1000, E1002, R104, A1005, A1007, L1009, and/or A1010 include any amino acid substitutions. By way of further example, SEQ ID NO: 439 sets forth two identified Tregitope mutations within SEQ ID NO: 17.

In aspects, mutations are made to specific anchoring residues of the spike polypeptide of SEQ ID NO: 3, or a fragment thereof, provided said fragment contains one or more of SEQ ID NOS: 8-17, 42-93, 246-370, 422, 423, 432, 434-439, and 794-833, including mutations to specific anchoring residues of the tolerizing epitopes identified therein (e.g., one or more of SEQ ID NOS: 8-17, 42-93, 246-370, 422, 423, 432, 434-439, and 794-833), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. Further, by mutating TCR contacts, including mutations to specific TCR contacts of the tolerizing epitopes identified therein (e.g., one or more of SEQ ID NOS: 8-17, 42-93, 246-370, 422, 423, 432, 434-439, and 794-833), T cell recognition of such tolerizing epitopes can be disrupted. Any amino acid substitution to TCR contacts disrupt TCR recognition.

In aspects, the present disclosure relates to detolerized SARS-CoV-2 nucleocapsid polypeptides of SEQ ID NO: 371 or fragments thereof, wherein one or more of SEQ ID NOS: 94, 95, 406-421, and 474-487 is modified or removed from said detolerized nucleocapsid polypeptide of SEQ ID NO: 371 or fragment thereof. For example, in aspects, modification or removal of one or more of the identified regulatory T cell epitopes (e.g., one or more of SEQ ID NOS: 94, 95, 406-421, and 474-487) comprises deletion of all or some of the amino acids of the one or more regulatory T cell epitopes of SEQ ID NOS: 94, 95, 406-421, and 474-487. In aspects, said modification or removal of the one or more regulatory T cell epitopes of SEQ ID NOS: 94, 95, 406-421, and 474-487 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 94, 95, 406-421, and 474-487 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 94, 95, 406-421, and 474-487 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 94, 95, 406-421, and 474-487), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 94, 95, 406-421, and 474-487), T cell recognition of such tolerizing epitopes can be disrupted. Any amino acid substitution to TCR contacts disrupt TCR recognition.

In aspects, the present disclosure relates to detolerized SARS-CoV-2 ORF3a polypeptides of SEQ ID NO: 372 or fragments thereof, wherein one or more of SEQ ID NOS: 96-101, 424, and 756-774 is modified or removed from said detolerized ORF3a polypeptide of SEQ ID NO: 372 or fragment thereof. For example, in aspects, modification or removal of one or more of the identified regulatory T cell epitopes (e.g., one or more of SEQ ID NOS: 96-101, 424, and 756-774) comprises deletion of all or some of the amino acids of the one or more regulatory T cell epitopes of SEQ ID NOS: 96-101, 424, and 756-774. In aspects, said modification or removal of the one or more regulatory T cell epitopes of SEQ ID NOS: 96-101, 424, and 756-774 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 96-101, 424, and 756-774 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 96-101, 424, and 756-774 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 96-101, 424, and 756-774), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 96-101, 424, and 756-774), T cell recognition of such tolerizing epitopes can be disrupted. Any amino acid substitution to TCR contacts disrupt TCR recognition.

In aspects, the present disclosure relates to detolerized SARS-CoV-2 ORF6 polypeptides of SEQ ID NO: 373 or fragments thereof, wherein one or more of SEQ ID NOS: 102, 103, and 775 is modified or removed from said detolerized ORF6 polypeptide of SEQ ID NO: 373 or fragment thereof. For example, in aspects, modification or removal of one or more of the identified regulatory T cell epitopes (e.g., one or more of SEQ ID NOS: 102, 103, and 775) comprises deletion of all or some of the amino acids of the one or more regulatory T cell epitopes of SEQ ID NOS: 102, 103, and 775. In aspects, said modification or removal of the one or more regulatory T cell epitopes of SEQ ID NOS: 102, 103, and 775 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 102, 103, and 775 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 102, 103, and 775 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 102, 103, and 775), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 102, 103, and 775), T cell recognition of such tolerizing epitopes can be disrupted. Any amino acid substitution to TCR contacts disrupt TCR recognition.

In aspects, the present disclosure relates to detolerized SARS-CoV-2 ORF7 polypeptides of SEQ ID NO: 374 or fragments thereof, wherein one or more of SEQ ID NOS: 104-107, 425, and 776-783 is modified or removed from said detolerized ORF7 polypeptide of SEQ ID NO: 374 or fragment thereof. For example, in aspects, modification or removal of one or more of the identified regulatory T cell epitopes (e.g., one or more of SEQ ID NOS: 104-107, 425, and 776-783) comprises deletion of all or some of the amino acids of the one or more regulatory T cell epitopes of SEQ ID NOS: 104-107, 425, and 776-783. In aspects, said modification or removal of the one or more regulatory T cell epitopes of SEQ ID NOS: 104-107, 425, and 776-783 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 104-107, 425, and 776-783 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 104-107, 425, and 776-783 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 104-107, 425, and 776-783), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 104-107, 425, and 776-783), T cell recognition of such tolerizing epitopes can be disrupted. Any amino acid substitution to TCR contacts disrupt TCR recognition.

In aspects, the present disclosure relates to detolerized SARS-CoV-2 the ORF1ab non-structural protein 2 (NSP2) protein polypeptides of SEQ ID NO: 375 or fragments thereof, wherein one or more of SEQ ID NOS: 108-115, 427, and 554-575 is modified or removed from said detolerized the ORF1ab non-structural protein 2 (NSP2) protein polypeptide of SEQ ID NO: 375 or fragment thereof. For example, in aspects, modification or removal of one or more of the identified regulatory T cell epitopes (e.g., one or more of SEQ ID NOS: 108-115, 427, and 554-575) comprises deletion of all or some of the amino acids of the one or more regulatory T cell epitopes of SEQ ID NOS: 108-115, 427, and 554-575. In aspects, said modification or removal of the one or more regulatory T cell epitopes of SEQ ID NOS: 108-115, 427, and 554-575 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 108-115, 427, and 554-575 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 108-115, 427, and 554-575 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 108-115, 427, and 554-575), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 108-115, 427, and 554-575), T cell recognition of such tolerizing epitopes can be disrupted. Any amino acid substitution to TCR contacts disrupt TCR recognition.

In aspects, the present disclosure relates to detolerized SARS-CoV-2 the ORF1ab non-structural protein 3 (NSP3) protein polypeptides of SEQ ID NO: 376 or fragments thereof, wherein one or more of SEQ ID NOS: 116-135, 428, 429, and 576-655 is modified or removed from said detolerized the ORF1ab non-structural protein 3 (NSP3) protein polypeptide of SEQ ID NO: 376 or fragment thereof. For example, in aspects, modification or removal of one or more of the identified regulatory T cell epitopes (e.g., one or more of SEQ ID NOS: 116-135, 428, 429, and 576-655) comprises deletion of all or some of the amino acids of the one or more regulatory T cell epitopes of SEQ ID NOS: 116-135, 428, 429, and 576-655. In aspects, said modification or removal of the one or more regulatory T cell epitopes of SEQ ID NOS: 116-135, 428, 429, and 576-655 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 116-135, 428, 429, and 576-655 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 116-135, 428, 429, and 576-655 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 116-135, 428, 429, and 576-655), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 116-135, 428, 429, and 576-655), T cell recognition of such tolerizing epitopes can be disrupted. Any amino acid substitution to TCR contacts disrupt TCR recognition.

In aspects, the present disclosure relates to detolerized SARS-CoV-2 the ORF1ab non-structural protein 4 (NSP4) protein polypeptides of SEQ ID NO: 377 or fragments thereof, wherein one or more of SEQ ID NOS: 136-141, 391, 392, and 656-684 is modified or removed from said detolerized the ORF1ab non-structural protein 4 (NSP4) protein polypeptide of SEQ ID NO: 377 or fragment thereof. For example, in aspects, modification or removal of one or more of the identified regulatory T cell epitopes (e.g., one or more of SEQ ID NOS: 136-141, 391, 392, and 656-684) comprises deletion of all or some of the amino acids of the one or more regulatory T cell epitopes of SEQ ID NOS: 136-141, 391, 392, and 656-684. In aspects, said modification or removal of the one or more regulatory T cell epitopes of SEQ ID NOS: 136-141, 391, 392, and 656-684 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 136-141, 391, 392, and 656-684 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 136-141, 391, 392, and 656-684 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 136-141, 391, 392, and 656-684), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 136-141, 391, 392, and 656-684), T cell recognition of such tolerizing epitopes can be disrupted. Any amino acid substitution to TCR contacts disrupt TCR recognition.

In aspects, the present disclosure relates to detolerized SARS-CoV-2 the ORF1ab non-structural protein 6 (NSP6) protein polypeptides of SEQ ID NO: 378 or fragments thereof, wherein one or more of SEQ ID NOS: 142-149 and 685-708 is modified or removed from said detolerized the ORF1ab non-structural protein 6 (NSP6) protein polypeptide of SEQ ID NO: 378 or fragment thereof. For example, in aspects, modification or removal of one or more of the identified regulatory T cell epitopes (e.g., one or more of SEQ ID NOS: 142-149 and 685-708) comprises deletion of all or some of the amino acids of the one or more regulatory T cell epitopes of SEQ ID NOS: 142-149 and 685-708. In aspects, said modification or removal of the one or more regulatory T cell epitopes of SEQ ID NOS: 142-149 and 685-708 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 142-149 and 685-708 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 142-149 and 685-708 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 142-149 and 685-708), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 142-149 and 685-708), T cell recognition of such tolerizing epitopes can be disrupted. Any amino acid substitution to TCR contacts disrupt TCR recognition.

In aspects, the present disclosure relates to detolerized SARS-CoV-2 the ORF1ab non-structural protein 7 (NSP7) protein polypeptides of SEQ ID NO: 379 or fragments thereof, wherein one or more of SEQ ID NOS: 150-153, 393-405, and 709-718 is modified or removed from said detolerized the ORF1ab non-structural protein 7 (NSP7) protein polypeptide of SEQ ID NO: 379 or fragment thereof. For example, in aspects, modification or removal of one or more of the identified regulatory T cell epitopes (e.g., one or more of SEQ ID NOS: 150-153, 393-405, and 709-718) comprises deletion of all or some of the amino acids of the one or more regulatory T cell epitopes of SEQ ID NOS: 150-153, 393-405, and 709-718. In aspects, said modification or removal of the one or more regulatory T cell epitopes of SEQ ID NOS: 150-153, 393-405, and 709-718 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 150-153, 393-405, and 709-718 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 150-153, 393-405, and 709-718 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 150-153, 393-405, and 709-718), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 150-153, 393-405, and 709-718), T cell recognition of such tolerizing epitopes can be disrupted. Any amino acid substitution to TCR contacts disrupt TCR recognition.

In aspects, the present disclosure relates to detolerized SARS-CoV-2 the ORF1ab non-structural protein 8 (NSP8) protein polypeptides of SEQ ID NO: 380 or fragments thereof, wherein one or more of SEQ ID NOS: 154-155 and 719-725 is modified or removed from said detolerized the ORF1ab non-structural protein 8 (NSP8) protein polypeptide of SEQ ID NO: 380 or fragment thereof. For example, in aspects, modification or removal of one or more of the identified regulatory T cell epitopes (e.g., one or more of SEQ ID NOS: 154-155 and 719-725) comprises deletion of all or some of the amino acids of the one or more regulatory T cell epitopes of SEQ ID NOS: 154-155 and 719-725. In aspects, said modification or removal of the one or more regulatory T cell epitopes of SEQ ID NOS: 154-155 and 719-725 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 154-155 and 719-725 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 154-155 and 719-725 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 154-155 and 719-725), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 154-155 and 719-725), T cell recognition of such tolerizing epitopes can be disrupted. Any amino acid substitution to TCR contacts disrupt TCR recognition.

In aspects, the present disclosure relates to detolerized SARS-CoV-2 the ORF1ab non-structural protein 9 (NSP9) protein polypeptides of SEQ ID NO: 381 or fragments thereof, wherein one or more of SEQ ID NOS: 156-159 and 726-730 is modified or removed from said detolerized the ORF1ab non-structural protein 9 (NSP9) protein polypeptide of SEQ ID NO: 381 or fragment thereof. For example, in aspects, modification or removal of one or more of the identified regulatory T cell epitopes (e.g., one or more of SEQ ID NOS: 156-159 and 726-730) comprises deletion of all or some of the amino acids of the one or more regulatory T cell epitopes of SEQ ID NOS: 156-159 and 726-730. In aspects, said modification or removal of the one or more regulatory T cell epitopes of SEQ ID NOS: 156-159 and 726-730 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 156-159 and 726-730 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 156-159 and 726-730 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 156-159 and 726-730), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 156-159 and 726-730), T cell recognition of such tolerizing epitopes can be disrupted. Any amino acid substitution to TCR contacts disrupt TCR recognition.

In aspects, the present disclosure relates to detolerized SARS-CoV-2 the ORF1ab RNA-dependent RNA polymerase polypeptides of SEQ ID NO: 382 or fragments thereof, wherein one or more of SEQ ID NOS: 160-165, 431, and 731-755 is modified or removed from said detolerized ORF1ab RNA-dependent RNA polymerase polypeptide of SEQ ID NO: 382 or fragment thereof. For example, in aspects, modification or removal of one or more of the identified regulatory T cell epitopes (e.g., one or more of SEQ ID NOS: 160-165, 431, and 731-755) comprises deletion of all or some of the amino acids of the one or more regulatory T cell epitopes of SEQ ID NOS: 160-165, 431, and 731-755. In aspects, said modification or removal of the one or more regulatory T cell epitopes of SEQ ID NOS: 160-165, 431, and 731-755 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 160-165, 431, and 731-755 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 160-165, 431, and 731-755 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 160-165, 431, and 731-755), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 160-165, 431, and 731-755), T cell recognition of such tolerizing epitopes can be disrupted. Any amino acid substitution to TCR contacts disrupt TCR recognition.

In aspects, the present disclosure relates to detolerized SARS-CoV-2 the ORF1ab helicase protein polypeptides of SEQ ID NO: 383 or fragments thereof, wherein one or more of SEQ ID NOS: 166-173 and 515-542 is modified or removed from said detolerized the ORF1ab helicase protein polypeptide of SEQ ID NO: 383 or fragment thereof. For example, in aspects, modification or removal of one or more of the identified regulatory T cell epitopes (e.g., one or more of SEQ ID NOS: 166-173 and 515-542) comprises deletion of all or some of the amino acids of the one or more regulatory T cell epitopes of SEQ ID NOS: 166-173 and 515-542. In aspects, said modification or removal of the one or more regulatory T cell epitopes of SEQ ID NOS: 166-173 and 515-542 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 166-173 and 515-542 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 166-173 and 515-542 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 166-173 and 515-542), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 166-173 and 515-542), T cell recognition of such tolerizing epitopes can be disrupted. Any amino acid substitution to TCR contacts disrupt TCR recognition.

In aspects, the present disclosure relates to detolerized SARS-CoV-2 the ORF1ab 3′-to-5′ exonuclease polypeptides of SEQ ID NO: 384 or fragments thereof, wherein one or more of SEQ ID NOS: 174-175 and 496-505 is modified or removed from said detolerized the ORF1ab 3′-to-5′ exonuclease polypeptides of SEQ ID NO: 384 or fragment thereof. For example, in aspects, modification or removal of one or more of the identified regulatory T cell epitopes (e.g., one or more of SEQ ID NOS: 174-175 and 496-505) comprises deletion of all or some of the amino acids of the one or more regulatory T cell epitopes of SEQ ID NOS: 174-175 and 496-505. In aspects, said modification or removal of the one or more regulatory T cell epitopes of SEQ ID NOS: 174-175 and 496-505 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 174-175 and 496-505 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 174-175 and 496-505 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 174-175 and 496-505), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 174-175 and 496-505), T cell recognition of such tolerizing epitopes can be disrupted. Any amino acid substitution to TCR contacts disrupt TCR recognition.

In aspects, the present disclosure relates to detolerized SARS-CoV-2 the ORF1ab endoRNase protein polypeptides of SEQ ID NO: 385 or fragments thereof, wherein one or more of SEQ ID NOS: 176-179 and 506-514 is modified or removed from said detolerized the ORF1ab endoRNase protein polypeptide of SEQ ID NO: 385 or fragment thereof. For example, in aspects, modification or removal of one or more of the identified regulatory T cell epitopes (e.g., one or more of SEQ ID NOS: 176-179 and 506-514) comprises deletion of all or some of the amino acids of the one or more regulatory T cell epitopes of SEQ ID NOS: 176-179 and 506-514. In aspects, said modification or removal of the one or more regulatory T cell epitopes of SEQ ID NOS: 176-179 and 506-514 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 176-179 and 506-514 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 176-179 and 506-514 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 176-179 and 506-514), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 176-179 and 506-514), T cell recognition of such tolerizing epitopes can be disrupted. Any amino acid substitution to TCR contacts disrupt TCR recognition.

In aspects, the present disclosure relates to detolerized SARS-CoV-2 the ORF1ab 2′O-ribose methyltransferase protein polypeptides of SEQ ID NO: 386 or fragments thereof, wherein one or more of SEQ ID NOS: 180-185, 433, and 543-551 is modified or removed from said detolerized the ORF1ab 2′O-ribose methyltransferase protein polypeptide of SEQ ID NO: 386 or fragment thereof. For example, in aspects, modification or removal of one or more of the identified regulatory T cell epitopes (e.g., one or more of SEQ ID NOS: 180-185, 433, and 543-551) comprises deletion of all or some of the amino acids of the one or more regulatory T cell epitopes of SEQ ID NOS: 180-185, 433, and 543-551. In aspects, said modification or removal of the one or more regulatory T cell epitopes of SEQ ID NOS: 180-185, 433, and 543-551 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 180-185, 433, and 543-551 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 180-185, 433, and 543-551 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 180-185, 433, and 543-551), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 180-185, 433, and 543-551), T cell recognition of such tolerizing epitopes can be disrupted. Any amino acid substitution to TCR contacts disrupt TCR recognition.

In aspects, the present disclosure relates to detolerized SARS-CoV-2 the ORF10 protein polypeptides of SEQ ID NO: 388 or fragments thereof, wherein one or more of SEQ ID NOS: 488 and 489 is modified or removed from said detolerized the ORF10 protein polypeptide of SEQ ID NO: 388 or fragment thereof. For example, in aspects, modification or removal of one or more of the identified regulatory T cell epitopes (e.g., one or more of SEQ ID NOS: 488 and 489) comprises deletion of all or some of the amino acids of the one or more regulatory T cell epitopes of SEQ ID NOS: 488 and 489. In aspects, said modification or removal of the one or more regulatory T cell epitopes of SEQ ID NOS: 488 and 489 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 488 and 489 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 488 and 489 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 488 and 489), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 488 and 489), T cell recognition of such tolerizing epitopes can be disrupted. Any amino acid substitution to TCR contacts disrupt TCR recognition.

In aspects, the present disclosure relates to detolerized SARS-CoV-2 the ORF1ab 3C-like proteinase polypeptides of SEQ ID NO: 389 or fragments thereof, wherein one or more of SEQ ID NOS: 490-495 is modified or removed from said detolerized the ORF1ab 3C-like proteinase polypeptide of SEQ ID NO: 389 or fragment thereof. For example, in aspects, modification or removal of one or more of the identified regulatory T cell epitopes (e.g., one or more of SEQ ID NOS: 490-495) comprises deletion of all or some of the amino acids of the one or more regulatory T cell epitopes of SEQ ID NOS: 490-495. In aspects, said modification or removal of the one or more regulatory T cell epitopes of SEQ ID NOS: 490-495 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 490-495 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 490-495 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 490-495), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 490-495), T cell recognition of such tolerizing epitopes can be disrupted. Any amino acid substitution to TCR contacts disrupt TCR recognition.

In aspects, the present disclosure relates to detolerized SARS-CoV-2 the ORF1ab non-structural protein 10 (NSP10) polypeptides of SEQ ID NO: 390 or fragments thereof, wherein one or more of SEQ ID NOS: 552 and 553 is modified or removed from said detolerized the ORF1ab non-structural protein 10 (NSP10) polypeptide of SEQ ID NO: 390 or fragment thereof. For example, in aspects, modification or removal of one or more of the identified regulatory T cell epitopes (e.g., one or more of SEQ ID NOS: 552 and 553) comprises deletion of all or some of the amino acids of the one or more regulatory T cell epitopes of SEQ ID NOS: 552 and 553. In aspects, said modification or removal of the one or more regulatory T cell epitopes of SEQ ID NOS: 552 and 553 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 552 and 553 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 552 and 553 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 552 and 553), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 552 and 553), T cell recognition of such tolerizing epitopes can be disrupted. Any amino acid substitution to TCR contacts disrupt TCR recognition.

In aspects, the present disclosure relates to detolerized SARS-CoV-2 the ORF8 protein polypeptides of SEQ ID NO: 387 or fragments thereof, wherein one or more of SEQ ID NOS: 426 and 784-793 is modified or removed from said detolerized the ORF8 protein polypeptide of SEQ ID NO: 387 or fragment thereof. For example, in aspects, modification or removal of one or more of the identified regulatory T cell epitopes (e.g., one or more of SEQ ID NOS: 426 and 784-793) comprises deletion of all or some of the amino acids of the one or more regulatory T cell epitopes of SEQ ID NOS: 426 and 784-793. In aspects, said modification or removal of the one or more regulatory T cell epitopes of SEQ ID NOS: 426 and 784-793 comprises deletion of some or all of the amino acids of the one or more regulatory T cell epitopes and adding one or more amino acids at the site of deletion of the regulatory T cell epitope amino acids. In aspects, said modification or removal of the one or more regulatory T cell epitopes of SEQ ID NOS: 426 and 784-793 from an antigen or polypeptide sequence comprises mutating the one or more regulatory T cell epitopes (for example, but not limited to, introduction one or more point mutations into the one or more regulatory T cell epitopes by site-directed mutagenesis or other recombinant techniques). In aspects, said modification or removal of the one or more regulatory T cell epitopes of SEQ ID NOS: 426 and 784-793 from an antigen or polypeptide sequence comprises introducing one or more amino acids into the one or more regulatory T cell epitope sequences, which in aspects will disrupt the one or more regulatory T cell epitope sequences, such that the immunogenicity of the sequences is enhanced. In aspects, the number of said added one or more amino acids at the site of modification/removal need not correspond to the number of amino acids deleted from the previously existing regulatory T cell epitope amino acids. In aspects, such modification or removal include mutations to specific anchoring residues of the tolerizing epitopes identified therein (e.g., one or more of SEQ ID NOS: 426 and 784-793), and the binding of such epitopes can be disrupted. Such mutations for the anchoring residues must select replacement amino acids which are dis-favorable for binding. Any amino acid substitution identified by EpiMatrix as disruptive (reduces predicted T cell epitope content) is viable. In aspects, such mutations can be determined by HLA binding assays, as is known in the art. Further, by mutating TCR contacts, including mutations to specific TCR contacts of the tolerizing epitopes identified therein (e.g., one or more of SEQ ID NOS: 426 and 784-793), T cell recognition of such tolerizing epitopes can be disrupted. Any amino acid substitution to TCR contacts disrupt TCR recognition.

In aspects, instant disclosure is directed to a polypeptide as shown below in Tables 6-8 and Tables 9-11, with the mutations to the full length spike of COVID-19 and the ectodomain of spike of COVID-19, respectively, italicized, underlined, and bolded.

TABLE 6
Full Length Spike Opt 1 (SEQ ID NO: 842)

MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVL [symbol] FELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVLIAIVMVTIMLCCMTSCCSCLKGCCSCGSCCKFDED
DSEPVLKGVKLHYT

TABLE 7
Full Length Spike Opt 2 (SEQ ID NO: 843)

MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQL [symbol] RAAE [symbol] RASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYT

TABLE 8
Full Length Spike Opt 3 (SEQ ID NO: 844)

MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVL [symbol] FELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQL [symbol] RAAE [symbol] RASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYT

TABLE 9
Ectodomain Spike Opt 1 (SEQ. ID NO: 845)

MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVL [symbol] FELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVKNLNESLIDLQELGKYEQYIKWP

TABLE 10
Ectodomain Spike Opt 2 (SEQ ID NO: 846)

MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSHAYTMSLGAENSVAYSNNSIAIPTNFTISVTTEILPVSMTKTSV
DCTMYICGDSTECSNLLLQYGSFCTQLNRALTGIAVEQDKNTQEVFAQV
KQIYKTPPIKDFGGFNFSQILPDPSKPSKRSFIEDLLFNKVTLADAGFI
KQYGDCLGDIAARDLICAQKFNGLTVLPPLLTDEMIAQYTSALLAGTIT
SGWTFGAGAALQIPFAMQMAYRFNGIGVTQNVLYENQKLIANQFNSAIG
KIQDSLSSTASALGKLQDVVNQNAQALNTLVKQLSSNFGAISSVLNDIL
SRLDKVEAEVQIDRLITGRLQSLQTYVTQQL [symbol] RAAE [symbol] RASANLAATKMS
ECVLGQSKRVDFCGKGYHLMSFPQSAPHGVVFLHVTYVPAQEKNFTTAP
AICHDGKAHFPREGVFVSNGTHWFVTQRNFYEPQIITTDNTFVSGNCDV
VIGIVNNTVYDPLQPELDSFKEELDKYFKNHTSPDVDLGDISGINASVV
NIQKEIDRLNEVKNLNESLIDLQELGKYEQYIKWP

TABLE 11
Ectodomain Spike Opt 3 (SEQ ID NO: 847)

MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVL [symbol] FELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQL [symbol] RAAE [symbol] RASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWP

In aspects, instant disclosure is directed to a polypeptide as shown below in Tables 12-19, with the mutations to the glycoprotein of COVID-19 (Tables 12-15) and the spike of COVID-19 (Tables 16-19).

TABLE 12
Surface Glycoprotein Opt1 (SEQ ID NO: 441)

MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVGVLFELLHAPATVCGPKKSTNLVKNKCVN
FNFNGLTGTGVLTESNKKFLPFQQFGRDIADTTDAVRDPQTLEILDITP
CSFGGVSVITPGTNTSNQVAVLYQDVNCTEVPVAIHADQLTPTWRVYST
GSNVFQTRAGCLIGAEHVNNSYECDIPIGAGICASYQTQTNSPRRARSV
ASQSIIAYTMSLGAENSVAYSNNSIAIPTNFTISVTTEILPVSMTKTSV
DCTMYICGDSTECSNLLLQYGSFCTQLNRALTGIAVEQDKNTQEVFAQV
KQIYKTPPIKDFGGFNFSQILPDPSKPSKRSFIEDLLFNKVTLADAGFI
KQYGDCLGDIAARDLICAQKFNGLTVLPPLLTDEMIAQYTSALLAGTIT
SGWTFGAGAALQIPFAMQMAYRFNGIGVTQNVLYENQKLIANQFNSAIG
KIQDSLSSTASALGKLQDVVNQNAQALNTLVKQLSSNFGAISSVLNDIL
SRLDKVEAEVQIDRLITGRLQSLQTYVTQQLIRAAEIRASANLAATKMS
ECVLGQSKRVDFCGKGYHLMSFPQSAPHGVVFLHVTYVPAQEKNFTTAP
AICHDGKAHFPREGVFVSNGTHWFVTQRNFYEPQIITTDNTFVSGNCDV
VIGIVNNTVYDPLQPELDSFKEELDKYDKNHTSPDVDLGDISGINASVV
NIQKEIDRLVEVAKNLNESLIDLQELGKYEQYIKWP

TABLE 13
Surface Glycoprotein Opt3 (SEQ ID NO: 442)

MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVGVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVIAHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLGRAAEGRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKIEDRLNEVAKNLNESLIDLQELGKYEQYIKWP

TABLE 14
Surface Glycoprotein Opt1 (SEQ ID NO: 443)

MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVGVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCCKFDEDDSPEVLKGVKLHYT

TABLE 15
Surface Glycoprotein Opt3 (SEQ ID NO: 444)

MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVGVLSFELLHAPATVCGPKKSTNLVKNKCV
NFNFNGLTGTGVLTESNKKFLPFQQRGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDLCLGDIAARDLICAQKFNGLTVLPPLLTDEMIAQYTSALLAGT
ITSGWTFGAGAALQIPFAMQMAYRFNGIGVTQNVLYENQKLIANQFNSA
IGKIQDSLSSTASALGKLQDVVNQNAQALNTLVKQLSSNFGAISSVLND
ILSRLDKVEAEVQIDRLITGRLQSLQTYVTQQLRGRAAEGRASANLAAT
KMSECVLGQSKRVDFCGKGYHLMSFPQSAPHGVVFLHVTYVPAQEKNFT
TAPAICHDGKAHFPREGVFVSNGTHWFVTQRNFYEPQIITTDNTFVSGN
CDVVIGIVNNTVYDPLQPELDSFKEELDKYFKNHTSPDVDLGDISGINA
SVVNIQKEIDRLNEVAKNLNESLIDLQELGKYEQYIKWPWYIWLGFIAG
LIAIVMVTIMLCCMTSCCSCLKGCCSCGSCCKFDEDDSEPVLKGVKLHY
T

TABLE 16
Spike (SEQ ID NO: 445)

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNERVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPGSASSVASQSIIAYTMSLG
AENSVAYSNNSIAIPTNFTISVTTEILPVSMTKTSVDCTMYICGDSTECS
NLLLQYGSFCTOLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALTLVKQL
SSNFGAISSVLNDILSRLDPPEAEVQIDRLITGRLQSLQTYVTQQLIRAA
EIRASANLAATKMSECVLGQSKRVDFCGKGYHLMSFPQSAPHGVVFLHVT
YVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDL
GDISGINASVVNIQKEIDRLNEVAKNLNESLIDLQELGKYEQGSGYIPEA
PRDGQAYVRKDGEWVLLSTFLGRSLEVLFQGPGHHHHHHHH

TABLE 17
Spike Opt1 (SEQ ID NO: 446)

MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVGVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPGSASS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDPPEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQGSGYIPEAPRDGQAYVR
KDGEWVLLSTFLGRSLEVLFQGPGHHHHHHHH

TABLE 18
Spike Opt2 (SEQ ID NO: 447)

MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPGSASS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDPPEAEVQIDRLITGRLQSLQTYVTQQLGRAAEGRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQGSGYIPEAPRDGQAYVR
KDGEWVLLSTFLGRSLEVLFQGPGHHHHHHHH

TABLE 19
Spike Opt3 (SEQ ID NO: 834)

MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQL [symbol] RAAE [symbol] RASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVKNLNESLIDLQELGKYEQYIKWP

In aspects, the 2019 COVID-19 antigens (e.g., peptides and polypeptides) of the instant disclosure can be isolated, synthetic, and/or recombinant. In aspects, the 2019 COVID-19 antigens (e.g., peptides and polypeptides) of the instant disclosure can be either in neutral (uncharged) or salt forms, and may be either free of or include modifications such as glycosylation, side chain oxidation, or phosphorylation. In aspects, the 2019 COVID-19 antigens (e.g., peptides and polypeptides) of the instant disclosure can be capped with an n-terminal acetyl and/or c-terminal amino group.

### (iv) Tregitope and Detolerized Antigen Polypeptides, Concatemers and Chimeric/Fusion Polypeptides

In aspects, the present disclosure provides a peptides or polypeptide chains derived from SARS-CoV-2 proteins (e.g., encoded proteins from a SARS-CoV-2 genome), including the envelope, membrane, spike, nucleocapsid, ORF3a, ORF6, ORF7a, ORFS, ORF10, ORF1ab non-structural protein 2 (NSP2), ORF1ab non-structural protein 3 (NSP3), ORF1ab non-structural protein 4 (NSP4), ORF1ab 3C-like proteinase, ORF1ab non-structural protein 6 (NSP6), ORF1ab non-structural protein 7 (NSP7), ORF1ab non-structural protein 8 (NSP8), ORF1ab non-structural protein 9 (NSP9), ORF1ab non-structural protein 10 (NSP10), ORF1ab RNA-dependent RNA polymerase, ORF1ab helicase, ORF1ab 3′-5′ exonuclease, ORF1ab endoRNase, and ORF1ab 2′O-ribose methyltransferase proteins of SARS-CoV-2. As explained in more detail in Example 1, T-cell epitopes of the present disclosure are highly conserved among known variants of their source proteins, and SARS-CoV-2 (taxid: 2697049), SARS-CoV-1 (taxid: 694009), MERS-CoV (taxid: 1335626), and human CoV (taxids: 11137, 443239, 277944 and 31631) antigen sequences isolated from human hosts were obtained from GenBank at the National Center for Biotechnology Information. SARS-CoV-2 epitopes were compared across sequences obtained from isolates with fully sequenced genomes isolated from December 2019 to December 2020 for T cell epitope mapping. SARS-CoV-2 Wuhan-Hu-1 (GenBank id: MN908947) was selected as the reference strain.

As further described in Example 1, T-cell epitopes of the present disclosure comprise at least one putative T cell epitope as identified by EpiMatrix™ analysis. EpiMatrix™ is a proprietary computer algorithm developed by EpiVax (Providence, R.I.), which is used to screen protein sequences for the presence of putative T cell epitopes. The algorithm uses matrices for prediction of 9- and 10-mer peptides binding to MHC molecules. Each matrix is based on position-specific coefficients related to amino acid binding affinities that are elucidated by a method similar to, but not identical to, the pocket profile method (Sturniolo, T. et al., Nat. Biotechnol., 17:555-561, 1999). Input sequences are, for example, parsed into overlapping 9-mer frames or 10-mer where each frame overlaps the last by 8 or 9 amino acids, respectively. Each of the resulting frames form the mutated peptide and the non-mutated peptide are then scored for predicted binding affinity with respect to MHC class I alleles (e.g., but not limited to, HLA-A and HLA-B alleles) and MHC class II alleles (e.g., but not limited to HLA-DRB1 alleles). Raw scores are normalized against the scores of a large sample of randomly generated peptides. The resulting “Z” scores are normally distributed and directly comparable across alleles. The resulting “Z” score is reported. In aspects, any 9-mer or 10-mer peptide with an allele-specific EpiMatrix™ Z-score in excess of 1.64, theoretically the top 5% of any given sample, is considered a putative T cell epitope.

As also further described in Example 1, peptides containing clusters of putative T cell epitopes are more likely to test positive in validating in vitro and in vivo assays. In aspects, the results of the initial EpiMatrix™ analysis are further screened for the presence of putative T cell epitope “clusters” using a second proprietary algorithm known as Clustimer™ algorithm. The Clustimer™ algorithm identifies sub-regions contained within any given amino acid sequence that contains a statistically unusually high number of putative T cell epitopes. Typical T-cell epitope “clusters” range from about 9 to roughly 30 amino acids in length and, considering their affinity to multiple alleles and across multiple 9-mer frames, can contain anywhere from about 4 to about 40 putative T cell epitopes. Each epitope cluster identified an aggregate EpiMatrix™ score is calculated by summing the scores of the putative T cell epitopes and subtracting a correcting factor based on the length of the candidate epitope cluster and the expected score of a randomly generated cluster of the same length. EpiMatrix™ cluster scores in excess of +10 are considered significant. In aspects, the T-cell epitopes of the instant disclosure contain several putative T-cell epitopes forming a pattern known as a T-cell epitope cluster.

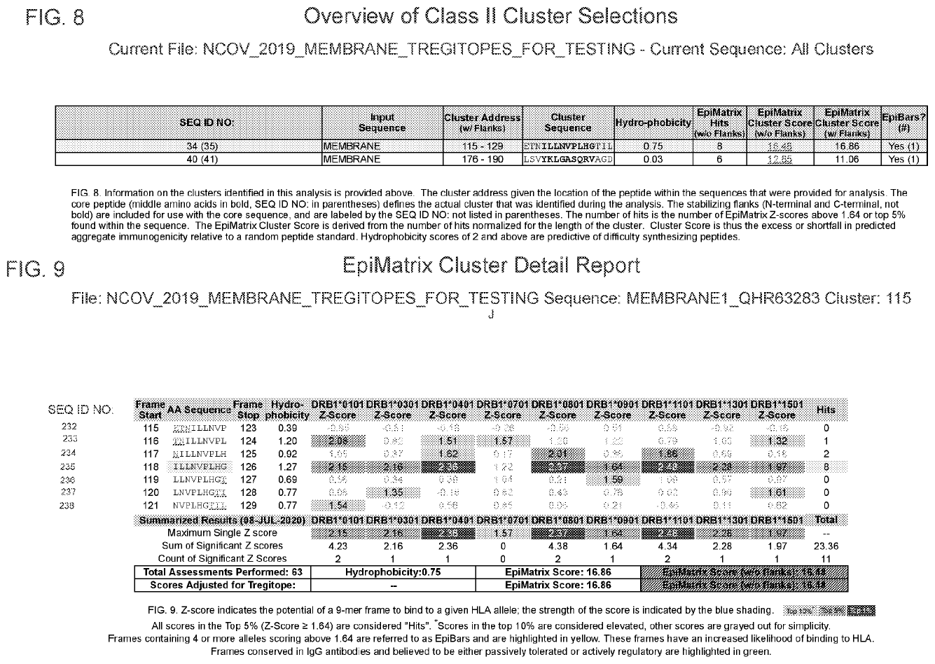

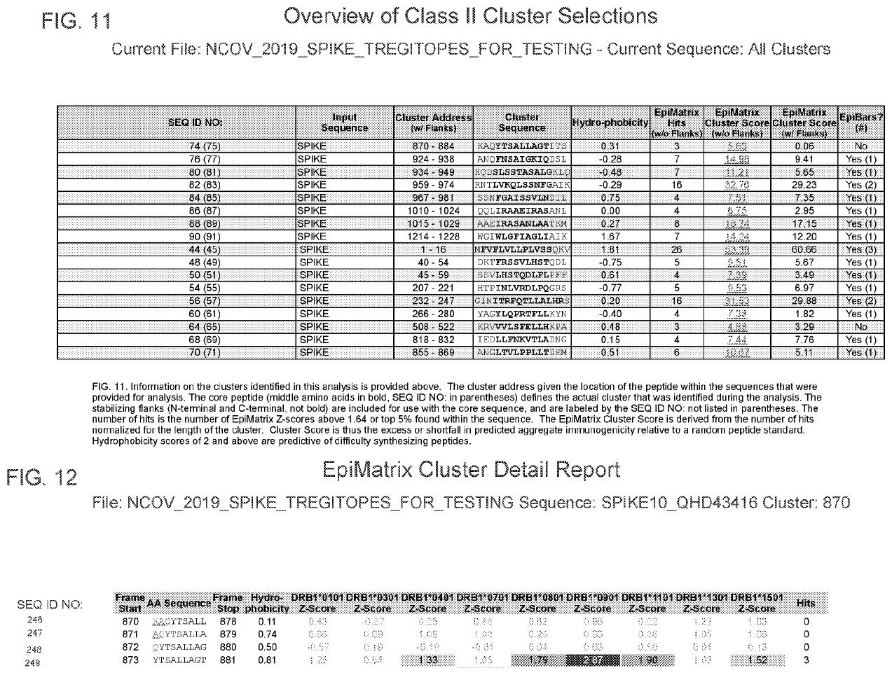

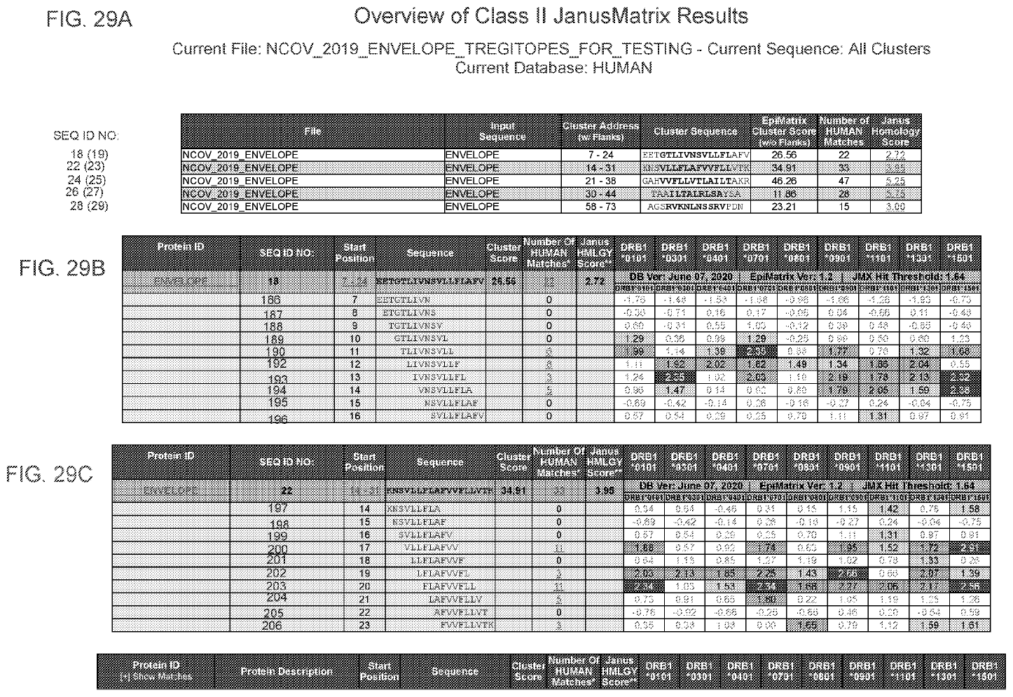

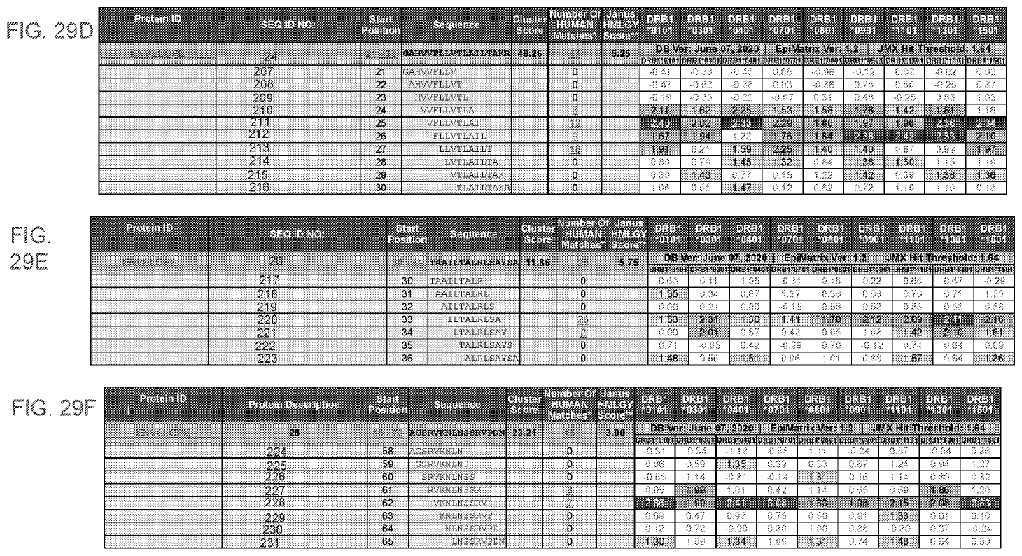

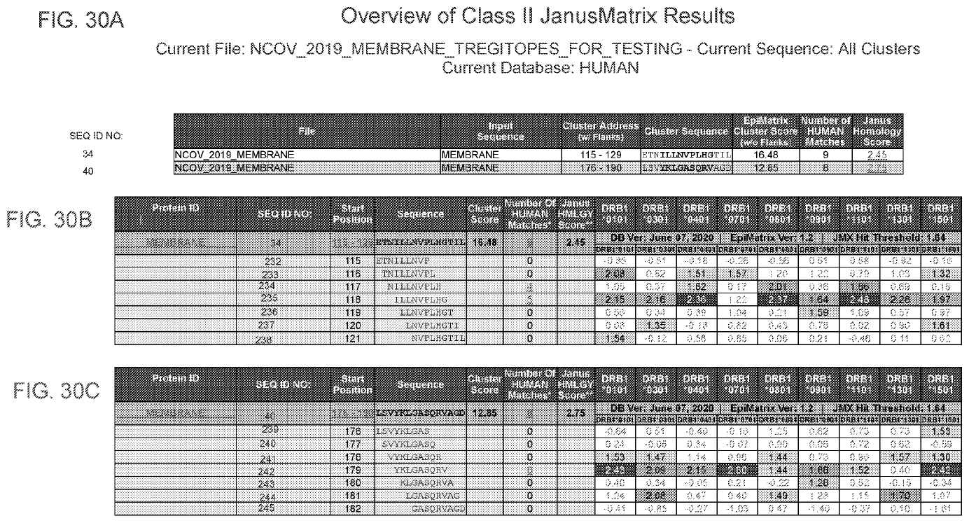

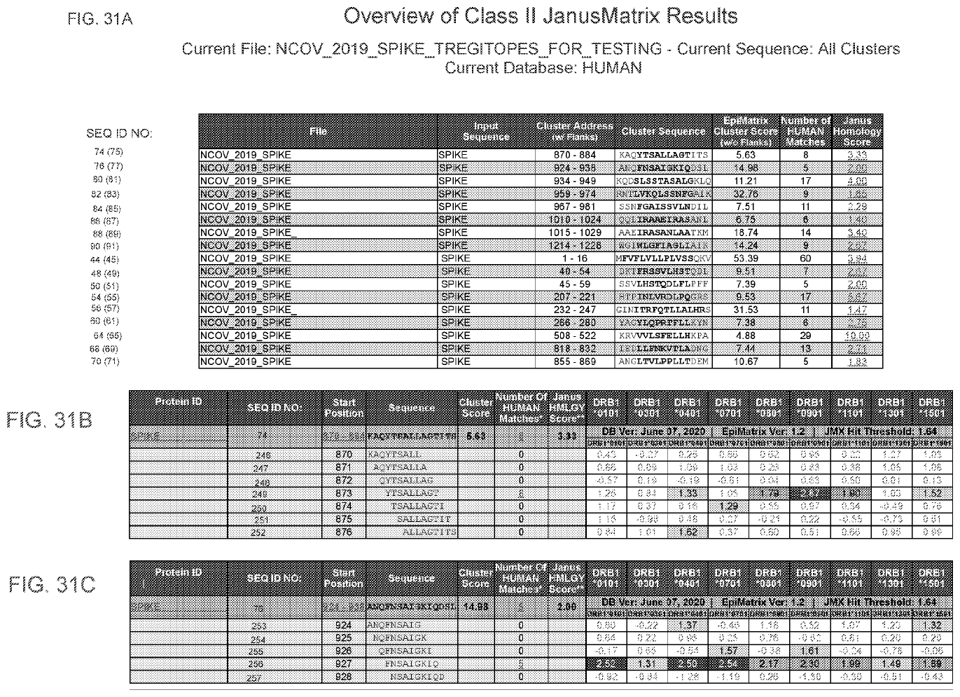

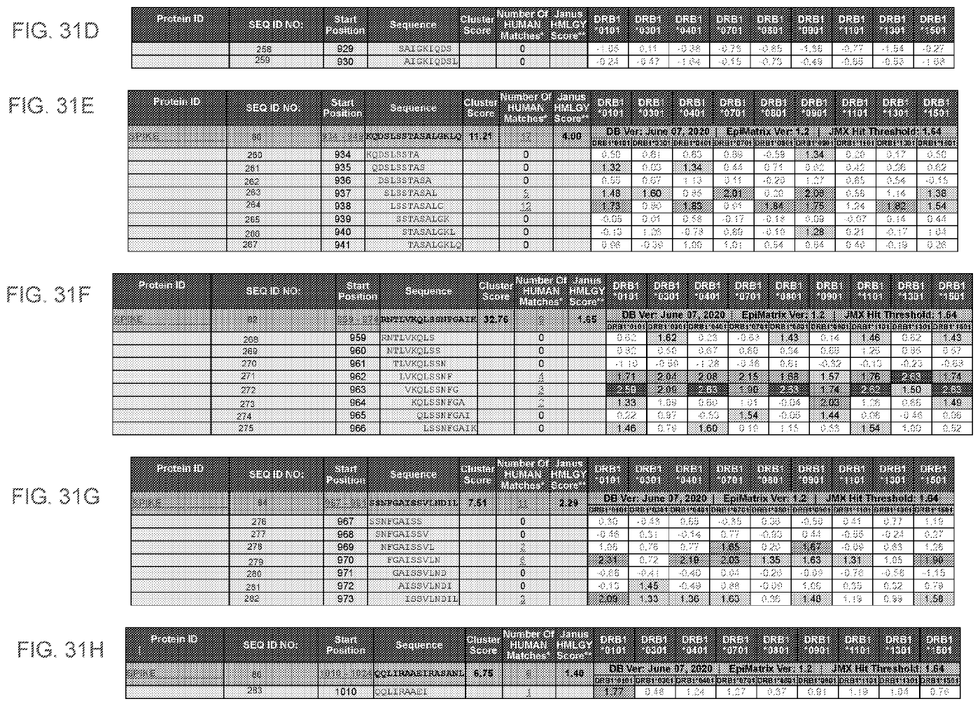

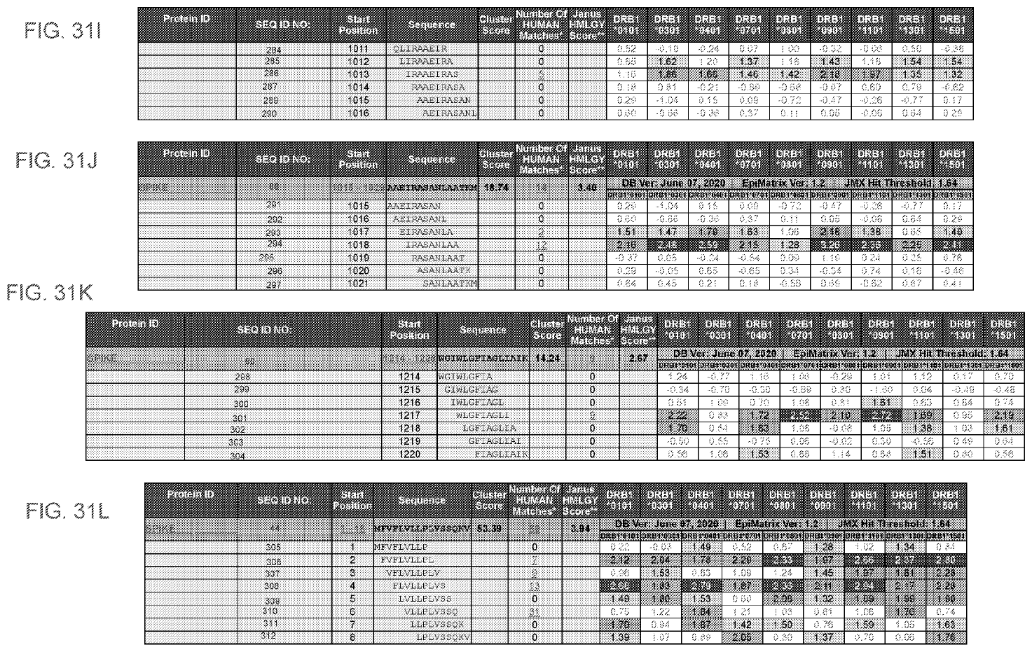

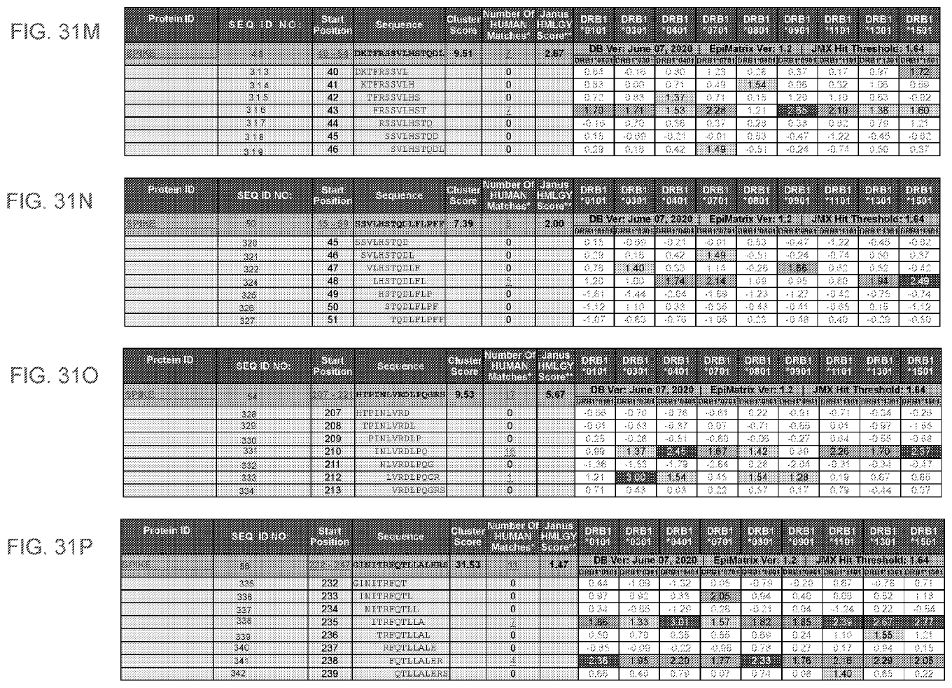

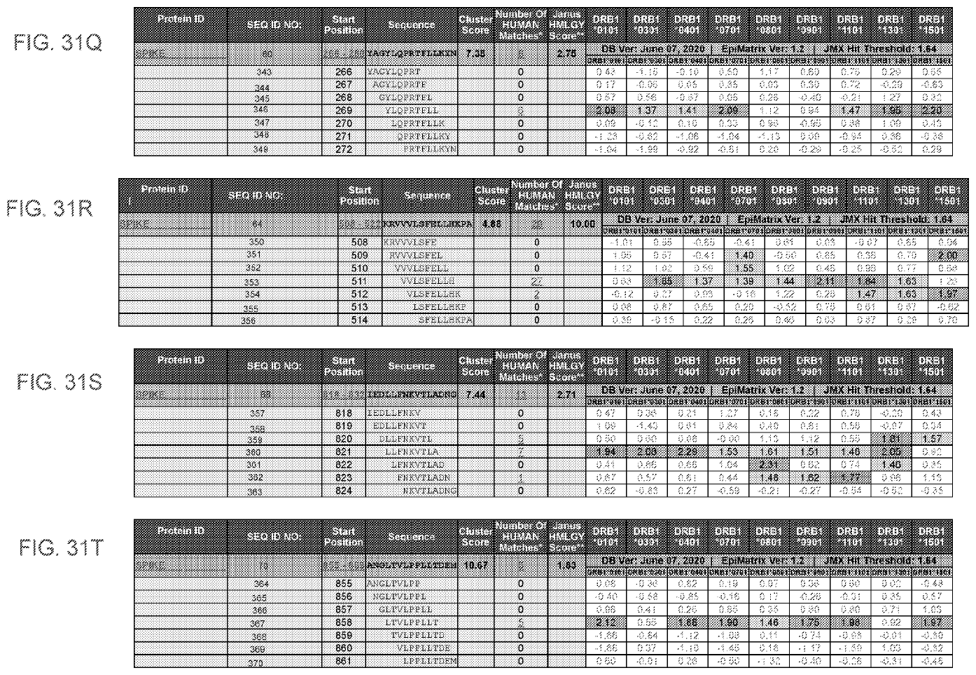

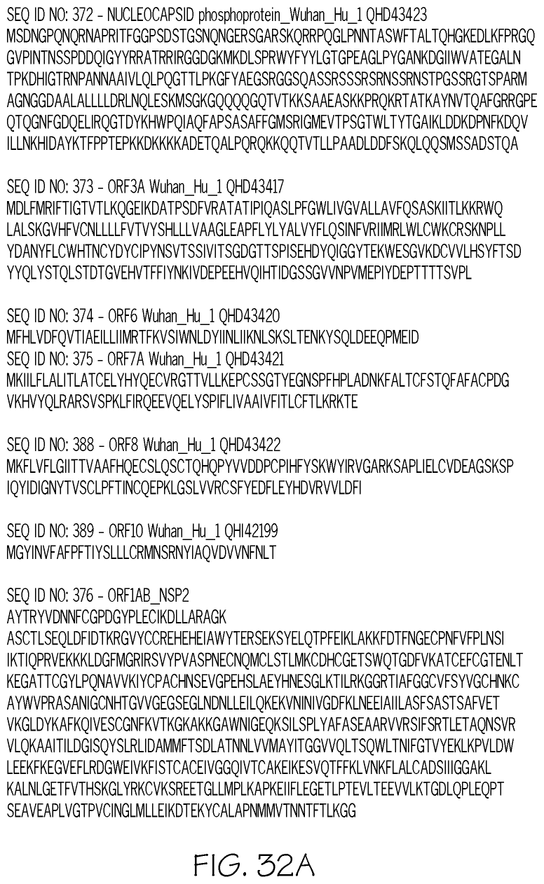

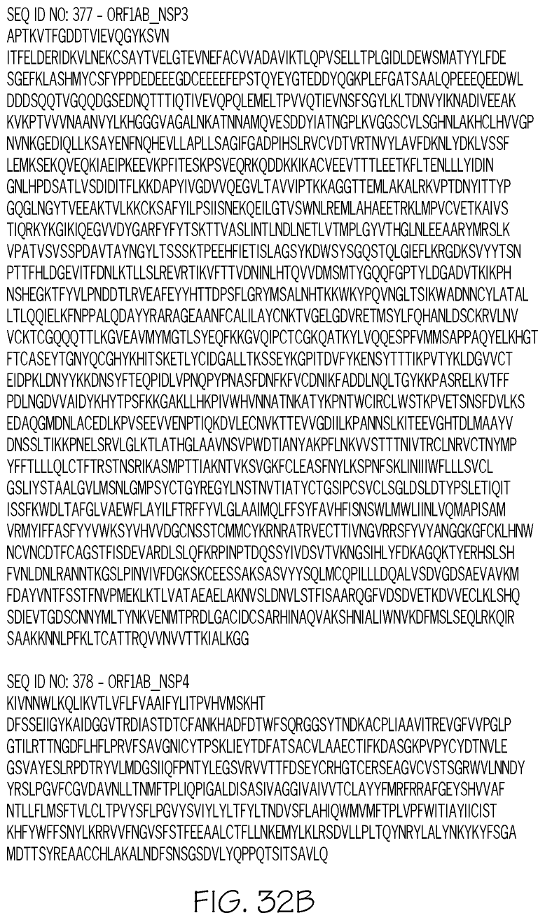

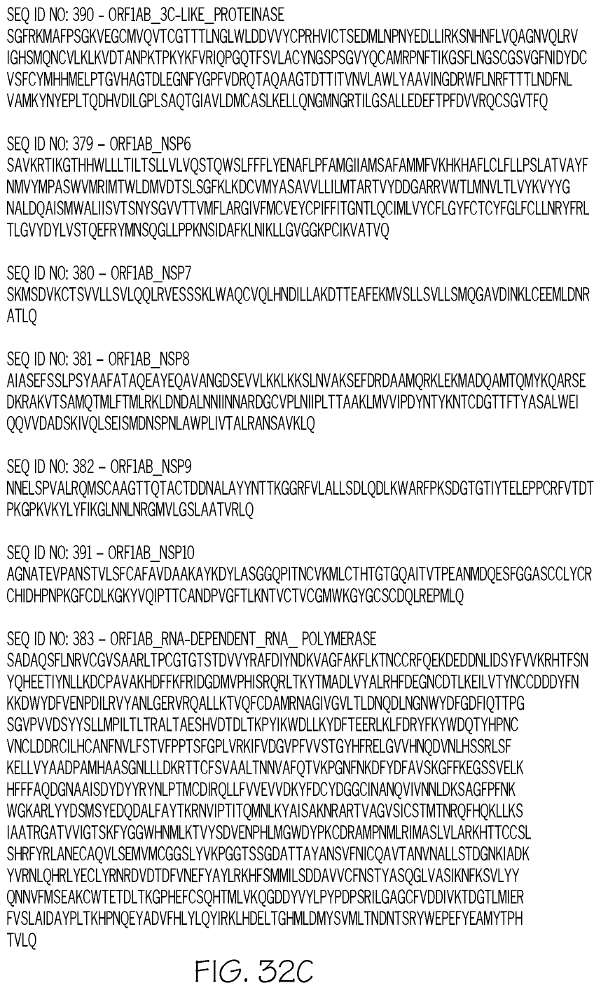

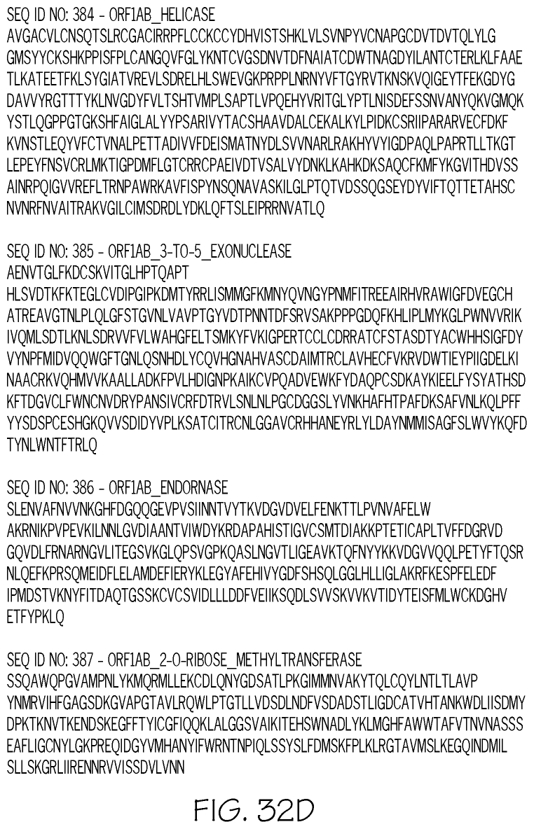

FIGS. 2-7 are EpiMatrix Cluster detail reports for identified MHC class II clusters of the envelope (SEQ ID NO: 1) of SARS-CoV-2 and relate to SEQ ID NOS: 18-31 and 186-231. FIGS. 8-10 are EpiMatrix Cluster detail reports for identified MHC class II clusters of the membrane (SEQ ID NO: 2) of SARS-CoV-2 and relate to SEQ ID NOS: 32-41 and 232-245. FIGS. 11-28 are EpiMatrix Cluster detail reports for identified MHC class II clusters of the spike (SEQ ID NO: 3) of SARS-CoV-2 and relate to SEQ ID NOS: 42-93 and 246-370. FIGS. 29A-F are JanusMatrix reports for identified MHC class II clusters of the envelope (SEQ ID NO: 1) of SARS-CoV-2 and relates to SEQ ID NOS: 186-231. FIGS. 30A-C are JanusMatrix reports for identified MHC class II clusters of the membrane (SEQ ID NO: 2) of SARS-CoV-2 and relates to SEQ ID NOS: 232-245. FIGS. 31A-T are JanusMatrix reports for identified MHC class II clusters of the spike (SEQ ID NO: 3) of SARS-CoV-2 and relates to SEQ ID NOS: 246-370.

Peptides containing clusters of putative T cell epitopes are more likely to test positive in validating in vitro and in vivo assays. The results of the initial EpiMatrix™ analysis are further screened for the presence of putative T cell epitope “clusters” using a second proprietary algorithm known as Clustimer™ algorithm. The Clustimer™ algorithm identifies sub-regions contained within any given amino acid sequence that contains a statistically unusually high number of putative T cell epitopes. Typical T-cell epitope “clusters” range from about 9 to roughly 30 amino acids in length and, considering their affinity to multiple alleles and across multiple 9-mer frames, can contain anywhere from about 4 to about 40 putative T cell epitopes. Each epitope cluster identified an aggregate EpiMatrix™ score is calculated by summing the scores of the putative T cell epitopes and subtracting a correcting factor based on the length of the candidate epitope cluster and the expected score of a randomly generated cluster of the same length. EpiMatrix™ cluster scores in excess of +10 are considered significant. In aspects, the Tregitopes of the instant disclosure contain several putative T cell epitopes forming a pattern known as a T cell epitope cluster.

Many of the most reactive T cell epitope clusters contain a feature referred to as an “EpiBar™.” An EpiBar™ is a single 9-mer frame that is predicted to be reactive to at least four different HLA alleles. In aspects, the Tregitopes of the present disclosure can comprise one or more EpiBars™.

The JanusMatrix system (EpiVax, Providence, R.I.) useful for screening peptide sequences for cross-conservation with a host proteome. JanusMatrix is an algorithm that predicts the potential for cross-reactivity between peptide clusters and the host genome or proteome, based on conservation of TCR-facing residues in their putative MHC ligands. The JanusMatrix algorithm first considers all the predicted epitopes contained within a given protein sequence and divides each predicted epitope into its constituent agretope and epitope. Each sequence is then screened against a database of host proteins. Peptides with a compatible MHC-facing agretope (i.e., the agretopes of both the input peptide and its host counterparty are predicted to bind the same MHC allele) and exactly the same TCR-facing epitope are returned. The JanusMatrix Homology Score suggests a bias towards immune tolerance. In the case of a therapeutic protein, cross-conservation between autologous human epitopes and epitopes in therapeutic may increase the likelihood that such a candidate will be tolerated by the human immune system. In the case of a vaccine, cross-conservation between human epitopes and the antigenic epitopes may indicate that such a candidate utilizes immune camouflage, thereby evading the immune response and making for an ineffective vaccine. When the host is, for example, a human, the peptide clusters are screened against human genomes and proteomes, based on conservation of TCR-facing residues in their putative HLA ligands. The peptides are then scored using the JanusMatrix Homology Score. In aspects, peptides with a JanusMatrix Homology Score above 3.0 indicate high tolerogenicity potential and as such may be very useful Tregitopes of the present disclosure.

FIGS. 29A-F are the overview of JanusMatrix results for select identified the Tregitopes, and 9-mers contained therein, identified in the envelope (SEQ ID NO: 1) of SARS-CoV-2. FIGS. 30A-C are the overview of JanusMatrix results for select identified the Tregitopes, and 9-mers contained therein, identified in the membrane (SEQ ID NO: 2) of SARS-CoV-2. FIGS. 31A-T are the overview of JanusMatrix results for select identified the Tregitopes, and 9-mers contained therein, identified in the spike (SEQ ID NO: 3) of SARS-CoV-2. With respect to a given EpiMatrix Hit (a 9-mer contained within the input sequence which is predicted to bind to a specific allele), a Janus Matrix match is a 9-mer derived from the search database (e.g., the human genome) which is predicted to bind to the same allele as the EpiMatrix Hit and shares TCR facing contacts with the EpiMatrix Hit. Further, the Janus Homology Score** represents the average depth of coverage in the search database for each EpiMatrix hit in the input sequence. For example, an input peptide with eight EpiMatrix hits, all of which have one match in the search database, has a Janus Homology Score of 1. An input peptide with four EpiMatrix Hits, all of which have two matches in the search database, has a Janus Homology Score of 2. The JanusMatrix Homology Score considers all constituent 9-mers in any given peptide, including flanks.

In aspects, peptides of the present disclosure bind to at least one and preferably two or more common HLA class II molecules with at least a moderate affinity (e.g., in aspects, <1000 μM IC₅₀, <500 μM IC₅₀, <400 μM IC₅₀, <300 μM IC₅₀, or <200 μM IC₅₀ in HLA binding assays based on soluble HLA molecules). In aspects, Tregitopes of the present disclosure are capable of being presented at the cell surface by APCs in the context of at least one and, in other aspects, two or more alleles of the HLA. In this context, the Tregitope-HLA complex can be recognized by naturally occurring T_{Regs} (in aspects, including natural T_{Regs} and/or adaptive T_{Regs}) having TCRs that are specific for the Tregitope-HLA complex and circulating in normal control subjects. In aspects, the recognition of the Tregitope-HLA complex can cause the matching regulatory T cell to be activated and to secrete regulatory cytokines and chemokines.

In other aspects, the detolerized antigens from the Tregitopes retain the peptide's HLA affinity, but are disrupted in the recognition by the T_{Regs}, including natural T_{Regs} and/or adaptive T_{Regs}. In other aspects, peptides include SARS-CoV-2 proteins or polypeptides wherein the Tregitopes are deleted, partially deleted and/or mutated.

In aspects, the present disclosure is directed to a polypeptide (of a Tregitope or a detolerized antigen thereof) having a sequence comprising, consisting of, or consisting essentially of one or more of SEQ ID NOS: 4-370, 391-440, and 448-833, including fragments thereof (e.g., but not limited to 9-mers, 12-mers, 15 mers, etc.). The phrase “consisting essentially of” is intended to mean that a polypeptide according to the present disclosure, in addition to having the sequence according to any of SEQ ID NOS: 4-370, 391-440, and 448-833 or a variant thereof, contains additional amino acids or residues that may be present at either terminus of the peptide and/or on a side chain that are not necessarily forming part of the peptide that functions as an MHC ligand and provided they do not substantially impair the activity of the peptide to function as a Tregitope. In certain aspects, such polypeptides can be capped with an N-terminal acetyl and/or C-terminal amino group.

In aspects, the instant disclosure is directed to a peptide or polypeptide comprising, consisting, or consisting essentially of an amino acid sequence of SEQ ID NOS: 4-370, 391-440, and 448-833 (and/or fragments or variants thereof), and optionally 1 to 12 additional amino acids distributed in any ratio on the N terminus and/or C-terminus of the polypeptide of SEQ ID NOS: 4-370, 391-440, and 448-833. In aspects, the instant disclosure is directed to a peptide or polypeptide have a core amino acid sequence comprising, consisting of, or consisting essentially of one or more peptides or polypeptides having an amino acid sequence of SEQ ID NOS: 4-370, 391-440, and 448-833, and optionally having extensions of 1 to 12 amino acids on the C-terminal and/or the N-terminal of the core amino acid sequence, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio). In aspects, the instant disclosure is directed to a peptide or polypeptide having a core sequence comprising, consisting of, or consisting essentially of one or more peptides or polypeptides having an amino acid sequence of SEQ ID NOS: 4-370, 391-440, and 448-833 (and/or fragments and variants thereof), optionally with extensions of 1 to 12 amino acids on the C-terminal and/or the N-terminal, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio), provided that the polypeptide with the flanking amino acids is still able to bind to the same HLA molecule (i.e., retain MHC binding propensity) as said polypeptide core sequence without said flanking amino acids. In aspects, said polypeptide with the flanking amino acids is still able to bind to the same HLA molecule (i.e., retain MHC binding propensity) and retain the same TCR specificity as said polypeptide core sequence without said flanking amino acids. In aspects, said flanking amino acid sequences are those that also flank the peptides or polypeptides included therein in the naturally occurring protein, for example, as described below:
For a peptide or polypeptide have a core sequence comprising, consisting of, or consisting essentially of one or more peptides or polypeptides having an amino acid sequence of SEQ ID NOS: 6, 7, 18-31, 186-231, and 448-459 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino add sequence of SEQ ID NOS: 6, 7, 18-31, 186-231, and 448-459 in the amino acid sequence of the envelope (SEQ ID NO: 1) of SARS-CoV-2. In some aspects, the peptides may include mutations to SEQ ID NO: 1 or a fragment thereof may include V62, L65, S67, and/or V70, (including V62A; V62G; V62N; V62Q; V62S; V62T; and/or S67Q); K63, N64, N66, S68, and/or R69; T11, L12, V14, N15, S16, V17, L19, F20, A22, F23, V24, V25, F26, L27, L28, V29, T30, L31, A32, I33, L34, A36, R38, and/or A41; and, L12, I13, V14, N15, S16, V17, L18, L19, F20, L21, A22, F23, V24, V25, F26, L27, L28, V29, T30, L31, A32, I33, L34T35, L37, L39, and/or C40.
For a peptide or polypeptide have a core sequence comprising, consisting of, or consisting essentially of one or more peptides or polypeptides having an amino acid sequence of SEQ ID NOS: 4, 5, 17, 32-41, 232-245, 440, and 450-471 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS. 4, 5, 17, 32-41, 232-245, 440, and 450-471 in the amino acid sequence of the membrane (SEQ ID NO: 2) of SARS-CoV-2. In some aspects, peptides may include mutations to SEQ ID NO: 2 or a peptide fragment thereof including I118, N121, P123, and/or G126 (including I118A; I118G; I118N; I118Q; I118S; I118T; N121P; P123Q; P123G; and/or G126P); L119, L120, V122, L124, and/or H125; Y179, G182, S184, and/or V187 (including Y179A; Y179N; Y1790; Y1795; Y179T; S184G; S184Q; and/or S184T); and, K180, L181, A183, Q185, and/or R186.
For a peptide or polypeptide have a core sequence comprising, consisting of, or consisting essentially of one or more peptides or polypeptides having an amino acid sequence of SEQ ID NOS. 8-17, 42-93, 246-370, 422, 423, 432, 434-439, and 794-833 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of 8-17, 42-93, 246-370, 422, 423, 432, 434-439, and 794-833 in the amino acid sequence of the spike (SEQ ID NO: 3) of SARS-CoV-2. In some aspects, peptides may include mutations to SEQ ID NO: 3 or a peptide fragment thereof including F28, S31, L33, T36, D38, and/or L41 (including F28G; F28A; F28N; F28T; F28S; F28Q, S31G; S31T and/or L33Q); R29, S30, V32, H34, S35, Q37, L39, and/or F40; I195, V198, D200 and/or Q203 (including I195A; I195G; I195N; I195S; I195T; I195Q; V198G; V198T; V198N; Q203E; Q203G; and/or Q203T); N196, L197, R199, L201, and/or P202; I220, F223, T225, and/or A228 (including I220A; I220G; I220N; I220Q; I220S; I220T; and/or T2250); T221, R222, Q224, L226, and/or L227; Y254, P257, Y259, and/or L262 (including Y254A; Y254G; Y254N; Y254Q; Y254S; Y254T; T259G; and/or T2590); L255, Q256, R258, F260, and/or L261; V496, S499, E501, and/or H504 (including V496A; V496G; V496N; V496Q; V496S; V496T; S499G; S499Q; and/or S499T); V497, L498, F500, L502, and/or L503; L806, N809, V811, and/or A814 (including L806A; L806G; L806N; L806Q; L806S; L806T; and/or N809G); L807, F808, K810, T812, and/or L813; L843, L846, P848, and/or T851 (including L843A; L843G; L843N; L843Q; L843S; L843T; L846G; L846T; and/or P8480); T844, V845, P847, L849, and/or L850; F912, A915, G917, 0920, L923, I926, S928, and/or G931 (including F912A; F912G; F912N; F912Q; F912S; F912T; A915G; L923A; L923G; L923N; L923Q; L923S; L923T, and/or T926G); N913, S914, I916, K918, I919, S924, S925, A927, A929, and/or L930; F955, I958, S960, and/or N963 (including F955A; F955G; F955N; F955Q; F955S; F955T, 1958G; S960G; S960Q; and/or S960T); G956, A957, S959, V961, and/or L962; I998, A1001, I1003, S1006, N1008, and/or A1011 (including I998A; I998G; I998N; I998Q; 1998S; 1998T; A1001G; A1001T; I1003A; I1003G; I1003N; I1003Q; I1003S; I1003T and/or N1008Q); and, R999, A1000, E1002, R104, A1005, A1007, L1009, and/or A1010.
For a peptide or polypeptide have a core sequence comprising, consisting of, or consisting essentially of one or more peptides or polypeptides having an amino acid sequence of SEQ ID NOS: 94, 95, 406-421, and 474-487 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ. ID NOS: 94, 95, 406-421, and 474-487 in the amino acid sequence of the nucleocapsid (SEQ ID NO: 371) of SARS-CoV-2.
For a peptide or polypeptide have a core sequence comprising, consisting of, or consisting essentially of one or more peptides or polypeptides having an amino acid sequence of SEQ ID NOS: 96-101, 424, and 756-774 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 96-101, 424, and 756-774 in the amino acid sequence of the ORF3a protein (SEQ ID NO: 372) of SARS-CoV-2.
For a peptide or polypeptide have a core sequence comprising, consisting of, or consisting essentially of one or more peptides or polypeptides having an amino acid sequence of SEQ ID NOS: 102, 103, and 775 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 102, 103, and 775 in the amino acid sequence of the ORF6 protein (SEQ ID NO: 373) of SARS-CoV-2.
For a peptide or polypeptide have a core sequence comprising, consisting of, or consisting essentially of one or more peptides or polypeptides having an amino acid sequence of SEQ ID NOS: 104-107, 425, and 776-783 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 104-107, 425, and 776-783 in the amino acid sequence of the ORF7a protein (SEQ ID NO: 374) of SARS-CoV-2.
For a peptide or polypeptide have a core sequence comprising, consisting of, or consisting essentially of one or more peptides or polypeptides having an amino acid sequence of SEQ ID NOS: 108-115, 427, and 554-575 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 108-115, 427, and 554-575 in the amino acid sequence of the ORF1ab non-structural protein 2 (NSP2) (SEQ ID NO: 375) of SARS-CoV-2.
For a peptide or polypeptide have a core sequence comprising, consisting of, or consisting essentially of one or more peptides or polypeptides having an amino acid sequence of SEQ. ID NOS: 116-135, 428, 429, and 576-655 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 116-135, 428, 429 and 576-655 in the amino acid sequence of the ORF1ab non-structural protein 3 (NSP3) (SEQ ID NO: 376) of SARS-CoV-2.
For a peptide or polypeptide have a core sequence comprising, consisting of, or consisting essentially of one or more peptides or polypeptides having an amino acid sequence of SEQ ID NOS: 136-141, 391, 392, and 656-684 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 136-141, 391, 392, and 656-684 in the amino acid sequence of the ORF1ab non-structural protein 4 (NSP4) (SEQ ID NO: 377) of SARS-CoV-2.
For a peptide or polypeptide have a core sequence comprising, consisting of, or consisting essentially of one or more peptides or polypeptides having an amino acid sequence of SEQ ID NOS: 142-149 and 685-708 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 142-149 and 685-708 in the amino acid sequence of the ORF1ab non-structural protein 6 (NSP6) (SEQ ID NO: 378) of SARS-CoV-2.
For a peptide or polypeptide have a core sequence comprising, consisting of, or consisting essentially of one or more peptides or polypeptides having an amino acid sequence of SEQ ID NOS: 150-153, 393-405, and 709-718 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 150-153, 393-405, and 709-718 in the amino acid sequence of the ORF1ab non-structural protein 7 (NSP7) (SEQ ID NO: 379) of SARS-CoV-2.
For a peptide or polypeptide have a core sequence comprising, consisting of, or consisting essentially of one or more peptides or polypeptides having an amino acid sequence of SEQ ID NOS: 154455 and 719-725 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 154-155 and 719-725 in the amino acid sequence of the ORF1ab non-structural protein 8 (NSP8) (SEQ ID NO: 380) of SARS-CoV-2,
For a peptide or polypeptide have a core sequence comprising, consisting of, or consisting essentially of one or more peptides or polypeptides having an amino acid sequence of SEQ ID NOS: 156-159 and 726-730 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 156-159 and 726-730 in the amino acid sequence of the ORF1ab non-structural protein 9 (NSP9) (SEQ ID NO: 381) of SARS-CoV-2.
For a peptide or polypeptide have a core sequence comprising, consisting of, or consisting essentially of one or more peptides or polypeptides having an amino acid sequence of SEQ. ID NOS: 160-165, 431, and 731-755 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 160-165, 431, and 731-755 in the amino acid sequence of the ORF1ab RNA-dependent RNA polymerase protein (SEQ ID NO: 382) of SARS-CoV-2.
For a peptide or polypeptide have a core sequence comprising, consisting of, or consisting essentially of one or more peptides or polypeptides having an amino acid sequence of SEQ ID NOS: 166-173 and 515-542 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 166-173 and 515-542 in the amino acid sequence of the ORF1ab helicase protein (SRI ID NO: 383) of SARS-CoV-2.
For a peptide or polypeptide have a core sequence comprising, consisting of, or consisting essentially of one or more peptides or polypeptides having an amino acid sequence of SEQ ID NOS: 174475 and 496-505 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 174-175 and 496-505 in the amino acid sequence of the ORF1ab 3′-5′ exonuclease protein (SEQ ID NO: 384) of SARS-CoV-2.
For a peptide or polypeptide have a core sequence comprising, consisting of, or consisting essentially of one or more peptides or polypeptides having an amino acid sequence of SEQ ID NOS: 176-179 and 506-514 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ. ID NOS: 176-179 and 506-514 in the amino acid sequence of the ORF1ab endoRNase protein (SEQ ID NO: 385) of SARS-CoV-2.
For a peptide or polypeptide have a core sequence comprising, consisting of, or consisting essentially of one or more peptides or polypeptides having an amino acid sequence of SEQ ID NOS: 180-185, 433, and 543-551 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 180-185, 433, and 543-551 in the amino acid sequence of the ORF1ab 2′O-ribose methyltransferase protein (SEQ ID NO: 386) of SARS-CoV-2.
For a peptide or polypeptide have a core sequence comprising, consisting of, or consisting essentially of one or more peptides or polypeptides having an amino acid sequence of SEQ ID NOS: 488 and 489 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 488 and 489 in the amino acid sequence of the ORF10 protein (SEQ ID NO: 388) of SARS-CoV-2.
For a peptide or polypeptide have a core sequence comprising, consisting of, or consisting essentially of one or more peptides or polypeptides having an amino acid sequence of SEQ ID NOS: 490-495 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 490-495 in the amino acid sequence of the ORF1ab 3C-like proteinase (SEQ ID NO: 389) of SARS-CoV-2.
For a peptide or polypeptide have a core sequence comprising, consisting of, or consisting essentially of one or more peptides or polypeptides having an amino acid sequence of SEQ ID NOS: 552 and 553 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 552 and 553 in the amino acid sequence of the ORF1ab non-structural protein 10 (NSP10) (SEQ ID NO: 390) of SARS-CoV-2.
For a peptide or polypeptide have a core sequence comprising, consisting of, or consisting essentially of one or more peptides or polypeptides having an amino acid sequence of SEQ ID NOS: 426 and 784-793 and/or a detolerized antigen thereof (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 426 and 784-793 in the amino acid sequence of the ORFS protein (SEQ ID NO: 389) of SARS-CoV-2.

In aspects, said flanking amino acid sequences as described herein may serve as a MHC stabilizing region. The use of a longer peptide may allow endogenous processing by patient cells and may lead to more effective antigen presentation and induction of T cell responses. In aspects, the peptides or polypeptides can be in either neutral (uncharged) or salt forms, and may be either free of or include modifications such as glycosylation, side chain oxidation, or phosphorylation. In certain aspects, the peptides can be capped with an N-terminal acetyl and/or C-terminal amino group. In aspects, said flanking amino acid sequences as described herein may serve as a MHC stabilizing region. The use of a longer peptide may allow endogenous processing by patient cells and may lead to more effective antigen presentation and induction of T cell responses. In aspects, the peptides or polypeptides can be in either neutral (uncharged) or salt forms, and may be either free of or include modifications such as glycosylation, side chain oxidation, or phosphorylation. In certain aspects, such polypeptides can be capped with an N-terminal acetyl and/or C-terminal amino group.

In aspects, the peptides or polypeptides of the instant disclosure can be isolated, recombinant, and/or synthetic. In aspects, the instant disclosure is directed to a polypeptide comprising an amino acid sequence having at least 75%, 80%, 85%, 90%, or 95% homology to any one of SEQ ID NOS: 4-370, 391-440, and 448-833 (and/or fragments thereof), including detolerized antigens thereof, wherein said polypeptide is still able to bind to a same HLA molecule (i.e., retain MHC binding propensity) and/or retain the same TCR specificity, and/or retain anti-SARS-CoV-2 activity. As used herein, two polypeptides (or a region of the polypeptides) are substantially homologous or identical when the amino acid sequences are at least about 45-55%, typically at least about 70-75%, more typically at least about 80-85%, more typically greater than about 90%, and more typically greater than 95% or more homologous or identical. To determine the percent homology or identity of two amino acid sequences, or of two nucleic acid sequences, the sequences are aligned for optimal comparison purposes (e.g., gaps can be introduced in the sequence of one polypeptide or nucleic acid molecule for optimal alignment with the other polypeptide or nucleic acid molecule). The amino acid residues or nucleotides at corresponding amino acid positions or nucleotide positions are then compared. When a position in one sequence is occupied by the same amino acid residue or nucleotide as the corresponding position in the other sequence, then the molecules are homologous at that position. As is known in the art, the percent identity between the two sequences is a function of the number of identical positions shared by the sequences, taking into account the number of gaps, and the length of each gap, which need to be introduced for optimal alignment of the two sequences. Sequence homology for polypeptides is typically measured using sequence analysis software. As used herein, amino acid or nucleic acid “homology” is equivalent to amino acid or nucleic acid “identity”. In aspects, the percent homology between the two sequences is a function of the number of identical positions shared by the sequences (e.g., percent homology equals the number of identical positions/total number of positions×100).

In aspects, the present disclosure also encompasses polypeptides having a lower degree of identity but having sufficient similarity so as to perform one or more of the same functions performed by a polypeptide of the instant disclosure (e.g., a polypeptide having a sequence comprising, consisting of, or consisting essentially of one or more of SEQ ID NOS: 4-370, 391-440, and 448-833 and/or fragments and variants thereof, and optionally 1 to 12 additional amino acids distributed in any ratio on the N terminus and/or C-terminus of the polypeptide of SEQ ID NOS: 4-370, 391-440, and 448-833. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu, Met, and Ile; interchange of the hydroxyl residues Ser and Thr, exchange of the acidic residues Asp and Glu, substitution between the amide residues Asn and Gln, exchange of the basic residues His, Lys and Arg and replacements among the aromatic residues Trp, Phe and Tyr. Guidance concerning which amino acid changes are likely to be phenotypically silent are found (Bowie J U et al., (1990), Science, 247(4948):130610, which is herein incorporated by reference in its entirety).

In aspects, functional variants of a polypeptide having a sequence (or a core sequence) comprising, consisting of, or consisting essentially of one or more of SEQ ID NOS: 4-370, 391-440, and 448-833 as disclosed herein may contain one or more conservative substitutions, and in aspects one or more non-conservative substitutions, at amino acid residues which are not believed to be essential for functioning (e.g., retain MHC binding propensity and/or TCR specificity, and/or retain anti-SARS-CoV-2 activity) of the instantly-disclosed polypeptides. For example, in aspects, a variant polypeptide having a sequence (or a core sequence) comprising, consisting of, or consisting essentially of one or more of SEQ ID NOS: 4-370, 391-440, and 448-833, or fragments thereof as disclosed herein, may contain one or more conservative substitutions (and in aspects, a nonconservative substitution) in one or more HLA contact residues, provided HLA binding is preserved. MHC binding assays are well known in the art. In aspects, such assays may include the testing of binding affinity with respect to MHC class I and class II alleles in in vitro binding assays, with such binding assays as are known in the art. Examples include, e.g., the soluble binding assays as disclosed in U.S. Pat. No. 7,884,184 or PCT/US2020/020089, both of which are herein incorporated by reference in their entireties. Additionally, in aspects, a fully functional variant polypeptide having a sequence (or a core sequence) comprising, consisting of, or consisting essentially of one or more of SEQ ID NOS: 4-370, 391-440, and 448-833 as disclosed herein do not contain mutations at one or more critical residues or regions, such as TCR contact residues.

In aspects, the TCR-binding epitope (which can be referred to as TCR binding residues, TCR facing epitope, TCR facing residues, or TCR contacts) for a 9-mer identified epitope (which may be a 9-mer fragment of one or more of SEQ ID NOS: 4-370, 391-440, and 448-833 as disclosed herein) that bind to a MHC class II molecule are at position 2, 3, 5, 7, and 8 of the identified epitope, while the MHC-binding agretope (which can be referred to as MHC contacts, MHC facing residues, MHC-binding residues, or MHC-binding face) for a 9-mer identified epitope (which may be a 9-mer fragment of one or more of SEQ ID NOS: 4-370, 391-440, and 448-833 as disclosed herein) that bind to a MHC class II molecule are at position 1, 4, 6, and 9, both as counted from the amino terminal.

In aspects, the TCR binding epitope for a 9-mer identified epitope (which may be a 9-mer fragment of one or more of SEQ ID NOS: 44-370, 391-440, and 448-833 or as disclosed herein) that binds to a MHC class I molecule are at position 4, 5, 6, 7, and 8 of the identified epitope, while the MHC binding agretope for a 9-mer identified epitope (which may be a 9-mer fragment of one or more of SEQ ID NOS: 4-370, 391-440, and 448-833 as disclosed herein) that bind to a MHC class I molecule are at position 1, 2, 3, and 9, both as counted from the amino terminal.

In aspects, the TCR binding epitope for a 10-mer identified epitope that bind to a MHC class I molecule are at position 4, 5, 6, 7, 8, and 9 of the identified epitope (which may be a 10-mer fragment of one or more of SEQ ID NOS: 4-370, 391-440, and 448-833 as disclosed herein), while the MHC binding agretope for a 10-mer identified epitope (which may be a 10-mer fragment of one or more of SEQ ID NOS: 4-370, 391-440, and 448-833 as disclosed herein) that bind to a MHC class I molecule are at position 1, 2, 3, 9, and 10, both as counted from the amino terminal.

In aspects, the TCR-binding epitope for a 9-mer identified epitope (which may be a 9-mer fragment of one or more of SEQ ID NOS: 4-370, 391-440, and 448-833 as disclosed herein) that bind to a MHC class II molecule are at any combination of residues at positions 2, 3, 5, 7, and 8 (e.g., but not limited to, positions 3, 5, 7 and 8; positions 2, 5, 7, and 8; positions 2, 3, 5, and 7, etc.) of the identified epitope, while the MHC binding agretope for a 9-mer identified epitope (which may be a 9-mer fragment of one or more of SEQ ID NOS: 4-370, 391-440, and 448-833 as disclosed herein) is the complementary face to the TCR facing residues, both as counted from the amino terminal.

In aspects, the TCR binding epitope for 9-mer identified epitope (which may be a 9-mer fragment of one or more of SEQ ID NOS: 4-370, 391-440, and 448-833 as disclosed herein) that bind to a MHC class I molecule are at positions 4, 5, 6, 7, and 8; 1, 4, 5, 6, 7 and 8; or 1, 3, 4, 5, 6, 7, and 8 of the identified epitope, while the MHC binding agretope for a 9-mer identified epitope (which may be a 9-mer fragment of one or more of SEQ ID NOS: 4-370, 391-440, and 448-833 as disclosed herein) is the complementary face to the TCR facing residues, both as counted from the amino terminal.

In aspects, the TCR-binding epitope for a 10-mer identified epitope (which may be a 10-mer fragment of one or more of SEQ ID NOS: 4-370, 391-440, and 448-833 as disclosed herein) that bind to a MHC class I molecule are at any combination of residues at positions 1, 3, 4, 5, 6, 7, 8, and 9 of the identified epitope, while the MHC binding agretope for a 10-mer identified epitope (which may be a 10-mer fragment of one or more of SEQ ID NOS: 4-370, 391-440, and 448-833 as disclosed herein) is the complementary face to the TCR facing residues, both as counted from the amino terminal.

Based on the above, it should be understood that in aspects in which one or more 9-mers and/or 10-mer epitopes that are contained within a longer polypeptide and are predicted to bind one or more Class I or Class II MHC molecules and are occurring in close proximity to each other in a naturally occurring sequence (e.g., wherein position 1 of each pair of binding 9-mers and/or 10-mers fall within, e.g., 3 amino acids of each other), such epitopes may be combined to form an Epitope Cluster. In a given cluster, any given amino acid may be, with respect to a given 9-mer epitope or 10-mer epitope, MHC facing and, with respect to another 9-mer epitope, TCR facing.

In aspects, the present disclosure also includes fragments of the instantly-disclosed polypeptides. In aspects, the present disclosure also encompasses fragments of the variants of the instantly-disclosed polypeptides and concatemeric polypeptides as described herein. In aspects, as used herein, a fragment comprises at least about nine contiguous amino acids. In aspects, the present disclosure also encompasses fragments of the variants of the T-cell epitopes described herein. Useful fragments (and fragments of the variants of the polypeptides and concatemeric polypeptides described herein) include those that retain one or more of the biological activities, particularly: MHC binding propensity and/or TCR specificity, and/or anti-SARS-CoV-2 activity. Biologically active fragments are, for example, about 9, 10, 11, 12, 1, 14, 15, 16, 20, 25, 30, 40, 50, 60, 70, 80, 90, 100 or more amino acids in length, including any value or range there between. Fragments can be discrete (not fused to other amino acids or polypeptides) or can be within a larger polypeptide. Several fragments can be comprised within a single larger polypeptide. In aspects, a fragment designed for expression in a host can have heterologous pre- and pro-polypeptide regions fused to the amino terminus of the polypeptide fragment and an additional region fused to the carboxyl terminus of the fragment.

In aspects, the instantly disclosed polypeptides and concatemeric polypeptides of the present disclosure can include allelic or sequence variants (“mutants”) or analogs thereof, or can include chemical modifications (e.g., pegylation, glycosylation). In aspects, a mutant retains the same function, particularly MHC binding propensity and/or TCR specificity, and/or anti-SARS-CoV-2 activity. In aspects, a mutant can provide for enhanced binding to MHC molecules. In aspects, a mutant can lead to enhanced binding to TCRs. In another instance, a mutant can lead to a decrease in binding to MHC molecules and/or TCRs. Also contemplated is a mutant that binds, but does not allow signaling via the TCR.

In aspects, one or more peptides or polypeptides of the instant disclosure (e.g., but not limited to, a peptide or polypeptide comprising, consisting, or consisting essentially of an amino acid sequence of SEQ ID NOS: 4-370, 391-440, and 448-833 (and/or detolerized antigens, including detolerized variants, fragments or variants thereof) may be joined to, linked to (e.g., fused in-frame, chemically-linked, or otherwise bound), and/or inserted into a heterologous polypeptide. In aspects, the one or more peptides or polypeptides of the instant disclosure may be joined to, linked to (e.g., fused in-frame, chemically linked, or otherwise bound), and/or inserted into a heterologous polypeptide as a whole, although it may be made up from a joined to, linked to (e.g., fused in-frame, chemically linked, or otherwise bound), and/or inserted amino acid sequence, together with flanking amino acids of the heterologous polypeptide.

In aspects, one or more peptides or polypeptides of the instant disclosure may be joined to, linked to (e.g., fused in-frame, chemically linked, or otherwise bound), and/or inserted into a heterologous polypeptide (e.g. but not limited to, a heterologous antibody (which can be IgG, IgM, IgA, IgD or IgE molecules or antigen-specific antibody fragments thereof (including, but not limited to, a Fab, F(ab′)₂, Fv, disulfide linked Fv, scFv, single domain antibody, closed conformation multispecific antibody, disulfide-linked scfv, diabody)). As previously described, the term “heterologous polypeptide” is intended to mean that the one or more peptides of the instant disclosure are heterologous to, or not included naturally, in the heterologous polypeptide. In aspects, one or more of the instantly-disclosed polypeptides (Tregitopes or detolerized antigens thereof) may be inserted into the heterologous polypeptide (e.g., through recombinant techniques, mutagenesis, or other known means in the art), may be added to the C-terminus (with or without the use of linkers, as is known in the art), and/or added to the N-terminus (with or without the use of linkers, as is known in the art) of the heterologous polypeptide. For example, protein engineering by mutagenesis can be performed using site-directed mutagenesis techniques, or other mutagenesis techniques known in the art (see e.g., James A. Brannigan and Anthony J. Wilkinson, 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety). In aspects, the one or more peptides may be inserted into or replace amino acids in a Fc domain as disclosed in U.S. Pat. Nos. 7,442,778, 7,645,861, 7,655,764, 7,655,765, and/or 7,750,128 (each of which are herein incorporated by reference in their entirety). In aspects, the one or more peptides or polypeptides of the instant disclosure may be joined to, linked to (e.g., fused in-frame, chemically linked, or otherwise bound), and/or inserted into a heterologous polypeptide as a whole, although it may be made up from a joined to, linked to (e.g., fused in-frame, chemically linked, or otherwise bound), and/or inserted amino acid sequence, together with flanking amino acids of the heterologous polypeptide. In aspects, the peptides or polypeptides can be in either neutral (uncharged) or salt forms, and may be either free of or include modifications such as glycosylation, side chain oxidation, or phosphorylation. In certain aspects, the peptides or polypeptides can be capped with an N-terminal acetyl and/or C-terminal amino group.

In aspects, the present disclosure is directed to polypeptide (which, in aspects, may be an isolated, synthetic, or recombinant) having a sequence comprising one or more of SEQ ID NOS: 4-370, 391-440, and 448-833 (and/or fragments or variants thereof), wherein said one or more of SEQ ID NOS: 4-370, 391-440, and 448-833 is not naturally included in the polypeptide and/or said one or more of SEQ ID NOS: 4-370, 391-440, and 448-833 is not located at its natural position in the polypeptide. In aspects of the above-described polypeptides, the polypeptides may be isolated, synthetic, or recombinant. In aspects, the peptides or polypeptides can be in either neutral (uncharged) or salt forms, and may be either free of or include modifications such as glycosylation, side chain oxidation, or phosphorylation. In certain aspects, the peptides or polypeptides can be capped with an N-terminal acetyl and/or C-terminal amino group.

The manner of producing the polypeptides of the present disclosure will vary widely, depending upon the nature of the various elements comprising the molecule. For example, an isolated polypeptide can be purified from cells that naturally express it, purified from cells that have been altered to express it (recombinant), or synthesized using known protein synthesis methods. The synthetic procedures may be selected so as to be simple, provide for high yields, and allow for a highly purified stable product. For example, polypeptides of the instant disclosure can be produced either from a nucleic acid disclosed herein, or by the use of standard molecular biology techniques, such as recombinant techniques, mutagenesis, or other known means in the art. An isolated polypeptide can be purified from cells that naturally express it, purified from cells that have been altered to express it (recombinant), or synthesized using known protein synthesis techniques. In aspects, a polypeptide of the instant disclosure is produced by recombinant DNA or RNA techniques. In aspects, a polypeptide of the instant disclosure can be produced by expression of a recombinant nucleic acid of the instant disclosure in an appropriate host cell. For example, a nucleic acid molecule encoding the polypeptide is cloned into an expression cassette or expression vector, the expression cassette or expression vector introduced into a host cell and the polypeptide expressed in the host cell. The polypeptide can then be isolated from the cells by an appropriate purification scheme using standard protein purification techniques. Alternatively a polypeptide can be produced by a combination of ex vivo procedures, such as protease digestion and purification. Further, polypeptides of the instant disclosure can be produced using site-directed mutagenesis techniques, or other mutagenesis techniques known in the art (see e.g., James A. Brannigan and Anthony J. Wilkinson, 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety).

A chimeric or fusion polypeptide composition can be produced by standard recombinant DNA or RNA techniques as are known in the art. For example, DNA or RNA fragments coding for the different polypeptide sequences may be ligated together in-frame in accordance with conventional techniques. In another embodiment, the fusion gene can be synthesized by conventional techniques including automated DNA synthesizers. Alternatively, polymerase chain reaction (PCR) amplification of nucleic acid fragments can be carried out using anchor primers which give rise to complementary overhangs between two consecutive nucleic acid fragments which can subsequently be annealed and re-amplified to generate a chimeric nucleic acid sequence (Short Protocols in Molecular Biology: A Compendium of Methods from Current Protocols in Molecular Biology, (2^{ND}, 1992), F M Asubel et al. (eds), Green Publication Associates, New York, N.Y. (Publ), ISBN: 9780471566355, which is herein incorporated by reference in its entirety). Further, one or more peptides, polypeptides or concatemeric of the instant disclosure (e.g., but not limited to, a peptide or polypeptide comprising, consisting, or consisting essentially of an amino acid sequence of SEQ ID NOS: 4-1676, 1713-2595, and 2605-2638 (and/or fragments or variants thereof), and optionally 1 to 12 additional amino acids distributed in any ratio on the N terminus and/or C-terminus of the polypeptide of SEQ ID NOS: 4-1676, 1713-2595, and 2605-2638) can be inserted into a heterologous polypeptide or inserted into a non-naturally occurring position of a polypeptide through recombinant techniques, synthetic polymerization techniques, mutagenesis, or other standard techniques known in the art. For example, protein engineering by mutagenesis can be performed using site-directed mutagenesis techniques, or other mutagenesis techniques known in the art (see e.g., James A. Brannigan and Anthony J. Wilkinson, 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety).

In aspects, the polypeptides, concatemeric polypeptides, and chimeric or fusion polypeptides can be purified to homogeneity or partially purified. It is understood, however, that preparations in which the T-cell epitope compounds and compositions are not purified to homogeneity are useful. The critical feature is that the preparation allows for the desired function of the composition, even in the presence of considerable amounts of other components. Thus, the present disclosure encompasses various degrees of purity. In one embodiment, the language “substantially free of cellular material” includes preparations of the polypeptides, concatemeric polypeptides, and chimeric or fusion polypeptides having less than about 30% (by dry weight) other proteins (e.g., contaminating protein), less than about 20% other proteins, less than about 10% other proteins, less than about 5% other proteins, less than about 4% other proteins, less than about 3% other proteins, less than about 2% other proteins, less than about 1% other proteins, or any value or range therebetween.

In aspects, when a polypeptide, concatemeric polypeptide, and chimeric or fusion polypeptide of the present disclosure is recombinantly produced, the composition can also be substantially free of culture medium, for example, culture medium represents less than about 20%, less than about 10%, or less than about 5% of the volume of the polypeptides, concatemeric polypeptides, and chimeric or fusion polypeptides preparation. The language “substantially free of chemical precursors or other chemicals” includes preparations of the polypeptides, concatemeric polypeptides, and chimeric or fusion polypeptides in which it is separated from chemical precursors or other chemicals that are involved in the T-cell epitope's synthesis. The language “substantially free of chemical precursors or other chemicals” can include, for example, preparations of the polypeptides, concatemeric polypeptides, and chimeric or fusion polypeptides having less than about 30% (by dry weight) chemical precursors or other chemicals, less than about 20% chemical precursors or other chemicals, less than about 10% chemical precursors or other chemicals, less than about 5% chemical precursors or other chemicals, less than about 4% chemical precursors or other chemicals, less than about 3% chemical precursors or other chemicals, less than about 2% chemical precursors or other chemicals, or less than about 1% chemical precursors or other chemicals.

In aspects, the present disclosure also includes pharmaceutically acceptable salts of the T-cell epitope compounds and compositions (including one or more of e.g., peptides or polypeptides as disclosed herein; concatemeric peptides as disclosed herein; chimeric or fusion polypeptide compositions as disclosed herein (which in aspects may be isolated, synthetic, and/or recombinant). “Pharmaceutically acceptable salt” means a salt that is pharmaceutically acceptable and that possesses the desired pharmacological activity of the parent peptide or polypeptide (e.g., peptides, polypeptides, concatemeric peptides, and/or chimeric or fusion polypeptides as disclosed herein). As used herein, “pharmaceutically acceptable salt” refers to derivative of the instantly-disclosed polypeptides, concatemeric polypeptides, and/or chimeric or fusion polypeptides, wherein such compounds are modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc

In aspects, the present disclosure is directed to a concatemeric polypeptide or peptide that comprises at one or more of the instantly-disclosed polypeptides or peptides linked, fused, or joined together (e.g., fused in-frame, chemically linked, or otherwise bound) to an additional peptide or polypeptide. Such additional peptide or polypeptide may be one or more of the instantly disclosed polypeptides or peptides, or may be an additional peptide or polypeptide of interest. In aspects a concatemeric peptide is composed of 3 or more, 4 or more, 5 or more 6 or more 7 or more, 8 or more, 9 or more of the instantly-disclosed peptides or polypeptides. In other aspects, the concatemeric peptides or polypeptides include 1000 or more, 1000 or less, 900 or less, 500 or less, 100 or less, 75 or less, 50 or less, 40 or less, 30 or less, 20 or less or 100 or less peptide epitopes. In yet other embodiments, a concatemeric peptide has 3-100, 5-100, 10-100, 15-100, 20-100, 25-100, 30-100, 35-100, 40-100, 45-100, 50-100, 55-100, 60-100, 65-100, 70-100, 75-100, 80-100, 90-100, 5-50, 10-50, 15-50, 20-50, 25-50, 30-50, 35-50, 40-50, 45-50, 100-150, 100-200, 100-300, 100-400, 100-500, 50-500, 50-800, 50-1,000, or 100-1,000 of the instantly disclosed peptides or polypeptides linked, fused, or joined together. Each peptide or polypeptide of the concatemeric polypeptide may optionally have one or more linkers, which may optionally be cleavage sensitive sites, adjacent to their N and/or C terminal end. In such a concatemeric peptide, two or more of the peptide epitopes may have a cleavage sensitive site between them. Alternatively, two or more of the peptide epitopes may be connected directly to one another or through a linker that is not a cleavage sensitive site. In certain aspects, such concatemeric polypeptides can be capped with an N-terminal acetyl and/or C-terminal amino group. In aspects of above-described concatemeric peptides or polypeptides, the concatemeric peptides or polypeptides may be isolated, synthetic, or recombinant. In aspects, the concatemeric peptides or polypeptides can be in either neutral (uncharged) or salt forms, and may be either free of or include modifications such as glycosylation, side chain oxidation, or phosphorylation. In certain aspects, the concatemeric polypeptides can be capped with a B-terminal acetyl and/or C-terminal amino group.

In aspects, the compositions of the present disclosure comprise one or more peptides (e.g., but not limited to, a peptide or polypeptide comprising, consisting, or consisting essentially of an amino acid sequence of SEQ ID NOS: 4-370, 391-440, and 448-833 or a detolerized variant thereof) incorporated as an internal sequence into an Fc domain as disclosed in U.S. Pat. Nos. 7,442,778, 7,645,861, 7,655,764, 7,655,765, and/or 7,750,128 (each of which are herein incorporated by reference in their entirety). Such an internal sequence may be added by insertion (i.e., between amino acids in the previously existing Fc domain) or by replacement of amino acids in the previously existing Fc domain (i.e., removing amino acids in the previously existing Fc domain and adding peptide amino acids). In the latter case, the number of peptide amino acids added need not correspond to the number of amino acids removed from the previously existing Fc domain; for example, in aspects, the compositions may comprise an added internal sequence of 9-21 amino acids, with a sequence of 1-21 amino acids removed from the native Fc domain. In aspects, the one or more Tregitopes are inserted at or replace (e.g., full or partial replacement) one or more preferred internal sites in the Fc domain as disclosed in U.S. Pat. Nos. 7,442,778, 7,645,861, 7,655,764, 7,655,765, and/or 7,750,128.

In aspects, the compositions of the present disclosure comprise a Tregitope peptide as described herein (e.g., but not limited to, a peptide or polypeptide comprising, consisting, or consisting essentially of an amino acid sequence of SEQ ID NOS: 4-370, 391-440, and 448-833 (and/or fragments or variants thereof) or a detolerized antigen peptide of a SARS-CoV-2 protein or polypeptide wherein one or more of the identified Tregitopes are deleted, partially deleted and/or mutated that is modified by attaching a reactive moiety to the peptide to create a modified peptide, wherein the reactive moiety of the modified peptide is capable of forming a bond with a reactive functionality on a blood component, wherein upon formation of a bond between the reactive moiety of the peptide and the reactive functionality on the blood component, a -blood component conjugate is formed, as disclosed in U.S. Pat. Nos. 6,849,714, 6,887,470, 7,256,253, and 7,307,148 (each of which are herein incorporated by reference in their entirety). In aspects, the Tregitope or detolerized antigen thereof in the -blood component conjugate retains all or most of its original biologic activity. In aspects, the bond formed between the reactive moiety of the one or more modified peptides and the blood component is a covalent bond. In aspects, the peptide sequence is independently selected from SEQ ID NOS: 4-370, 391-440, and 448-833 or a detolerized antigen variant thereof.

The peptide-blood component conjugates can extend the half-life of the modified polypeptides comprising Tregitopes or detolerized antigens in vivo, protect the modified polypeptides from rapid proteolytic degradation, protect the modified polypeptides from rapid clearance from circulation and/or rapid kidney excretion, allow for wide distribution of the peptide-blood component conjugates throughout the body of a subject, aid in delivery of modified polypeptides to appropriate immune cells (such as macrophages and APCs), allow the modified polypeptides to be processed by the endocytic pathway of certain immune cells (such as macrophages and APCs), and/or aid in the presentation of modified polypeptides as an antigen by said immune cells.

In aspects, the peptide component conjugates comprise a blood component which acts as a carrier protein (e.g., albumin), and further comprise a modified polypeptide, said modified polypeptide comprising one or more Tregitopes or detolerized antigens as described herein. The modified polypeptide comprises a reactive moiety that is attached to the polypeptide, with the reactive moiety being capable of forming a bond (e.g., a covalent linkage) with a reactive functionality on the blood component. Peptide-blood component conjugates may be formed by modifying a polypeptide comprising a Tregitope or detolerized antigen thereof by attaching a reactive moiety to the polypeptide to create a modified polypeptide, then forming a bond between reactive moiety of the modified polypeptide with a reactive functionality on a blood component, as disclosed in U.S. Pat. Nos. 6,849,714, 6,887,470, 7,256,253, and 7,307,148, herein incorporated by reference in their entireties. In aspects of above-described peptide-blood component conjugates and modified polypeptides comprising Tregitopes or detolerized antigen, the peptide-blood component conjugates and modified polypeptides may be isolated, synthetic, or recombinant.

In aspects, the blood components of the peptide-blood component conjugates may be either fixed or mobile, as disclosed in U.S. Pat. Nos. 6,849,714, 6,887,470, 7,256,253, and 7,307,148. Fixed blood components are non-mobile blood components and include tissues, membrane receptors, interstitial proteins, fibrin proteins, collagens, platelets, endothelial cells, epithelial cells and their associated membrane and membranous receptors, somatic body cells, skeletal and smooth muscle cells, neuronal components, osteocytes and osteoclasts and all body tissues, especially those associated with the circulatory and lymphatic systems. Mobile blood components are blood components that do not have a fixed situs for any extended period of time, generally not exceeding 5, more usually one minute. These blood components are not membrane-associated and are present in the blood for extended periods of time and are present in a minimum concentration of at least 0.1 μg/ml. Mobile blood components include serum albumin, transferrin, ferritin and immunoglobulins such as IgM and IgG. The half-life of mobile blood components is at least about 12 hours.

In aspects of the peptide-blood component conjugates, the blood component is albumin, such as serum albumin, human serum albumin, recombinant albumin, and recombinant human serum albumin. Albumin is a preferred blood component because it contains an Fc neonatal binding domain that will carry the peptide-albumin conjugate into the appropriate cells, such as macrophages and APCs. Further, albumin contains a cysteine at amino acid 34 (Cys34) (the location of the amino acid in the amino acid sequence of human serine albumin), containing a free thiol with a pKa of approximately 5, which may serve as a preferred reactive functionality of albumin. Cys34 of albumin is capable of forming a stable thioester bond with maleimidopropionamido (MPA), which is a preferred reactive moiety of a modified Tregitope peptide.

In aspects, reactive functionalities on the blood component of the peptide-blood component conjugates or on the blood components that are capable of forming a conjugate with the instantly-disclosed modified polypeptides are groups on blood components, including mobile and fixed proteins, to which reactive groups on modified therapeutic peptides react to form covalent bonds. As disclosed in U.S. Pat. Nos. 6,849,714, 6,887,470, 7,256,253, and 7,307,148, such functionalities usually include hydroxyl groups for bonding to ester reactive groups, thiol groups for bonding to maleimides, imidates and thioester groups; amino groups for bonding to activated carboxyl, phosphoryl or any other acyl groups on reactive groups. In aspects, the reactive functionality of the blood component is an amino group, a hydroxyl group, or a thiol group. In aspects, the reactive functionality of the blood component is a component of a side group of an amino acid in a polypeptide and/or protein, wherein the reactive functionality is near the surface of the polypeptide and/or protein. In aspects, the reactive functionality of the blood component is a thiol group of a free cysteine residue of a proteinaceous blood component. In aspects, the reactive functionality is a free thiol group of the cysteine at amino acid 34 (Cys³⁴) of serine albumin. In aspects, the reactive functionality of the blood component is a thiol with a pKa of approximately 5 in a physiological environment, such as plasma. In aspects, the reactive functionality of the blood component is a thiol with a pKa of approximately 5.5 in a physiological environment, such as plasma. In aspects, the reactive functionality of the blood component is a thiol with a pKa of 3-7 in a physiological environment, such as plasma. In aspects, the reactive functionality of the blood component is a thiolate anion. In aspects, the reactive functionality is a thiolate anion of the cysteine at amino acid 34 (Cys³⁴) of serine albumin.

In aspects, the modified polypeptides of the peptide-blood component conjugates and the modified polypeptides used to form the peptide-blood component conjugates comprise a reactive moiety that is attached to the polypeptide, with the reactive moiety being capable of forming a bond (e.g., a covalent linkage) with a reactive functionality on the blood component. In aspects, the reactive group is capable of reacting with an amino group, a hydroxyl group, or a thiol group on blood component to form a covalent bond therewith. In aspects, the reactive moiety is placed at a site such that when the modified polypeptide is bonded to the blood component, the modified peptide retains a substantial proportion of the parent compound's activity. In aspects, the reactive moiety may be a succinimidyl or maleimido group. In aspects, the reactive moiety may be attached to an amino acid positioned in the less therapeutically active region of amino acids of the polypeptide to be modified. In aspects, the reactive moiety is attached to the amino terminal amino acid of the modified polypeptide. In aspects, the reactive moiety is attached to the carboxy terminal amino acid of the modified polypeptide. In aspects, the reactive moiety is attached to an amino acid positioned between the amino terminal amino acid and the carboxy terminal amino acid of the modified polypeptide. In aspects, the reactive group may be attached to the polypeptide (to be modified) either via a linking group, or optionally without using a linking group. Further, one or more additional amino acids (e.g., one or more lysines) may be added to the polypeptide to facilitate the attachment of the reactive group. Linking groups are chemical moieties that link or connect reactive groups of blood components to polypeptides comprising one or more Tregitopes or detolerized antigens as described herein. Linking groups may comprise one or more alkyl groups, alkoxy group, alkenyl group, alkynyl group or amino group substituted by alkyl groups, cycloalkyl group, polycyclic group, aryl groups, polyaryl groups, substituted aryl groups, heterocyclic groups, and substituted heterocyclic groups. Linking groups may also comprise poly ethoxy aminoacids such as AEA ((2-amino)ethoxy acetic acid) or a preferred linking group AEEA ([2-(2-amino)ethoxy)]ethoxy acetic acid). In aspects, linking groups may comprise a polyethyleneglycol linker (e.g., but not limited to, PEG2 or PEG12).

As should be understood, modified polypeptides may be administered in vivo such that conjugation with blood components occurs in vivo, or they may be first conjugated to blood components in vitro and the resulting peptidase-stabilized polypeptide administered in vivo. Further, as disclosed in in U.S. Pat. Nos. 6,849,714, 6,887,470, 7,256,253, and 7,307,148, a peptidase-stabilized polypeptide is a modified polypeptide that has been conjugated to a blood component via a covalent bond formed between the reactive group of the modified peptide and the functionalities of the blood component, with or without a linking group. Such reaction is preferably established by covalent bonding of a polypeptide modified with a maleimide link (e.g. prepared from GMBS, MPA or other maleimides) to a thiol group on a mobile blood protein such as serum albumin or IgG. Peptidase-stabilized polypeptides are more stable in the presence of peptidases in vivo than a non-stabilized peptide. A peptidase-stabilized therapeutic peptide generally has an increased half-life of at least 10-50% as compared to a non-stabilized peptide of identical sequence. Peptidase-stability is determined by comparing the half-life of the unmodified therapeutic peptide in serum or blood to the half-life of a modified counterpart therapeutic peptide in serum or blood. Half-life is determined by sampling the serum or blood after administration of the modified and non-modified peptides and determining the activity of the peptide. In addition to determining the activity, the length of therapeutic peptide may also be measured.

In aspects, the modified polypeptides of the peptide-blood component conjugates and the modified polypeptides used to form the peptide-blood component conjugates comprise one or more Tregitopes or detolerized antigens thereof as disclosed herein. In aspects, the one or more Tregitopes or detolerized antigens thereof of the modified polypeptides have a sequence comprising, consisting of, or consisting essentially of one or more of SEQ ID NOS: 4-370, 391-440, and 448-833 (and fragments and variants thereof) as essentially disclosed herein or a SARS-CoV-2 protein or polypeptide wherein the Tregitopes as described herein are deleted, partially deleted and/or mutated. In aspects, the one or more peptides of the modified polypeptide may optionally have one or more linkers, which may optionally be cleavage sensitive sites, adjacent to their N and/or C terminal end. In such a modified polypeptide, two or more of the peptides may have a cleavage sensitive site between them. Alternatively, two or more of the peptides may be connected directly to one another or through a linker that is not a cleavage sensitive site. In aspects, the modified polypeptide comprising the one or more Tregitopes and/or the detolerized antigens contained therein can be in either neutral (uncharged) or salt forms, and may be either free of or include modifications such as glycosylation, side chain oxidation, or phosphorylation. In certain aspects, the modified polypeptide can be capped with an N-terminal acetyl and/or C-terminal amino group. In aspects, the one or more Tregitopes included in the modified polypeptide can be capped with an N-terminal acetyl and/or C-terminal amino group.

In aspects, the reactive moiety of the modified peptide is a soft electrophile. In aspects, the reactive moiety of the modified peptide is an electrophile that is selective for thiols. In a preferred embodiment, the reactive moiety attached to create the modified peptide is maleimide. In aspects, the reactive moiety is maleimide propionic acid. In a preferred embodiment, the reactive moiety attached of the modified Tregitope peptide is maleimide, the blood component is albumin, and the reactive functionality on the albumin is a free thiol or thiolate anion of Cys³⁴ of albumin. When the reactive moiety of the modified peptide a maleimide, the blood component is albumin, and the reactive functionality of the albumin is a free thiol or thiolate anion of Cys³⁴ of albumin, a stable thioester linkage between the maleimide group and the sulfhydryl is formed which cannot be cleaved under physiological conditions. In aspects, the modified peptide contains a linker, wherein the reactive moiety is attached to the peptide through the linker. In aspects, the modified peptide binds to the blood component in a 1:1 molar ratio.

The manner of producing the modified peptides of the present disclosure will vary widely, depending upon the nature of the various elements comprising the molecule. The synthetic procedures may be selected so as to be simple, provide for high yields, and allow for a highly purified stable product. Normally, the reactive moiety will be created as the last stage, for example, with a carboxyl group, esterification to form an active ester will be the last step of the synthesis.

In aspects, the present disclosure is also directed to a method of synthesizing the modified Tregitope peptides or modified detolerized antigen peptides as described herein, with processes as disclosed in U.S. Pat. Nos. 6,849,714, 6,887,470, 7,256,253, and 7,307,148. In aspects, the method comprises the following steps. In the first step, the one or more peptide sequence can be made as essentially disclosed herein. In the second step, if the polypeptide does not contain a cysteine, then the polypeptide may be synthesized from the carboxy terminal amino acid and the reactive moiety is added to the carboxy terminal amino acid. Alternatively, a terminal lysine (or one or more lysines) may added to the carboxy terminal amino acid and the reactive moiety is added to the terminal lysine. In the third step, if the polypeptide contains only one cysteine, then the cysteine is reacted with a protective group prior to addition of the reactive moiety to an amino acid in a less therapeutically active region of the polypeptide. In the fourth step, if the polypeptide contains two cysteines as a disulfide bridge, then the two cysteines are oxidized and the reactive moiety is added to the amino terminal amino acid, or to the carboxy terminal amino acid, or to an amino acid positioned between the carboxy terminal amino acid and the amino terminal amino acid of the polypeptide. In the fifth step, if the polypeptide contains more than two cysteines as disulfide bridges, the cysteines are sequentially oxidized in the disulfide bridges and the peptide is purified prior to the addition of the reactive moieties to the carboxy terminal amino acid.

In aspects, the present disclosure is also directed to a method of synthesizing the peptide-blood component conjugate. In a first step, reactive maleimidopropionamido (MPA) is added via an N-terminal lysine on the polypeptide comprising one or more Tregitopes to create a modified polypeptide. In aspects, one or more lysines are present on the N-terminus of the polypeptide, optionally present at the N-terminus of a sequence selected from the group of SEQ. ID NOS: 4-370, 391-440, and 448-833 or detolerized antigens thereof as disclosed herein. Optionally, polyethyleneglycol linker, such as PEG2 or PEG12, is present between the one or more lysines and a Tregitope or detolerized antigen sequence, or at the N-terminus of a Tregitope or detolerized antigen sequence. In aspects, a lysosomal cleavage site, such as a Cathepsin B site, optionally consisting (sequentially from N-terminus to C-terminus) of valine and citrulline, is present between the PEG2 or PEG12 moiety and the peptide sequence. The lysosomal cleavage site (such as Cathepsin B site) may be incorporated to provide a lysosomal protease site, allowing the peptide to be released into the lysosomal compartment. In aspects, lysosomal cleavage site (such as Cathepsin B site) is present to provide a lysosomal protease site, allowing the peptide to be released into cells, preferably into the early endosome. In a preferred embodiment, the lysosomal cleavage site (such as Cathepsin B site) is present to provide a lysosomal protease site, allowing the peptide to be released into cells, such as into a membrane-enclosed vesicle (such as the early endosome, late endosome, or lysosome), such that the peptide may be processed for antigen presentation. In aspects, the peptide is presented as antigen by immune cells, such as macrophages or antigen-presenting cells, preferably presented as an MHC class II antigen. In aspects, a lysosomal cleavage site, such as a Cathepsin B site, optionally consisting (sequentially from N-terminus to C-terminus) of valine and citrulline, is present between the PEG2 moiety and the peptide sequence, and/or between one or more peptide. In aspects, one or more peptide may be present on the construct, optionally more proximate to the C-terminus than the linker. In aspects, one or more lysosomal cleavage sites are present between multiple peptide (for example, such that a single lysosomal cleavage site separates two Tregitopes or detolerized antigens, or such that one lysosomal cleavage site is present between a first and second peptide, and another lysosomal cleavage site is present between a second and third peptide, and so on). In aspects, a norleucine (Nle) residue is present at the C-terminus as a means to quantitate the amount of peptide incorporated into the final peptide-blood component conjugate, for example for evaluation by mass spectrometry. In aspects, the C-terminus of the polypeptide is capped with a c-terminal amino group. In a second step, a maleimide-based chemistry is used to covalently link the modified polypeptide to a blood component, preferably serum albumin, in a 1:1 molar ratio. The second step may be performed in vivo or ex vivo, as described further below and in the examples of the present disclosure.

In aspects, the formation of the peptide-blood component conjugate protects the Tregitope or detolerized antigen, when present in vivo, from rapid degradation by peptidases, rapid clearance from circulation, and/or rapid kidney excretion. In aspects, the formation of the peptide-blood component conjugate significantly extends the half-life of the peptide in vivo. In aspects, the formation of the peptide-blood component conjugate allows wide distribution of the peptide-blood component conjugate throughout the body of a subject. In aspects, the peptide-blood component conjugate does not cross the blood-brain barrier when present in the plasma of a subject. In aspects, the peptide-blood component conjugate aid in delivery of peptides to appropriate immune cells, such as macrophages and/or antigen-presenting cells (APCs). In aspects, upon delivery of peptides to appropriate immune cells, such as macrophages and/or APCs, the peptides are encompassed in a membrane-bound vesicle, preferably a vesicle in the endocytic pathway such as an early endosome, late endosome, or lysosome. In aspects, the peptides, once processed by the appropriate immune cells, such as macrophages and/or APCs, are presented as MHC class II antigens.

In aspects, the Tregitope or detolerized antigen in the peptide-blood component conjugate has a plasma half-life in vivo of up to 12 hours. In aspects, the Tregitope or detolerized antigen in the peptide-blood component conjugate has a plasma half-life in vivo of up to 1 day. In aspects, the Tregitope or detolerized antigen in the peptide-blood component conjugate has a plasma half-life in vivo of up to 40-48 hours. In aspects, the Tregitope or detolerized antigen in the peptide-blood component conjugate has a plasma half-life in vivo of up to 60 hours. In aspects, the Tregitope or detolerized antigen in the peptide-blood component conjugate has a plasma half-life in vivo of up to 15 days.

In aspects, the modified polypeptide comprising one or more Tregitopes and/or detolerized antigens is administered to a subject, wherein upon administration, the modified polypeptide reacts in vivo with a reactive functionality of a circulating blood component. In aspects, the peptide is administered to a human subject, and the blood component is human albumin, preferably the circulating albumin of the human subject.

In aspects, the modified polypeptides used to form the peptide-blood component conjugates is capable of forming a bond ex vivo with a reactive functionality on a blood component, wherein upon formation of a bond between the reactive moiety of the modified polypeptide and the reactive functionality on the blood component, a peptide-blood component conjugate is formed, as disclosed in U.S. Pat. Nos. 6,849,714, 6,887,470, 7,256,253, and 7,307,148. In aspects, the modified polypeptide as disclosed herein is configured to covalently attach to a reactive functionality of a blood component outside of the body. In aspects, the blood component is albumin. In aspects, the blood component is selected from the group of recombinant albumin, human recombinant albumin, and albumin from a genomic source.

### (v) Nucleic Acids

In aspects, the present disclosure also provides for nucleic acids (e.g., DNAs (including cDNA, RNAs (such as, but limited to mRNA), vectors, viruses, or hybrids thereof, all of which may be isolated, synthetic, or recombinant) that encode in whole or in part one or more one or more peptides, polypeptides (including Tregitopes and detolerized antigens as disclosed herein), concatemeric peptides, and/or chimeric or fusion polypeptides of the present disclosure as described herein. In aspects, the nucleic acid further comprises, or is contained within, an expression cassette, a plasmid, and expression vector, or recombinant virus, wherein optionally the nucleic acid, or the expression cassette, plasmid, expression vector, or recombinant virus is contained within a cell, optionally a human cell or a nonhuman cell, and optionally the cell is transformed with the nucleic acid, or the expression cassette, plasmid, expression vector, or recombinant virus. In aspects, cells are transduced, transfected, or otherwise engineered to contain within one or more of e.g., polypeptides of the present disclosure; isolated, synthetic, or recombinant nucleic acids, expression cassettes, plasmids, expression vectors, or recombinant viruses as disclosed herein; and/or isolated, synthetic, or recombinant chimeric or fusion polypeptide compositions as disclosed herein. In aspects, the cell can be a mammalian cell, bacterial cell, insect cell, or yeast cell. In aspects, the nucleic acid molecules of the present disclosure can be inserted into vectors and used, for example, as expression vectors or gene therapy vectors. Gene therapy vectors can be delivered to a subject by, e.g., intravenous injection, local administration (U.S. Pat. No. 5,328,470) or by stereotactic injection (Chen S H et al., (1994), Proc Natl Acad Sci USA, 91(8):3054-7, which are herein incorporated by reference in their entirety). Similarly, the nucleic acid molecules of the present disclosure can be inserted into plasmids. The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. Such pharmaceutical compositions can be included in a container, pack, or dispenser together with instructions for administration. In aspects of the above nucleic acids (e.g., DNAs, RNAs, vectors, viruses, or hybrids thereof) that encode in whole or in part at least one or more peptides, polypeptides, concatemeric peptides, and/or chimeric or fusion polypeptides as described herein, the nucleic acids encode one or more peptides or polypeptides of the instant disclosure as described above (e.g., but not limited to, a peptide or polypeptide comprising, consisting, or consisting essentially of an amino acid sequence of SEQ ID NOS: 4-370, 391-440, and 448-833, detolerized antigens of proteins or peptides derived from SARS-CoV-2 wherein one or more Tregitopes are deleted, partially deleted and/or mutated (and/or fragments or variants thereof), and optionally 1 to 12 additional amino acids distributed in any ratio on the N terminus and/or C-terminus of the polypeptide; as well as any concatemeric peptides described herein. In aspects, the present disclosure is directed to a vector comprising a nucleic acid of the present disclosure encoding one or more polypeptides of the present disclosure or chimeric or fusion polypeptide composition of the present disclosure. In aspects, the present disclosure is directed to a cell comprising a vector of the present disclosure. In aspects, the cell can be a mammalian cell, bacterial cell, insect cell, or yeast cell.

The nucleic acid of the instant disclosure may be DNAs (including but not limited to cDNA) or RNAs (including but not limited to mRNA), single- or double-stranded. The nucleic acid is typically DNA or RNA (including mRNA). The nucleic acid may be produced by techniques well known in the art, such as synthesis, or cloning, or amplification of the sequence encoding the immunogenic polypeptide; synthesis, or cloning, or amplification of the sequence encoding the cell membrane addressing sequence; ligation of the sequences and their cloning/amplification in appropriate vectors and cells. The nucleic acids provided herein (whether RNAs, DNAs, vectors, viruses or hybrids thereof) that encode in whole or in part one or more peptides, polypeptides, concatemeric peptides, and/or chimeric or fusion polypeptides as described herein can be isolated from a variety of sources, genetically engineered, amplified, synthetically produced, and/or expressed/generated recombinantly. Recombinant polypeptides generated from these nucleic acids can be individually isolated or cloned and tested for a desired activity. Any recombinant expression system can be used, including e.g. in vitro, bacterial, fungal, mammalian, yeast, insect or plant cell expression systems. In aspects nucleic acids provided herein are synthesized in vitro by well-known chemical synthesis techniques (as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066, all of which are herein incorporated by reference in their entirety). Further, techniques for the manipulation of nucleic acids provided herein, such as, e.g., subcloning, labeling probes (e.g., random-primer labeling using Klenow polymerase, nick translation, amplification), sequencing, hybridization and the like are well described in the scientific and patent literature (see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993), all of which are herein incorporated by reference in their entirety).

A further object of the disclosure relates to a nucleic acid molecule encoding one or more peptides, polypeptides, concatemeric peptides, and/or chimeric or fusion polypeptides as described herein. The nucleic acid may be used to produce the one or more peptides, polypeptides, concatemeric peptides, and/or chimeric or fusion polypeptides as described herein in vitro or in vivo, or to produce cells expressing the polypeptide on their surface, or to produce vaccines wherein the active agent is the nucleic acid or a vector containing the nucleic acid. The nucleic acid may be, e.g., DNA, cDNA, PNA, CNA, RNA, either single- and/or double-stranded, or native or stabilized forms of polynucleotides as are known in the art.

As previously mentioned, the nucleic acid molecules according to the present disclosure may be provided in the form of a nucleic acid molecule per se such as naked nucleic acid molecules; a plasmid, a vector; virus or host cell etc., either from prokaryotic or eukaryotic origin. Vectors include expression vectors that contain a nucleic acid molecule of the invention. An expression vector capable of expressing a polypeptide can be prepared. Expression vectors for different cell types are well known in the art and can be selected without undue experimentation. Generally, the (e.g., cDNA, or RNA, including mRNA) is inserted into an expression vector, such as a plasmid, in proper orientation and correct reading frame for expression. If necessary, the DNA (e.g., cDNA, or RNA, including mRNA) may be linked to the appropriate transcriptional and translational regulatory control nucleotide sequences recognized by the desired host (e.g., bacteria), although such controls are generally available in the expression vector. The vector is then introduced into the host bacteria for cloning using standard techniques. The vectors of the present invention may, for example, comprise a transcriptional promoter, and/or a transcriptional terminator, wherein the promoter is operably linked with the nucleic acid molecule, and wherein the nucleic acid molecule is operably linked with the transcription terminator. One or more peptides or polypeptides of the present disclosure may be encoded by a single expression vector. Such nucleic acid molecules may act as vehicles for delivering peptides/polypeptides to the subject in need thereof, in vivo, in the form of, e.g., DNA/RNA vaccines.

In aspects, the vector may be a viral vector comprising a nucleic acid as defined above. The viral vector may be derived from different types of viruses, such as, Swinepox, Fowlpox, Pseudorabies, Aujezky's virus, salmonella, vaccinia virus, BHV (Bovine Herpes Virus), HVT (Herpes Virus of Turkey), adenovirus, TGEV (Transmissible Gastroenteritidis Coronavirus), Erythrovirus, and SIV (Simian Immunodeficiency Virus). Other expression systems and vectors may be used as well, such as plasmids that replicate and/or integrate in yeast cells.

The instant disclosure also relates to a method for preparing a peptide, polypeptide, concatemeric peptide, and/or chimeric or fusion polypeptide of the instant disclosure, the method comprising culturing a host cell containing a nucleic acid or vector as defined above under conditions suitable for expression of the nucleic acid and recovering the polypeptide. As indicated above, the proteins and peptides may be purified according to techniques known per se in the art.

### (vi) Pharmaceutical Compositions

In aspects, the present disclosure provides a novel class of Tregitopes and/or detolerized antigens, which comprise a peptide or polypeptide chain derived from SARS-CoV-2 proteins, including the envelope, membrane, spike, nucleocapsid, ORF3a, ORF6, ORF7a, ORF1ab non-structural protein 2 (NSP2), ORF1ab non-structural protein 3 (NSP3), ORF1ab non-structural protein 4 (NSP4), ORF1ab non-structural protein 6 (NSP6), ORF1ab non-structural protein 7 (NSP7), ORF1ab non-structural protein 8 (NSP8), ORF1ab non-structural protein 9 (NSP9), ORF1ab RNA-dependent RNA polymerase, ORF1ab helicase, ORF1ab 3′-5′ exonuclease, ORF1ab endoRNase, and ORF1ab 2′O-ribose methyltransferase proteins of SARS-CoV-2 as well as the detolerized mutants thereof as described herein.

In aspects, the present disclosure provides compositions, including polypeptides (Tregitopes or detolerized antigens thereof as disclosed herein; nucleic acids, expression cassettes, plasmids, expression vectors, recombinant viruses, or cells (all of which in aspects may be isolated, synthetic, or recombinant) as disclosed herein; isolated, synthetic, or recombinant chimeric or fusion polypeptide compositions as disclosed herein; and/or pharmaceutical compositions or formulations as disclosed herein. In aspects, the compositions include one or more of the regulatory Tregitopes of SEQ ID NOS: 4-370, 391-440, and 448-833 (including fragments thereof, variants thereof, and fragments of such variants thereof, provided said fragments and/or variants retain MHC binding propensity and/or TCR specificity). In aspects the detolerized antigen compositions include SARS-CoV-2 derived proteins and polypeptides with one or more of the disclosed deletions, partial delectation and/or mutations to the Tregitopes as set forth in SEQ ID NOS: 4-370, 391-440, and 448-833 (including fragments thereof, variants thereof, and fragments of such variants thereof, provided said fragments and/or variants demonstrate reduced T cell binding and/or MHC binding). In certain aspects, the Tregitopes or detolerized antigen peptides can be capped with an N-terminal acetyl and/or C-terminal amino group.

In aspects, the Tregitope compositions and the detolerizing antigen compositions of the present disclosure as disclosed herein can be purified to homogeneity or partially purified. It is understood, however, that preparations in which the Tregitope compositions are not purified to homogeneity are useful. The critical feature is that the preparation allows for the desired function of the Tregitope, even in the presence of considerable amounts of other components. Thus, the present disclosure encompasses various degrees of purity. In one embodiment, the language “substantially free of cellular material” includes preparations of the Tregitope having less than about 30% (by dry weight) other proteins (e.g., contaminating protein), less than about 20% other proteins, less than about 10% other proteins, less than about 5% other proteins, less than about 4% other proteins, less than about 3% other proteins, less than about 2% other proteins, less than about 1% other proteins, or any value or range therebetween.

In aspects, when a composition of the present disclosure is recombinantly produced, said composition can also be substantially free of culture medium, for example, culture medium represents less than about 20%, less than about 10%, or less than about 5% of the volume of the polypeptide, nucleic acid, or chimeric or fusion polypeptide preparation. The language “substantially free of chemical precursors or other chemicals” includes preparations of the polypeptide, nucleic acid, or chimeric or fusion polypeptide in which it is separated from chemical precursors or other chemicals that are involved in the peptide's synthesis. The language “substantially free of chemical precursors or other chemicals” can include, for example, preparations of the Tregitope, nucleic acid, or chimeric or fusion polypeptide having less than about 30% (by dry weight) chemical precursors or other chemicals, less than about 20% chemical precursors or other chemicals, less than about 10% chemical precursors or other chemicals, less than about 5% chemical precursors or other chemicals, less than about 4% chemical precursors or other chemicals, less than about 3% chemical precursors or other chemicals, less than about 2% chemical precursors or other chemicals, or less than about 1% chemical precursors or other chemicals.

In aspects, the present disclosure also includes pharmaceutical compositions or formulations that include the peptides or variants or fragments thereof, including the Tregitopes and detolerized antigens as set forth herein. In aspects, pharmaceutical compositions or formulations generally comprise a composition of the present disclosure and a pharmaceutically acceptable carrier, excipient, and/or adjuvant. In aspects, said pharmaceutical compositions are suitable for administration. Pharmaceutically acceptable carriers and/or excipients are determined in part by the particular composition being administered, as well as by the particular method used to administer the composition. Accordingly, there is a wide variety of suitable formulations of pharmaceutical compositions for administering the instantly disclosed Tregitope compositions (see, e.g., Remington's Pharmaceutical Sciences, (18^{TH} Ed, 1990), Mack Publishing Co., Easton, Pa. Publ)). In aspects, the pharmaceutical compositions are generally formulated as sterile, substantially isotonic, and in full compliance with all Good Manufacturing Practice (GMP) regulations of the U.S. Food and Drug Administration.

The terms “pharmaceutically-acceptable,” “physiologically-tolerable,” and grammatical variations thereof, as they refer to compositions, carriers, excipients, and reagents, are used interchangeably and represent that the materials are capable of administration to or upon a subject without the production of undesirable physiological effects to a degree that would prohibit administration of the composition. For example, “pharmaceutically-acceptable excipient” means, for example, an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. A person of ordinary skill in the art would be able to determine the appropriate timing, sequence and dosages of administration for particular compositions of the present disclosure.

Examples of pharmaceutically acceptable carriers, excipients or diluents include, but are not limited to demineralized or distilled water; saline solution; vegetable based oils such as peanut oil, arachis oil, safflower oil, olive oil, cottonseed oil, maize oil, sesame oil, or coconut oil; silicone oils, including polysiloxanes, such as methyl polysiloxane, phenyl polysiloxane and methylphenyl polysolpoxane; volatile silicones; mineral oils such as light liquid paraffin oil, or heavy liquid paraffin oil; squalene; cellulose derivatives such as methylcellulose, ethylcellulose, carboxymethylcellulose, carboxymethylcellulose sodium salt, or hydroxypropyl methylcellulose; lower alkanols, for example ethanol or isopropanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrrolidone; agar; carrageenan; gum tragacanth or gum acacia; and petroleum jelly. Typically, the carrier or carriers will form from 10% to 99.9% by weight of the vaccine composition and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

In aspects, examples of such carriers or diluents include, but are not limited to, water, saline, Ringer's solutions, dextrose solution, and 5% human serum albumin. Liposomes and non-aqueous vehicles such as fixed oils can also be used. The use of such media and compounds for pharmaceutically active substances is well known in the art. Except insofar as any conventional media or compound is incompatible with the compositions of the present disclosure and as previously described above use thereof in the compositions is contemplated. Supplementary active compounds can also be incorporated into the compositions.

Examples of adjuvants include, but are not limited to, oil in water emulsions, aluminum hydroxide (alum), immunostimulating complexes, non-ionic block polymers or copolymers, cytokines (like IL-1, IL-2, IL-7, IFN-α, IFN-β, IFN-γ, etc.), saponins, monophosphoryl lipid A (MLA), muramyl dipeptides (MDP) and the like. Other suitable adjuvants include, for example, aluminum potassium sulfate, heat-labile or heat-stable enterotoxin(s) isolated from Escherichia coli, cholera toxin or the B subunit thereof, diphtheria toxin, tetanus toxin, pertussis toxin, Freund's incomplete or complete adjuvant, etc. Toxin-based adjuvants, such as diphtheria toxin, tetanus toxin and pertussis toxin may be inactivated prior to use, for example, by treatment with formaldehyde.

In aspects, compositions of the present disclosure are formulated to be compatible with its intended route of administration. The compositions of the present disclosure can be administered by parenteral, topical, intravenous, oral, subcutaneous, intra-arterial, intradermal, transdermal, rectal, intracranial, intrathecal, intraperitoneal, intranasal; vaginally; intramuscular route or as inhalants. In aspects, the compositions of the present disclosure can be injected directly into a particular tissue where deposits have accumulated, e.g., intracranial injection. In other aspects, intramuscular injection or intravenous infusion may be used for administration of the compositions of the present disclosure. In some methods, the compositions of the present disclosure are injected directly into the cranium. In some methods, the compositions of the present disclosure are administered as a sustained release composition or device, such as but not limited to a Medipad™ device.

In aspects, the compositions of the present disclosure can optionally be administered in combination with other agents that are at least partly effective in treating various medical conditions as described herein. For example, in the case of administration into the central nervous system of a subject, the compositions of the present disclosure can also be administered in conjunction with other agents that increase passage of the agents of the invention across the blood-brain barrier.

In aspects, solutions or suspensions used for parenteral, intradermal, or subcutaneous application can include, but are not limited to, the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. Examples of excipients can include starch, glucose, lactose, sucrose, gelatin, malt, rice, flour, chalk, silica gel, water, ethanol, DMSO, glycol, propylene, dried skim milk, and the like. The composition can also contain pH buffering reagents, and wetting or emulsifying agents.

In aspects, the parenteral preparation can be enclosed in ampoules, disposable syringes or multiple dose vials made of glass or plastic.

In aspects, pharmaceutical compositions or formulations suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition is sterile and should be fluid to the extent that easy syringeability exists. It is stable under the conditions of manufacture and storage and is preserved against the contaminating action of microorganisms such as bacteria and fungi. In aspects, the formulations may include aggregates, fragments, breakdown products and post-translational modifications, to the extent these impurities bind HLA and present the same TCR face to cognate T cells they are expected to function in a similar fashion to pure Tregitopes. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be preferable to include isotonic compounds, e.g., sugars, polyalcohols such as manitol, sorbitol, and sodium chloride in the composition. Prolonged absorption of the injectable compositions can be brought about by including in the composition a compound that delays absorption, e.g., aluminum monostearate and gelatin.

In aspects, sterile injectable solutions can be prepared by incorporating the compositions of the present disclosure in the required amount in an appropriate solvent with one or a combination of ingredients enumerated above, as required, followed by filtered sterilization. Generally, dispersions are prepared by incorporating the binding agent into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. Further, the compositions of the present disclosure can be administered in the form of a depot injection or implant preparation that can be formulated in such a manner as to permit a sustained or pulsatile release of the active ingredient.

In aspects, oral compositions generally include an inert diluent or an edible carrier and can be enclosed in gelatin capsules or compressed into tablets. In aspects, for the purpose of oral therapeutic administration, the binding agent can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. In aspects, the tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel or corn starch; a lubricant such as magnesium stearate or Sterotes; a glidant such as colloidal silicon dioxide; a sweetening compound such as sucrose or saccharin; or a flavoring compound such as peppermint, methyl salicylate or orange flavoring.

For administration by inhalation, the compositions of the present disclosure can be delivered in the form of an aerosol spray from pressured container or dispenser that contains a suitable propellant, e.g., a gas such as carbon dioxide, or a nebulizer.

In aspects, systemic administration of the compositions of the present disclosure can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the compositions may be formulated into ointments, salves, gels, or creams, and applied either topically or through transdermal patch technology as generally known in the art.

In aspects, the compositions of the present disclosure can also be prepared in the form of suppositories (e.g., with conventional suppository bases such as cocoa butter and other glycerides) or retention enemas for rectal delivery.

In aspects, the compositions of the present disclosure are prepared with carriers that protect the compositions against rapid elimination from the body, such as a controlled-release formulation, including implants and microencapsulated delivery systems. Biodegradable, biocompatible polymers can be used, such as, for example, ethylene vinyl acetate, polyanhydrides, polyglycolic acid, collagen, polyorthoesters, and polylactic acid. Methods for preparation of such formulations will be apparent to those skilled in the art. The materials can also be obtained commercially, e.g., from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art (U.S. Pat. No. 4,522,811, which is herein incorporated by reference in its entirety). In aspects, the compositions of the present disclosure can be implanted within or linked to a biopolymer solid support that allows for the slow release of the Tregitope compositions to the desired site.

In aspects, it is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of binding agent calculated to produce the desired therapeutic effect in association with the required pharmaceutical carrier. The specification for the dosage unit forms of the instant disclosure are dictated by and directly dependent on the unique characteristics of the binding agent and the particular therapeutic effect to be achieved, and the limitations inherent in the art of compounding such Tregitope compositions for the treatment of a subject.

In aspects, the one or more polypeptides (including Tregitopes and/or detolerized antigens) as disclosed herein can also be administered to the patient by ex vivo pulsing of isolated dendritic cells (DC) with peptides, followed by reinfusion of the pulsed cells into the patient. These can be prepared according to methods known to those skilled in the art (Butterfield, (2013), Front Immunol, 4:454 and Dissanayake et al., (2014), PLoS One, 9(3)1-10). These reinfusions may be administered by the above methods and compositions.

### (vii) Vaccine Compositions

The term “vaccine” as used herein includes an agent which may be used to cause, stimulate or amplify the immune system of animals (e.g., humans) against a pathogen. Vaccines of the invention are able to cause or stimulate or amplify an immune response against SARS-CoV-2 infection and related diseases caused by SARS-CoV-2, including COVID-19.

The term “immunization” includes the process of delivering an immunogen to a subject. Immunization may, for example, enable a continuing high level of antibody and/or cellular response in which T-lymphocytes can kill or suppress the pathogen in the immunized animal, such as a human, which is directed against a pathogen or antigen to which the animal has been previously exposed.

While the Tregitopes may be themselves ineffective as vaccines due to their immune-suppressive effects, the detolerized antigens thereof can be applied as a vaccine. Vaccines of the instant disclosure include an immunologically effective amount of a composition (including one or more of the detolerized antigens as set forth herein; nucleic acids, expression cassettes, plasmids, expression vectors, recombinant viruses, or cells (all of which in aspects may be isolated, synthetic, or recombinant) as disclosed herein; chimeric or fusion polypeptide compositions as disclosed herein (which in aspects may be isolated, synthetic, or recombinant) as described above, and in aspects in a pharmaceutically acceptable vehicle and optionally with additional excipients and/or an adjuvant. As a result of the vaccination with a detolerizing composition of the invention, animals, and in aspects humans, become at least partially or completely immune to SARS-CoV-2 infection and/or related diseases caused by SARS-CoV-2, including COVID-19, or resistant to developing moderate or severe SARS-CoV-2 infection and/or related diseases caused by SARS-CoV-2, including COVID-19. The instantly disclosed vaccines may be used to elicit a humoral and/or a cellular response, including CD4+ and CD8+T effector cell responses. Preferably, an animal subject, such as a human, is protected to an extent to which one to all of the adverse physiological symptoms or effects of SARS-CoV-2 infection and/or related diseases caused by SARS-CoV-2, including COVID-19, are significantly reduced, ameliorated or totally prevented.

In practice, the exact amount required for an immunologically effective dose may vary from subject to subject depending on factors such as the age and general condition of the subject, the nature of the formulation and the mode of administration. An appropriate “effective amount” may be determined by one of ordinary skill in the art using only routine experimentation. For instance, methods are known in the art for determining or titrating suitable dosages of a vaccine to find minimal effective dosages based on the weight of the animal subject, including human subject, concentration of the vaccine and other typical factors.

The dosage of the vaccine, concentration of components therein and timing of administering the vaccine, which elicit a suitable immune response, can be determined by methods such as by antibody titrations of sera, e.g., by ELISA and/or seroneutralization assay analysis and/or by vaccination challenge evaluation.

In aspects, the vaccine comprises a composition (including one or more of e.g., one or more of the detolerized SARS-CoV-2 polypeptides as disclosed herein; nucleic acids, expression cassettes, plasmids, expression vectors, recombinant viruses, or cells (all of which in aspects may be isolated, synthetic, or recombinant) as disclosed herein; chimeric or fusion polypeptide compositions as disclosed herein (which in aspects may be isolated, synthetic, or recombinant) as disclosed herein) in purified form, optionally in combination with any suitable excipient, carrier, adjuvant, and/or additional protein antigen.

In aspects, the vaccine comprises a nucleic acid as defined above, optionally in combination with any suitable excipient, carrier, adjuvant, and/or additional protein antigen. In aspects, the vaccine comprises a viral vector containing a nucleic acid as defined above. In aspects, the vaccine comprises one or more plasmid vectors.

The instantly-disclosed vaccine constructs including putative T-cell epitopes, for example a DNA or RNA epitope vaccine or an “epistring”/concatemeric polypeptide that consists of overlapping putative T-cell epitopes as disclosed herein (e.g., peptides or polypeptides as disclosed herein, which may be isolated, synthetic, or recombinant, such as polypeptides having an amino acid sequence comprising, consisting of, or consisting essentially of one or more of the detolerized SARS-CoV-2 polypeptides and nucleic acids (e.g., RNA mRNA, DNA, cDNA) encoding such concatemeric peptides; chimeric or fusion polypeptide compositions as disclosed herein), upon administration to a subject may initiate a strong T-cell mediated immune response but may not induce a humoral immune response. Therefore, aspects of a vaccine against SARS-CoV-2 infection and/or related diseases caused by SARS-CoV-2, including COVID-19, contains a combination of the putative T-cell epitopes (e.g., including one or more of the detolerized antigens as set forth herein) together with either live attenuated virus (LAV, for example live attenuated SARS-CoV-2) or inactivated virus (for example inactivated SARS-CoV-2). The vaccine composition (including both the putative T-cell epitopes and an LAV or inactivated virus) upon administration to a subject may induce both cellular and humoral immune responses, thereby conferring comprehensive immunity against SARS-CoV-2 infection and/or related diseases caused by SARS-CoV-2, including COVID-19, in the animals, including humans.

Vaccines may comprise other ingredients, known per se by one of ordinary skill in the art, such as pharmaceutically acceptable carriers, excipients, diluents, adjuvants, freeze drying stabilizers, wetting or emulsifying agents, pH buffering agents, gelling or viscosity enhancing additives, and preservatives, depending on the route of administration.

Examples of pharmaceutically acceptable carriers, excipients or diluents include, but are not limited to demineralized or distilled water; saline solution; vegetable based oils such as peanut oil, arachis oil, safflower oil, olive oil, cottonseed oil, maize oil, sesame oil, or coconut oil; silicone oils, including polysiloxanes, such as methyl polysiloxane, phenyl polysiloxane and methylphenyl polysolpoxane; volatile silicones; mineral oils such as light liquid paraffin oil, or heavy liquid paraffin oil; squalene; cellulose derivatives such as methylcellulose, ethylcellulose, carboxymethylcellulose, carboxymethylcellulose sodium salt, or hydroxypropyl methylcellulose; lower alkanols, for example ethanol or isopropanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrrolidone; agar; carrageenan; gum tragacanth or gum acacia; and petroleum jelly. Typically, the carrier or carriers will form from 10% to 99.9% by weight of the vaccine composition and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

Examples of adjuvants include, but are not limited to, oil in water emulsions, aluminum hydroxide (alum), immunostimulating complexes, non-ionic block polymers or copolymers, cytokines (like IL-1, IL-2, IL-7, IFN-α, IFN-β, IFN-γ, etc.), saponins, monophosphoryl lipid A (MLA), muramyl dipeptides (MDP) and the like. Other suitable adjuvants include, for example, aluminum potassium sulfate, heat-labile or heat-stable enterotoxin(s) isolated from Escherichia coli, cholera toxin or the B subunit thereof, diphtheria toxin, tetanus toxin, pertussis toxin, Freund's incomplete or complete adjuvant, etc. Toxin-based adjuvants, such as diphtheria toxin, tetanus toxin and pertussis toxin may be inactivated prior to use, for example, by treatment with formaldehyde.

Examples of freeze-drying stabilizer may be for example carbohydrates such as sorbitol, mannitol, starch, sucrose, dextran or glucose, proteins such as albumin or casein, and derivatives thereof.

Vaccines may additionally comprise at least one immunogen from at least one additional pathogen, e.g., a pig pathogen such as Actinobacillus pleuropneunomia; Adenovirus; Alphavirus such as Eastern equine encephalomyelitis viruses; Balantidium coli; Bordetella bronchiseptica; Brachyspira spp., preferably B. hyodyentheriae, B. pilosicoli, B. innocens, Brucella suis, preferably biovars 1, 2 and 3; Classical swine fever virus, Chlamydia and Chlamydophila spp., preferably C. pecorum and C. abortus; Clostridium spp., preferably Cl. difficile, Cl. perfringens types A, B and C, Cl. novyi, Cl. septicum, Cl. tetani; Digestive and respiratory Coronavirus; Cryptosporidium parvum; Eimeria spp.; Eperythrozoonis suis currently named Mycoplasma haemosuis; Erysipelothrix rhusiopathiae; Escherichia coli; Haemophilus parasuis, preferably subtypes 1, 7 and 14; Hemagglutinating encephalomyelitis virus; Isospora suis; Japanese Encephalitis virus; Lawsonia intracellulars; Leptospira spp., preferably Leptospira australis, Leptospira canicola, Leptospira grippotyphosa, Leptospira icterohaemorrhagicae, Leptospira interrogans, Leptospira Pomona and Leptospira tarassovi; Mannheimia haemolytica; Mycobacterium spp., preferably M. avium, M. intracellular and M. bovis: Mycoplasma hyponeumoniae; Parvovirus; Pasteurella multocida; Porcine circovirus; Porcine cytomegolovirus; Porcine parovirus, Porcine reproductive and respiratory syndrome virus: Pseudorabies virus; Rotavirus; Sagiyama virus; Salmonella spp., preferably S. thyhimurium and S. choleraesuis; Staphylococcus spp., preferably S. hyicus; Streptococcus spp., preferably Strep suis; Swine cytomegalovirus; Swine herpes virus; Swine influenza virus; Swinepox virus; Toxoplasma gondii; Vesicular stomatitis virus and virus of exanthema of swine; or other isolates and subtypes of porcine circovirus.

The vaccine compositions of the instant disclosure may be liquid formulations such as an aqueous solution, water-in-oil or oil-in-water emulsion, syrup, an elixir, a tincture, or a preparation for parenteral, subcutaneous, intradermal, intramuscular or intravenous administration (e.g., injectable administration), such as sterile suspensions or emulsions. Such formulations are known in the art and are typically prepared by dissolution of the antigen and other typical additives in the appropriate carrier or solvent systems. Liquid formulations also may include suspensions and emulsions that contain suspending or emulsifying agents.

The route of administration can be percutaneous, via mucosal administration, or via a parenteral route (intradermal, intramuscular, subcutaneous, intravenous, or intraperitoneal). Vaccine compositions according to the present disclosure may be administered alone, or can be co-administered or sequentially administered with other treatments or therapies.

The present disclosure also relates to methods of immunizing or inducing an immune response in animals (e.g., humans) comprising administering to said animal a peptide, polypeptide, concatemeric peptide, chimeric or fusion polypeptide, nucleic acid, cell, vector, pharmaceutical, or vaccine as described above.

The present disclosure also relates to methods of treating and/or preventing SARS-CoV-2 infection and/or related diseases caused by SARS-CoV-2, including COVID-19, diseases in animals (e.g., humans) comprising administering to said animal a peptide, polypeptide, concatemeric peptide, chimeric or fusion polypeptide, nucleic acid, cell, vector, pharmaceutical, or vaccine as disclosed herein.

A vaccine of the present disclosure can conveniently be administered intranasally, transdermally (i.e., applied on or at the skin surface for systemic absorption), parenterally, ocularly, etc. The parenteral route of administration includes, but is not limited to, intramuscular, intravenous, and intraperitoneal routes and the like.

The dosage of the vaccines of the present disclosure will depend on the species, breed, age, size, vaccination history, and health status of the animal (e.g., human) to be vaccinated, as well as the route of administration, e.g., subcutaneous, intradermal, oral intramuscular or intravenous administration. The vaccines of the present disclosure can be administered as single doses or in repeated doses. The vaccines of the instant disclosure can be administered alone, or can be administered simultaneously or sequentially administered with one or more further compositions, such as other porcine immunogenic or vaccine compositions. Where the compositions are administered at different times, the administrations may be separate from one another or overlapping in time.

In one aspect, the vaccine compositions of the present disclosure are administered to a subject susceptible to or otherwise at risk for SARS-CoV-2 infection and/or related diseases caused by SARS-CoV-2, including COVID-19, to enhance the subject own immune response capabilities. The subject to which the vaccine is administered is, in one aspect, a human. The animal may be susceptible to infection by SARS-CoV-2 infection (or a closely related virus) and/or related diseases caused by SARS-CoV-2, including COVID-19.

The present disclosure also provides a container comprising an immunologically effective amount of a polypeptide, nucleic acid or vaccine as described above. The present disclosure also provides vaccination kits comprising an optionally sterile container comprising an immunologically effective amount of the vaccine, means for administering the vaccine to animals, and optionally an instruction manual including information for the administration of the immunologically effective amount of the composition for treating and/or preventing SARS-CoV-2 infection (or a closely related virus) and/or related diseases caused by SARS-CoV-2, including COVID-19.

### (viii) Methods of Use of Tregitope Compositions

In some aspects, the present disclosure concerns methods of using the Treitope peptides and compositions thereof as set forth herein. Stimulating regulatory T cells with Tregitope peptide or compositions thereof of the present disclosure can stimulate, induce, and/or expand corresponding naturally occurring T_{Reg} populations (in aspects, including natural T_{Regs} and/or adaptive T_{Regs}) and in aspects results in increased secretion of one or more of the following cytokines and chemokines: IL-10, IL-35, TGF-β, TNF-α and MCP1. This increased secretion of regulatory cytokines and chemokines is a hallmark of regulatory T cells. In aspects, stimulation can result in the increased expression of IL-2Rα by corresponding naturally occurring T_{Reg} populations (in aspects, including natural T_{Regs} and/or adaptive T_{Regs}) and deprivation of IL-2 to effector T cells. In further aspects, stimulation can result in increased perforin granzyme by corresponding naturally occurring T_{Reg} populations (in aspects, including natural T_{Regs} and/or adaptive T_{Regs}), which allows for such Treg populations to kill T effector cells and other immune stimulatory cells. In even further aspects, such stimulation can result in the generation of immune suppressive adenosine by corresponding naturally occurring T_{Reg} populations (in aspects, including natural T_{Regs} and/or adaptive T_{Regs}). In other aspects, such stimulation can result in corresponding naturally occurring T_{Reg} populations (in aspects, including natural T_{Regs} and/or adaptive T_{Regs}) binding to and removing costimulatory molecules on dendritic cells, resulting the inhibition of dendritic cell function. Further, in aspects, such stimulation can result in T_{Reg} induced upregulation of checkpoint molecules on dendritic cells and other cell populations, e.g. but not limited to endothelial cells, by corresponding naturally occurring T_{Reg} populations (in aspects, including natural T_{Regs} and/or adaptive T_{Regs}). In additional aspects, such stimulation can result in T_{Reg} stimulation of B-regulatory cells. B-regulatory cells (“B-regs”) are cells that are responsible for the anti-inflammatory effect, which is characterized by the expression of CD1d, CD5, and the secretion of IL-10. B-regs are also identified by expression of Tim-1 and can be induced through Tim-1 ligation to promote tolerance. The ability of being B-regs was shown to be driven by many stimulatory factors such as toll-like receptors, CD40-ligand and others. However, full characterization of B-regs is ongoing. B-regs also express high levels of CD25, CD86, and TGF-β. The increased secretion of such regulatory cytokines and chemokines by regulatory T cells, as well as other activities described above, are hallmarks of regulatory T cells. In aspects, regulatory T cells activated by the Tregitope compositions of the present disclosure may express a CD4+CD25+FOXP3 phenotype. In aspects, regulatory T cells activated by the Tregitope compositions of the present disclosure may express a CD4+CD25+Foxp3+ phenotype. Regulatory T cells activated by the Tregitope compositions of the present disclosure directly suppress T-effector immune responses ex vivo as measured by decreased antigen-specific Th1- or Th2-associated cytokine levels, principally INF-γ, IL-4, and IL-5, and by decreased proliferation and/or effector function of antigen-specific T effector cells as measured by CFSE dilution and/or cytolytic activity. In aspects, regulatory T cells activated by the Tregitope compositions of the present disclosure directly suppress T effector immune responses in vivo, as measured by decreased antigen-specific Th1- or Th2-associated cytokine levels (as measured by Elisa assay), decreased antigen-specific T effector cell levels (as measured by EliSpot assay), decreased cytolytic activity, and/or decreased antibody titers for protein antigens.

In aspects, natural regulatory T cells activated by the Tregitope compositions of the present disclosure stimulate the development of adaptive T_{Reg} cells. In aspects, co-incubating peripheral T cells with the Tregitope compositions of the present disclosure in the presence of antigen results in the expansion of antigen-specific CD4+/CD25+ T cells, upregulates the expression of the Foxp3 gene or Foxp3 protein in those cells and suppresses the activation of antigen-specific T effector cells in vitro. In aspects, the Tregitope compositions of the present disclosure may result in the activation and/or expansion of T regulatory type 1 (Tr1) cells. Tr1 cells have strong immunosuppressive capacity in several immune-mediated diseases (Roncarolo and Battaglia, 2007, Nat Rev Immunol 7, 585-598; Roncarolo et al., 2011, Immunol Rev 241, 145-163; Pot et al., 2011, Semin Immunol 23, 202-208). The secretion of high levels of IL-10, and the killing of myeloid antigen-presenting cells (APCs) via Granzyme B are the main mechanisms of Tr1-mediated suppression (Groux et al., 1997, Nature 389, 737-742; Magnani et al., 2011 Eur J Immunol 41, 1652-1662). Tr1 cells are distinguished from T helper (T_{H})1, T_{H}2, and T_{H}17 cells by their unique cytokine profile and the regulatory function. Tr1 cells have been shown secrete higher levels of IL-10 than IL-4 and IL-17, the hallmark cytokines of T_{H}2 and T_{H}17 cells, respectively. Tr1 cells can also secrete low levels of IL-2 and, depending on the local cytokine milieu, can produce variable levels of IFN-γ, together, the key T_{H}1 cytokines (Roncarolo et al., 2011, Immunol Rev 241, 145-163). FOXP3 is not a biomarker for Tr1 cells since its expression is low and transient upon activation. IL-10-producing Tr1 cells express ICOS (Haringer et al., 2009, J Exp Med 206, 1009-1017) and PD-1 (Akdis et al., 2004, J Exp Med 199, 1567-1575), but these markers are not specific (Maynard et al., 2007, Nat Immunol 8, 931-941). CD49b, the α2 integrin subunit of the very-late-activation antigen (VLA)-2, has been proposed as a marker for IL-10-producing T cells (Charbonnier et al., 2006, J Immunol 177, 3806-3813); but it is also expressed by human T_{H}17 cells (Boisvert et al., 2010, Eur J Immunol 40, 2710-2719). Moreover, murine CD49b⁺ T cells secrete IL-10 (Charbonnier et al., 2006, J Immunol 177, 3806-3813) but also pro-inflammatory cytokines (Kassiotis et al., 2006, J Immunol 177, 968-975). Lymphocyte activation gene-3 (LAG-3), a CD4 homolog that binds with high affinity to MHC class II molecules, is expressed by murine IL-10-producing CD4⁺ T cells (Okamura et al., 2009, Proc Natl Acad Sci USA 106, 13974-13979), but also by activated effector T cells (Workman and Vignali, 2005, J Immunol 174, 688-695; Bettini et al., 2011, J Immunol 187, 3493-3498; Bruniquel et al., 1998, Immunogenetics 48, 116-124; Lee et al., 2012, Nat Immunol 13, 991-999) and by FOXP3⁺ regulatory T cells (Tregs) (Camisaschi et al., 2010, J Immunol 184, 6545-6551). It was recently shown that human Tr1 cells express CD226 (DNAM-1), which is involved in the specific killing of myeloid APCs (Magnani et al., 2011 Eur J Immunol 41, 1652-1662). In further aspects, Tregitope compositions of the present disclosure may result in the activation and/or expansion of TGF-β secreting Th3 cells, regulatory NKT cells, regulatory CD8⁺ T cells, double negative regulatory T cells. “Th3 cells” refer to cells having the following phenotype CD4⁺FoxP3⁺ and capable of secreting high levels TGF-β upon activation, amounts of IL-4 and IL-10 and no IFN-γ or IL-2. These cells are TGF-β derived. “Regulatory NKT cells” refers to cells having the following phenotype at rest CD161⁺CD56⁺CD16⁺ and a Vα24/Vβ11 TCR. “Regulatory CD8+ T cells” refers to cells having the following phenotype at rest CD8⁺CD122⁺ and capable of secreting highs levels of IL-10 upon activation. “Double negative regulatory T cells” refers to cells having the following phenotype at rest TCRαβ⁺CD4⁻CD8⁻.

In aspects, the Tregitope compositions of the present disclosure are useful for regulating immune response to monoclonal antibodies, protein therapeutics, self-antigens promoting autoimmune response, allergens, transplanted tissues, and in other applications where tolerance is the desired outcome.

In aspects, the Tregitopes of the present disclosure can bind MHC class II molecules, engage TCR in context of MHC class II molecules and activate naturally occurring T_{Regs} (in aspects, including natural T_{Regs} and/or adaptive T_{Regs}).

Suppressing an Immune Response in a Subject in Need Thereof. In aspects, the present disclosure is directed to a method of stimulating, inducing, and/or expanding regulatory T-cells by administering or introducing or contact with an amino acid sequence of SEQ ID NOS: 4-370, 391-440, and 448-833, either directly or through introduction of a nucleic acid encoding such and providing or allowing for transcription and translation thereof.

In aspects, the present disclosure is directed to a method of stimulating/inducing, regulatory T-cells (e.g., naturally occurring T_{Regs} (in aspects, including natural T_{Regs} and/or adaptive T_{Regs})) to suppress an immune response in a subject in need thereof by administering to the subject a therapeutically effect amount of a Tregitope composition of the present disclosure. In aspects, the immune response is the result of one or more therapeutic treatments with at least one therapeutic protein, treatment with a vaccine (particularly in situations in which an adverse event results from the vaccination), or treatment with at least one antigen. In another aspect, the administration of a Tregitope composition of the present disclosure shifts one or more antigen presenting cells to a regulatory phenotype, one or more dendritic cells to a regulatory phenotype, decreases CD11c and HLA-DR expression in the dendritic cells or other antigen presenting cells.

In aspects, the present disclosure is directed to a method for repressing/suppressing an immune response in a subject, comprising administering a therapeutically effective amount of Tregitope composition of the present disclosure wherein the Tregitope composition represses/suppresses the immune response. In aspects, the Tregitope composition represses/suppresses an innate immune response. In aspects, the Tregitope composition represses/suppresses an adaptive immune response. In aspects, the Tregitope composition represses/suppresses an effector T cell response. In aspects, the Tregitope composition represses/suppresses a memory T cell response. In aspects, the Tregitope composition represses/suppresses helper T cell response. In aspects, the Tregitope composition represses/suppresses B cell response. In aspects, the Tregitope composition represses/suppresses an NKT cell response.

In aspects, the present disclosure is directed to a method of suppressing an immune response, specifically an antigen-specific immune response in a subject, through the administration of a therapeutically effective amount of a Tregitope composition of the present disclosure, wherein said Tregitope composition activates naturally occurring T_{Regs} (in aspects, including natural T_{Regs} and/or adaptive T_{Regs}, and in aspects CD4⁺/CD25⁺/FoxP3⁺ regulatory T-cells) or suppresses the activation of CD4⁺ T-cells, the proliferation of CD4⁺ and/or CD8⁺ T-cells, and/or suppresses the activation or proliferation of β-cells or NKT Cells. In aspects, a Tregitope composition of the present disclosure may be either covalently bound, non-covalently bound, or in admixture with a specific target antigen. In particular aspects, one or more of e.g., polypeptides (Treg activating regulatory T-cell epitope, Tregitope, Tregitope peptide, or T-cell epitope polypeptide, which in aspects may be isolated, synthetic, or recombinant) and/or chimeric or fusion polypeptide compositions of the presently disclosed Tregitope compositions may be either covalently bound, non-covalently bound, or in admixture with a specific target antigen. In aspects, an administered Tregitope composition of the present disclosure that is covalently bound, non-covalently bound, or in admixture with a specific target antigen results in the diminution of immune response against the target antigen.

In aspects, the target antigen may be an autologous protein or protein fragment. In aspects, the target antigen may be an allergen. In aspects, the target antigen may be an allogenic protein or protein fragments. In aspects, the target antigen may be a biologic medicine or fragments thereof. In aspects, the suppressive effect is mediated by natural T_{Regs}. In aspects, the suppressive effect is mediated by an adaptive T_{Regs}. In aspects, the one or more Tregitopes included in the Tregitope composition of the present disclosure suppresses an effector T cell response. In aspects, the one or more Tregitopes of the presently disclosed Tregitope composition suppresses an innate immune response. In aspects, the one or more Tregitopes of the presently disclosed Tregitope composition suppresses an adaptive immune response. In aspects, the one or more Tregitopes of the presently disclosed Tregitope composition suppresses helper T cell response. In aspects, the one or more Tregitopes of the presently disclosed Tregitope composition suppresses a memory T cell response. In aspects, the one or more Tregitopes of the presently disclosed Tregitope composition suppresses a β cell response. In aspects, the one or more Tregitopes of the presently disclosed Tregitope composition suppresses a NKT cell response.

Designing Small Molecule Therapeutics. In one aspect, the present disclosure provides methods of using a Tregitope composition of the present disclosure for the purpose of designing small molecule therapeutics. In one aspect, Tregitope-specific T cells are stimulated three times with pools of small molecule mixtures at a concentration of 1 μg/ml and autologous dendritic cells (DC) at 2-week intervals, followed by stimulation with heterologous DC and antigens. T cells (1.25×10⁵) and DC (0.25×10⁵) are added per well in round-bottom, 96-well plates. T cell medium is made by supplementing 500 ml of RPMI medium 1640 with 50 ml of FCS (HyClone Laboratories, Inc., Logan, Utah), penicillin, and streptomycin (GIBCO Laboratories, Gaithersburg, Md.); 20 mM Hepes (GIBCO); and 4 mL 1 N NaOH solution. The IL-2 concentration is initially 0.1 nM and gradually is increased to 1 nM during subsequent rounds of stimulation. T cell clones are derived by limiting dilution by using 0.6×10⁵ Epstein-Barr virus-transformed B cells (100 Gray) and 1.3×10⁵ heterologous peripheral blood mononuclear cells (33 Gray) as feeder cells and 1 μg/ml Difco™ phytohemagglutinin (Bacterius Ltd, Houston, Tex.) in medium containing 2 nM IL-2. Small molecules pools that stimulate the Tregitope specific T cells are then tested as individual molecules.

Cloning T Cell Receptors. In aspects, the present disclosure provides methods of using a Tregitope composition of the present disclosure for the purpose of cloning T cell receptors. Cloning of Tregitope-specific T cells can be conducted by techniques known to one of skill in the art. For example, isolated PBMCs are stimulated with Tregitopes at 10 μg/ml RPMI media containing 20% HSA. IL-2 is added (10 U/ml final concentration) every other day starting on day 5. T cells are stained with tetramer pools on day 11 or 12. For each pool, 2-3×10⁵ cells are incubated with 0.5 mg of PE-labeled tetramer in 50 ml of culture medium (10 mg/ml) at 37° C. for 1 to 2 h, and then stained with anti-CD4-FITC (BD PharMingen™, San Diego, Calif.) for 15 min at room temperature. Cells are washed and analyzed with a Becton Dickinson FACSCalibur™ flow cytometer (Becton Dickinson, San Jose, Calif.). Tetramers loaded with the corresponding single peptides are generated for those pools that give positive staining, and analysis is done on day 14 or 15. Cells that are positive for a particular tetramer are single-cell sorted into 96-well U-bottom plates by using a Becton Dickinson FACSVantage™ (San Jose, Calif.) on the same or following day. Sorted cells are expanded with 1.5-3×10⁵ unmatched, irradiated (5000 rad) PBMC per well as feeders with 2.5 mg/ml PHA and 10 U/ml IL-2 added 24 h later. Specificity of cloned T cells is confirmed by staining with tetramers (loaded with cognate peptide or control peptide, HA307-319) and T cell proliferation assays with 10 mg/ml of specific peptide (Novak E J et al., J Immunol, 166(11):6665-70). In aspects, total RNA is extracted with an RNeasy Mini Kit (Qiagene, Hilden, Del.) from the Tregitope specific T cell lines generated as described above. One microgram of total RNA is used to clone the TCR cDNAs by a rapid amplification of cDNA end (RACE) method using a GeneRacer® kit (Invitrogen, Carlsbad, Calif.). Before synthesizing the single-strand cDNA, the RNA is de-phosphorylated, de-capped, and ligated with an RNA oligonucleotide according to the instruction manual of 5′ RACE GeneRacer® kit. SuperScript II RT® (Life Technologies Corp, Carlsbad, Calif.) and GeneRacer® Oligo-dT are used for reverse transcription of the RNA Oligo-ligated mRNA to single-strand cDNAs. 5′ RACE is performed by using GeneRacer® 5′ (GeneRacer® Kit) as 5′ primer and gene-specific primer TCRCAR (5′-GTT AAC TAG TTC AGC TGG ACC ACA GCC GCA GC-3′; SEQ ID NO: 839) or TCRCB1R (5′-CGG GTT AAC TAG TTC AGA AAT CCT TTC TCT TGA CCA TGG C-3′; SEQ ID NO: 840), or TCRCBR2 (5′-CTA GCC TCT GGA ATC CTT TCT CTT G-3′; SEQ ID NO: 841) as 3′ primers for TCR α, β1, or β2 chains, respectively. The polymerase chain reaction (PCR) products are cloned into pCR2.1 TOPO vector (Invitrogen, Carlsbad, Calif.) and then transformed into One Shot TOP10 Competent Escherichia coli (Invitrogen, Carlsbad, Calif.). Plasmid DNAs are prepared from 96 individual clones from each construct for TCRα, β1, and β2 chains. Full-length insert of all the plasmids is sequenced to determine the vα/vβ usage (Zhao Y et al., (2006), J Immunother, 29(4):398-406, herein incorporated by reference in its entirety).

Methods of Preventing or Treating a Medical Condition. The present disclosure is directed to, for example methods of preventing or treating one or more medical conditions in a subject comprising administering a Tregitope composition of the present disclosure, and preventing or treating the medical condition in a subject by said step of administering. The medical condition can be, for example, primary immunodeficiencies, immune-mediated thrombocytopenia, Kawasaki disease, hematopoietic stem cell transplantation in patients older than 20 years, chronic B-cell lymphocytic leukemia, and pediatric HIV type 1 infections. Specific examples include: (Hematology) aplastic anemia, pure red cell aplasia, Diamond-Blackfan anemia, autoimmune hemolytic anemia, hemolytic disease of the newborn, acquired factor VIII inhibitors, acquired von Willebrand disease, immune-mediated neutropenia, refractoriness to platelet transfusion, neonatal alloimmune/autoimmune thrombocytopenia, posttransfusion purpura, thrombotic thrombocytopenia purpura/hemolytic uremic syndrome; Infectious diseases, solid organ transplantation, surgery, trauma, burns, and HIV infection; (Neurology) epilepsy and pediatric intractable Guillain-Barré syndrome, chronic inflammatory demyelinating polyneuropathy, myasthenia gravis, Lambert-Eaton myasthenic syndrome, multifocal motor neuropathy, multiple sclerosis; (Obstetrics) recurrent pregnancy loss; (Pulmonology) asthma, chronic chest symptoms, rheumatology, rheumatoid arthritis (adult and juvenile), systemic lupus erythematosus, systemic vasculitides, dermatomyositis, polymyositis, inclusion-body myositis, wegener granulomatosis; (Miscellaneous) adrenoleukodystrophy, amyotrophic lateral sclerosis, Behçet syndrome, acute cardiomyopathy, chronic fatigue syndrome, congential heart block, cystic fibrosis, autoimmune blistering dermatosis, diabetes mellitus, acute idiopathic dysautonomia, acute disseminated encephalomyelitis, endotoxemia, hemolytic transfusion reaction, hemophagocytic syndrome, acute lymphoblastic leukemia, lower motor neuron syndrome, multiple myeloma, human T-cell lymphotrophic virus-1-associated myelopathy, nephritic syndrome, membranous nephropathy, nephrotic syndrome, euthyroid ophthalmopathy, opsoclonus-myoclonus, recurrent otitis media, paraneoplastic cerebellar degeneration, paraproteinemic neuropathy, parvovirus infection (general), polyneuropathy, organomegaly, endocrinopathy, M-protein, and skin changes (POEMS) syndrome, progressive lumbosacral plexopathy, lyme radiculoneuritis, Rasmussen syndrome, Reiter syndrome, acute renal failure, thrombocytopenia (nonimmune), streptococcal toxic shock syndrome, uveitis, and Vogt-Koyanagi-Harada syndrome.

In a particular aspect, the present invention is directed to, for example, methods of treating allergy, autoimmune disease, transplant-related disorders such as graft versus host disease, enzyme or protein deficiency disorders, hemostatic disorders (e.g., Hemophilia A, B, or C), cancers (particularly tumor associated autoimmunity), infertility, or infections (viral, bacterial, or parasitic). The Tregitope composition of the present disclosure can be used with in conjunction with other proteins or compounds used for treating a subject with a medical condition in order to reduce adverse events or enhance the efficacy of the co-administered compound.

Application to Allergy. Allergen-specific regulatory T cells play an important role in controlling the development of allergy and asthma. Naturally occurring T_{Regs} (in aspects, including natural T_{Regs} and/or adaptive T_{Regs}, and in aspects CD4⁺/CD25⁺/FoxP3⁺ regulatory T-cells) have been shown to inhibit the inappropriate immune responses involved in allergic diseases. A number of recent studies indicate that regulatory T cells play an important role in controlling the overdevelopment of T-helper type 2 biased immune responses in susceptible individuals, not only in animal models, but in humans as well. Recent studies indicate that T_{regs} also suppress T cell co-stimulation by the secretion of TGF-β and IL-10, suggesting an important role of T_{regs} in the regulation of allergic disorders. Impaired expansion of natural or adaptive regulatory T cells leads to the development of allergy, and treatment to induce allergen-specific T_{regs} would provide curative therapies for allergy and asthma. One strategy for both the prevention and therapy of asthma is the induction of T_{regs}. Animals can be protected from developing asthma by immune stimulation leading to Th1 or T_{reg} responses. Accordingly, Tregitope compositions of the present disclosure are useful in methods for the prevention or treatment of allergy and/or asthma. As such, in aspects, the present disclosure is directed to a method of preventing or treating allergy and/or asthma in a subject, the method comprising administering a therapeutically-effective amount of a Tregitope composition of the present disclosure, and preventing or treating allergy and/or asthma in a subject by said step of administering.

Application to Transplantation. The Tregitope compositions of the present disclosure are useful to induce tolerance during the transplantation process, by promoting the development of cells that specifically down regulate immune responses against donor cells. Induction of Ag-specific T_{Reg} cells for treating organ-specific autoimmunity is an important therapeutic development, avoiding generalized immune suppression. In murine models of bone marrow transplantation, T_{Regs} promote donor bone marrow engraftment and decrease the incidence and severity of graft versus host disease without abrogating the beneficial graft versus tumor immunologic effect. These findings, in concert with observations that T_{Regs} in mice and humans share phenotypic and functional characteristics, have led to active investigations into the use of these cells to decrease complications associated with human hematopoietic cell transplantation. An imbalance of T_{Regs} and effector T cells contributes to the development of graft versus host disease, however, the mechanisms of immunoregulation, in particular, the allorecognition properties of T_{Regs}, their effects on and interaction with other immune cells, and their sites of suppressive activity, are not well understood.

Accumulating evidence from both humans and experimental animal models has implicated the involvement of T_{Regs} in the development of graft versus host disease (GVHD). The demonstration that T_{Regs} can separate GVHD from graft versus tumor (GVT) activity suggests that their immunosuppressive potential could be manipulated to reduce GVHD without detrimental consequence on GVT effect. Although a variety of T lymphocytes with suppressive capabilities have been reported, the two best-characterized subsets are the naturally arising, intrathymic-generated T_{Regs} (natural T_{Regs}) and the peripherally generated, inducible T_{Regs} (inducible T_{Regs}). Accordingly, Tregitope compositions of the present disclosure are useful in methods for inducing tolerance during the transplantation process. As such, in aspects, the present disclosure is directed to a method of inducing tolerance during the transplantation process in a subject, the method comprising administering a therapeutically-effective amount of a Tregitope composition of the present disclosure, and inducing tolerance during the transplantation process in a subject by said step of administering.

Application as a Tolerizing Agent and to Autoimmunity. In aspects, Tregitope compositions of the present disclosure can be used as a tolerizing agents for immunogenic compounds (protein therapeutics) (Weber C A et al., (2009), Adv Drug Deliv, 61(11):965-76). This discovery has implications for the design of protein therapeutics. Thus, administration of an immunogenic compound (e.g., protein therapeutic, such as but not limited to monoclonal antibody, autologous cytokine, or foreign protein) in conjunction with a Tregitope composition of the present disclosure suppresses adverse T effector immune responses. In vivo, T_{Regs} act through dendritic cells to limit autoreactive T-cell activation, thus preventing their differentiation and acquisition of effector functions. By limiting the supply of activated pathogenic cells, T_{Regs} prevent or slow down the progression of autoimmune diseases. This protective mechanism appears, however, insufficient in autoimmune individuals, likely because of a shortage of T_{Regs} cells and/or the development and accumulation of T_{Reg}-resistant pathogenic T cells over the long disease course. Thus, restoration of self-tolerance in these patients may require purging of pathogenic T cells along with infusion of T_{Regs} with increased ability to control ongoing tissue injury. Organ-specific autoimmune conditions, such as thyroiditis and insulin-dependent diabetes mellitus have been attributed to a breakdown of this tolerance mechanism (Mudd P A et al., (2006), Scand J Immunol, 64(3):211-8). Accordingly, Tregitope compositions of the present disclosure are useful in methods for the prevention or treatment of autoimmunity. As such, in aspects, the present disclosure is directed to a method of preventing or treating autoimmunity in a subject, the method comprising administering a therapeutically-effective amount of a Tregitope composition of the present disclosure, and preventing and/or treating autoimmunity in a subject by said step of administering.

Application to Hepatitis B (HBV) infection. Chronic HBV is usually either acquired (by maternal fetal transmission) or can be a rare outcome of acute HBV infection in adults. Acute exacerbations of chronic hepatitis B (CH-B) are accompanied by increased cytotoxic T cell responses to hepatitis B core and e antigens (HBcAg/HBeAg). In a recent study, the SYFPEITHI T cell epitope mapping system was used to predict MHC class II-restricted epitope peptides from the HBcAg and HbeAg (Feng I C et al., (2007), J Biomed Sci, 14(1):43-57). MHC class II tetramers using the high scoring peptides were constructed and used to measure T_{Reg} and CTL frequencies. The results showed that T_{Reg} cells specific for HBcAg declined during exacerbations accompanied by an increase in HBcAg peptide-specific cytotoxic T cells. During the tolerance phase, FOXp3-expressing T_{Reg} cell clones were identified. These data suggest that the decline of HbcAg T_{Reg} T cells accounts for the spontaneous exacerbations on the natural history of chronic hepatitis B virus infection. Accordingly, Tregitope compositions of the present disclosure are useful in methods for the prevention or treatment of chronic hepatitis B viral infection. As such, in aspects, the present disclosure is directed to a method of preventing or treating a viral infection (e.g., HBV infection) in a subject, the method comprising administering a therapeutically-effective amount of a Tregitope composition of the present disclosure, and preventing and/or treating said viral infection in a subject by said step of administering.

Ex Vivo Expansion and/or Stimulation of T-Regulatory Cells Using Tregitope Compositions. In aspects, the present disclosure provides ex vivo methods for the expansion of regulatory T-cells. In one embodiment, the invention provides a method of expanding regulatory T-cells in a biological sample, the method comprising: (a) providing a biological sample from a subject; (b) isolating regulatory T-cells from the biological sample; and contacting the isolated regulatory T-cells with an effective amount of a Tregitope composition of the present disclosure under conditions wherein the T-regulatory cells increase in number to yield an expanded regulatory T-cells, thereby expanding the regulatory T-cells in the biological sample. In aspects, the method further comprises the step of administration of the expanded regulatory T-cells to a subject. In aspects, the subject administered the expanded regulatory T-cells is the same individual from which the original biological sample was obtained, e.g., by autologous transplantation of the expanded Tregitope (Ruitenberg J J et al., (2006), BMC Immunol, 7:11).

In aspects, the present disclosure provides ex vivo methods for stimulation of regulatory T-cells in a biological sample, the method comprising: (a) providing a biological sample from a subject; and (b) isolating regulatory T-cells from the biological sample; and contacting the isolated regulatory T-cells with an effective amount of a Tregitope composition of the present disclosure under conditions wherein the T-regulatory cells are stimulated to alter one or more biological function, thereby stimulating the regulatory T-cells in the biological sample. In aspects, the method further comprises the step of administration of the stimulated regulatory T-cells to a subject. In aspects, the subject to which the stimulated regulatory T-cells are administered is the same subject from which the original biological sample was obtained, e.g., by autologous transplantation of the expanded Tregitope.

Ex Vivo Pulsing of Antigen Presenting Cells using Tregitope Compositions. In aspects, the present disclosure provides ex vivo methods for antigen presenting cells (e.g., dendritic cells, macrophages, etc.) in a biological sample, the method comprising: (a) providing a biological sample from a subject; and (b) isolating antigen presenting cells from the biological sample; and contacting the isolated antigen presenting with an effective amount of a Tregitope composition of the present disclosure under conditions wherein the antigen presenting cells are stimulated to alter one or more biological function (e.g., to present the Tregitopes and/or skew the antigen presenting cells to a be tolerogenic (which, in aspects, can further include cytokine treatment of the antigen presenting cells to induce such a tolerogenic state), thereby stimulating the antigen presenting cells in the biological sample. In aspects, the method further comprises the step of administration of the stimulated antigen presenting cells to a subject. In aspects, the subject to which the stimulated antigen presenting cells are administered is the same subject from which the original biological sample was obtained, e.g., by autologous transplantation of the stimulated antigen presenting cells.

In Vitro Uses of Tregitope Compositions. In aspects, the present disclosure provides the use of a Tregitope composition of the present disclosure as reagents in the study of regulatory T-cell function in in vitro studies and experimental models.

Kits. The methods described herein can be performed, e.g., by utilizing pre-packaged kits comprising at least one Tregitope composition of the present disclosure, which can be conveniently used, e.g., in clinical settings to treat subjects exhibiting symptoms or family history of a medical condition described herein. In one embodiment, the kit further comprises instructions for use of the at least one Tregitope composition of the instant disclosure to treat subjects exhibiting symptoms or family history of a medical condition described herein.

### (ix) Methods of Treatment with the Detolerized Antigens

In some aspects, the present disclosure concerns methods of use of the detolerized antigens as set forth herein, including proteins or peptides of SARS-CoV-2 wherein one or more of the Tregitopes as identified herein are removed and/or mutated to avoid, reduce or abolish T_{Reg} cell engagement. Stimulating T-cells with detolerized antigens or compositions thereof can stimulate, induce, and/or expand a corresponding naturally occurring immune response, e.g., stimulating, inducing, and/or expanding a corresponding naturally occurring immune response to a SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19, including CD4+ and/or CD8+ T cell responses, and in aspects results in increased secretion of one or more cytokines and chemokines. In aspects, T-cells activated by the T-cell epitope compounds and compositions of the present disclosure stimulate cell-mediated immunity against SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19, in a subject.

In aspects, T cells activated by the detolerized antigens or compositions thereof of the present disclosure can stimulate cell-mediated immunity against SARS-CoV-2 infection (or a closely related virus) and/or related diseases caused by SARS-CoV-2, including COVID-19 in a subject.

In aspects, the present disclosure is directed to a method of stimulating, inducing, and/or expanding an immune response, e.g., against SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19, in a subject in need thereof by administering to the subject a therapeutically effect amount of a detolerized antigen or composition thereof as set forth herein.

In aspects, the present disclosure is directed to a method of preventing, treating, or ameliorating a disease by SARS-CoV-2 infection (or a closely related virus such as Severe Acute Respiratory Syndrome (SARS) or Middle East respiratory syndrome coronavirus (MERS-CoV))) such as COVID-19, in a subject in need thereof by administering to the subject a therapeutically effect amount of a detolerized antigen or composition thereof as set forth herein.

Although any assay, methods, and materials similar or equivalent to those described herein can be used in the practice or testing of the present disclosure, the preferred methods and materials are described. Other features, objects, and advantages of the present disclosure will be apparent from the description and the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All references cited herein are incorporated herein by reference in their entirety and for all purposes to the same extent as if each individual publication, patent, or patent application was specifically and individually indicated to be incorporated by reference in its entirety for all purposes.

### (x) Aspects

A 1st aspect, either alone or in combination with any or all other aspects herein, is directed to a polypeptide consisting of an amino acid sequence selected from the group consisting of SEQ ID NOS: 4-370, 391-440, and 448-833, and/or fragments and variants thereof, and optionally 1 to 12 additional amino acids distributed in any ratio on the N terminus and/or C-terminus of the polypeptide of SEQ ID NOS: 4-370, 391-440, and 448-833.

A 2^{nd} aspect, either alone or in combination with any or all other aspects herein, is directed to a polypeptide consisting essentially of an amino acid sequence selected from the group consisting of SEQ ID NOS: 4-370, 391-440, and 448-833, and/or fragments and variants thereof, and optionally 1 to 12 additional amino acids distributed in any ratio on the N terminus and/or C-terminus of the polypeptide of SEQ ID NOS: 4-370, 391-440, and 448-833.

A 3rd aspect, either alone or in combination with any or all other aspects herein, is directed to a polypeptide comprising an amino acid sequence selected from the group consisting of SEQ ID NOS: 4-370, 391-440, and 448-833, and/or fragments and variants thereof, and optionally 1 to 12 additional amino acids distributed in any ratio on the N terminus and/or C-terminus of the polypeptide of SEQ ID NOS: 4-370, 391-440, and 448-833.

A 4th aspect, either alone or in combination with any or all other aspects herein, is directed to a polypeptide according to any one of claims 1-3, wherein said variant or fragment of an amino acid sequence selected from the group consisting of SEQ ID NOS: 4-370, 391-440, and 448-833 retains MHC binding propensity and TCR specificity, and/or retains anti-SARS-CoV-2 activity.

A 5th aspect, either alone or in combination with any or all other aspects herein, is directed to a polypeptide consisting of an amino acid sequence having at least 75%, 80%, 85%, 90%, or 95% homology to any one of SEQ ID NOS: 4-370, 391-440, and 448-833, and fragments thereof, wherein said polypeptide retains MHC binding propensity and the same TCR specificity, and/or retains anti-SARS-CoV-2 activity.

A 6th aspect, either alone or in combination with any or all other aspects herein, is directed to a polypeptide consisting essentially of an amino acid sequence having at least 75%, 80%, 85%, 90%, or 95% homology to any one of SEQ ID NOS: 4-370, 391-440, and 448-833, and fragments thereof, wherein said polypeptide retains MHC binding propensity and the same TCR specificity, and/or retains anti-SARS-CoV-2 activity.

A 7th aspect, either alone or in combination with any or all other aspects herein, is directed to a polypeptide comprising an amino acid sequence having at least 75%, 80%, 85%, 90%, or 95% homology to any one of SEQ ID NOS: 4-370, 391-440, and 448-833, and fragments thereof, wherein said polypeptide retains MHC binding propensity and the same TCR specificity, and/or retains anti-SARS-CoV-2 activity.

An 8th aspect, either alone or in combination with any or all other aspects herein, is directed to a protein or peptide derived from the SARS-CoV-2 virus wherein one or more Tregitopes is deleted, partially deleted and/or mutated such that the protein or peptide is a detolerzed antigen and cannot engage, stimulate or activate a T_{Reg} cell when administered.

A 9^{th} aspect, either alone or in combination with any or all other aspects herein, is directed to the protein or peptide of the 8^{th} aspect, wherein the deleted, partially deleted and/or mutated Tregitope is selected from an amino acid sequence as set forth in SEQ ID NOS: 4-370, 391-440, and 448-833.

A 10^{th} aspect, either alone or in combination with any or all other aspects herein, is directed to a polypeptide according to any of aspects 8 or 9, wherein the amino acid sequence is mutated in an anchoring amino acid to the MHC and/or in a T-cell receptor binding epitope to detolerize the polypeptide.

An 11th aspect, either alone or in combination with any or all other aspects herein, is directed to a detolerized antigen of the SARS-CoV-2 envelope of SEQ ID NO: 1, wherein the detolerized antigen comprises a deletion, partial deletion and/or mutation to one or more of SEQ ID NOS: 6, 7, 18-31, 186-231, and/or 448-459.

A 12^{th} aspect, either alone or in combination with any or all other aspects herein is directed to the detolerized antigen of aspect 11, wherein SEQ ID NO: 7 includes one or more mutations at positions K63, N64, N66, S68, R69, V62, L65, S67, and/or V70 in relation to SEQ ID NO: 1.

A 13^{th} aspect, either alone or in combination with any or all other aspects herein is directed to the detolerized antigen of aspect 11, wherein SEQ ID NO: 6 includes one or more mutations at positions T11, L12, V14, N15, S16, V17, L19, F20, A22, F23, V24, V25, F26, L27, L28, V29, T30, L31, A32, I33, L34, A36, R38, and/or A41 in relation to SEQ ID NO: 1.

A 14^{th} aspect, either alone or in combination with any or all other aspects herein is directed to the detolerized antigen of aspect 11, wherein SEQ ID NO: 6 includes one or more mutations at positions L12, I13, V14, N15, S16, V17, L18, L19, F20, L21, A22, F23, V24, V25, F26, L27, L28, V29, T30, L31, A32, I33, L34T35, L37, L39, and/or C40 in relation to SEQ ID NO: 1.

A 15^{th} aspect either alone or in combination with any or all other aspects herein, is directed to a detolerized antigen of the SARS-CoV-2 membrane of SEQ ID NO: 2, wherein the detolerized antigen comprises a deletion, partial deletion and/or mutation to one or more of SEQ ID NOS: 4, 5, 17, 32-41, 232-245, 440, and 450-471.

A 16^{th} aspect, either alone or in combination with any or all other aspects herein is directed to the detolerized antigen of aspect 15, wherein SEQ ID NO: 4 includes one or more mutations at positions I118, N121, P123, and/or G126 in relation to SEQ ID NO: 2.

A 17^{th} aspect, either alone or in combination with any or all other aspects herein is directed to the detolerized antigen of aspect 16, wherein the mutations to SEQ ID NO: 4 includes one or more of I118A; I118G; I118N; I118Q; I118S; I118T; N121P; P123Q; P123G; and/or G126P in relation to SEQ ID NO: 2.

An 18^{th} aspect, either alone or in combination with any or all other aspects herein is directed to the detolerized antigen of aspect 15, wherein SEQ ID NO: 4 includes one or more mutations at positions L119, L120, V122, L124, and/or H125 in relation to SEQ ID NO:

2.

A 19^{th} aspect, either alone or in combination with any or all other aspects herein, is directed to the detolerized antigen of aspect 15, wherein SEQ ID NO: 440 includes one or more mutations at positions Y179, G182, S184, and/or V187 in relation to SEQ ID NO: 2.

A 20^{th} aspect, either alone or in combination with any or all other aspects herein, is directed to the detolerized antigen of aspect 15, wherein SEQ ID NO: 440 includes one or more mutations at positions K180, L181, A183, Q185, and/or R186 in relation to SEQ ID NO: 2.

A 21st aspect, either alone or in combination with any or all other aspects herein, is directed to detolerized antigen of the SARS-CoV-2 spike of SEQ ID NO: 3, wherein the detolerized antigen comprises a deletion, partial deletion and/or mutation to one or more of SEQ ID NOS: 8-17, 42-93, 246-370, 422, 423, 432, 434-439, and 794-833.

A 22nd aspect, either alone or in combination with any or all other aspects herein is directed to the detolerized antigen of aspect 21, wherein SEQ ID NO: 8 includes one or more mutations at positions F28, S31, L33, T36, D38, and/or L41 in relation to SEQ ID NO: 3.

A 23rd aspect, either alone or in combination with any or all other aspects herein is directed to the detolerized antigen of aspect 22, wherein the mutations to SEQ ID NO: 8 includes one or more of F28G; F28A; F28N; F28T; F28S; F28Q, S31G; S31T and/or L33Q in relation to SEQ ID NO: 3.

An 24^{th} aspect, either alone or in combination with any or all other aspects herein is directed to the detolerized antigen of aspect 21, wherein SEQ ID NO: 8 includes one or more mutations at positions R29, S30, V32, H34, S35, Q37, L39, and/or F40 in relation to SEQ ID NO: 3.

A 25th aspect, either alone or in combination with any or all other aspects herein is directed to the detolerized antigen of aspect 21, wherein SEQ ID NO: 9 includes one or more mutations at positions I195, V198, D200 and/or Q203 in relation to SEQ ID NO: 3.

A 26th aspect, either alone or in combination with any or all other aspects herein is directed to the detolerized antigen of aspect 25, wherein the mutations to SEQ ID NO: 9 includes one or more of I195A; I195G; I195N; I195S; I195T; I195Q; V198G; V198T; V198N; Q203E; Q203G; and/or Q203T in relation to SEQ ID NO: 3.

An 27^{th} aspect, either alone or in combination with any or all other aspects herein is directed to the detolerized antigen of aspect 21, wherein SEQ ID NO: 9 includes one or more mutations at positions N196, L197, R199, L201, and/or P202 in relation to SEQ ID NO:

3.

A 28th aspect, either alone or in combination with any or all other aspects herein is directed to the detolerized antigen of aspect 21, wherein SEQ ID NO: 10 includes one or more mutations at positions 1220, F223, T225, and/or A228 in relation to SEQ ID NO: 3.

A 29th aspect, either alone or in combination with any or all other aspects herein is directed to the detolerized antigen of aspect 28, wherein the mutations to SEQ ID NO: 10 includes one or more of I220A; I220G; I220N; I220Q; I220S; I220T; and/or T225Q in relation to SEQ ID NO: 3.

An 30^{th} aspect, either alone or in combination with any or all other aspects herein is directed to the detolerized antigen of aspect 21, wherein SEQ ID NO: 10 includes one or more mutations at positions T221, R222, Q224, L226, and/or L227 in relation to SEQ ID NO: 3.

A 31st aspect, either alone or in combination with any or all other aspects herein is directed to the detolerized antigen of aspect 21, wherein SEQ ID NO: 11 includes one or more mutations at positions Y254, P257, Y259, and/or L262 in relation to SEQ ID NO: 3.

A 32nd aspect, either alone or in combination with any or all other aspects herein is directed to the detolerized antigen of aspect 31, wherein the mutations to SEQ ID NO: 11 includes one or more of Y254A; Y254G; Y254N; Y254Q; Y254S; Y254T; T259G; and/or T259Q in relation to SEQ ID NO: 3.

An 33rd aspect, either alone or in combination with any or all other aspects herein is directed to the detolerized antigen of aspect 21, wherein SEQ ID NO: 11 includes one or more mutations at positions L255, Q256, R258, F260, and/or L261 in relation to SEQ ID NO: 3.

A 34th aspect, either alone or in combination with any or all other aspects herein is directed to the detolerized antigen of aspect 21, wherein SEQ ID NO: 12 includes one or more mutations at positions V496, S499, E501, and/or H504 in relation to SEQ ID NO: 3.

A 35th aspect, either alone or in combination with any or all other aspects herein is directed to the detolerized antigen of aspect 34, wherein the mutations to SEQ ID NO: 12 includes one or more of V496A; V496G; V496N; V496Q; V496S; V496T; S499G; S499Q; and/or S499T in relation to SEQ ID NO: 3.

An 36th aspect, either alone or in combination with any or all other aspects herein is directed to the detolerized antigen of aspect 21, wherein SEQ ID NO: 12 includes one or more mutations at positions V497, L498, F500, L502, and/or L503 in relation to SEQ ID NO: 3.

A 37th aspect, either alone or in combination with any or all other aspects herein is directed to the detolerized antigen of aspect 21, wherein SEQ ID NO: 13 includes one or more mutations at positions L806, N809, V811, and/or A814 in relation to SEQ ID NO: 3.

A 38th aspect, either alone or in combination with any or all other aspects herein is directed to the detolerized antigen of aspect 37, wherein the mutations to SEQ ID NO: 13 includes one or more of L806A; L806G; L806N; L806Q; L806S; L806T; and/or N809G in relation to SEQ ID NO: 3.

An 39th aspect, either alone or in combination with any or all other aspects herein is directed to the detolerized antigen of aspect 21, wherein SEQ ID NO: 13 includes one or more mutations at positions L807, F808, K810, T812, and/or L813 in relation to SEQ ID NO: 3.

A 40th aspect, either alone or in combination with any or all other aspects herein is directed to the detolerized antigen of aspect 21, wherein SEQ ID NO: 14 includes one or more mutations at positions L843, L846, P848, and/or T851 in relation to SEQ ID NO: 3.

A 41st aspect, either alone or in combination with any or all other aspects herein is directed to the detolerized antigen of aspect 40, wherein the mutations to SEQ ID NO: 14 includes one or more of L843A; L843G; L843N; L843Q; L843S; L843T; L846G; L846T; and/or P848Q in relation to SEQ ID NO: 3.

An 42nd aspect, either alone or in combination with any or all other aspects herein is directed to the detolerized antigen of aspect 21, wherein SEQ ID NO: 14 includes one or more mutations at positions T844, V845, P847, L849, and/or L850 in relation to SEQ ID NO: 3.

A 43rd aspect, either alone or in combination with any or all other aspects herein is directed to the detolerized antigen of aspect 21, wherein SEQ ID NO: 15 includes one or more mutations at positions F912, A915, G917, Q920, L923, T926, S928, and/or G931 in relation to SEQ ID NO: 3.

A 44th aspect, either alone or in combination with any or all other aspects herein is directed to the detolerized antigen of aspect 43, wherein the mutations to SEQ ID NO: 15 includes one or more of F912A; F912G; F912N; F912Q; F912S; F912T; A915G; L923A; L923G; L923N; L923Q; L923S; L923T, and/or T926G in relation to SEQ ID NO: 3.

An 45th aspect, either alone or in combination with any or all other aspects herein is directed to the detolerized antigen of aspect 21, wherein SEQ ID NO: 15 includes one or more mutations at positions N913, S914, I916, K918, I919, S924, S925, A927, A929, and/or L930 in relation to SEQ ID NO: 3.

A 46th aspect, either alone or in combination with any or all other aspects herein is directed to the detolerized antigen of aspect 21, wherein SEQ ID NO: 16 includes one or more mutations at positions F955, I958, S960, and/or N963 in relation to SEQ ID NO: 3.

A 47th aspect, either alone or in combination with any or all other aspects herein is directed to the detolerized antigen of aspect 46, wherein the mutations to SEQ ID NO: 16 includes one or more of F955A; F955G; F955N; F955Q; F955S; F955T, I958G; S960G; 5960Q; and/or S960T in relation to SEQ ID NO: 3.

An 48th aspect, either alone or in combination with any or all other aspects herein is directed to the detolerized antigen of aspect 21, wherein SEQ ID NO: 16 includes one or more mutations at positions G956, A957, S959, V961, and/or L962 in relation to SEQ ID NO: 3.

A 49th aspect, either alone or in combination with any or all other aspects herein is directed to the detolerized antigen of aspect 21, wherein SEQ ID NO: 17 includes one or more mutations at positions I998, A1001, I1003, S1006, N1008, and/or A1011 in relation to SEQ ID NO: 3.

A 50th aspect, either alone or in combination with any or all other aspects herein is directed to the detolerized antigen of aspect 49, wherein the mutations to SEQ ID NO: 17 includes one or more of I998A; I998G; I998N; I998Q; I998S; I998T; A1001G; A1001T; I1003A; I1003G; I1003N; I1003Q; I1003S; I1003T and/or N1008Q in relation to SEQ ID NO: 3.

An 51st aspect, either alone or in combination with any or all other aspects herein is directed to the detolerized antigen of aspect 21, wherein SEQ ID NO: 17 includes one or more mutations at positions R999, A1000, E1002, R104, A1005, A1007, L1009, and/or A1010 in relation to SEQ ID NO: 3. A 52nd aspect, either alone or in combination with any or all other aspects herein, is directed to a nucleic acid encoding a polypeptide consisting of an amino acid sequence selected from the group consisting of SEQ ID NOS: 4-370, 391-440, and 448-833. and/or fragments and variants thereof, and optionally 1 to 12 additional amino acids distributed in any ratio on the N terminus and/or C-terminus of the polypeptide of SEQ ID NOS: 4-370, 391-440, and 448-833.

A 53rd aspect, either alone or in combination with any or all other aspects herein, is directed to a nucleic acid encoding a polypeptide consisting essentially of an amino acid sequence selected from the group consisting of SEQ ID NOS: 4-370, 391-440, and 448-833, and/or fragments and variants thereof, and optionally 1 to 12 additional amino acids distributed in any ratio on the N terminus and/or C-terminus of the polypeptide of SEQ ID NOS: 4-370, 391-440, and 448-833.

A 54th aspect, either alone or in combination with any or all other aspects herein, is directed to a nucleic acid encoding a polypeptide comprising an amino acid sequence selected from the group consisting of SEQ ID NOS: 4-370, 391-440, and 448-833, and/or fragments and variants thereof, and optionally 1 to 12 additional amino acids distributed in any ratio on the N terminus and/or C-terminus of the polypeptide of SEQ ID NOS: 4-370, 391-440, and 448-833.

An 558th aspect, either alone or in combination with any or all other aspects herein, is directed to a nucleic acid encoding the polypeptide of aspect 8 to 51.

A 56th aspect, either alone or in combination with any or all other aspects herein, is directed to a vector comprising the nucleic acid according to any one of aspects 52-55.

A 57th aspect, either alone or in combination with any or all other aspects herein, is directed to a plasmid comprising the vector according to aspect 56.

A 58th aspect, either alone or in combination with any or all other aspects herein, is directed to a cell comprising the vector according to aspect 56.

A 59th aspect, either alone or in combination with any or all other aspects herein, is directed to a pharmaceutical composition comprising a polypeptide according to any one of the aspects 1 to 51 and a pharmaceutically-acceptable carrier and/or excipient.

A 60th aspect, either alone or in combination with any or all other aspects herein, is directed to a pharmaceutical composition comprising a nucleic acid according to any one of the aspects 52 to 55 and a pharmaceutically-acceptable carrier and/or excipient.

A 61st aspect, either alone or in combination with any or all other aspects herein, is directed to a pharmaceutical composition comprising a vector according to the 56^{th} aspect and a pharmaceutically-acceptable carrier and/or excipient.

A 62nd aspect, either alone or in combination with any or all other aspects herein, is directed to pharmaceutical composition comprising a plasmid according to the 57th aspect and a pharmaceutically-acceptable carrier and/or excipient.

A 63rd aspect, either alone or in combination with any or all other aspects herein, is directed to vaccine comprising a polypeptide according to any one of aspects 1-51 and a pharmaceutically-acceptable excipient, carrier, and/or adjuvant.

A 64th aspect, either alone or in combination with any or all other aspects herein, is directed to vaccine comprising a nucleic acid according to any one of aspects 52-56 and a pharmaceutically-acceptable excipient, carrier, and/or adjuvant.

A 65th aspect, either alone or in combination with any or all other aspects herein, is directed to vaccine comprising a vector according to the 56th aspect and a pharmaceutically-acceptable excipient, carrier, and/or adjuvant.

A 66th aspect, either alone or in combination with any or all other aspects herein, is directed to vaccine comprising a plasmid according to the 57th aspect and a pharmaceutically-acceptable excipient, carrier, and/or adjuvant.

A 67th aspect, either alone or in combination with any or all other aspects herein, is directed to chimeric or fusion polypeptide comprising a polypeptide of any one of aspects 1-51, wherein said polypeptide is joined, linked, or inserted into a heterologous polypeptide.

A 68th aspect, either alone or in combination with any or all other aspects herein, is directed to a method for suppressing an immune response in a subject in need thereof, the method comprising administering to the subject a therapeutically effective amount of at least one polypeptide according to any one of aspects 1-7.

A 69th aspect, either alone or in combination with any or all other aspects herein, is directed to the method of the 68th aspect, wherein the immune response is a result of treatment with at least one or more therapeutic treatments with at least one therapeutic protein, treatment with a vaccine or treatment with at least one antigen.

A 70th aspect, either alone or in combination with any or all other aspects herein, is directed to the method of the 68th aspect, wherein the polypeptide is administered to isolated dendritic cells ex vivo, and said dendritic cells are the re-introduced to the subject.

A 71st aspect, either alone or in combination with any or all other aspects herein, is directed to the method of the 68th aspect, wherein the administration of the polypeptide shifts one or more antigen presenting cells to a regulatory phenotype.

A 72nd aspect, either alone or in combination with any or all other aspects herein, is directed to the method of the 68th aspect, wherein the administration of the polypeptide shifts one or more dendritic cells to a regulatory phenotype.

A 73rd aspect, either alone or in combination with any or all other aspects herein, is directed to the method of the 72nd aspect, wherein the regulatory phenotype is characterized by a decrease in CD11c and HLA-DR expression in the dendritic cells or other antigen presenting cells.

A 74th aspect, either alone or in combination with any or all other aspects herein, is directed to the method of the 68th aspect, wherein the administration of the polypeptide shifts one or more T cells to a regulatory phenotype.

A 75th aspect, either alone or in combination with any or all other aspects herein, is directed to the method of the 74^{th} aspect, wherein the administration of the polypeptide shifts one or more CD4+ T cells to a regulatory phenotype.

A 76th aspect, either alone or in combination with any or all other aspects herein, is directed to the method of the 74^{th} aspect, wherein the administration of the polypeptide shifts one or more CD8+ T cells to a regulatory phenotype.

A 77th aspect, either alone or in combination with any or all other aspects herein, is directed to the method of the 74^{th} aspect, wherein the administration of the polypeptide shifts one or more B cells to a regulatory phenotype.

A 78th aspect, either alone or in combination with any or all other aspects herein, is directed to the method of the 68th aspect, wherein the administration of the one or more polypeptides activates CD4⁺/CD25⁺/FoxP3⁺ regulatory T-cells.

A 79th aspect, either alone or in combination with any or all other aspects herein, is directed to the method of the 68th, wherein the administration of the one or more polypeptides suppresses activation of CD4⁺ T-cells.

A 80th aspect, either alone or in combination with any or all other aspects herein, is directed to the method of the 68th aspect, wherein the administration of the one or more polypeptides suppresses activation or proliferation of CD4⁺ and/or CD8⁺ T-cells.

A 81st aspect, either alone or in combination with any or all other aspects herein, is directed to the method of the 68th aspect, wherein the administration of the one or more polypeptides suppresses activation or proliferation of B-cells.

A 82nd aspect, either alone or in combination with any or all other aspects herein, is directed to the method of the 69^{th} aspect, wherein the administration of the one or more polypeptides suppresses an immune response selected from the group consisting of an innate immune response, an adaptive immune response, an effector T cell response, a memory T cell response, a helper T cell response, a B cell response, a nKT cell response, or any combination thereof.

A 83rd aspect, either alone or in combination with any or all other aspects herein, is directed to method for enhancing an immune response in a subject in need thereof, the method comprising administering to the subject a therapeutically effective amount of a polypeptide according to any one of aspects 8-51.

A 84th aspect, either alone or in combination with any or all other aspects herein, is directed to the method of the 83rd aspect, wherein the polypeptide reduces T_{Reg} activation in the subject.

A 85th aspect, either alone or in combination with any or all other aspects herein, is directed to the method of the 83rd aspect, wherein the polypeptide retains MHC binding.

A 86 aspect, either alone or in combination with any or all other aspects herein, is directed to the method of the 83rd aspect, wherein the polypeptide stimulates CD4+ and/or CD8+ T cells.

A 87th aspect, either alone or in combination with any or all other aspects herein, is directed to method for affecting immunity against a SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19 in a subject in need thereof, the method comprising administering to the subject a therapeutically effective amount of a polypeptide according to any one of aspects 1-51.

A 88th aspect, either alone or in combination with any or all other aspects herein, is directed to method for affecting immunity against a SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19 in a subject in need thereof, the method comprising administering to the subject a therapeutically effective amount of a nucleic acid according to any one of aspects 52-56.

A 89th aspect, either alone or in combination with any or all other aspects herein, is directed to method for affecting immunity against a SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19 in a subject in need thereof, the method comprising administering to the subject a therapeutically effective amount of a vector according to the 57th^{t} aspect.

A 90th aspect, either alone or in combination with any or all other aspects herein, is directed to method for affecting immunity SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19 in a subject in need thereof, the method comprising administering to the subject a therapeutically effective amount of a vector according to the 58th aspect.

A 91st aspect, either alone or in combination with any or all other aspects herein, is directed to method for affecting immunity against SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19 in a subject in need thereof, the method comprising administering to the subject a therapeutically effective amount of a pharmaceutical composition according to any one of aspects 59-62.

A 92nd aspect, either alone or in combination with any or all other aspects herein, is directed to a method for affecting immunity against SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19 in a subject in need thereof, the method comprising administering to the subject a therapeutically effective amount of a vaccine composition according to any one of aspects 63-66.

A 93rd aspect, either alone or in combination with any or all other aspects herein, is directed to a method according to any one of aspects 87-92, wherein the step of administration additionally includes administration of an SARS-CoV-2 virus, wherein the virus is a live attenuated virus or inactivated virus.

A 94th aspect, either alone or in combination with any or all other aspects herein, is directed to method for affecting an immune response against SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19 in a subject in need thereof, the method comprising administering to the subject a therapeutically effective amount of one or more of a polypeptide according to any one of aspects 1-51.

A 95th aspect, either alone or in combination with any or all other aspects herein, is directed to a composition comprising an effective amount of one or more isolated peptides and/or fragments and variants thereof according to any one of aspects 1-51 and one or more immune stimulating T-cell epitope polypeptides.

A 96th aspect, either alone or in combination with any or all other aspects herein, is directed to the composition according to the 95^{th} aspect, wherein said one or more immune stimulating T-cell epitope polypeptides is one or more therapeutic protein, treatment with a vaccine or treatment with at least one antigen.

A 97th aspect, either alone or in combination with any or all other aspects herein, is directed to the composition according to the 95^{th} aspect, wherein the polypeptide is either covalently bound, non-covalently bound or in admixture with a specific target antigen for use in the diminution of immune response against the target antigen.

A 98th aspect, either alone or in combination with any or all other aspects herein, is directed to The composition according to the 97th aspect, wherein the suppressive effect is mediated by a natural T_{Reg} or an adaptive T_{Reg} or a viral homolog of the natural T_{Reg}.

A 99th aspect, either alone or in combination with any or all other aspects herein, is directed to the composition according to the 98^{th} aspect, wherein any of effector T cells, helper T cells, or B cells are subject to the suppressive effect of the regulatory T cell epitope.

A 100th aspect, either alone or in combination with any or all other aspects herein, is directed to pharmaceutical composition comprising one or more polypeptides, wherein each polypeptide comprised an amino acid sequence selected from the group consisting of SEQ ID NOS: 4-370, 391-440, and 448-833, and/or fragments and variants thereof, and optionally 1 to 12 additional amino acids distributed in any ratio on the N terminus and/or C-terminus of the polypeptide of SEQ ID NOS: 4-370, 391-440, and 448-833.

A 101st aspect, either alone or in combination with any or all other aspects herein, is directed to pharmaceutical composition comprising the Tregitope composition according to the 100^{th} aspect and a pharmaceutically acceptable carrier.

A 102nd aspect, either alone or in combination with any or all other aspects herein, is directed to method for affecting regulatory T-cells in a subject in need thereof, the method comprising administering to the subject a therapeutically effective amount of a composition of the 100^{th} aspect.

A 103rd aspect, either alone or in combination with any or all other aspects herein, is directed to kit for affecting an immune response in a subject, wherein the kit comprises a composition according to the 100^{th} aspect.

A 104th aspect, either alone or in combination with any or all other aspects herein, is directed to the kit of the 100^{th} aspect, further comprising an effective amount of an antigen or allergen.

A 105th aspect, either alone or in combination with any or all other aspects herein, is directed to method for enhancing the immunogenicity of a vaccine delivery vector containing a virus, comprising administrating to a subject the polypeptide of aspects 8-51.

A 106th aspect, either alone or in combination with any or all other aspects herein, is directed to polypeptide composition comprising one or more T-cell epitope polypeptides linked to a heterologous polypeptide, wherein the T-cell epitope polypeptide consists of an amino acid sequence selected from the group consisting of SEQ ID NOS: 4-370, 391-440, and 448-833, and/or fragments and variants thereof, and optionally 1 to 12 additional amino acids distributed in any ratio on the N terminus and/or C-terminus of the polypeptide of SEQ ID NOS: 4-370, 391-440, and 448-833.

A 107th aspect, either alone or in combination with any or all other aspects herein, is directed to the polypeptide composition of the 106th aspect, wherein the T-cell epitope polypeptide is linked to the N-terminus of the heterologous polypeptide.

A 108th aspect, either alone or in combination with any or all other aspects herein, is directed to the polypeptide composition of the 106th aspect, wherein the T-cell epitope polypeptide is linked to the C-terminus of the heterologous polypeptide.

A 109th aspect, either alone or in combination with any or all other aspects herein, is directed to the polypeptide composition of the 106th aspect, wherein the heterologous polypeptide comprises a biologically active molecule and wherein the biologically active molecule is selected from the group consisting of an immunogenic molecule, a T-cell epitope, a viral protein, and a bacterial protein.

A 110th aspect, either alone or in combination with any or all other aspects herein, is directed to the polypeptide composition of the 106th aspect, wherein the heterologous polypeptide is operatively linked to the T-cell epitope polypeptide.

A 111th aspect, either alone or in combination with any or all other aspects herein, is directed to method of affecting regulatory T-cells in a subject comprising administrating to the subject a therapeutically effective amount of a polypeptide composition of the 106th aspect.

### EXEMPLIFICATION

The examples that follow are not to be construed as limiting the scope of the invention in any manner. In light of the present disclosure, numerous embodiments within the scope of the claims will be apparent to those of ordinary skill in the art.

### (1) In-Silico Identification of a Tregitope Composition

T cells specifically recognize epitopes presented by antigen presenting cells (APCs) in the context of MHC (Major Histocompatibility Complex) Class II molecules. These T-helper epitopes can be represented as linear sequences comprising 7 to 30 contiguous amino acids that fit into the MHC Class II binding groove. A number of computer algorithms have been developed and used for detecting Class II epitopes within protein molecules of various origins (De Groot A S et al., (1997), AIDS Res Hum Retroviruses, 13(7):539-41; Schafer J R et al., (1998), Vaccine, 16(19):1880-4; De Groot A S et al., (2001), Vaccine, 19(31):4385-95; De Groot A S et al., (2003), Vaccine, 21(27-30):4486-504). These “in silicas” predictions of T-helper epitopes have been successfully applied to the design of vaccines and the de-immunization of therapeutic proteins, e.g., antibody-based drugs, Fc fusion proteins, anticoagulants, blood factors, bone morphogenetic proteins, engineered protein scaffolds, enzymes, growth factors, hormones, interferons, interleukins, and thrombolytics (Dimitrov D S, (2012), Methods Mol Biol, 899:1-26).

The Conservatrix system (EpiVax, Providence, R.I.) is an algorithm useful for identifying 9-mer polypeptide sequences from a larger set of data. The Conservatrix system parses input sequences into 9-mer sequences that are conserved amongst multiple inputted whole sequences, such as multiple strains of the same pathogen, for even the most mutable of potential vaccine targets. These 9-mer sequences may be searched for identically matched 9-mer sequences across data sets.

The EpiMatrix™ system (EpiVax, Providence, R.I.) is a set of predictive algorithms encoded into computer programs useful for predicting class I and class II HLA ligands and T cell epitopes. The EpiMatrix™ system uses 20×9 coefficient matrices in order to model the interaction between specific amino acids (20) and binding positions within the HLA molecule (9). In order to identify putative T cell epitopes resident within any given input protein, the EpiMatrix™ System first parses the input protein into a set of overlapping 9-mer frames where each frame overlaps the last by eight amino acids. Each frame is then scored for predicted affinity to one or more common alleles of the human HLA molecule; typically DRB1*0101, DRB1*0301, DRB1*0401, DRB1*0701, DRB1*0801, DRB1*1101, DRB1*1301, and DRB1*1501 (Mack et al., (2013), Tiss Antig, 81(4):194-203). Briefly, for any given 9-mer peptide specific amino acid codes (one for each of 20 naturally occurring amino acids) and relative binding positions (1-9) are used to select coefficients from the predictive matrix. Individual coefficients are derived using a proprietary method similar to, but not identical to, the pocket profile method first developed by Sturniolo (Sturniolo T et al., 1999, Nat Biotechnol, 17(6):555-61). Individual coefficients are then summed to produce a raw score. EpiMatrix™ raw scores are then normalized with respect to a score distribution derived from a very large set of randomly generated peptide sequences. The resulting “Z” scores are normally distributed and directly comparable across alleles.

EpiMatrix™ peptide scoring. It was determined that any peptide scoring above 1.64 on the EpiMatrix™ “Z” scale (approximately the top 5% of any given peptide set) has a significant chance of binding to the MHC molecule for which it was predicted. Peptides scoring above 2.32 on the scale (the top 1%) are extremely likely to bind; most published T cell epitopes fall within this range of scores. Previous studies have also demonstrated that EpiMatrix™ accurately predicts published MHC ligands and T cell epitopes.

Identification of promiscuous T cell Epitope Clusters. Potential T cell epitopes are not randomly distributed throughout protein sequences but instead tend to “cluster.” T cell epitope “clusters” range from 9 to roughly 30 amino acids in length and, considering their affinity to multiple alleles and across multiple frames, contain anywhere from 4 to 40 binding motifs. Following epitope mapping, the result set produced by the EpiMatrix™ algorithm is screened for the presence of T cell epitope clusters and EpiBars™ by using a proprietary algorithm known as Clustimer™. Briefly, the EpiMatrix™ scores of each 9-mer peptide analyzed are aggregated and checked against a statistically derived threshold value. High scoring 9mers are then extended one amino acid at a time. The scores of the extended sequences are then re-aggregated and compared to a revised threshold value. The process is repeated until the proposed extension no longer improves the overall score of the cluster. Tregitope(s) identified in the present studies will be identified by the Clustimer-™ algorithm as T cell epitope clusters. They are predicted to contain significant numbers of putative T cell epitopes and EpiBars™ indicating a high potential for MHC binding and T cell reactivity.

Identification of tolerogenic T cell Epitope Clusters. The JanusMatrix system (EpiVax, Providence, R.I.) useful for screening peptide sequences for cross-conservation with a host proteome. JanusMatrix is an algorithm that predicts the potential for cross-reactivity between peptide clusters and the host genome or proteome, based on conservation of TCR-facing residues in their putative MHC ligands. The JanusMatrix algorithm first considers all the predicted epitopes contained within a given protein sequence and divides each predicted epitope into its constituent agretope and epitope. Each sequence is then screened against a database of host proteins. Peptides with a compatible MHC-facing agretope (i.e., the agretopes of both the input peptide and its host counterparty are predicted to bind the same MHC allele) and exactly the same TCR-facing epitope are returned. The JanusMatrix Homology Score suggests a bias towards immune tolerance. In the case of a therapeutic protein, cross-conservation between autologous human epitopes and epitopes in therapeutic may increase the likelihood that such a candidate will be tolerated by the human immune system. In the case of a vaccine, cross-conservation between human epitopes and the antigenic epitopes may indicate that such a candidate utilizes immune camouflage, thereby evading the immune response and making for an ineffective vaccine. When the host is, for example, a human, the peptide clusters are screened against human genomes and proteomes, based on conservation of TCR-facing residues in their putative HLA ligands. The peptides are then scored using the JanusMatrix Homology Score. In aspects, peptides with a JanusMatrix Homology Score above 3.0 indicate high tolerogenicity potential and as such may be very useful Tregitopes of the present disclosure.

### Example 1. Identification of a Tregitope Composition

EpiMatrix analysis results for Tregitopes include a Z score that indicates the potential of a 9-mer frame to bind to a given HLA allele. All scores in the top 5% are considered “hits”, while non-hits (*) below 10% will be masked. JanusMatrix analysis results for Tregitopes include the count of HUMAN JanusMatrix matches found in the search database. With respect to a given EpiMatrix Hit (a 9-mer contained within the input sequence which is predicted to bind to a specific allele), a Janus Matrix match is a 9-mer derived from the search database (e.g., the human genome) which is predicted to bind to the same allele as the EpiMatrix Hit and shares TCR facing contacts with the EpiMatrix Hit. Further, the Janus Homology Score** represents the average depth of coverage in the search database for each EpiMatrix hit in the input sequence. For example, an input peptide with eight EpiMatrix hits, all of which have one match in the search database, has a Janus Homology Score of 1. An input peptide with four EpiMatrix Hits, all of which have two matches in the search database, has a Janus Homology Score of 2. The JanusMatrix Homology Score considers all constituent 9-mers in any given peptide, including flanks.

Tregitopes of the present disclosure comprise at least one putative T cell epitope as identified by EpiMatrix™ analysis. EpiMatrix™ is a proprietary computer algorithm developed by EpiVax (Providence, R.I.), which is used to screen protein sequences for the presence of putative T cell epitopes. Input sequences are parsed into overlapping 9-mer frames where each frame overlaps the last by 8 amino acids. Each of the resulting frames is then scored for predicted binding affinity with respect to a panel of eight common Class II HLA alleles (DRB1*0101, DRB1*0301, DRB1*0401, DRB1*0701, DRB1*0801, DRB1*1101, DRB1*1301, and DRB1*1501). Raw scores are normalized against the scores of a large sample of randomly generated peptides. The resulting “Z” score is reported. In aspects, any 9-mer peptide with an allele-specific EpiMatrix™ Z-score in excess of 1.64, theoretically the top 5% of any given sample is considered a putative T cell epitope. FIGS. 2-7 are EpiMatrix Cluster detail reports for select identified MHC class II clusters of the envelope (SEQ ID NO: 1) of SARS-CoV-2. FIGS. 8-10 are EpiMatrix Cluster detail reports for select identified MHC class II clusters of the membrane (SEQ ID NO: 2) of SARS-CoV-2. FIGS. 11-28 are EpiMatrix Cluster detail reports for select identified MHC class II clusters of the spike (SEQ ID NO: 3) of SARS-CoV-2.

FIGS. 29A-C are the overview of JanusMatrix results for select identified the Tregitopes, and 9-mers contained therein, identified in the envelope (SEQ ID NO: 1) of SARS-CoV-2. FIGS. 30A-C are the overview of JanusMatrix results for select identified the Tregitopes, and 9-mers contained therein, identified in the membrane (SEQ ID NO: 2) of SARS-CoV-2. FIGS. 30A-T are the overview of JanusMatrix results for select identified the Tregitopes, and 9-mers contained therein, identified in the spike (SEQ ID NO: 3) of SARS-CoV-2.

According to EpiMatrix analysis, each of the identified Tregitopes of SEQ ID NOS: 4-370, 391-440, and 448-833 have at least four EpiMatrix hits. According to JanusMatrix analysis, each of the identified Tregitopes of SEQ ID NOS: 4-370, 391-440, and 448-833 are conserved in at least five human proteins. Each of the identified Tregitopes of SEQ ID NOS: 4-370, 391-440, and 448-833 are expected to be predicted by JanusMatrix to have a homology score above 2.

### (2) Methods for the Assessment of Tregitope Binding to Soluble MHC.

Synthesis of peptides. The Tregitopes of the present disclosure (e.g., but not limited to, a peptide or polypeptide comprising, consisting, or consisting essentially of an amino acid sequence of SEQ ID NOS: 4-370, 391-440, and 448-833 (and/or fragments or variants thereof), and optionally 1 to 12 additional amino acids distributed in any ratio on the N terminus and/or C-terminus of the polypeptide of SEQ ID NOS: 4-370, 391-440, and 448-833, can be produced by direct chemical synthesis or by recombinant methods (J Sambrook et al., Molecular Cloning: A Laboratory Manual, (2^{ED}, 1989), Cold Spring Harbor Laboratory Press, Cold Springs Harbor, N.Y. (Publ)). Every peptide undergoes rigorous quality control characterization before release to determine purity, mass, and correct sequence. Peptides will be assessed for purity by reversed phase high-pressure liquid chromatography (RP-HPLC). Peptides are ≥90% pure, and each preparation will undergo Amino Acid Analysis to ensure that the equivalent molar amounts are used in assays for consistency and reproducibility between different lots of peptides, and will also allow for reliable comparison studies between peptide efficacy. Peptides will also be assessed for mass and correct sequence using tandem mass spectrometry and MS CheckT analysis. In certain aspects, the Tregitopes can be capped with an n-terminal acetyl and/or c-terminal amino group. HPLC, mass spectrometry and UV scan (ensuring purity, mass and spectrum, respectively) analysis of the selected Tregitopes will indicate >80% purity.

HLA Binding Assay. Binding activity will be analyzed at EpiVax (Providence, R.I.) and may be conducted for any Tregitope disclosed herein (e.g., but not limited to, a peptide or polypeptide comprising, consisting, or consisting essentially of an amino acid sequence of SEQ ID NOS: 4-370, 391-440, and 448-833 (and/or fragments or variants thereof), and optionally 1 to 12 additional amino acids distributed in any ratio on the N terminus and/or C-terminus of the polypeptide of SEQ ID NOS: 4-370, 391-440, and 448-833. The binding assay that will be used (Steere A C et al., (2006), J Exp Med, 2003(4):961-71) will yield an indirect measure of peptide-MHC affinity. Soluble HLA molecules will be loaded onto a 96-well plate with the unlabeled experimental Tregitopes and labeled control peptide. Once the binding mixture reached steady equilibrium (at 24 hours), the HLA-Tregitope complexes will be captured on an ELISA plate coated with anti-human DR antibody and detected with a Europium-linked probe for the label (PerkinElmer, Waltham, Mass.). Time-resolved fluorescence measuring bound labeled control peptide will be assessed by a SpectraMax® M5 unit (Spectramax, Radnor, Pa.). Binding of experimental Tregitopes will be expressed as the percent inhibition of the labeled control peptide (experimental fluorescence/control fluorescence multiplied by 100). The percent inhibition values for each experimental Tregitope (across a range of molar concentrations) will be used to calculate the concentration at which it inhibits 50% of the labeled control Tregitope's specific binding, i.e., the Tregitope's IC₅₀.

Select experimental Tregitopes will be solvated in DMSO. The diluted Tregitope will then be mixed with binding reagents in aqueous buffering solution, yielding a range of final concentrations from 100,000 nM down to 100 nM. The select Tregitopes (SEQ ID NO: 14) will then be assayed against a panel of eight common Class II HLA alleles: DRB1*0101, DRB1*0301, DRB1*0401, DRB1*0701, DRB1*0801, DRB1*1101, DRB1*1301, and DRB1*1501. From the percent inhibition of labeled control peptide at each concentration, IC₅₀ values will be derived for each Tregitope/allele combination using linear regression analysis.

In this assay, the experimental Tregitopes are considered to bind with very high affinity if they inhibit 50% of control peptide binding at a concentration of 100 nM or less, high affinity if they inhibit 50% of control peptide binding at a concentration between 100 nM and 1,000 nM, and moderate affinity if they inhibit 50% of control peptide binding at a concentration between 1,000 nM and 10,000 nM. Low affinity peptides inhibit 50% of control peptide binding at concentrations between 10,000 nM and 100,000 nM. Peptides that fail to inhibit at least 50% of control peptide binding at any concentration below 100,000 nM and do not show a dose response are considered non-binders (NB).

### Example 2. Peptide Characterization by Binding to HLA Class II Molecules

Soluble MHC binding assays may be performed on any of the instantly disclosed Tregitopes (e.g., but not limited to, a peptide or polypeptide comprising, consisting, or consisting essentially of an amino acid sequence of SEQ ID NOS: 4-370, 391-440, and 448-833 (and/or fragments or variants thereof), and optionally 1 to 12 additional amino acids distributed in any ratio on the N-terminus and/or C-terminus of the polypeptide of SEQ ID NOS: 4-370, 391-440, and 448-833. Soluble MHC binding assays will be performed on selected Tregitopes of the instant disclosure according to the methods described previously. IC₅₀ values (nM) will be derived from a six-point inhibition curve. A summary of HLA binding results for Tregitopes will be presented. EpiMatrix™ Predictions, calculated IC₅₀ values, and results classifications will be reported for each Tregitope and HLA allele. Binding curves will be generated for certain Tregitopes against the selected Class II HLA alleles, such as for the HLA DRB1*0801 assay and the HLA DRB1*1501 assay.

### (3) Methods for Assessing the Phenotype of Peptide-Exposed APC

Surface expression of Class II HLA (HLA-DR) and CD86 by professional antigen presenting cells (APCs) is one way APCs modulate T cell response. Expression of Class II HLA surface marker has been previously demonstrated for down-regulated in response to Tregitopes, and in particular to, the control Tregitope 167 (21^{st} Century Biochemicals, Marlboro, Mass.). Additionally, reduced expression of surface marker CD86 correlates positively with enhanced T_{Reg} function (Zheng Y et al., J Immunol, 2004, 172(5):2778-84). In this assay, candidate Tregitopes, including the selected Tregitopes, will be tested for their ability to down-regulate the expression of Class II HLA and the co-stimulatory molecule CD86 on the surface of professional APCs, specifically dendritic cells.

Tregitopes of the instant disclosure (e.g., but not limited to, a peptide or polypeptide comprising, consisting, or consisting essentially of an amino acid sequence of SEQ ID NOS: 4-370, 391-440, and 448-833 (and/or fragments or variants thereof), and optionally 1 to 12 additional amino acids distributed in any ratio on the N terminus and/or C-terminus of the polypeptide of SEQ ID NOS: 4-370, 391-440, and 448-833, will be individually tested for regulatory potential using a proprietary APC phenotyping assay previously developed at EpiVax (EpiVax, Providence, R.I.). Previously harvested and frozen PBMC will be thawed and suspended in chRPMI by conventional means. Under the direction and guidance of the Inventors from EpiVax, HLA typing will be conducted on small, extracted samples of cellular material, provided by EpiVax, by Hartford Hospital (Hartford, Conn.). On assay day 0, 0.5×10⁶ cells will be extracted, screened for the presence of surface marker CD11c (a marker specific to dendritic cells) and will be analyzed for the presence of surface markers HLA-DR and CD86 by flow cytometry. The remaining cells will be plated (4.0×10⁶ cell per ml in chRPMI plus 800 ul media) and will be stimulated (50 μg/mL) with one of the selected peptides or positive and negative controls including buffer only (negative control), Tregitope 167 (positive control) (21^{st} Century Biochemicals, Marlboro, Mass.), Flu-HA 306-318 (negative control) (21^{ST} Century Biochemicals, Marlboro, Mass.) and Ova 323-339 (negative control) (21^{st} Century Biochemicals, Marlboro, Mass.). Plated cells will be incubated for seven days at 37° C. On assay day 7, incubated cells will be screened by flow cytometry for the presence of surface marker CD11c. CD11c positive cells will be then analyzed for the presence of surface markers HLA-DR and CD86. The experimental peptides will be tested in samples drawn from five different human donors.

Leukocyte Reduction Filters will be obtained from the Rhode Island Blood Center (Providence, R.I.) to filter white blood cells from whole blood obtained from healthy donors. After the whole blood is run through the filters, the filters will be flushed in the opposite direction to push collected white blood cells out of the filter. The white blood cells will then be isolated using a conventional Ficoll™ separation gradient (GE Healthcare). The collected white blood cells will be thereafter frozen for future use. When needed for use in an assay, the frozen white blood cells will be thawed using conventional methods. For the GvHD studies discussed below, PBMCs will be obtained (e.g., from HemaCare, Van Nuys, Calif.) and the experiments will be performed.

Exposure to putative Tregitopes on the phenotypes of dendritic cells will be measured by multiple means. First, for each experimental condition, dot-plots, contrasting surface expression of CD11c and HLA-DR, will be produced. Dot-plots of cells exposed to all control and experimental peptides will be overlaid onto dot-plots produced from control cells exposed to only the culture media. The overlay will provide an effective method to visually observe shifts in HLA-DR distribution between Tregitope stimulated and unstimulated CD11c-high cells (data not shown). Observed shifts in the distribution of HLA-DR will be reported as a qualitative measure. Next, the change in intensity of HLA-DR expression for the CD11c-high segment of each dot-plot will be calculated. Percent change in intensity of HLA-DR expression equals Mean Florescence Index (MFI) of HLA-DR expression for peptide exposed cells minus MFI of HLA-DR expression for media exposed cells divided by MFI of HLA-DR expression for media exposed cells, times 100 (^{HLA-DR}MFI_{peptide}−^{HLA-DR}MFI_{media}/^{HLA-DR}MFI_{media}*100). Next, the percent change in the percentage of HLA-DR-low cells present among the CD11c high population will be calculated for each peptide relative to media control. Percent change in the percentage of HLA-DR-low cells will be calculated, and equals the percent of HLA-DR-low for peptide exposed cells minus the percent of HLA-DR-low for media exposed cells divided by percent of HLA-DR-low for media exposed cells times 100 (^{HLA-DR-low}%_{peptide}−^{HLA-DR-low}%_{media}/^{HLA-DR-low}%_{media}*100). In this assay, a negative change in observed HLA-DR MFI and a positive change in percentage of HLA-DR-low cells present in the CD11c-high population indicates reduced expression of HLA and a shift to a regulatory APC phenotype.

A similar process will be used to assess the impact Tregitope exposure on surface expression of CD86, which is a costimulatory molecule known to promote T cell activation. First, for each experimental condition, dot plots contrasting surface expression of CD11c and CD86 will be produced. Dot plots of cells exposed to all control and experimental Tregitopes will be overlaid onto dots plots produced from control cells exposed to only the culture media. The overlay provides an effective method to visually observe shifts in CD86 distribution between Tregitope stimulated and un-stimulated CD11c-high cells. Observed shifts in the distribution of CD86 will be reported as a qualitative measure. Next, the change in intensity of CD86-high expression for the CD11c-high segment of each dot plot will be calculated. Percent change in intensity of CD86-high expression equals Mean Florescence Index (MFI) of CD86 expression for peptide exposed cells minus MFI of CD86-high expression for media exposed cells divided by MFI of CD86 expression for media exposed cells, times 100 (^{CD86-high}MFI_{peptide}−CD^{86-high}MFI_{media}/^{CD86-high}MFI_{media}*100). Next, the percent change in the percentage of CD86-low cells present among the CD11c high population will be calculated. Percent change in the percentage of CD86-high cells equals the percent of CD86-high for peptide exposed cells minus the percent of CD86-high for media exposed cells divided by percent of CD86-high for media exposed cells, times 100 (^{CD86-low}% I_{peptide}−^{CD86-low}%_{media}/^{CD86-low}%_{media}*100). In this assay, a negative change in observed CD86 MFI and a positive change in percentage of CD86-low cells present in the CD11c-high population indicates reduced expression of CD86 and a shift to a regulatory APC phenotype.

### Example 3. Characterization of Peptide Exposed APC

Dendritic cell phenotyping assays will be performed on Tregitopes of the instant disclosure (e.g., but not limited to, a peptide or polypeptide comprising, consisting, or consisting essentially of an amino acid sequence of SEQ ID NOS: 4-370, 391-440, and 448-833 (and/or fragments or variants thereof), and optionally 1 to 12 additional amino acids distributed in any ratio on the N-terminus and/or C-terminus of the polypeptide of SEQ ID NOS: 4-370, 391-440, and 448-833) according to the methods described previously.

Dot plots representing the surface expression of CD11 vs HLA-DR will be analyzed on assay day 7 across the five donors in the presence of various peptide stimulants. It is expected that downward movement of the CD11c+/HLA-DR+ population will apparent in the samples treated with the Tregitopes of the instant disclosure as compared to media control indicating an acquired regulatory phenotype.

Dot plots representing the surface expression of CD11c vs CD86 will be analyzed on assay day 7 across the five donors in the presence of various peptide stimulants. It is expected that an increase in CD86-low cells present in the samples treated with Tregitopes of the instant disclosure as compared to media control, which indicates a shift to the acquired regulatory phenotype. It is further expected that exposure to claimed Tregitopes will result in decreased expression of HLA-DR in all subjects that will be tested.

### (4) Methods for Assessing Peptide Effects on Proliferation of Regulatory T Cells

Previous studies performed by EpiVax (Providence, R.I.) demonstrated increased proliferation of regulatory T cells following exposure to known Tregitope, including positive control Tregitope. Tregitopes of the instant disclosure (e.g., but not limited to, a peptide or polypeptide comprising, consisting, or consisting essentially of an amino acid sequence of SEQ ID NOS: 4-370, 391-440, and 448-833 (and/or fragments or variants thereof), and optionally 1 to 12 additional amino acids distributed in any ratio on the N-terminus and/or C-terminus of the polypeptide of SEQ ID NOS: 4-370, 391-440, and 448-833), may be tested for their ability to induce proliferation among CD4+CD25+FoxP3+ regulatory T cells. In this assay, candidate Tregitopes, including the Tregitopes of the instant disclosure, will be tested for their ability to induce proliferation among CD4+CD25+FoxP3+ regulatory T cells. Previously harvested and frozen PBMC will be thawed and suspended in conditioned chRPMI (3.3×10⁶ cells/mL) by conventional means. The donors to be evaluated will represent a diversity of HLA DRB1 supertypes. Cells will be stained with CFSE (Cat #: 65-0850-84, Affymetrix, Santa Clara, Calif.) and plated at 300,000 cells per well. Plates will be incubated overnight (37° C. in 5% CO₂). Each well contains 200 μL of media. On assay day 1, candidate Tregitope will be reconstituted in sterile DMSO yielding a final stock concentration of 10 mg/mL. Previous titration experiments performed at EpiVax (EpiVax, Providence, R.I.) have established that stimulation with 0.5 μg/ml Tetanus Toxoid (TT) (Astarte Biologics, Bothell, Wash.) elicits a measurable CD4+ effector memory T cells response in PBMC drawn from healthy control donors (Rhode Island Blood Center, Providence, R.I.). Tetanus Toxoid stock (100 μg/mL) (Astarte Biologics, Bothell, Wash.) will be diluted in conditioned chRPMI yielding a working concentration of 1 ug/mL (2× concentration). Plated cells (in 100 μL media) will then be stimulated with either 100 μL of conditioned chRPMI (negative control), 100 μL Tetanus Toxoid solution (2× solution, positive control) (Astarte Biologics, Bothell, Wash.), 100 μL of a dilution of 2991 μL Tetanus Toxoid solution plus 9 μL Tregitope solution, 100 μL of a dilution of 2997 μL Tetanus Toxoid solution plus 3 μL Tregitope solution, or 100 μL of a dilution of 6998.2 μL Tetanus Toxoid solution plus 1.8 μL Tregitope solution. All plates will then be incubated for six additional days. On assay day five, 100 μL of supernatant will be removed from each well and replaced with freshly conditioned chRPMI (for no TT control wells), or 100 μl of media with 2×TT (1 mg/mL) for the wells originally incubated with TT alone or TT+Tregitope. No extra Tregitope will be added.

Highly activated regulatory T cells displaying elevated levels of FoxP3, CD25, Granzyme B and proliferation will be selected. The gating strategy for highly activated regulatory T cells and CD4+ effector T cells will be as follows: cells will first be gated to eliminate aggregates and dead cells, and live cells will be gated for CD4+ T cells and all subsequent analysis will be done on this population; CD4+ T cells will be gated for elevated CD25, FoxP3, and low CFSE (proliferation). It is expected that proliferating and activated CD4+ T cell populations will be highly correlated.

### Example 4. Tregitopes Induces a Population of Highly Proliferative, Activated Regulatory T Cells

Regulatory T cell proliferation assays will be performed on the Tregitopes of the present disclosure (e.g., but not limited to, a peptide or polypeptide comprising, consisting, or consisting essentially of an amino acid sequence of SEQ ID NOS: 4-370, 391-440, and 448-833 (and/or fragments or variants thereof), and optionally 1 to 12 additional amino acids distributed in any ratio on the N-terminus and/or C-terminus of the polypeptide of SEQ ID NOS: 4-370, 391-440, and 448-833) according to the methods described previously. It is expected that such data will demonstrates that the Tregitopes of the present disclosure strongly induces a population of highly proliferative, activated regulatory T cells.

### (5) Methods for Assessing Peptide Effects on Proliferation of CD4+ Effector T Cells

CD4+ effector memory T cells contained within PBMC cell populations can be induced to proliferate in response to stimulation with known T cell epitopes.

The purpose of this experiment is to establish the ability of Tregitopes of the instant disclosure (e.g., but not limited to, a peptide or polypeptide comprising, consisting, or consisting essentially of an amino acid sequence of SEQ ID NOS: 4-370, 391-440, and 448-833 (and/or fragments or variants thereof), and optionally 1 to 12 additional amino acids distributed in any ratio on the N-terminus and/or C-terminus of the polypeptide of SEQ ID NOS: 4-370, 391-440, and 448-833) to suppress the proliferation of antigen stimulated CD4+ effector memory T cells by either direct (engagement and activation of T_{Reg}) or indirect (modulation of APC phenotype) means.

Previous studies performed by EpiVax (Providence, R.I.) demonstrated increased proliferation of regulatory T cells following exposure to known Tregitope, including positive control Tregitope. In this assay, candidate Tregitopes, including the Tregitopes of the instant disclosure, will be tested for their ability to induce proliferation among CD4+CD25+FoxP3+ regulatory T cells. Previously harvested and frozen PBMC will be thawed and suspended in conditioned chRPMI (3.3×10⁶ cells/mL) by conventional means. The donors that will be evaluated will represent a diversity of HLA DRB1 supertypes. Cells will be stained with CFSE (Cat #: 65-0850-84, Affymetrix, Santa Clara, Calif.) and plated at 300,000 cells per well. Plates were incubated overnight (37° C. in 5% CO₂). Each well contains 200 μL of media. On assay day 1, Tregitope peptide will be reconstituted in sterile DMSO yielding a final stock concentration of 10 mg/mL. Previous titration experiments performed at EpiVax (EpiVax, Providence, R.I.) have established that stimulation with 0.5 μg/ml Tetanus Toxoid (TT) (Astarte Biologics, Bothell, Wash.) elicits a measurable CD4+ effector memory T cells response in PBMC drawn from healthy control donors (Rhode Island Blood Center, Providence, R.I.). Tetanus Toxoid stock (100 μg/mL) (Astarte Biologics, Bothell, Wash.) will be diluted in conditioned chRPMI yielding a working concentration of 1 μg/mL (2× concentration). Plated cells (in 100 μL media) will then be stimulated with either 100 μL of conditioned chRPMI (negative control), 100 μL Tetanus Toxoid solution (2× solution, positive control) (Astarte Biologics, Bothell, Wash.), 100 μL of a dilution of 2991 μL Tetanus Toxoid solution plus 9 μL Tregitope solution, 100 μL of a dilution of 2997 μL Tetanus Toxoid solution plus 3 μL Tregitope solution, or 100 μL of a dilution of 6998.2 μL Tetanus Toxoid solution plus 1.8 μL Tregitope solution. All plates will then be incubated for six additional days. On assay day five, 100 μL of supernatant will be removed from each well and replaced with freshly conditioned chRPMI (for no TT control wells), or 100 μl of media with 2×TT (1 mg/mL) for the wells originally incubated with TT alone or TT+Tregitope. No extra Tregitope will be added.

On assay day seven, cells will be removed from incubation. Cells will first be gated to eliminate aggregates and dead cells, and live cells were gated for CD4+ T cells and all subsequent analysis will be done on this population. CD4+ T cells will be gated for elevated CD25, FoxP3, and low CFSE (proliferation). The activated T effector population will be identified as the CD4+/CD25-high/FoxP3-intermediate (CD4⁺/CD25^{hi}/FoxP3^{int}). Proliferation of CD4+/Foxp3-low/CD25-high (CD4⁺/Foxp3^{lo}/CD25^{hi}) T cells will be estimated from the dilution of the CFSE stain (Cat #: 65-0850-84, Affymetrix, Santa Clara, Calif.) and % proliferation determined by the CFSE-low)(CFSE^{lo}) population.

### Example 5. Peptide SEQ ID NO: 7 Suppressed Proliferation and Activation of CD4+ Effector T Cells

The change in activation (as will be demonstrated by: absolute values with media only controls subtracted; normalized values with media only controls subtracted; absolute values with media only controls not subtracted; and normalized values with media only controls not subtracted) and proliferation (as will be demonstrated by: absolute values with media only controls subtracted; normalized values with media only controls subtracted; absolute values with media only controls not subtracted; and normalized values with media only controls not subtracted) of CD4+ effector cells when the proliferation stimulant (Tetanus Toxoid) is co-delivered with Tregitope will be measured and the proliferative response of CD4+ T cells, comprised mainly of T effector memory cells, will be characterized.

T cell proliferation assays will be performed on the Tregitopes of the present disclosure according to the methods described previously. It is expected that, in the various donors, Tregitope peptide will strongly suppress a population of activated effector CD4+ T cells (CD4+/CD25-high/FoxP3-intermediate, shown as CD4⁺/CD25^{hi}/FoxP3^{int}) reacting to Tetanus Toxoid in a dose-dependent manner.

### (6) Methods for Assessing Peptide Effects on CD8+ Effector T Cells.

It was previously shown that CD8+ effector memory T cells contained within PBMC cell populations can be induced to proliferate in response to stimulation with known class I T cell epitopes. The results of this assay will establish the ability of the instantly-disclosed Tregitopes (e.g., but not limited to, a peptide or polypeptide comprising, consisting, or consisting essentially of an amino acid sequence of SEQ ID NOS: 4-370, 391-440, and 448-833 (and/or fragments or variants thereof), and optionally 1 to 12 additional amino acids distributed in any ratio on the N-terminus and/or C-terminus of the polypeptide of SEQ ID NOS: 4-370, 391-440, and 448-833) to suppress the proliferation of antigen stimulated CD8+ T effector memory T cells by either direct (engagement and activation of T_{Reg}) or indirect (modulation of APC phenotype) means.

T cell proliferation assays will be performed on the Tregitopes of the present disclosure (e.g., but not limited to, a peptide or polypeptide comprising, consisting, or consisting essentially of an amino acid sequence of SEQ ID NOS: 4-370, 391-440, and 448-833 (and/or fragments or variants thereof), and optionally 1 to 12 additional amino acids distributed in any ratio on the N-terminus and/or C-terminus of the polypeptide of SEQ ID NOS: 4-370, 391-440, and 448-833) according to the methods described previously. PBMCs from two healthy donors will be thawed and suspended in conditioned chRPMI (3.3×10⁶ cells/mL) by conventional means. Cells will be stained with CFSE (Cat #: 65-0850-84, Affymetrix, Santa Clara, Calif.) and plated at 300,000 cells per well. Plates will be incubated overnight (37° C. in 5% CO₂). On assay day 1, Tregitopes of the instant disclosure will be reconstituted in sterile DMSO yielding a final stock concentration of 20 mg/mL. Intermediate solutions of Tregitopes of the instant disclosure at twice the final concentration in chRPMI will be prepared as described previously. Final concentration of Tregitopes of the instant disclosure will be tested from 2.5, 5, 10 and 20 μg/ml. As a CD8+ stimulating antigen, the CEF peptide pool, which consists of 23 MHC class I restricted viral epitopes derived from human cytomegalovirus, Epstein-Barr virus, and influenza virus, will be used. CEF peptides will be added to the wells (data shown for 2 μg/mL) with cells and media (control) or a Tregitope of the instant disclosure at 0, 1, 2 or 4 μg/ml. All plates will be incubated for six additional days. On assay day 5, 100 μL of supernatant will be removed from each well and will be replaced with freshly conditioned chRPMI.

Conventional methods will be used to stain cells for live/dead marker, extracellular markers CD4, CD8a and CD25, CD127, CD45RA and CCR7, and intracellular marker FoxP3. After FACS analysis, cells will be gated to eliminate aggregates and dead cells. On the live cells population, CD8a and CD4 cells will be gated separately and each population will be analyzed for proliferation (CFSE low population) or activation (CD25-high/FoxP3 low/intermediate) as explained previously.

### Example 6. Tregitopes of the Instant Disclosure Will Suppress Proliferation of CD8+ Effector T Cells

The potential inhibition of CD8+ T cell response by Tregitopes of the instant disclosure (e.g., but not limited to, a peptide or polypeptide comprising, consisting, or consisting essentially of an amino acid sequence of SEQ ID NOS: 4-370, 391-440, and 448-833 (and/or fragments or variants thereof), and optionally 1 to 12 additional amino acids distributed in any ratio on the N-terminus and/or C-terminus of the polypeptide of SEQ ID NOS: 4-370, 391-440, and 448-833) when PBMC from healthy donors are stimulated with CEF peptides mixture will be tested. It is expected that Tregitopes of the instant disclosure will strongly inhibit the CD8+ T cell proliferative response to CEF peptides, as well as activation, in a dose-dependent manner.

### (7) Generation of Tregitope-Blood Component Conjugates

Fusion of a Tregitope with a blood component conjugate, such as albumin, can be useful as a carrier protein for Tregitope payload. Tregitope-blood component conjugates can extend the half-life of Tregitopes in vivo, protect Tregitopes from rapid proteolytic degradation, protect Tregitopes from rapid clearance from circulation and/or rapid kidney excretion, allow for wide distribution of Tregitope-blood component conjugates throughout the body of a subject, aid in delivery of Tregitopes to appropriate immune cells (such as macrophages and APCs), allow the Tregitopes to be processed by the endocytic pathway of certain immune cells (such as macrophages and APCs), and aid in the presentation of Tregitopes as an antigen by said immune cells.

Tregitope-blood component conjugates may be formed by modifying a Tregitope peptide of the instant disclosure (e.g., but not limited to, a peptide or polypeptide comprising, consisting, or consisting essentially of an amino acid sequence of SEQ ID NOS: 4-370, 391-440, and 448-833 (and/or fragments or variants thereof), and optionally 1 to 12 additional amino acids distributed in any ratio on the N-terminus and/or C-terminus of the polypeptide of SEQ ID NOS: 4-370, 391-440, and 448-833) by attaching a reactive moiety to the Tregitope peptide to create a modified Tregitope peptide, then forming a bond between reactive moiety of the modified Tregitope peptide with a reactive functionality on a blood component, as disclosed in U.S. Pat. Nos. 6,849,714, 6,887,470, 7,256,253, and 7,307,148. Albumin is a preferred blood component because it contains an Fc neonatal binding domain that will carry the Tregitope-albumin conjugate into the appropriate cells, such as macrophages and APCs. Further, albumin contains a cysteine at amino acid 34 (Cys³⁴) (the location of the amino acid in the amino acid sequence of human serine albumin), containing a free thiol with a pKa of approximately 5, which may serve as a preferred reactive functionality of albumin. Cys³⁴ of albumin is capable of forming a stable thioester bond with maleimidopropionamido (MPA), which is a preferred reactive moiety of a modified Tregitope peptide. The stable thioester bond between albumin and the Tregitope peptide modified with MPA cannot be cleaved under physiological conditions.

The Tregitope peptide may be as disclosed herein, and in certain aspects is preferably selected from SEQ ID NOS: 4-370, 391-440, and 448-833, or a peptide or polypeptide comprising, consisting, or consisting essentially of an amino acid sequence of SEQ ID NOS: 4-370, 391-440, and 448-833 (and/or fragments or variants thereof), and optionally 1 to 12 additional amino acids distributed in any ratio on the N-terminus and/or C-terminus of the polypeptide of SEQ ID NOS: 4-370, 391-440, and 448-833. One or more lysines may be present on the N-terminus of the Tregitope peptide, such as, but not limited to, added onto to the N-terminus of peptides selected from SEQ ID NOS: 4-370, 391-440, and 448-833. A linker, such as a polyethylene glycol linker (e.g., PEG2 or PEG12), is present between the one or more lysines and the Tregitope sequence, or at the N-terminus of a Tregitope sequence. In aspects, a lysosomal cleavage site, such as a Cathepsin B site, optionally consisting (sequentially from N-terminus to C-terminus) of valine and citrulline, is present between the PEG2 moiety and the Tregitope sequence. A maleimide-based chemistry may be used to covalently link the modified Tregitope peptide to a blood component, preferably serum albumin, in a 1:1 molar ratio. Linking the modified Tregitope peptide to a blood component may be performed in vivo or ex vivo.

Cathepsin B is the first described member of the family of lysosomal cysteine proteases. Cathepsin B possesses both endopeptidase and exopeptidase activities, in the latter case acting as a peptidyldipeptidase. Cathepsin B was been included in the Tregitope peptide design to facilitate the proper cleavage of the Tregitope from Albumin once it is in the lysosomal compartment in the antigen presenting cells. The Valine-Citrulline is a cathepsin B cleavage site that has been previously used successfully and has been FDA approved in Antibody Drug conjugate (e.g., monomethyl auristatin E (MMAE) conjugate in the drug brentuximab vedotin). Our interest in incorporating the site is to provide cleavage sites that would allow the proper cleavage of the Tregitope from the human serum albumin for efficient MHC class II presentation once it is in the APC. The purpose of these experiments will be to determine whether the incorporation of the Cathepsin B site is essential to the design of the Tregitope composition.

### Example 7. Generation of a Tregitope-Albumin Conjugate by Ex Vivo Conjugation

Standard Fmoc (9-fluorenylmethoxycarbonyl) solid-phase peptide synthesis chemistry will be used for peptide synthesis. Synthesis will be performed on Intavis™ MultiPep™ automated peptide synthesizers. Amino acids will be added stepwise to the growing peptide chain (C-terminus to N-terminus; right to left), while attached to an insoluble polystyrene resin support. Amino acid building blocks, protected at their amino terminus by an Fmoc group, will be coupled to the growing chain after activation of the carboxylic acid terminus via one or more condensation reagents (e.g., Hexafluorophosphate Azabenzotriazole Tetramethyl Uronium (HATU), O-(1H-6-Chlorobenzotriazole-1-yl)-1,1,3,3-tetramethyluronium hexafluorophosphate (HCTU)). The reaction by-products at each addition will be removed by solvent washing (6×, Dimethylformamide (DMF)). Following each coupling and capping step, the Fmoc will be removed via piperidine deprotection of the peptide resin (performed 2×; 20% in DMF volume/volume with 0.1M HOBt to suppress Asp dehydration), the resin will be washed with DMF 6×, and the next amino acid will be added. A Cathepsin B cleavage site will be incorporated at the N-terminus of the Tregitope sequence.

For a PEG2 construct (“PEG2” or “P2”), after the desired Tregitope peptide is completed, a PEG2 moiety will be added to the N-terminus, followed by the addition of 4 lysines to the N-terminus. The PEG2 and Lysines will be incorporated to provide a potential docking area for the Cathepsin B. Additionally, the PEG2 and lysines (via the primary amine on the lysine side-chain) is predicted to increase the solubility of the final construct. The composition of the PEG2 construct is shown in Table 5 (below).

TABLE 5
PEG2 construct composition

HSA (Cys 34)- Maleimide linkage- KKKK-Peg2-Val-Cit (CatB cleavage site) _Tregitope_Nle

For a PEG12 construct (“PEG12” or “P12”), two additions of a PEG6 will be added after the Tregitope peptide synthesis. In this case, no lysines will be added. Increasing the PEG length will also provide a docking region for Cathepsin B and improve the solubility of the Tregitope. The composition of the PEG2 construct is shown Table 6 (below).

TABLE 6
PEG12 construct composition

HSA (Cys 34)-Maleimide linkage- Peg12-Val-Cit (CatB cleavage site) _Tregitope_Nle

Subsequently, a small amount of the peptide constructs will be removed from the resin and the peptide sample will be cleaved and deprotected by treatment with trifluoroacetic acid (TFA. 92.5% v/v) in the presence of TIS (triisopropylsilane, 5%) and water (2.5%) to scavenge side-chain protecting groups. Each crude, linear, peptide (˜3-5 mg) will be purified by preparative reversed phased HPLC (Gilson) using a 20 mm×50 mm YMC C18, 5 μm, Hydrosphere column. The peptides will be purified to >90% purity (determined via analytical HPLC) and the mass verified utilizing an ABI-SCIEX QSTAR XL Pro Qo-TOF mass spectrometer prior to the Cathepsin B evaluation. The remaining peptides (PEG2-Tregitope and PEG12-Tregitope) will be left on the resin for the addition of 3-maleimidoproprionic acid (MPA) at a later time.

Recombinant human Cathepsin B (catalog 953-CY of R&D Systems™) will be used to evaluate the cleavage of the Val-Cit site engineered into the Tregitope peptide. The activity assay protocol will be used according to the R&D Systems™'s recommendations with final assay conditions of 0.01 μg of rhCathepsin B and 10 uM of peptide substrate. After incubation of Cathepsin B with purified peptides (at RT for 15 min), the peptide will be evaluated by mass spec using the Qstar XL Pro™. It is predicted that the PEG2 peptide will not have successful cleavage, and further modification of the Cathepsin B protocol will not produce successful cleavage. For the PEG12 product, successful cleavage is predicted.

After evaluation of the cleavage of the Val-Cit site by Cathepsin B, the reactive moiety of 3-maleimidoproprionic acid (MPA) will be added to the N-terminus of the PEG2 and PEG12 peptides. Similar, to the amino acid building blocks, the MPA will be protected by an Fmoc group, and will be coupled to the growing chain after activation of the carboxylic acid terminus. The final MPA-Tregitope constructs will be removed from the resin and the peptide sample will be cleaved and deprotected by treatment with trifluoroacetic acid (TFA. 92.5% v/v) in the presence of TIS (triisopropylsilane, 5%) and water (2.5%). Each crude, linear, peptide (˜20 mg) will be purified by preparative reversed phased HPLC (Gilson™) using a 20 mm×50 mm YMC C18, 5 μm, Hydrosphere column. The MPA-peptides will be purified to >90% purity (determined via analytical HPLC) and the mass will be verified utilizing an ABI-SCIEX QSTAR XL Pro™ Qo-TOF mass spectrometer. A total of 15 mg of the MPA-P2 and MPA-P12 Tregitopes will be used in the subsequent conjugation to rHSA (Albucult-Novozyme™) to construct the final preformed HSA-Tregitope conjugate.

Ellman's Reagent (5,5′-dithio-bis-[2-nitrobenzoic acid]) will be used to estimate sulfhydryl groups in a sample by comparing to a standard curve of a sulfhydryl-containing compound such as cysteine. Ellman's test will be performed on rHSA (Sigma™, Albucult®) at multiple concentrations to ensure the accuracy of the analysis. Ellman's reagent (Sigma™), rHSA from Sigma™ lot RF-009 will be evaluated for free cysteine that would be available for conjugation with the maleimide.

Peptide will be solubilized in dH20, rHSA added (15 mg/ml) and 100 mM Phosphate buffer added to give a final pH of 8. The peptide will be added in a 10× molar excess to the HSA. Peptide/HSA will be incubated at room temperature for 2 h followed by incubation at 4° C. for approximately 24-30 hours.

After the conjugation step, the HSA-conjugate will then be dialyzed into PBS (pH 7.0) first at room temperature for 2 hours, followed by two changes to fresh PBS at 4° C. for 18-24 h. This process will remove excess peptide from the HSA and HSA-Tregitope conjugate preparation.

The Ellman's test will be performed on each conjugate to demonstrate conjugation of the peptide via the rHSA free Cysteine, and determine the efficiency of conjugation in the reaction. It is predicted that the HSA-conjugation preparation will not remove the reduced HSA (mercaptabumin), inherent in the preexisting preparation. The percentage remaining unreacted HSA will be determined HSA-MPA_P2-Tregitope construct, meaning after conjugation with the maleimide-Tregitope the determined percentage of the free cysteine will remain. Thus, it will be possible to calculate the percentage of total rHSA preparation that was reacted with the MPA_P2-Tregitope peptide.

### (8) Methods for Assessing Effect of Tregitope-Blood Component Conjugates on Immune Cells

A maleimide-based chemistry may be used to covalently link a Tregitope (e.g., but not limited to, a peptide or polypeptide comprising, consisting, or consisting essentially of an amino acid sequence of SEQ ID NOS: 4-370, 391-440, and 448-833 (and/or fragments or variants thereof), and optionally 1 to 12 additional amino acids distributed in any ratio on the N-terminus and/or C-terminus of the polypeptide of SEQ ID NOS: 4-370, 391-440, and 448-833) payload to recombinant HSA (rHSA) in a 1:1 stoichiometry. Maleimidopropionamido (MPA) forms a stable thiol ester conjugate with the available free Cys34 in HSA. HSA leverages the neonatal receptor (FcRn) recycling pathway, increasing the half-life of any conjugated payload, and potentially decreasing the need for repeat dosing. rHSA is also known to deliver conjugated payloads to the lymph nodes and is endocytosed by dendritic cells and other antigen presenting cells that express FcRn.

EpiVax designed an rHSA-Tregitope conjugate to contain cleavage sites between the Tregitopes. The cleavage sites are specific for an early endosomal protease, which enable the Tregitopes to be liberated from the rHSA molecule, increasing the efficiency of MHC class II presentation on the cell surface. The long and substantiated history of this FDA-Approved rHSA conjugation chemistry approach, as well as its successful manufacturing history, support its selection for delivery of the Tregitope payload.

Once Tregitope-blood component conjugates are formed, for example as described in Example 7 and subsection 7 of the examples section, as well as the detailed description, the Tregitope-blood component conjugates may be evaluated for their effectiveness in inhibiting effector T-cells and activating regulatory T-cells and their proliferation, for example in comparison with Tregitope peptides alone. Further, the Tregitope-blood component conjugates may be evaluated for their capacity to induce immune tolerance against certain antigens

### Example 8. Evaluation of the Inhibitory Effect of Tregitope-Albumin Delivery Vehicle

To determine the inhibitory effect of the Tregitope delivery vehicle, healthy donor PBMCs are used in a tetanus toxoid bystander suppression assay (TTBSA), and analysis is done on CD4 T-cell proliferation, activation of T cells, frequencies of T effector and T regulatory cells to determine the ratio of Treg/Teff.

So as to optimize the best combination of Tregitopes for translation to the clinic, the effect of combinations of Tregitopes for their ability to synergistically suppress effector T-cell responses in vitro is analyzed. To facilitate these comparisons, a high throughput in vitro assay is developed using human donor peripheral blood mononuclear cells (PBMCs). This assay, referred to as the Tetanus Toxoid Bystander Suppression Assay (TTBSA), takes advantage of the ability of Tregs to suppress T memory cells specific to Tetanus that are elicited in individuals with a history of Tetanus toxoid (TT) vaccination.

At day 0, PBMCs are incubated and stained with Carboxyfluorescein succinimidyl ester (CFSE) dye. At day 1, cells are stimulated with by adding media, Tetanus Toxoid, and either: 8, 6, or 24 μg/mL of a Tregitope; or 10, 40, or 100 μg/mL of a Tregitope-albumin conjugate. Tetanus Toxoid is used at a final concentration of 0.5 μg/ml, where the concentration is methodically titrated and optimized to measure the inhibitory capacity of Tregitopes. Negative controls, including media-only, are included. At day 7, LID cell population marker, extracellular stain, and intracellular stain are added to the cells. At day 8, a readout is taken. Cell sorting assays for analysis of activation markets (e.g., CFSE, CD25) and cell population markets (e.g., LID, CD2c, CD4, and FoxP3) are performed.

Incubation of donor PBMCs with TT stimulates expansion of T effector cells. Tregitopes are added to PBMC in vitro with TT, and activate CD25^{hi}FoxP3^{hi} regulatory T cells suppressing expansion of TT-specific T effector cells. Tregitopes significantly inhibit the proliferation (as is measured by CFSE dilution) and activation (as is measured by CD25 expression) of CD4+T effector cells in a dose dependent manner, and also slightly expand Tregs)(CD25⁺/FoxP3⁺/CD127^{lo}), which is suggested by an increase in the ratio of Treg/Teff cells. A reduction of effector T cell proliferation is a direct consequence of the activation of T regulatory cells and/or the conversion of TT-specific T effector to Treg, for example as is supported by the induction of Treg in vivo.

Using the TTBSA, each of a number of available Tregitopes individually and in pairwise combinations is examined for their potential to suppress CD4+ T cell proliferation. The most promising IgG-Tregitope peptides are selected for further testing. A certain Tregitope, Tregitope A, is the single Tregitope has the most suppressive activity in the TTBSA as compared to the other single Tregitopes. Combining Tregitope A with Tregitope C, an even greater suppressive effect on TT-specific T cell proliferation is observed. Conjugating A+C to rHSA improves their efficacy in vitro.

Using TTBSA, it is expected that HSA-Tregitope conjugates of the instant disclosure will inhibit CD4+ T cell proliferation and activation, and increase the ratio of Treg cells to Teff cells.

### Example 9. Evaluation of the Effectiveness of Preformed Conjugate HSA-Tregitope Therapeutics and Maleimide-Tregitope Peptide Therapeutics

The effect on the response to OVA immunization of preformed conjugate HSA-Tregitope therapeutics and a free-maleimide-Tregitope peptide is evaluated. The latter free-maleimide peptide forms a conjugation in vivo after injection via the reactive maleimide group to the free-Cys34 of the subject's endogenous HSA. 5 mgs of the MPA-P2 and MPA-P12 is used as free-MPA-Tregitope, with the unconjugated HSA in the sample being accounted for by calculating the molar ratio of conjugated to unconjugated HSA.

Mice (female C57BL/6) are immunized s.c. with 50 mg ovalbumin (OVA) on day 0 (CFA) and day 14 (IFA). The preformed HSA conjugate treatments is administered with the OVA in CFA on day 0. Test groups include OVA/HSA-P2-high and OCA/HSA-P2-low. Per injection OVA is 50 μg, and HSA at 800 μg, and HSA-P2H(high) conjugation is at 825 μg (˜20 μg Tregitope). HSA-P2L(low) conjugation is at 100 μg (˜3.7 μg Tregitope). Four control groups include PBS only, PBS/OVA, HSA/OVA, and Tregitope/OVA. A last arm is included to evaluate the utility of the free-maleimide Tregitope peptide and is administered by IV into tail vein. There are five mice per group.

Mice are sacrificed on Day 17. Upon sacrifice, cardiac bleeds and spleens are harvested for each animal. IFNγ/IL2 fluorospot assays, IFNγ/IL17 fluorospot assays, CD4 T cell proliferation, and T cell characterization are performed on the splenocytes stimulated with OVA. PHA is used as a positive control stimulation for spleen cell assays. All of the wells in PHA stimulation are confluent. An acceptance criteria is used wherein SFC (spot forming cells) after stimulation must be greater than 50 spots/10⁶ over negative control (media wells) and must also have a stimulation index greater than 2. According to both the IFNγ/IL2 fluorospot and IFNγ/IL17 fluorospot assays, it is expected that IFNγ production is inhibited by treatment, and the HSA-only control group is inhibited less compared than the treatment groups.

For T-cell proliferation and characterization assays, splenocyte samples are evaluated for induction of FoxP3 expression in TCR Tg cells and for the suppression of OVA specific T cell proliferation (in response to OVA peptide in vitro) by CFSE dilution. To detect FoxP3⁺ Tregs, a single-cell suspension of draining lymph nodes is incubated with 2.4G2 mAb (anti-CD16/32, ATCC) for 15 minutes to block FcR then is stained with anti CD3, CD4, CD25 and anti-clonotypic KJ1-26 for 40 minutes at 4° C. KJ1-26 is specific for clonotypic TCR expressed by D011.10 transgenic mice. Cells are then be permeabilized and stained for FoxP3 nuclear expression and acquired on a Thermo Attune NxT Autosampler™ for FACS analysis. The CD4⁺CD25⁺FoxP3⁺KJ1-26⁺ live cell gate population is established to determine the number and proportion of OVA-Specific T regulatory cells compared to PBS or HSA alone.

Antigen-specific T cell proliferation is evaluated by CFSE dilution. Draining lymph nodes are harvested, are stained with cell proliferation dye CFSE, and a single-cell suspension is prepared at 2×10⁶ cells/mL. Cells are added to 96-well plates at 1004 per well in the presence of 10 μg/ml concentration of OVA 323-339 (New England Peptide, Gardner, Mass., USA). Cells are stimulated for 72 hours and harvested for staining with CD3a, CD4, CD8, CD54RA, CCR7, CD25, CD127, IFNγ HLA-II, CD69, CD154, IL-17, IL-21 for 40 minutes at 4° C. Cells are be fixed, permeabilized and stained for FoxP3 expression and analyzed by flow cytometry. There is an expected increase of OVA-specific KJ1-26⁺CD4⁺CD25⁺FoxP3⁺ adaptive (converted) T regulatory cells in mice treated with free maleimide-Tregitopes and HSA-Tregitope conjugates as compared to mice treated with rHSA. Free maleimide-Tregitopes and HSA-Tregitope conjugates are expected to more effectively reduce OVA-specific proliferation of KJ1-26⁺CD4⁺ T effector cells as compared to rHSA alone.

Anti-OVA antibodies in serum from the bleeds harvested on day 17 are evaluated in serum by ELISA, including a serial dilution plot and a standard ELISA to determine antibody concentrations. Mice treated with HSA-conjugates and free maleimide are expected to lower serum antibody titers compared to no treatment, as indicated by absorbance at different dilutions, as well as comparison of absorbance over a standard curve.

### Example 7: T Cell Responses to SARS-CoV-2 in COVID-19 Convalescents

Materials and Methods

Peptide synthesis. Synthetic peptides are manufactured using 9-fluoronylmethoxycarbonyl (Fmoc) chemistry by 21st Century Biochemicals (Marlboro, Mass.). Peptide purity was >90% as ascertained by analytical reversed phase HPLC. Peptide mass was confirmed by tandem mass spectrometry.

SARS-CoV-2 convalescent donors. Convalescent patients were recruited by Sanguine Biosciences, a clinical services group that identified, consented and enrolled participants. Inclusion criteria included subjects (i) willing and able to provide written informed consent and photo identification, (ii) aged 18-60, both male or female, (iii) confirmed COVID-19 diagnosis (recovered) with date of diagnosis a minimum of 30 days from blood collection, and (iv) positive COVID-19 PCR based-kit documented by time-stamped medical record and/or diagnostic test report and test kit used identified. Exclusion criteria included subjects who (i) are pregnant or nursing, (ii) have a known history of HIV, hepatitis or other infectious diseases, (iii) have autoimmune diseases, (iv) in vulnerable patient population (prisoners, mentally impaired), (v) have medical conditions impacting their ability to donate blood (i.e. anemia, acute illness) (vi) received immunosuppressive therapy or steroids within the last 6 months, (vii) received an investigational product in the last 30 days, (viii) experienced excess blood loss including blood donation defined as 250 mL in the last month or 500 mL in the last two months, or (ix) had a positive COVID-19 PCR test, but were asymptomatic. Samples were collected in accordance with NIH regulations and with IRB approval.

Healthy unexposed donors. Samples were obtained from leukocyte reduction filters from the Rhode Island Blood Center for unrelated studies prior to the SARS-CoV-2 outbreak in December 2019. Samples were collected in accordance with NIH regulations and with IRB approval.

PBMC culture. Thawed whole PBMCs (normal healthy donors) will be rested overnight and expanded by antigen stimulation (including the detolerized SARS-CoV-2 polypeptides of the instant disclosure) over nine days at 37° C. under a 5% CO₂ atmosphere. In a 48-well plate, 5×10\hat{ }6 cells in 150 μl RPMI medium supplemented with human AB serum will be stimulated with pools of peptides at 10 μg/ml on Day 1. Three days later, IL-2 will be added to 10 ng/ml and the culture volume raised to 300 μl. On Day 7, cells will be supplemented with 10 ng/ml IL-2 by half media replacement. Two days later, PBMCs will be collected and washed in preparation to measure immune recall responses.

FluoroSpot Assay. Interferon-gamma (IFNg) Fluorospot assays will be performed ex vivo and following culture using kits purchased from Mabtech and performed according to the manufacturer's specifications. Peptides will be added individually at 10 μg/ml and pooled at 10 μg/ml (8 peptides, 1.25 μg/mL) to triplicate wells containing 250,000 PBMCs (ex vivo) or 100,000 PBMCs (cultured) in RPMI medium supplemented with 10% human AB serum. Triplicate wells will be plated with ConA (10 μg/ml) as a positive control, and six wells containing no antigen stimulus were used for background determination. Cells will incubated for 40-48 hours at 37° C. under a 5% CO2 atmosphere. Plates will be developed according to the manufacturer's directions using FITC-labeled anti-IFN-γ detection antibody.

Raw spot counts will be recorded by ZelINet Consulting, Inc. using a FluoroSpot reader system (iSpot Spectrum, AID, Strassberg, Germany) with software version 7.0, build 14790, where fluorescent spots will be counted utilizing separate filters for FITC, Cy3, and Cy5. Camera exposure and gain settings will be adapted for each filter to obtain high quality spot images preventing over- or underexposure. Fluorophore-specific spot parameters will be defined using spot size, spot intensity and spot gradient (fading of staining intensity from center to periphery of spot), and a spot separation algorithm will be applied for optimal spot detection.

Results will be calculated as the average number of spots in the peptide wells, adjusted to spots per one million cells. Responses meeting the following criteria are positive when the number of spots is (i) at least twice background, (ii) greater than 50 spot forming cells per well above background (1 response per 20,000 PBMCs), and (iii) statistically different (p<0.05) from the media-only control by the Student's t test.

Results: It is expected that the detolerized SARS-CoV-2 polypeptides of the instant disclosure of the instant disclosure will be recognized by T cells raised in natural infection, stimulate Th1 cytokine production, may stimulate pre-existing immunity to common cold coronaviruses, and may boost memory immunity in clinical trials.

### EQUIVALENTS

While the instant disclosure has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains, and as fall within the scope of the appended claims.

1-76. (canceled)

77. A polypeptide comprising, an amino acid sequence having at least 80%, identity to any one of SEQ ID NOS: 4-370, 391-440, and 448-833, or any one of SEQ ID NOS: 4-370, 391-440, and 448-833 further comprising 1 to 12 additional amino acids distributed in any ratio on the N terminus and/or C-terminus, wherein the polypeptide retains MHC binding propensity and the same TCR specificity, and/or retains anti-SARS-CoV-2 activity;
or a nucleic acid encoding said polypeptide.

78. A polypeptide according to claim 77 wherein the amino acid sequence is mutated in an anchoring amino acid to the MHC and/or in a T-cell receptor binding epitope to detolerize the polypeptide.

79. The polypeptide of claim 78, wherein the amino acid sequence comprises SEQ ID NO: 6, 7, 18-31, 186-231, and/or 448-459 with a mutation at one of positions V62, L65, S67, V70, K63, N64, N66, S68, R69, T11, L12, V14, N15, S16, V17, L19, F20, A22, F23, V24, V25, F26, L27, L28, V29, T30, L31, A32, I33, L34, A36, R38, A41, I13, L18, F20, L21, T35, L37, L39, and/or C40 in relation to SEQ ID NO: 1.

80. The polypeptide of claim 79, wherein the mutation comprises one or more of V62A, V62G, V62N, V62Q, V62S, V62T, and/or S67Q in relation to SEQ ID NO: 1.

81. The polypeptide of claim 77, wherein the amino acid sequence comprises SEQ ID NO: 4, 5, 17, 32-41, 232-245, 440, and 450-471 with a mutation at one of positions I118, N121, P123, G126, L119, L120, V122, L124, H125, Y179, G182, S184, V187, K180, L181, A183, Q185, and/or R186 in relation to SEQ ID NO: 2.

82. The polypeptide of claim 81, wherein the mutation comprises one or more of I118A, I118G, I118N, I118Q, I118S, I118T, N121P, P123Q, P123G, G126P, Y179A, Y179N, Y179Q, Y1795, Y179T, S184G, S184Q, and/or S184T in relation to SEQ ID NO: 2.

83. The polypeptide of claim 77, wherein the amino acid sequence comprises SEQ ID NO: 8-17, 42-93, 246-370, 422, 423, 432, 434-439, and 794-833 with a mutation at one of positions F28, S31, L33, T36, D38, L41, R29, S30, V32, H34, S35, Q37, L39, F40, I195, V198, D200, Q203, N196, L197, R199, L201, P202, I220, F223, T225, A228, T221, R222, Q224, L226, L227, Y254, P257, Y259, L262, L255, Q256, R258, F260, L261, V496, S499, E501, H504, V497, L498, F500, L502, L503, L806, N809, V811, A814, L807, F808, K810, T812, L813, L843, L846, P848, T851, T844, V845, P847, L849, L850 F912, A915, G917, Q920, L923, T926, S928, G931, N913, S914, I916, K918, I919, S924, S925, A927, A929, L930, F955, I958, S960, N963, G956, A957, S959, V961, L962, I998, A1001, I1003, S1006, N1008, A1011, R999, A1000, E1002, R104, A1005, A1007, L1009, and/or A1010 in relation to SEQ ID NO: 3.

84. The polypeptide of claim 83, wherein the mutation comprises one or more of F28G, F28A, F28N, F28T, F28S, F28Q, S31G, S31T, L33Q, I195A, I195G, I195N, I195S, I195T, I195Q, V198G, V198T, V198N, Q203E, Q203G, Q203T, I220A, I220G, I220N, I220Q, I220S, I220T, T225Q, Y254A, Y254G, Y254N, Y254Q, Y254S, Y254T, T259G, T259Q, V496A, V496G, V496N, V496Q, V496S, V496T, S499G, S499Q, S499T, L806A, L806G, L806N, L806Q, L806S, L806T, N809G, L843A, L843G, L843N, L843Q, L843S, L843T, L846G, L846T, P848Q, F912A, F912G, F912N, F912Q, F912S, F912T, A915G, L923A, L923G, L923N, L923Q, L923S, L923T, T926G, F955A, F955G, F955N, F955Q, F955S, F955T, I958G, 5960G, S960Q, S960T, I998A, I998G, I998N, I998Q, I998S, I998T, A1001G, A1001T, I1003A, I1003G, I1003N, I1003Q, I1003S, I1003T and/or N1008Q in relation to SEQ ID NO: 3.

85. The polypeptide of claim 77, wherein said nucleic acid is housed in a vector.

86. The polypeptide of claim 77, wherein said nucleic acid is housed in a plasmid.

87. The polypeptide of claim 77, wherein said nucleic acid is expressed by a cell.

88. The polypeptide of claim 77, wherein the polypeptide is joined, linked, or inserted into a heterologous polypeptide.

89. A pharmaceutical composition comprising:
at least one polypeptide or nucleic acid according to 77; and
a pharmaceutically-acceptable carrier and/or excipient.

90. The pharmaceutical composition according to claim 88 configured as a vaccine, said vaccine further comprising a carrier, and/or adjuvant.

91. A method for suppressing an immune response in a subject in need thereof, the method comprising administering to the subject a therapeutically effective amount of at least one polypeptide or nucleic acid according to claim 77.

92. The method according to claim 91, wherein the immune response is a result of treatment with at least one or more therapeutic treatments with at least one therapeutic protein, treatment with a vaccine or treatment with at least one antigen.

93. The method according to claim 91, wherein the polypeptide is administered to isolated dendritic cells ex vivo, and said dendritic cells are the re-introduced to the subject.

94. The method according to claim 91, wherein the administration of the polypeptide shifts one or more antigen presenting cells to a regulatory phenotype.

95. The method according to claim 91, wherein the administration of the polypeptide shifts one or more dendritic cells to a regulatory phenotype.

96. The method according to claim 95, wherein the regulatory phenotype is characterized by a decrease in CD11c and HLA-DR expression in the dendritic cells or other antigen presenting cells.
